(19) United States
(12) Patent Application Publication
(10) Pub. No.: US 2008/0085284 A1
(43) Pub. Date: Apr. 10, 2008

(54) Construction of a Comparative Database and Identification of Virulence Factors Comparison of Polymorphic Regions in Clinical Isolates of Infectious Organisms

(76) Inventors: Villoo Morawala Patell, Bangalore (IN); K.R. Rajyashri, Bangalore (IN); Marc Rodrigue, Marcy (FR); Guy Vernet, Marcy (FR)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 11/632,108

(22) PCT Filed:
(86) PCT No.: PCT/IB04/02598

(51) Int. Cl. A61K 31/70; A61K 39/00; A61P 43/00; C07H 21/04; C12Q 1/68

(52) U.S. Cl. 424/184.1; 435/6; 514/44; 536/23.7; 536/24.33; 707/102

(57) ABSTRACT

The present invention is directed to novel nucleotide sequences to be used for diagnosis, identification of the strain, typing of the strain and giving orientation to its potential degree of virulence, infectivity and/or latency for all infectious diseases more particularly tuberculosis. The present invention also includes method for the identification and selection of polymorphisms associated with the virulence' and/or infectivity in infectious diseases more particularly in tuberculosis by a comparative genomic analysis of the sequences of different clinical isolates/strains of infectious organisms. The regions of polymorphisms, can also act as potential drug targets and vaccine targets. More particularly, the invention also relates to identifying virulence factors of M. tuberculosis strains and other infectious organisms to be included in a diagnostic DNA chip allowing identification of the strain, typing of the strain and finally giving orientation to its potential degree of virulence. Although the present invention has been illustrated with specific reference to the polymorphic region in the Mycobacterium tuberculosis, the said invention is not to be understood and construed as being limited to Tuberculosis but is applicable to all infectious diseases.

### FIELD OF INVENTION

The present invention is directed to novel nucleotide sequences to be used for diagnosis, identification of the strain, typing of the strain and giving orientation to its potential degree of virulence, infectivity and/or latency for all infectious diseases including tuberculosis. The present invention also includes method for the identification and selection of polymorphisms associated with the virulence and/or infectivity in infectious diseases by a comparative genomic analysis of the sequences of different clinical isolates/strains of infectious organisms. The regions of polymorphisms, can also act as potential drug targets and vaccine targets. More particularly, the invention also relates to identifying virulence factors of M. tuberculosis strains and other infectious organisms to be included in a diagnostic DNA chip allowing identification of the strain, typing of the strain and finally giving orientation to its potential degree of virulence.

Although the present invention has been illustrated with specific reference to the polymorphic region in the Mycobacterium tuberculosis, the said invention is not to be understood and construed as being limited to Tuberculosis but is applicable to all infectious diseases.

### BACKGROUND OF THE INVENTION

Microbial pathogens use a variety of complex strategies to subvert host cellular functions to ensure their multiplication and survival. Some pathogens that have co-evolved or have had a long-standing association with their hosts utilize finely tuned host-specific strategies to establish a pathogenic relationship.

During infection, pathogens encounter different conditions, and respond by expressing virulence factors that are appropriate for the particular environment, host, or both.

Although antibiotics have been effective tools in treating infectious disease, the emergence of drug resistant pathogens is becoming problematic in the clinical setting. New antibiotic or antipathogenic molecules are therefore needed to combat such drug resistant pathogens. Accordingly, there is a need in the art for screening methods aimed not only at identifying and characterizing potential antipathogenic agents, but also for identifying and characterizing the virulence factors that enable pathogens to infect and debilitate their hosts.

The mycobacteria are rod-shaped, acid-fast, aerobic bacilli that do not form spores. Several species of mycobacteria are pathogenic to humans and/or animals, and factors associated with their virulence. Tuberculosis is a worldwide health problem, which causes approximately 3 million deaths each year, yet little is known about the molecular basis of tuberculosis pathogenesis. The disease is caused by infection with Mycobacterium tuberculosis; tubercle bacilli are inhaled and then ingested by alveolar macrophages. As is the case with most pathogens, infection with M. tuberculosis does not always result in disease. The infection is often arrested by developing cell-mediated immunity (CMI) resulting in the formation of microscopic lesions, or tubercles, in the lung. If CMI does not limit the spread of M. tuberculosis, caseous necrosis, bronchial wall erosion, and pulmonary cavitations may occur. The factors that determine whether infection with M. tuberculosis results in disease are not well understood.

The tuberculosis complex is a group of four mycobacterial species that are so closely related genetically that it has been proposed treat they or combined into a single species. Three important members of the complex are Mycobacterium tuberculosis, the major cause of human tuberculosis; Mycobacterium africanum, a major cause of human tuberculosis in some populations; and Mycobacterium bovis, the cause of bovine tuberculosis. None of these mycobacteria is restricted to being pathogenic for a single host species. For example, M. bovis causes tuberculosis in a wide range of animals including humans in which it causes a disease that is clinically indistinguishable from that caused by M. tuberculosis. Human tuberculosis is a major cause of mortality throughout the world, particularly in less developed countries. It accounts for approximately eight million new cases of clinical disease and three million deaths each year. Bovine tuberculosis, as well as causing a small percentage of these human cases, is a major cause of animal suffering and large economic costs in the animal industries.

Antibiotic treatment of tuberculosis is very expensive and requires prolonged administration of a combination of several anti-tuberculosis drugs. Treatment with single antibiotics is not advisable as tuberculosis organisms can develop resistance to the therapeutic levels of all antibiotics that are effective against them. Strains of M. tuberculosis that are resistant to one or more anti-tuberculosis drugs are becoming more frequent and treatment of patients infected with such strains is expensive and difficult. In a small but increasing percentage of human tuberculosis cases the tuberculosis organisms have become resistant to the two most useful antibiotics, isoniazid and rifampicin. Treatment of these patients presents extreme difficulty and in practice is often unsuccessful. In the current situation there is clearly an urgent need to develop new methods for detecting virulent strains of mycobacteria and to develop tuberculosis therapies.

There is a recognized vaccine for tuberculosis, which is an attenuated form of M. bovis known as BCG. This is very widely used but it provides incomplete protection. The development of BCG was completed in 1921 but the reason for its avirulence was and has continued to remain unknown. Methods of attenuating tuberculosis strains to produce a vaccine in a more rational way have been investigated but have not been successful for a variety of reasons. However, in view of the evidence that dead M. bovis BCG was less effective in conferring immunity than live BCG, there exists a need for attenuated strains of mycobacteria that can be used in the preparation of vaccines.

A variety of compounds have been proposed as virulence factors for tuberculosis but, despite numerous investigations, good evidence to support these proposals is lacking. Nevertheless, the discovery of a virulence factor or factors for tuberculosis is very important and is an active area of current research. Such a discovery would not only enable the possible development of a new generation of tuberculosis vaccines but might also provide a target for the design or discovery of new or improved anti-tuberculosis drugs or therapies.

Present methods for the identification and characterization of mycobacteria in samples from human and animal diseases are by Zeil-Neilson staining, in-vitro and in vivo culture, biochemical testing and serological typing. These methods are generally slow and do not readily discriminate between closely related mycobacterial strains and species particularly, for example,Mycobacterium paratuberculosis and Mycobacterium avium. Mycobacteria are widespread in the environment, and rapid methods do not exist for the identification of specific pathogenic strains from amongst the many environmental strains, which are generally non-pathogenic. Difficulties with existing methods of mycobacterial identification and characterization have increased relevance for the analysis of microbial isolates from Crohn's disease (Regional Ileitis) in humans and Johne's disease in animals (particularly cattle, sheep and goats) as well as for M. avium strains from AIDS patients with mycobacterial superinfections. Although recognition of the causative agents of human leprosy and tuberculosis are clear, clinico-pathological forms of each disease exist, such as the tuberculoid form of leprosy, in which mycobacterial tissue abundance is low and identification correspondingly difficult. Improvements in the specific recognition and characterization of mycobacteria may also increase in relevance if current evidence linking diseases such as rheumatoid arthritis to mycobacterial antigens is substantiated. Emerging drug resistance to mycobacteria including M. avium isolates from AIDS patients, any Mycobacterium tuberculosis from TB patients is an increasing problem.

There is no data or technical information in the prior art, which permits to select specifically potential new targets and protective antigens for new drugs and vaccine compositions to treat and prevent infectious diseases, particularly tuberculosis. Furthermore, there is a need for the development of new tools for the selection of genes which encode for essential proteins or regulatory nucleotidic sequences in the survival or infection of mycobacterium species and useful for the design of anti-tuberculosis drugs and vaccines based on the knowledge of comparative mycobacterial genomics.

A method of using DNA probes for the precise identification of mycobacteria and discrimination between closely related mycobacterial strains and species by genotype characterization is essential. The method of genotypic analysis is further applicable to the rapid identification of phenotypic properties such as drug resistance and pathogenicity.

The invention aids in fulfilling these needs in the art. The method according to the invention has the advantage to reduce drastically the number of potential new targets and protective antigens by giving for the first time an exhaustive description of conserved SNPs in the tuberculosis. The isolated polynucleotides described in the present invention, which are highly conserved in genomic sequences of both virulent and avirulent, are by this characteristic essential for the survival or the virulence of these mycobacteria in the host. The identification of antigens and potentially therapeutic targets has been made by a method of comparative genomic analysis.

### PRIOR ART

Patent application WO 02074903 describes a method of selection of purified nucleotidic sequences or polynucleotides encoding proteins or part of proteins carrying at least an essential function for the survival or the virulence of mycobacterium species by a comparative genomic analysis of the sequence of the genome of M. tuberculosis aligned on the genome sequence of M. leprae and M. tuberculosis and M. leprae marker polypeptides of nucleotides encoding the polypeptides, and methods for using the nucleotides and the encoded polypeptides are disclosed.

U.S. Pat. No. 6,228,575 provides oligonucleotide based arrays and methods for speciating and phenotyping organisms, for example, using oligonucleotide sequences based on the Mycobacterium tuberculosis, rpoB gene. The groups or species to which an organism belongs may be determined by comparing hybridization patterns of target nucleic acid from the organism to hybridization patterns in a database.

Patent application No. WO9954487 and U.S. Pat. No. 6,492,506 describes a method for isolating a polynucleotide of interest that is present or is expressed in a genome of a first mycobacterium strain and that is absent or altered in a genome of a second mycobacterium strain which is different from the first mycobacterium strain using a bacterial artificial chromosome (BAC) vector. This invention further relates to a polynucleotide isolated by this method and recombinant BAC vector used in this methQd. In addition the present invention comprises method and kit for detecting the presence of a mycobacteria in a biological sample.

U.S. Pat. No. 5,783,386 describes polynucleotides associated with virulence in mycobacteria, and particularly a fragment of DNA isolated from M. bovis that contains a region encoding a putative sigma factor. Also provided are methods for a DNA sequence or sequences associated with virulence determinants in mycobacteria, and particularly in M. tuberculosis and M. bovis. In addition, the invention provides a method for producing strains with altered virulence or other properties, which can themselves be used to identify and manipulate individual genes.

U.S. Pat. No. 5,955,077 relates to novel antigens from mycobacteria capable of evoking early (within 4 days) immunological responses from T-helper cells in the form of gamma-interferon release in memory immune animals after rechallenge infection with mycobacteria of the tuberculosis complex. The antigens of the invention are believed useful especially in vaccines, but also in diagnostic compositions, especially for diagnosing infection with virulent mycobacteria. Also disclosed are nucleic acid fragments encoding the antigens as well as methods of immunizing animals/humans and methods of diagnosing tuberculosis.

U.S. Pat. No. 6,596,281 describes two genes for proteins of M. tuberculosis have been sequenced. The DNAs and their encoded polypeptides can be used for immunoassays and vaccines. Cocktails of at least three purified recombinant antigens, and cocktails of at least three DNAs encoding them can be used for improved assays and vaccines for bacterial pathogens and parasites.

U.S. Pat. No. 5,700,683 provides specific genetic deletions that result in an avirulent phenotype of a mycobacterium. These deletions may be used as phenotypic markers of providing a means for distinguishing between disease-producing and non-disease producing mycobacteria.

U.S. Pat. No. 5,225,324 relates to a family of DNA insertion sequences (ISMY) of mycobacterial origin and other DNA probes which may be used a probes in assay methods for the identification of mycobacteria and the differentiation between closely related mycobacterial strains and species. The use of ISMY, and of proteins and peptides encoded by ISMY, in vaccines, pharmaceutical preparations and diagnostic test kits is also disclosed.

WO0066157 patent application provides for polypeptides encoded by open reading frames present in the genome of Mycobacterium tuberculosis but absent from the genome of BCG and diagnostic and prophylactic methodologies using these polypeptides.

U.S. Pat. No. 6,458,366 discloses compounds and methods for diagnosing tuberculosis. The compounds provided include polypeptides that contain at least one antigenic portion of one or more M. tuberculosis proteins, and DNA sequences encoding such polypeptides. Diagnostic kits containing such polypeptides or DNA sequences and a suitable detection reagent may be used for the detection of M. tuberculosis infection in patients and biological samples. Antibodies directed against such polypeptides are also provided.

S. T. Cole has sequences the complete genome sequence of the best-characterized strain of Mycobacterium tuberculosis, H37Rv. The sequence has been analyzed in order to improve our understanding of the biology of this slow-growing pathogen and to help the conception of new prophylactic and therapeutic interventions. [Nature 393, 537-544 (1998)]

In a multicomponent analysis to determine the association of polymorphism to the degree of virulence and infectivity is in progress. These polymorphisms constitute a set of putative virulence markers that are being validated in 120 clinical isolates of tuberculosis. The study results in a set of virulence markers, which could be used in predicting the degree of virulence and infectivity of Mycobacterium infections.

There is no data or technical information in the prior art, which permits to select specifically potential new targets and protective antigens for new drugs and vaccine compositions to treat and prevent infectious diseases including mycobacterial diseases, particularly tuberculosis and leprosy.

### SUMMARY OF THE INVENTION

The object of the present invention is to identify genes which encode for essential proteins or regulatory nucleotidic sequences in the survival or infection of mycobacterium species as also all infectious diseases and which could be useful for the design of drugs and vaccines based on the knowledge of comparative genomics.

Yet another object of the present invention is to provide for the identification of strains including mycobacterium in disease samples, for the specific recognition of pathogenic strains, for precisely distinguishing closely related strains including mycobacterial strains and for defining virulence and resistance patterns.

The method according to the invention has the advantage to reduce drastically the number of potential new targets and protective antigens by giving for the first time an exhaustive description of conserved SNPs in different M. tuberculosis strains, which cause tuberculsosis. The isolated polynucleotides described in the present invention, which are highly conserved in genomic sequences of virulent strains are essential for the survival or the virulence of these strains, in particular mycobacteria, in the host. The identification of antigens and potentially therapeutic targets has been made by a method of comparative genomic analysis.

The invention is directed to identifying virulence factors in M. tuberculosis & other infectious diseases, using both strands of DNA, RNA and/or proteins associated with the virulence factors, allowing identification of the strain, typing of the strain and finally giving orientation to its potential degree of virulence, infectivity and/or latency.

Accordingly this invention provides a nucleotide sequences for diagnosis, identification of the strain, typing of the strain and giving orientation to its potential degree of virulence, infectivity and/or latency of all infectious diseases having a SEQ ID nos 1 to 2531.

The invention is further directed to a method comprising of aligning the genomic sequences of different mycobacteria species to

a. Select a polynucleotide sequence highly conserved amongst the virulent strains and corresponds to an essential gene for the survival or the virulence of mycobacterium species

b. Select polymorphisms between virulent and avirulent strains to identify genes and regions conferring virulence to the former strains

c. And optionally, testing the polynucleotide selected for its capacity of virulence or involved in the survival of a mycobacterium species said testing being based on the activation or inactivation of said polynucleotide in a bacterial host or said testing being based on the activity of the product of expression of said polynucleotide in vivo or in vitro.

The invention further comprises of identification of following polymorphisms, having potential to be used as reagents and in diagnostics, drug and vaccine development for infectious diseases:

i. Identical nucleotide in. virulent strains/species, but a different nucleotide in avirulent strains/species at the same position

ii. Some of the virulent strains differ in the nucleotide sequence at specific positions and share the nucleotide sequence with that of avirulent strains.

Yet another object of the present invention is to provide for the identification of strains including mycobacterium in disease samples, for the specific recognition of pathogenic strains, for precisely distinguishing closely related strains including mycobacterial strains and for defining virulence and resistance patterns.

The method according to the invention has the advantage to reduce drastically the number of potential new targets and protective antigens by giving for the first time an exhaustive description of conserved SNPs in different M. tuberculosis strains, which cause tuberculsosis. The isolated polynucleotides described in the present invention, which are highly conserved in genomic sequences of virulent strains are essential for the survival or the virulence of these strains, in particular mycobacteria, in the host. The identification of antigens and potentially therapeutic targets has been made by a method of comparative genomic analysis.

The invention is directed to identifying virulence factors in M. tuberculosis & other infectious diseases, using both strands of DNA, RNA and/or proteins associated with the virulence factors, allowing identification of the strain, typing of the strain and finally giving orientation to its potential degree of virulence, infectivity and/or latency.

Accordingly this invention provides a nucleotide sequences for diagnosis, identification of the strain, typing of the strain and giving orientation to its potential degree of virulence, infectivity and/or latency of all infectious diseases having a SEQ ID nos 1 to 2531.

The invention is further directed to a method comprising of aligning the genomic sequences of different mycobacteria species to

a. Select a polynucleotide sequence highly conserved amongst the virulent strains and corresponds to an essential gene for the survival or the virulence of mycobacterium species

b. Select polymorphisms between virulent and avirulent strains to identify genes and regions conferring virulence to the former strains

c. And optionally, testing the polynucleotide selected for its capacity of virulence or involved in the survival of a mycobacterium species said testing being based on the activation or inactivation of said polynucleotide in a bacterial host or said testing being based on the activity of the product of expression of said polynucleotide in vivo or in vitro.

The invention further comprises of identification of following polymorphisms, having potential to be used as reagents and in diagnostics, drug and vaccine development for infectious diseases:

i. Identical nucleotide in virulent strains/species, but a different nucleotide in avirulent strains/species at the same position

ii. Some of the virulent strains differ in the nucleotide sequence at specific positions and share the nucleotide sequence with that of avirulent strains.

The invention relates to the identification and analysis of Non-synonymous SNPs to predict conservative and non-conservative amino acid substitutions. The effect of the substitution on the function of the proteins encoded provided a powerful insight in predicting SNPs correlating with virulence and infectivity in infectious diseases for example M. tuberculosis.

The invention further relates to proteins, RNA, DNA and metabolites encoded by the region carrying the polymorphisms in tuberculosis and other infectious disease causing organisms; which can be utilized for developing drugs and vaccines effective against tuberculosis and other infectious diseases, plays a important role in gene therapy, RNAi technology and imaging.

The invention is also directed to a process for the production of recombinant polypeptides and chimeric polypeptides comprising them, antibodies generated against these polypeptides, immunogenic or vaccine compositions comprising at least one polypeptide useful as protective antigens or capable to induce a protective response in vivo or in vitro against mycobacterium infections, immunotherapeutic compositions comprising at least such a polypeptide according to the invention, and the use of such nucleic acids and polypeptides in diagnostic methods, vaccines, kits, or antimicrobial therapy.

SEQ ID Nos. 1 to 1829 are single nucleotide polymorphisms.

SEQ ID Nos. 1830 to 2286 is an insertion/deletion (indel)

SEQ ID No 2287 to 2531 are regions of long polymorphism.

The present invention also includes primer sequences for amplifying the region around the polymorphism SEQ ID nos 1 to 2531

The nucleotide sequences flanking the polymorphisms of SEQ ID Nos. 1 to 2531 to a length of 35 nucleotides on either side are used in reagents and in diagnostics, drug development, RNAi, gene therapy and other such technologies.

SEQ ID Nos 1 to 2531 are used as targets for drug design using bioinformatics and other tools, drug development, for gene therapy and vaccine development. This invention also includes the use of proteins, RNA, DNA and metabolites encoded by the region carrying the polymorphisms having a SEQ ID Nos. 1 to 2531 for RNAi technology and antisense technologies.

This invention also includes a database for identification and selection of the polymorphisms having SEQ ID nos. 1 to 2531.

### BRIEF DESCRIPTION OF THE FIGURES AND TABLES

FIG. 1 describes Entity Relationship Model.

FIG. 2 illustrates the identification of SNPs in M. tuberculosis strains H37Rv, CDC1551 and M. bovis BCG. A total of 1829 SNP's have been identified in the three genomes. Of these 1825 SNPs are identical in H37Rv and CDC1551, with a different nucleotide in BCG. 1579 of these are in ORFs while the rest (246) are in non-coding regions. The SNPs in the ORF are categorized into synonymous, non-synonymous SNPs. The latter are further categorized on the basis of the change in primary structure of the protein that results - conservative for no-change and non-servative for changed primary structure of protein encoded.

FIG. 3 illustrates the identification of indels in M. tuberculosis strains H37Rv, CDC1551 and M. bovis BCG. A total of 794 indels have been identified in the three genomes. Of these, 237 are present in both H37Rv and CDC1551 with respect to BCG, 178 in ORF and 59 are outside the ORF.

FIG. 4 illustrates Identification of long plymorphisms in M. tuberculosis strains H37Rv, CDC1551 and M. bovis BCG. 136 polymorphisms are present in the three genomes, 30 of them being identical to CDC1551 and H37Rv. 22 of these polymorphisms are present in the ORFs while 8 are outside the ORF.

FIG. 5 display shows a region of 10 kb of the BCG genome with three types of annotations: BCG ORF's, SNP's in H37Rv, and SNP's in CDC1551.

FIG. 6 shows the comparative genomics browser displaying BCG in the upper panel and H37Rv in the bottom panel. The segments labeled MUM-* are the perfect matches generated by the MUMmer tool, and the vertical lines show the alignment of the MUM segments in both genomes. The color coding of the ORF's is used to indicate the length of the ORF. This is very helpful to researchers because if an ORF in H37 aligns with an ORF in BCG but they have different colors, then there is a mutation that makes them have different lengths (see for example the genes in the MUTM-1280 region).

FIG. 7.1-7.25 are the primers used for the amplification to encompass the regions of polymorphisms.

Table 1 gives the list of Single Nucleotide Polymorphisms in Mycobacterium tuberculosis/M. bovis BCG.

Table 2 gives the list of Insertions/deletions (Indels) in Mycobacterium tuberculosis/M. bovis BCG.

Table 3 gives the list of long polymorphisms in Mycobacterium tuberculosis/M. bovis BCG.

Table 4 lists Polymorphisms in genes involved in cell wall synthesis.

Table 5 lists Polymorphisms in transcription factors.

Table 6 lists Polymorphisms in genes involved in lipid metabolism

Table 7 lists Polymorphisms in genes encoding membrane transport proteins

Table 8 lists Polymorphisms in genes implicated in virulence

### DETAILED DESCRIPTION OF THE INVENTION

The Mycobacterium tuberculosis complex consists of six species—M. tuberculosis, M. bovis, M. caitotti, M. microtii and M. africanum. Of these, the genomes of two different strains of M. tuberculosis, which are virulent and infective to humans, have been completely sequenced, while the complete genome of M. bovis BCG, which is non-virulent and non-infective has also been sequenced. Only partial sequences are available for the other species. All Mycobacterium sequences available in the NCBI, EMBL, GENBANK, Sanger and TIGR databases were retrieved and compiled.

The total numbers of sequences retrieved are as follows:

| Species name | No of sequences retrieved |
|---|---|
| Mycobacterium africanum | 16 |
| Mycobacterium canetti | 03 |
| Mycobacterium microtii | 24 |
| Mycobacterium tuberculosis | 1274 |
| Mycobacterium bovis | 183 |

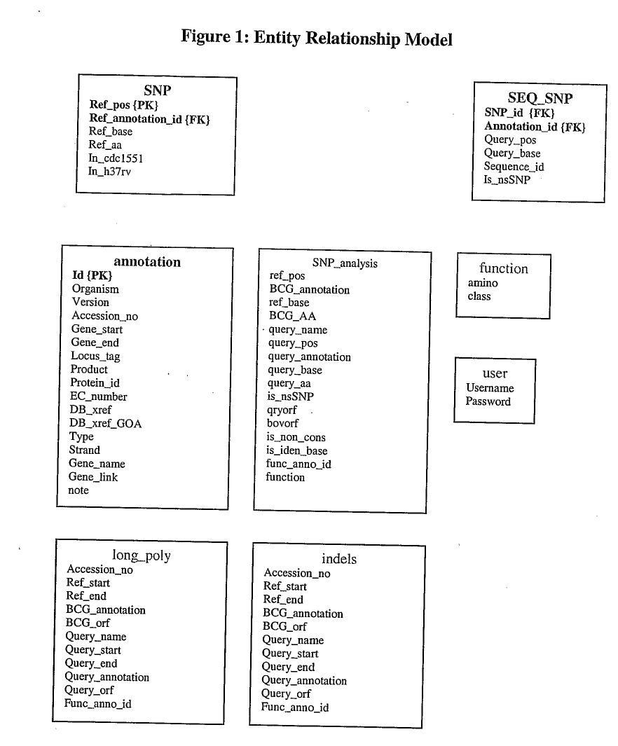

The complete genomes of Mycobacterium tuberculosis strains H37Rv (referred to as H37Rv) and CDC1551 (referred to as CDC1551) - both of which are virulent and infective to humans) and Mycobacterium bovis BCG (referred to as BCG)—non-virulent and non-infective in humans - were aligned and a database constructed. The structure of the database is given in FIG. 1.

Sequences were aligned using the pairwise alignment tool “MUMmer-3.08” (www.tigr.org).

The use of MUMmer required three distinct steps:

1. running MUMmer for each of the target genomes (CDC1551 and H37Rv) against the reference genome (BCG)

2. parsing the MUMmer output using to produce a list of polymorphisms, and loading these data into a polymorphism database.

3. generating feature files for visualization, and loading these features into a feature database.

BCG was chosen as the reference genome and compare the two tuberculosis strains, CDC1551 and H37Rv, against the reference. MUMmer uses fasta files as input and was run using the following command line:

| run-mummer1 bovis.fasta cdc1551.fasta BCG-CDC | | | |
|---|---|---|---|
| which takes the format, | | | |
| program | <reference> | <query> | <output> |

The BCG-CDC parameter provides the file name prefix for the output files, the bovis.fasta parameter is the reference fasta file, and the CDC1551.fasta parameter is the name of the query fasta sequence file.

The database is generated using the scripts:

Parsing MUMmer .align file to extract polymorphism data

The file is parsed to extract useful information and stored it in a much simpler tab-delimited text file format. A custom perl script named mum-parse.pl which uses the Perl module Parse::RecDescent to create a recursive descent parser based on the grammar contained in the custom file Muinmer. pm. is used to run the following command line:
$ perl./mum-parse.pl—mummer1—outfile=../mummer/BCG-CDC./mummer/BCG-CDC.align

This creates three output files:

1. BCG-CDC.gaps—this is the initial output file that simply lists the location of all exact matches in the two sequences.

2. BCG-CDC.errorgaps—this is a processed version of the gaps file.

3. BCG-CDC.align—this is the fully annotated file that is used to locate all polymorphisms.

Pairwise alignments of BCG-H37Rv and BCG-CDC1551 was done using the BCG genomic sequence as reference. Results of the alignment identified three types of polymorphisms:

1. SNPs—single nucleotide polymorphisms in one or more of the sequences aligned.

2. indels—insertion or deletion of one or more bases in the sequences aligned.

3. Long polymorphic regions—regions with numerous changes in the sequences aligned.

Inserting the Annotation of the Complete Genomes into the Database

The gene annotation downloaded from either genbank or EMBL is included into the database by running the following script
$/work/mtb/scripts annot.pl—seq=[filename]—dbname=[NAME]—user=[NAME]—password=[PASS]
filename indicates either genbank or the EMBL genes annotation file.
Inserting the Data into the DB

To insert the CDC1551 SNP's into the DB the following command is run:
$ perl/work/mtb/scripts/snp-insert.pl—snp=../mummer/BCG-CDC.snp—user=[NAME]—password=[PASS]—query_acc=NC_{—}002755

To insert the H37Rv SNP's into the DB run the following command is run:
$ perl/work/mtb/scripts/snp-insert.pl—snp=../mummer/BCG-H37.snp—user=[NAME]—password=[PASS]—query_acc=NC_{—}000962

To determine whether SNP's are synonymous or non-synonymous, whether they are within or outside an open reading frame is first determined. All SNP's that lie within an ORF are taken and the amino acid for that codon containing the SNP is determined.

To determine if the BCG locations lie within ORF's run the following command is run:
$ perl/work/mtb/scripts/snp-orf-ref.pl—ref_seq=../seqs/bovis.fasta—user=[NAME]—password=[PASS]

All BCG locations within ORF's must have their amino acids determined. To do so, the following command is run:
$ perl/work/mtb/scripts/ref-aa.pl—ref_seq=../seqs/bovis.fasta—user=[NAME]—password=[PASS]

Next, the H37Rv and CDC1551 locations are mapped. To assign the CDC1551 ORF's the following command is run:
$ perl/work/mtb/scripts/snp-orf2.pl—query_seq=../seqs/CDC1551.fasta—user=[NAME]—password=[PASS]

To assign the H37Rv ORF's the following command is run:
$ perl scripts/snp-orf2.pl—query_seq=../seqs/H37Rv.fasta—user=[NAME]—password=[PASS]

To determine whether the CDC1551 SNP's are synonymous or non-synonymous the following command is run:
$ cd/work/mtb/scripts
$ perl s/work/mtb/scripts/synomous.pl—bcg_file=../seqs/bovis.fasta—query_seq=../seqs/CDC1551.fasta—user=[NAME]—password=[PASS]

To determine whether the H37Rv SNP's are synonymous or non-synonymous the following command is run:
$ cd /work/mtb/scripts
$ perl/work/mtb/scripts/synomous.pl—bcg_file=../seqs/bovis.fasta—bcg_file=../seqs/H37Rv.fasta—user=[NAME]—password=[PASS]

A set of summary columns are used to coallesce all the SNP data in one place. To do this, the following command is run:
$ perl/work/mtb/scripts/compare-snps.pl—user=[NAME]—password=[PASS]

To insert data into the SNP analysis table the SNP data from the SNP, SEQ_SNP and gene ontology tables is fetched and entered into the SNP_analysis table. This step also identifies the conservative and non-conservative amino acids.

To do this, the following program is run:
$ run.sh/work/mtb/scripts/

The SNP data in the database is thus complete.

Analysis of SNPs

The SNPs identified were of two kinds:

i. Identical nucleotide in CDC1551 and H37Rv, but a different nucleotide in BCG at the same position.

ii. One of the three sequences is polymorphic; the nucleotide sequence of CDC1551 and H37Rv are different from each other and one of them is identical to the BCG sequence at identical positions.

The SNPs thus identified were categorized according to their location in Open Reading Frames. SNPs falling within the ORF of both BCG and H37Rv were identified. The results were validated by determining if the SNPs were present in the ORFs of BCG and CDC1551.

The SNPs falling in ORFs were further categorized into synonymous and non-synonymous SNPs. A SNP was said to cause a non-synonymous change if:

1) It occurs in an ORF

2) It occurs in the *same* ORF in the genome it is being compared to.

In some cases a SNP can be in one ORF in the reference sequence but in another ORF in the comparison sequence, e.g. due to a frame-shift mutation earlier in the sequence.

So before we assign SNP's to ‘Non Synonymous’ or ‘synonymous’ groupings all SNP's which either did not fall in an ORF, or fell into different ORF's on the reference and comparison sequences were eliminated. The BCG and H37 genomes have been annotated with respect to one another. However CDC1551 has not been so thoroughly annotated, so it was not possible to immediately assess if an ORF in BCG was the corresponding ORF in CDC. Therefore, a metric was devised to eliminate spurious comparisons.

The non-synonymous SNPs thus identified was analysed to predict conservative and non-conservative amino acid substitutions. The effect of the substitution on the function of the proteins encoded was predicted. This provides a powerful insight in predicting SNPs correlating with virulence and infectivity in M.tuberculosis.

Below is an example of the output obtained from the database.

[image]

The above figure describes the SNP details, which is as follows:

Bovis_pos—Bovis position having a SNP.

Bovis_ORF—Y es indicates that the SNP in bovis is in bovis ORF. No indicates not in ORF.

Bovis_base—Indicates the SNP detailSNP pos ition in bovis

Bovis_AA—Displays the bovis amino acid after the codon translation.

Qry_name—Displays the name of a strain, example H37Rv or microtii

Qry_pos—Displays the position of a SNP in either CDC1551 or H37Rv with respect to bovis SNP position.

Qry_ORF—Displays Yes if the SNP falls in the ORF of the query (H37Rv or CDC1551)

Qry_base—Displays the query SNP.

Qry_AA—Displays the amino acid of the query (H37Rv or CDC1551).

Is_nsSNP—Displays SNPs synonymous (S), non-synonymous (NS) and SNPs in non-coding region (NC).

Conservative_subst—Displays homologous substitution in H37rv and CDC1551.

Fun_annotation—Will display the functional annotation of the query.

A list of Single nucleotide polymorphisms identified in the manner described above is given in Table 1.

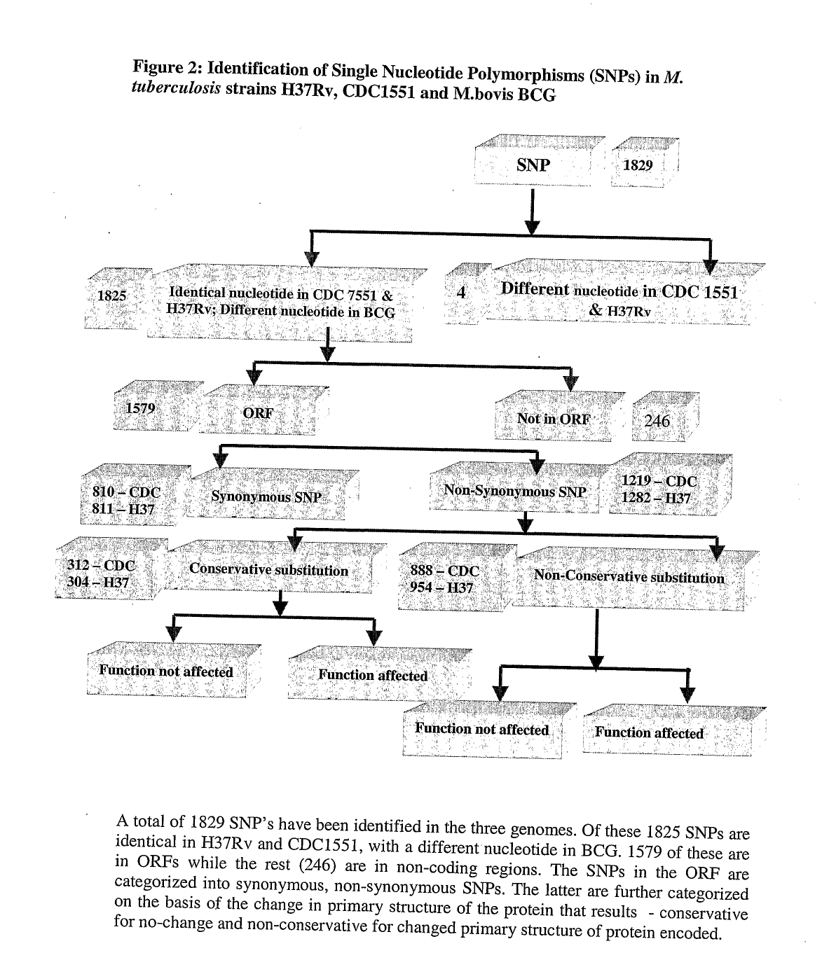

A total of 1829 have been identified in the three genomes. Of these 1825 SNPs consist of having the same nucleotide in H37Rv and CDC1551, with a different nucleotide in BCG. Of thel829 SNPs, 1579 are in ORFs while the rest (246) are in non-coding regions. 811 H37Rv SNPs and 810 CDC1551 SNPs are synonymous while 1282 H37Rv and 1219 CDC1551 SNPs are non-synonymous. Out of 1219 CDC1551 nsSNPs, 312 SNPs have conservative amino acid substitution, 888 have non-conservative substitution and 19 results in truncated proteins. Out of 1282 H37Rv non-synomous SNPs, 304 have conservative amino acid substitution, 954 have non-conservative substitution and 24 results in truncated proteins. (FIG. 2)

Analysis of Indels (Insertions and Deletions):

Indels are insertions and deletions in the sequence with respect to BCG sequence. These indels could be of one or more nucleotides. Considering BCG as reference sequence, the indels in the both the strains of M.tuberculosis, H37rv and CDC1551 were identified.

To insert the indels from the align file of the mummer output into the database, the following java program is run:
$ java/work/mtb/scripts/indel

To enter functional annotation from the gene ontology database into the indels table, the following program is run:
$ java/work/mtb/scripts/indfunction

The list of indels identified is given in Table 2.

A total of 794 indels have been identified in the three genomes. Of these, 237 (H37Rv) and 237 (CDC1551) indels are present in both H37Rv and CDC1551 with respect to BCG. Of these, 178 are in ORF and 59 are outside the ORF. (FIG. 2)

Analysis of Long polymorphs:

Long polymorphs are insertions or deletions of long stretches of nucleotides with respect to BCG sequence.

To insert the long polymorphs from the align file of the mummer output into the database, following java program is run:
$ java/work/mtb/scripts/indel

To enter the functional annotation from the gene ontology database into the long polymorph table, following java program is run:
$ java/work/mtb/scripts/indfunction

A table listing the long polymorphisms is given in Table 3.

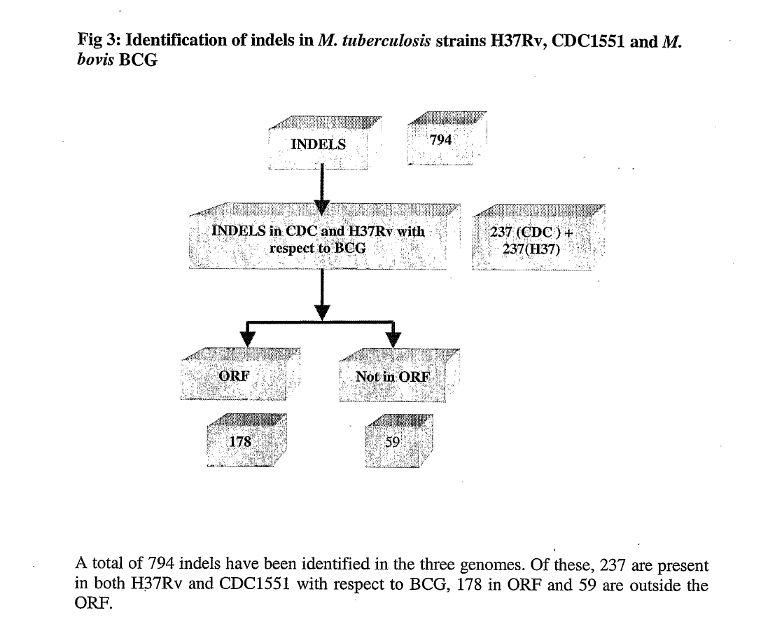

A total of 136 long polymorphisms have been identified in the three genomes. Of these, 30 (H37Rv) and 30 (CDC1551) indels are present in both H37Rv and CDC1551 with respect to BCG. Of these, 22 are in ORF and 8 are outside the ORF. (FIG. 3)

Functional Annotation of the Polymorphisms Identified

In order to identify polymorphisms with a putative functional association, a tool was built using the Gene Ontology DB (GO). The EMBL sequence DB has made putative GO assignments to most of the ORF's in the three TB genomes, so a local installation of GO was used together with the EMBL cross reference tables to identify TB polymorphisms based on their putative functional classification.

The annotation table consisting of the genbank features of the genes such as coding region, database reference and product information to name a few was constructed.

To inserts the gene ontology features such as term definition and name from the gene ontology database into the indels and long polymorph table, following program is run:
$ java/work/mtb/scripts/indfunctionl

The following are the list of attributes in the annotation table.

Accession no—This indicates the accession number of the sequences

Gene_start—This indicates the start of the coding region

Gene_end—This indicates the end of the coding region

Locus_tag—

db_xref—This indicates the gene indices representation of the gene

db_xref_GOA—This indicates the gene ontology identity of the gene product

id—This indicates the gene annotation

type—

strand—This indicates the forward or reverse strand of the sequence that is stored in the genbank

gene_name—This indicates the gene name

gene_link—This provides a hyperlink to the gene features form the genbank

note—This provides the general information and the protein information of the gene.

A front-end was constructed as an essential part of the database:

Front End of the Database:

The front-end displaying the results of alignment as follows:

The annotation table consists of genbank annotation about the genes in bovis, H37Rv and CDC1551. It specifies details including the coding region of a gene and its database reference.

The annotation id for the SNPs, indels and long polymorphs has been hyperlinked to obtain all the records pertaining to a particular gene.

The data pertaining to indels and long polymorphs have also been added to the front-end.

Description of the Queries:

The database is made queryable to retrieve the required features of SNPs, indels and long polymorphs respectively.

The main options to query the SNP information are:

Select SNPs

ALL—This displays all the records which satisfies the below features.

Identical in both queries—This query indicates that SNPs are present in BCG with respect to H37Rv and CDC1551.

Different bases in both queries—This query indicates different nucleotides in H37Rv and CDC1551.

Having SNPs in BCG-H37 only—This query specifies SNPs in BCG and H37Rv only and not in CDC1551.

Having SNPs in BCG-CDC only—This query specifies SNPS in BCG and CDC1551 only and not in H37Rv.

BCG-H37 SNPs—This query indicates, that SNPs are present in H37Rv with respect to BCG-position and may or may not be present in CDC1551 at that particular position.

BCG-CDC SNPs—This query indicates, that SNPs are present in CDC1551 with respect to BCG position and may or may not be present in H37Rv at that particular position.

The other options considered are:

Select BCG ORF—This provides an option to select the presence of BCG SNPs in BCG ORF or outside the BCG ORF.

Select query ORF—This provides an option to select the presence of query SNPs in query ORF or outside the query ORF.

Select synonymous—This provides an option to select if the SNP is synonymous or non-synonymous.

Select Conservative—This provides an option to select if the non-synonymous SNP results in conservative, non-conservative substitution or truncated protein.

Select function—This provides an option to select a required function, which includes cell wall synthesis, Transcription factor, Lipid metabolism, Membrane transport and Surface proteins.

An example of a query to extract SNP information from the database is shown below.

[image]

The result obtained from the above query is shown below:

[image]

The query has been designed in the similar way for both indels and long polymorphs.

The SNP analysis includes functional annotation id, which is hyperlinked to the functional annotation of the gene carrying the polymorphism. The functional annotation id consists of either one of the Swiss Prot, SPTREMBL or gene ontology id's. Similarly the indels and long polymorphs are also functionally annotated.

Genes with known involvement in virulence of Mycobacterium tuberculosis can also be accessed from the SNP database query or from the Long polymorphs database query respectively.

Polymorphisms involved in the following functions have been identified:

1. Cell wall synthesis

2. Transcription factor

3. Lipid metabolism

4. Membrane transport

5. Surface proteins.

6. Virulence genes

One such query for cell wall synthesis function is shown below

[image]

The output of the above query is shown below

[image]

The polymorphisms detected in genes involved in cell wall synthesis are listed in Table 4.

Visualization Tools

To increase the utility of the SNP data, two tools to visualize the Tuberculosis SNP data have been created: the first tool was based on the Generic Genome Browser developed at Cold Spring Harbor Lab (CSHL). This visualization tool could show a single TB genome along with any annotations, e.g. SNP locations for all other genomes.

The details of the browser is as follows:

The output displays the polymorphs in the region of interest.

Alternatively the output can be obtained by specifying the region of interest in the text box labeled as “landmark or region”. In case of SNP, the gene start and the gene end has to be specified and in case of indels or long polymorphs, the BCG start and BCG end must be specified.

By clicking the ruler at the region of interest across the genome, the view can be re-centered.

The display can also be zoomed in or out by selecting the required number of base pairs in the scroll down menu.

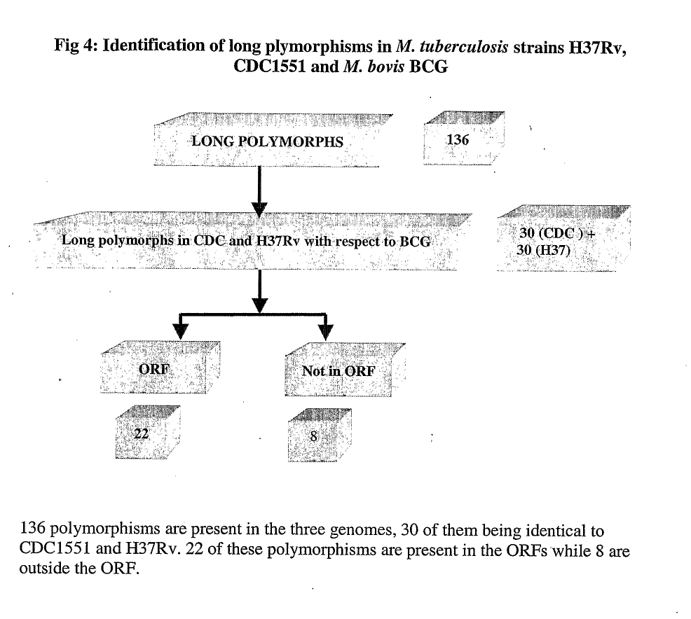

The required features can be displayed by selecting the options in the tracks checkbox as shown in FIG. 4

FIG. 4 display shows a region of 10 kb of the BCG genome with three types of annotations: BCG ORF's, SNP's in H37Rv, and SNP's in CDC1551.

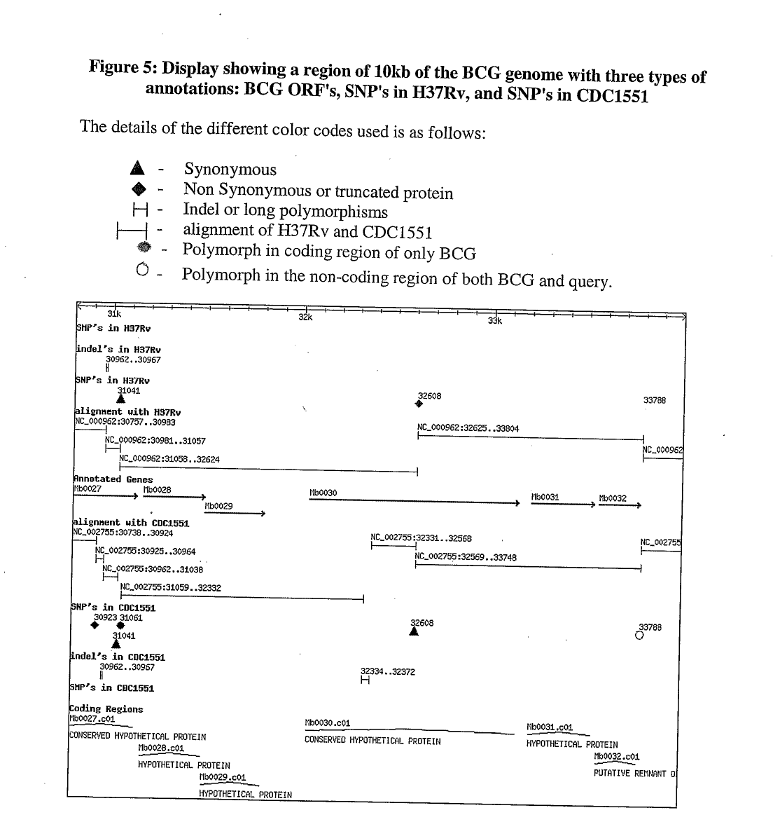

To compare multiple genomes, a second tool based on the WormBase synteny browser was built. This tool can visualize two TB genomes at one time and was very useful in validating the polymorphisms the CDC1551 genome as shown in FIG. 5.

FIG. 5 shows the comparative genomics browser displaying BCG in the upper panel and H37Rv in the bottom panel. The segments labeled MUM-* are the perfect matches generated by the MUMmer tool, and the vertical lines show the alignment of the MUM segments in both genomes. The color coding of the ORF's is used to indicate the length of the ORF. This is very helpful to researchers because if an ORF in H37 aligns with an ORF in BCG but they have different colors, then there is a mutation that makes them have different lengths (see for example the genes in the MUM-1280 region).

A methodical screening of all the regions of polymorphism identified above in clinical isolates with known disease profiles to further home-in on the polymorphisms associated with virulence and/or infectivity in M.tuberculosis is in progress.

2. Screening of Regions of Polymorphisms

A set of five Mycobacterium tuberculosis strains with known virulence is being screened for the polymorphisms identified above.

Strains chosen: The following strains have been chosen for the study:

a. H37Rv—a reference laboratory strain known to be infective to mice, but is only mildly infective in humans. It has undergone a number of passages in the lab since its isolation. It is the standard used in studies on tuberculosis in different laboratories across the world.

b. Beijing strain—a clinical isolate with known virulence and infectivity in humans. 70% of the patients with tuberculosis in certain areas of India and China are infected with this strain. The strain was isolated from a patient in the Western Indian state of Mumbai.

c. S.I—a mild South Indian strain with only mild virulence and infectivity in humans isolated from a patient residing in the South Indian state of Hyderabad.

d. N.I.F—Fatal North Indian strain isolated from Safderjung hospital, Delhi where the patient developed pulmonary tuberculosis died.

e. N.I.NF—a non-fatal North Indian strain isolated from Safderjung hospital, Delhi. Known clinical progression of disease in the patient.

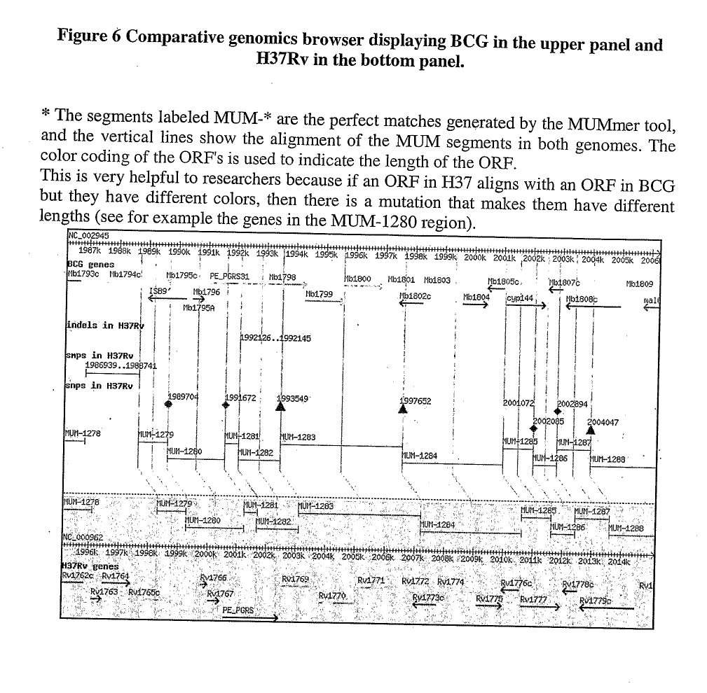

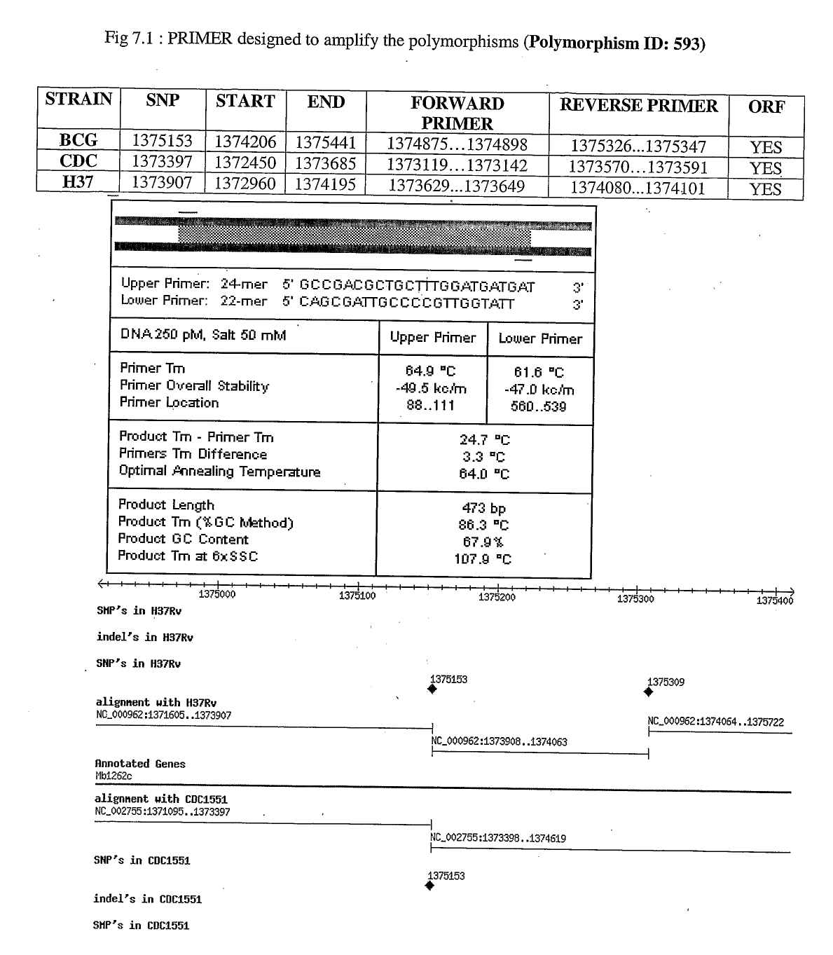

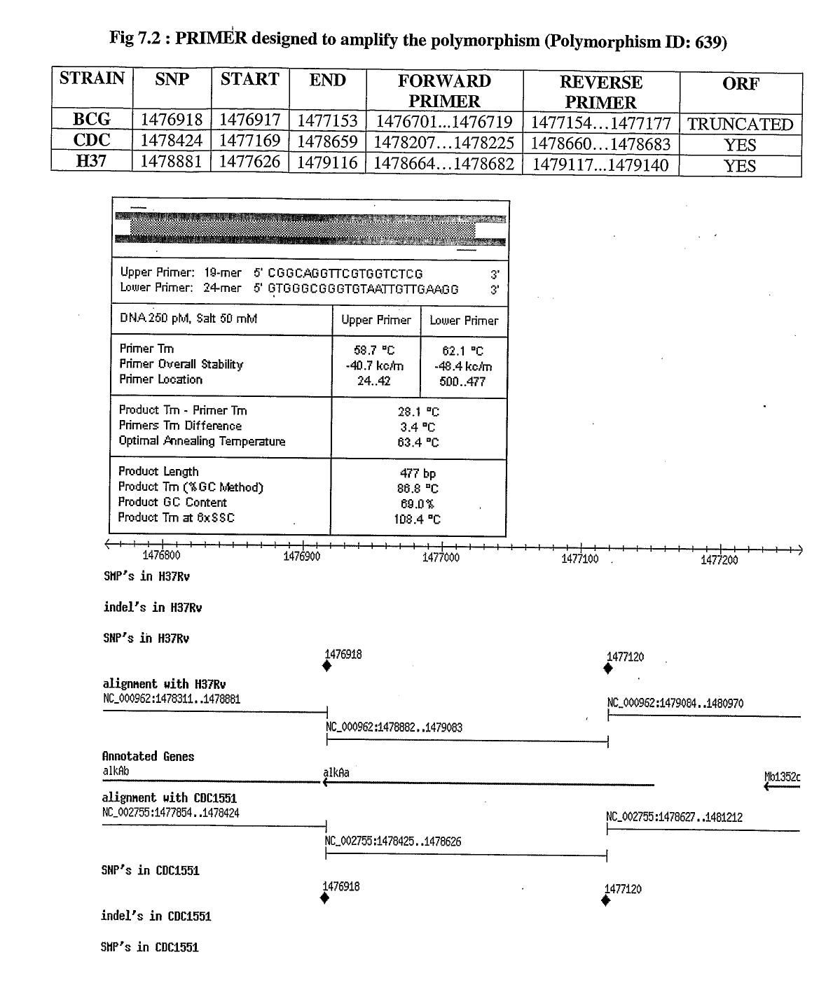

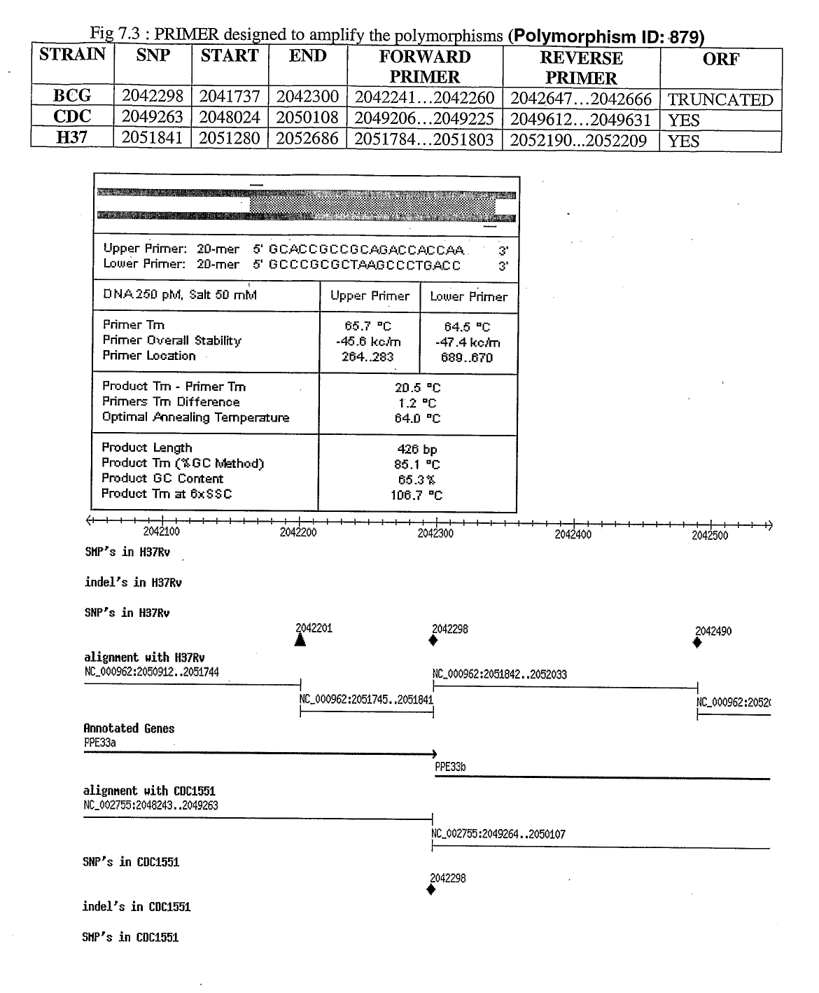

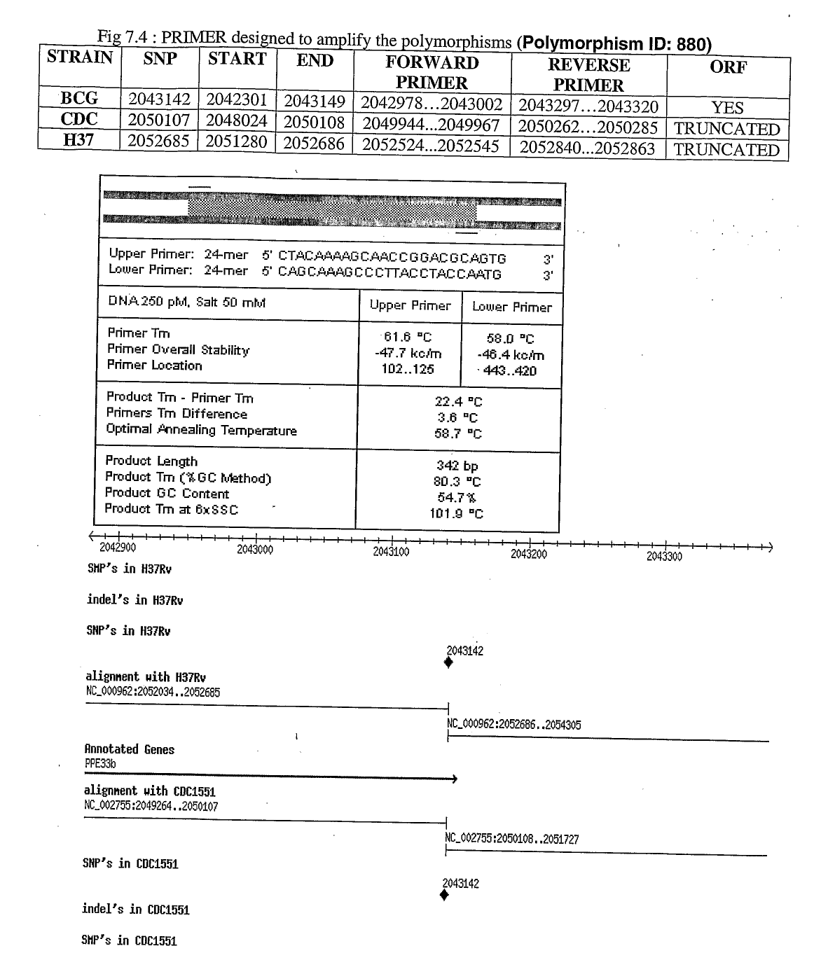

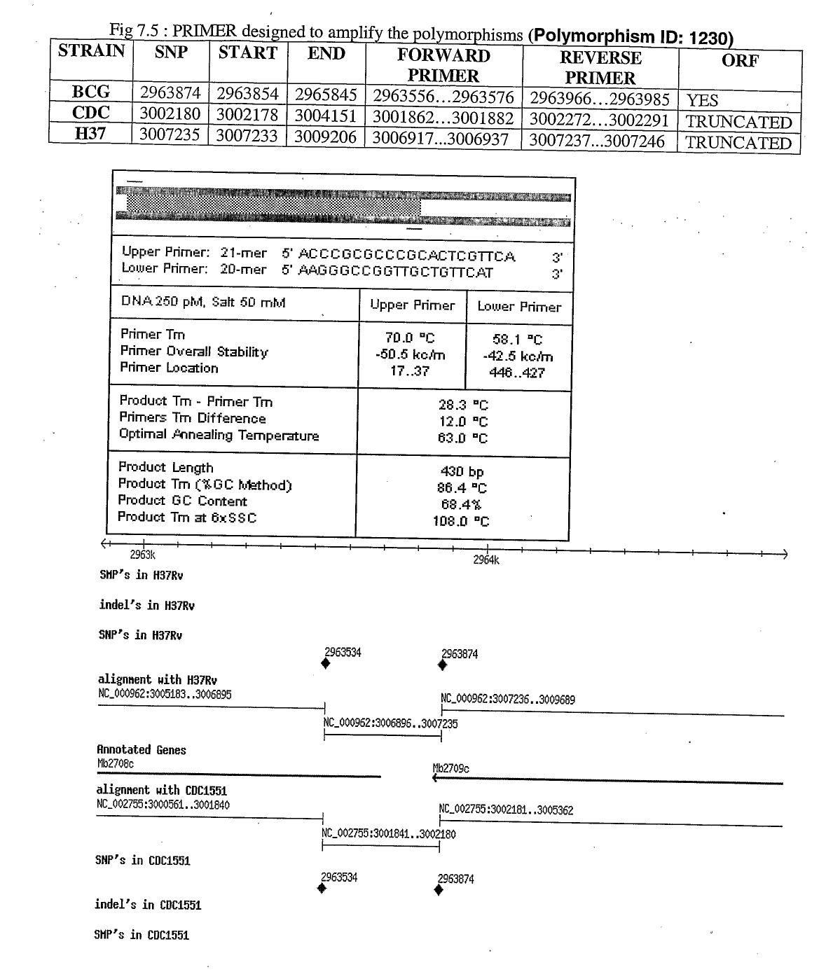

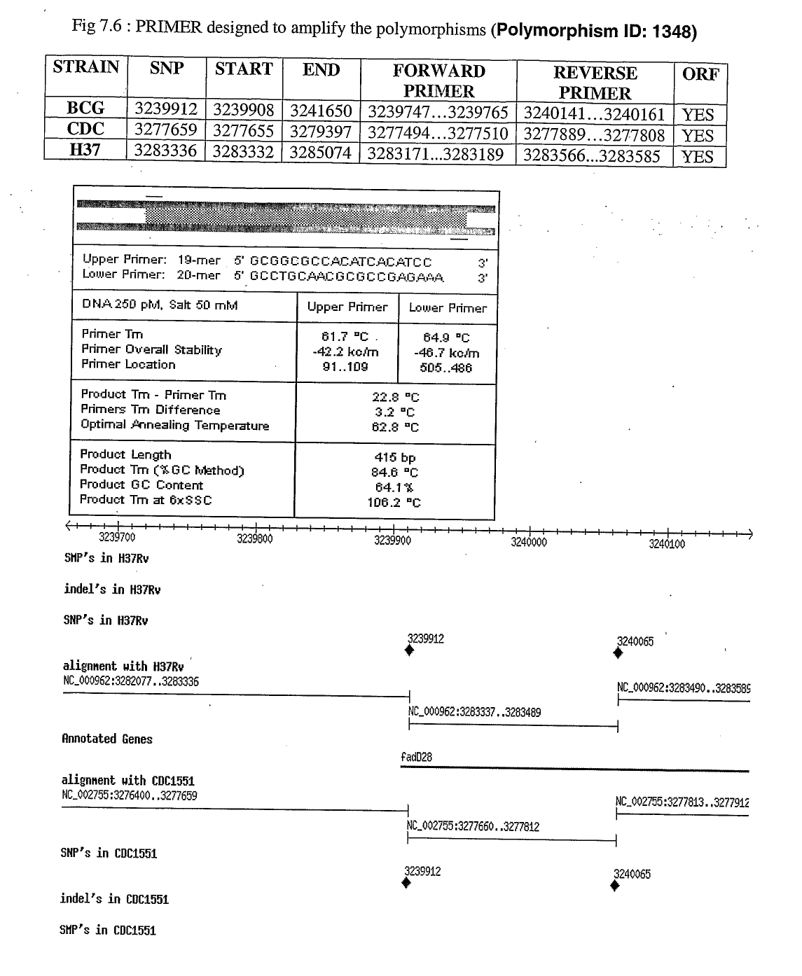

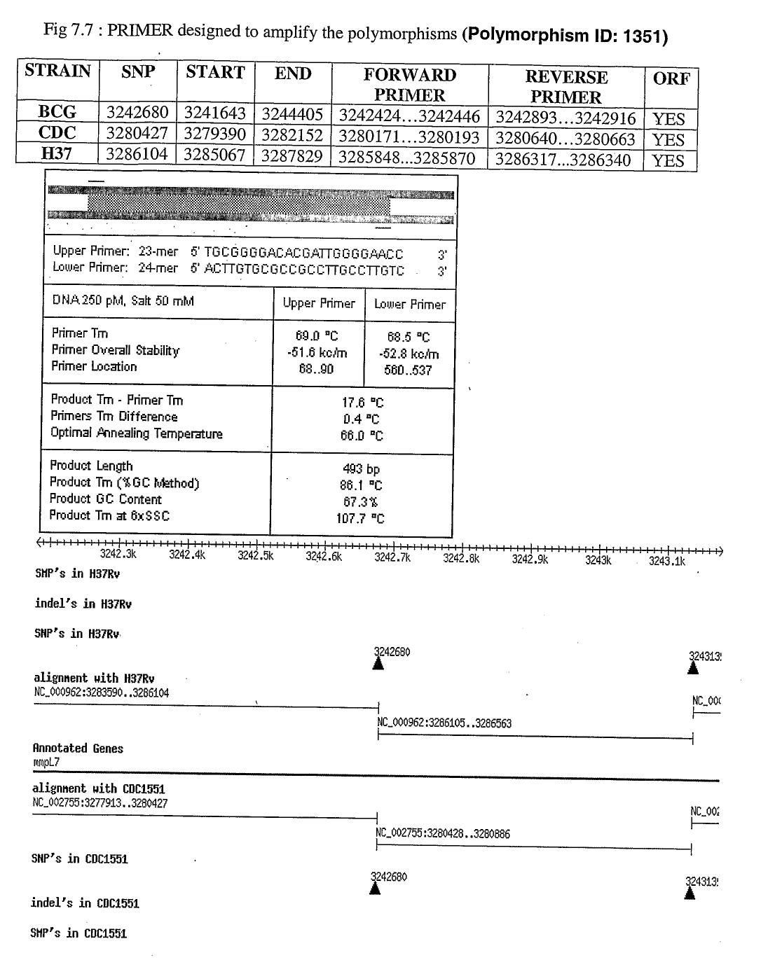

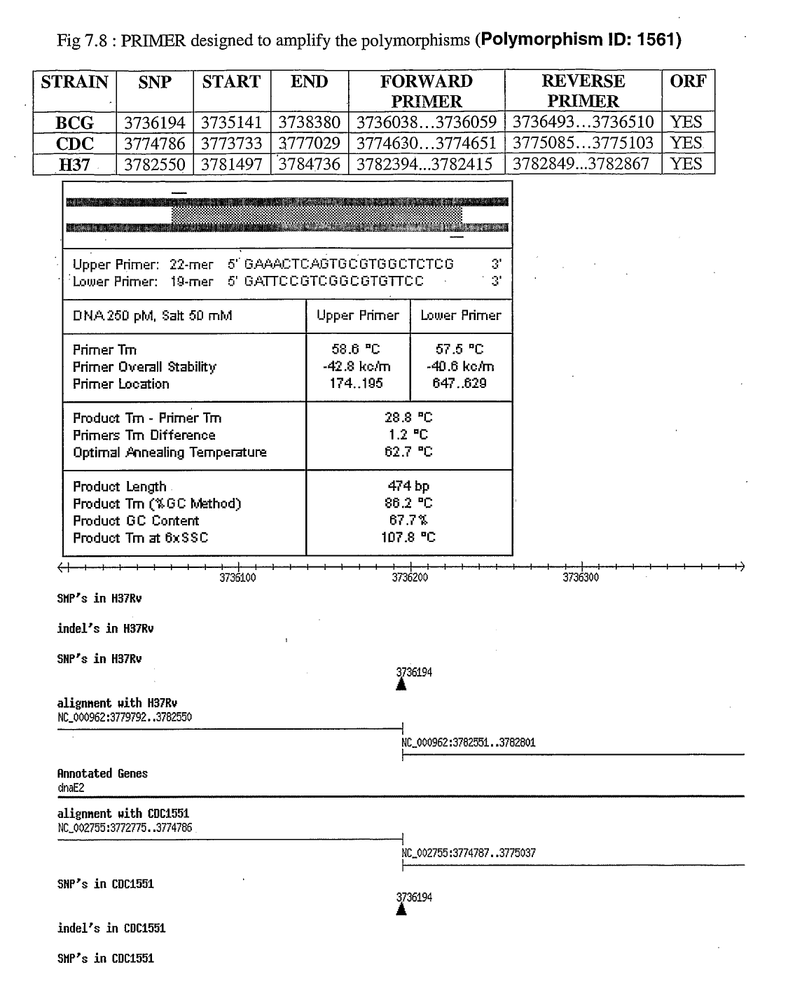

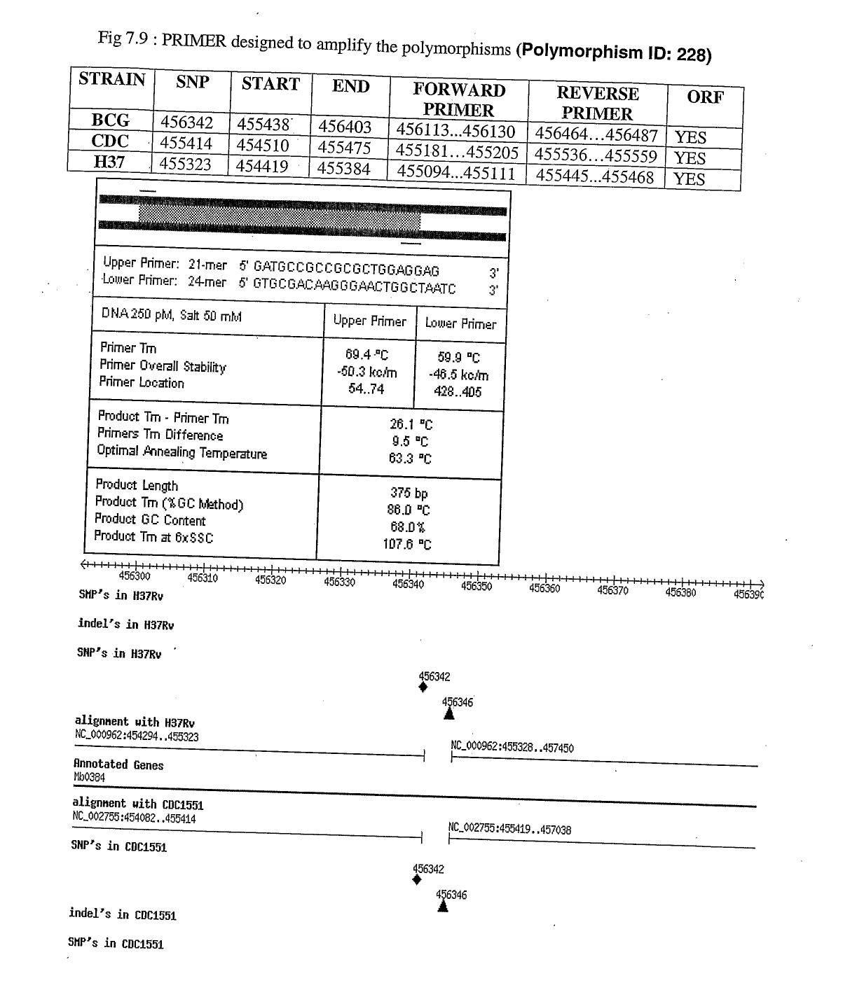

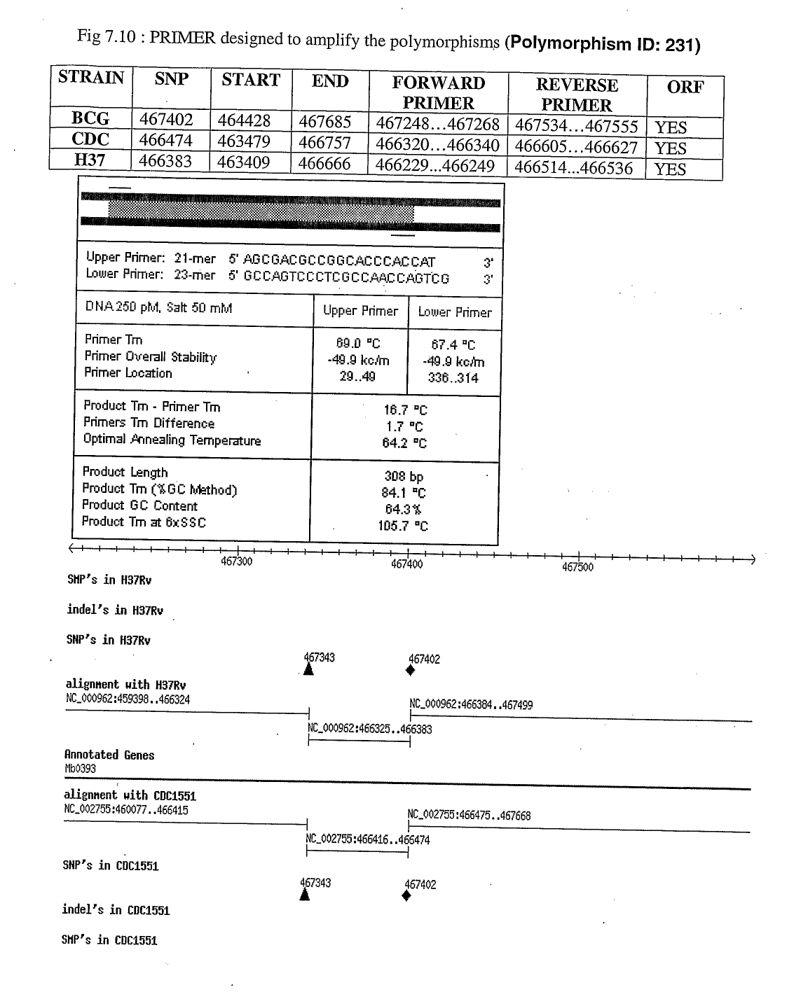

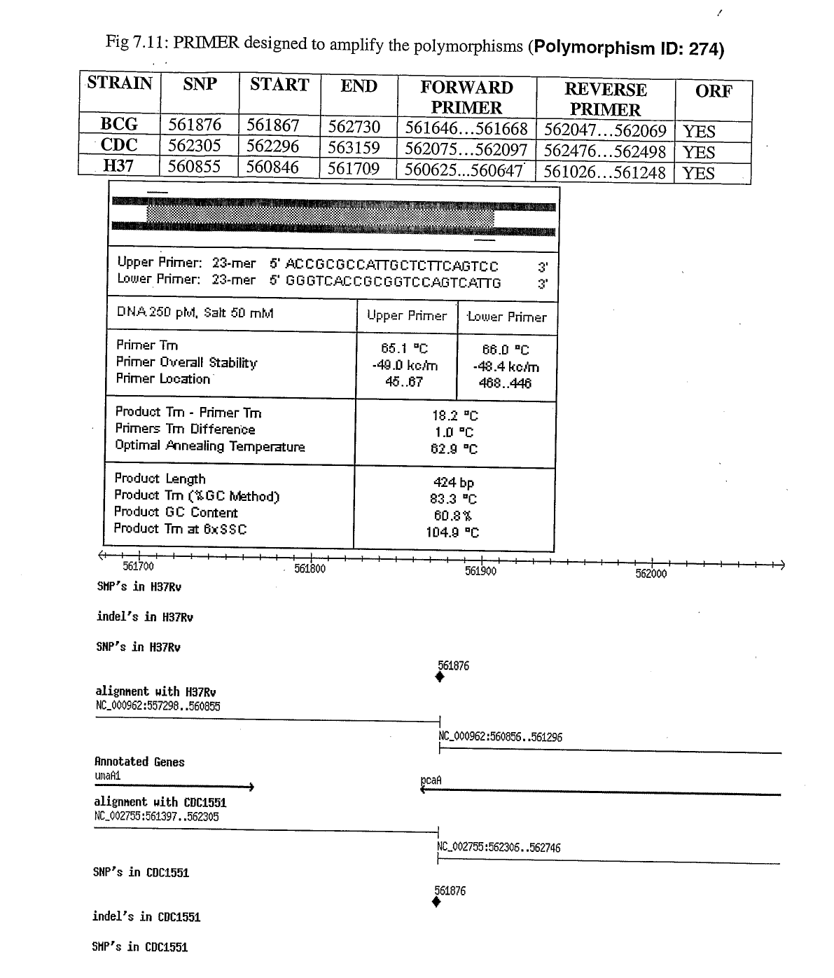

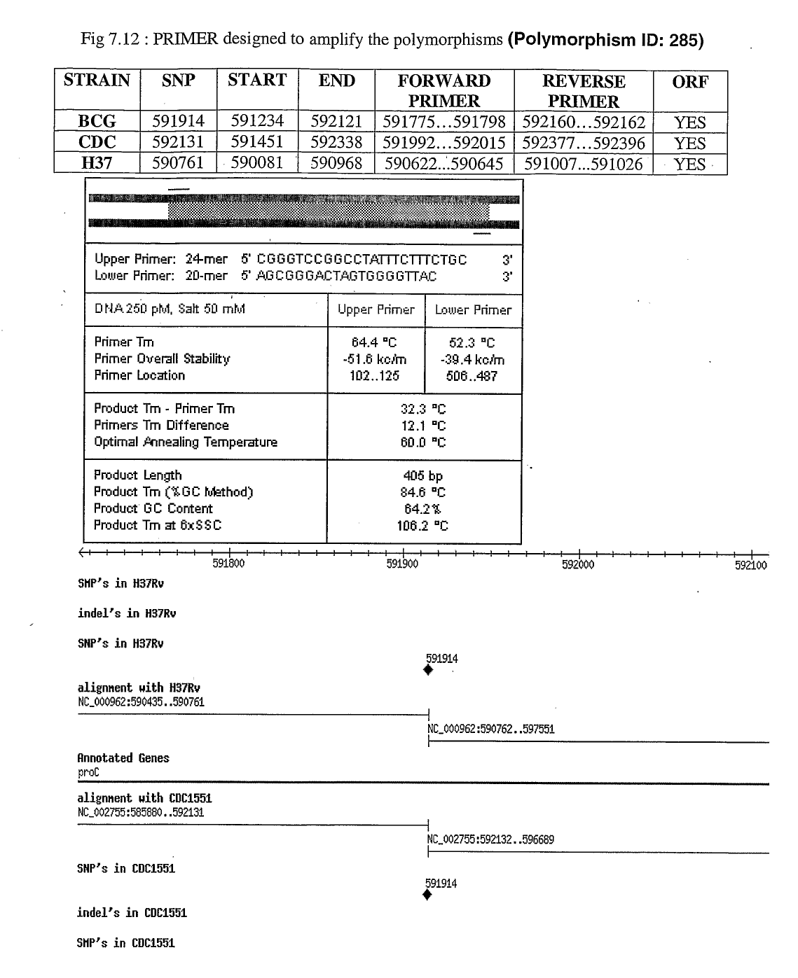

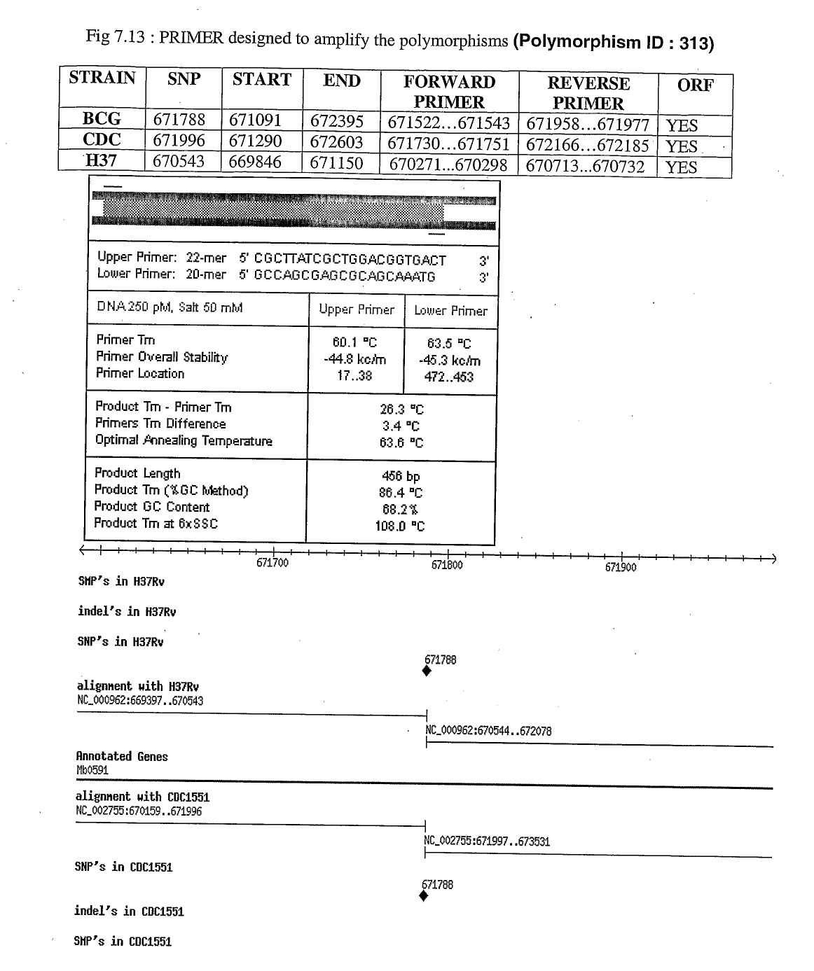

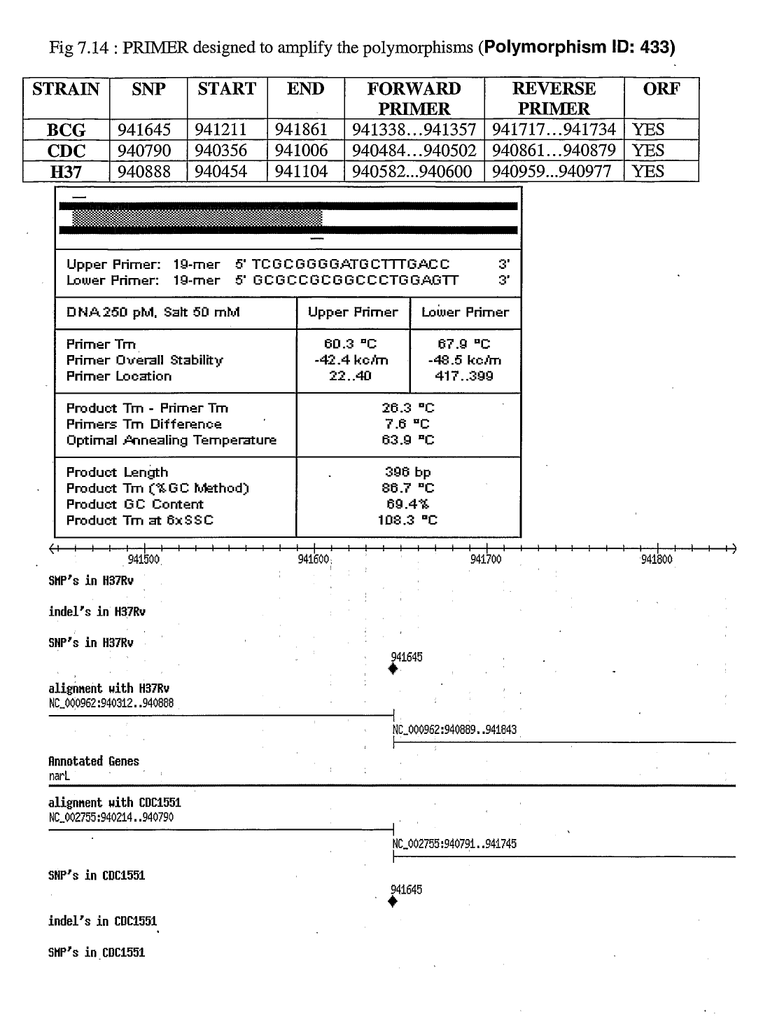

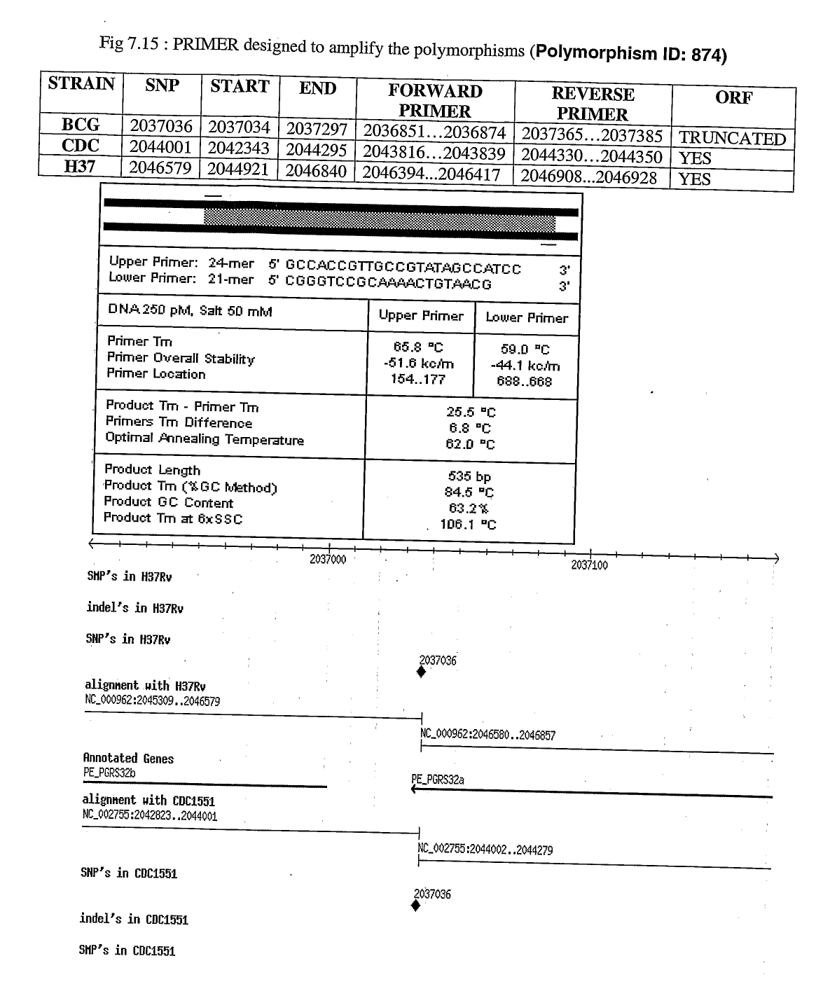

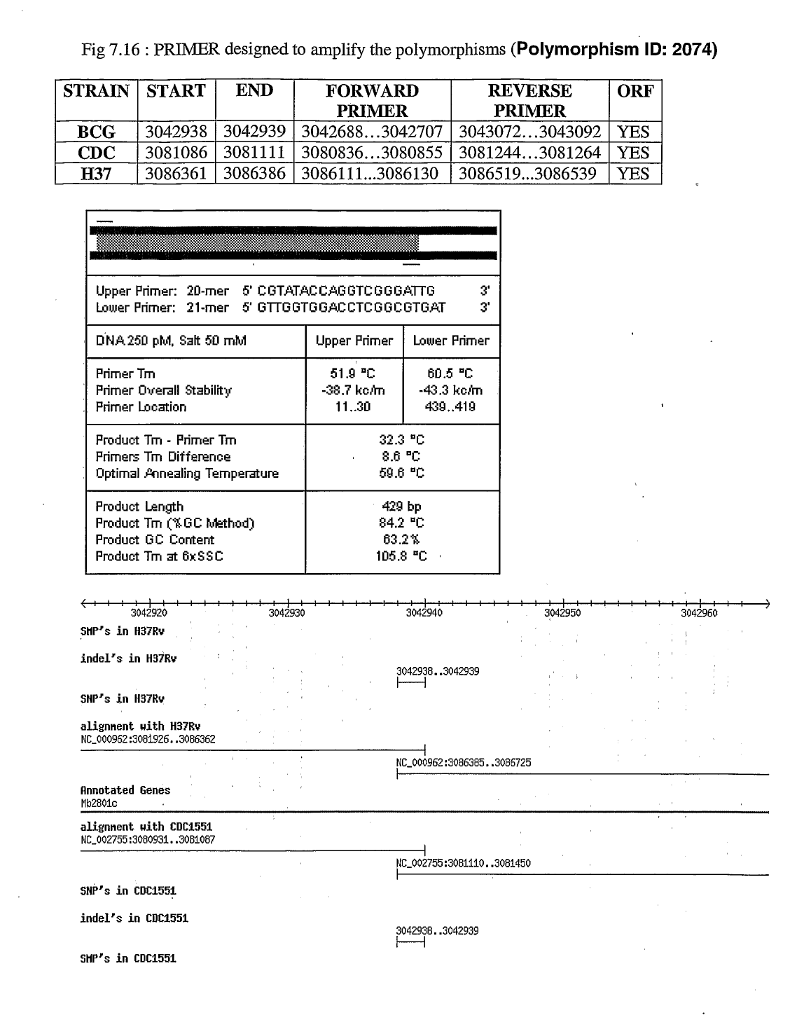

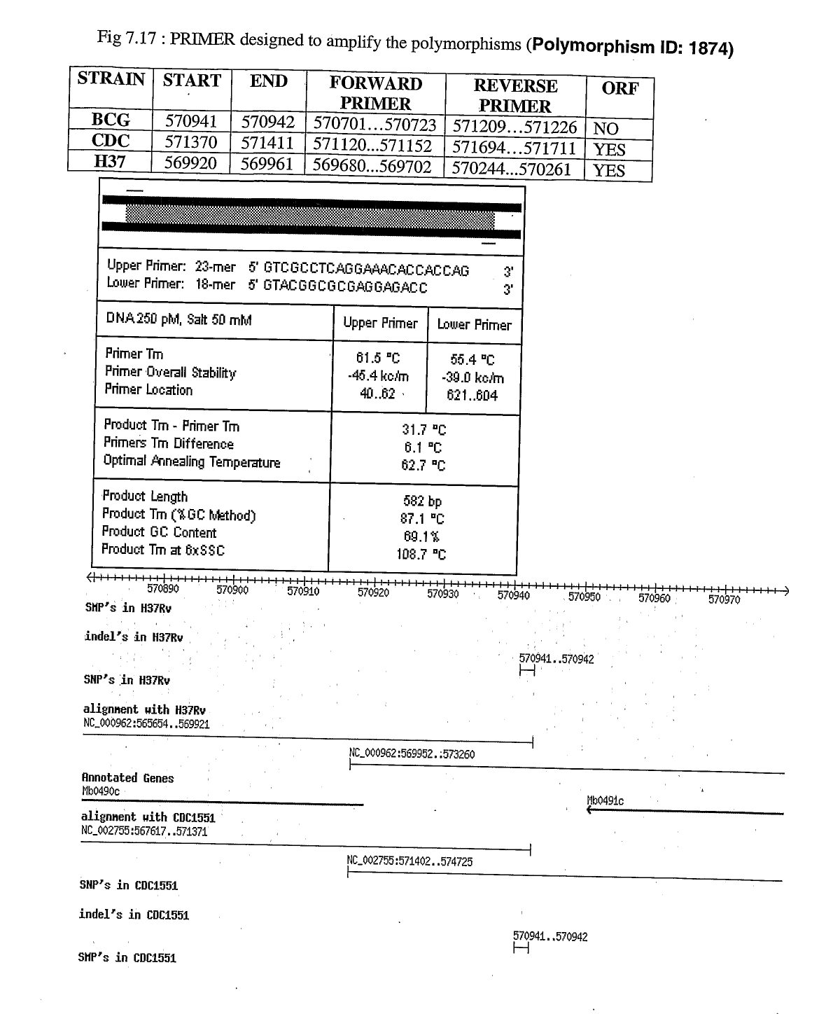

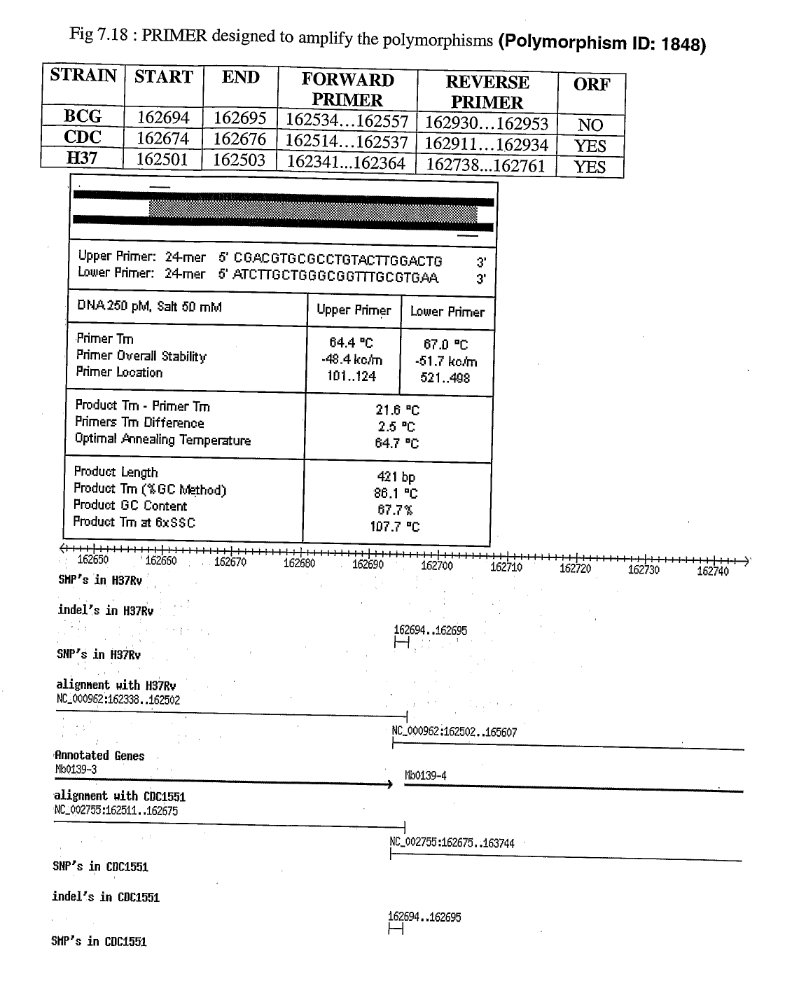

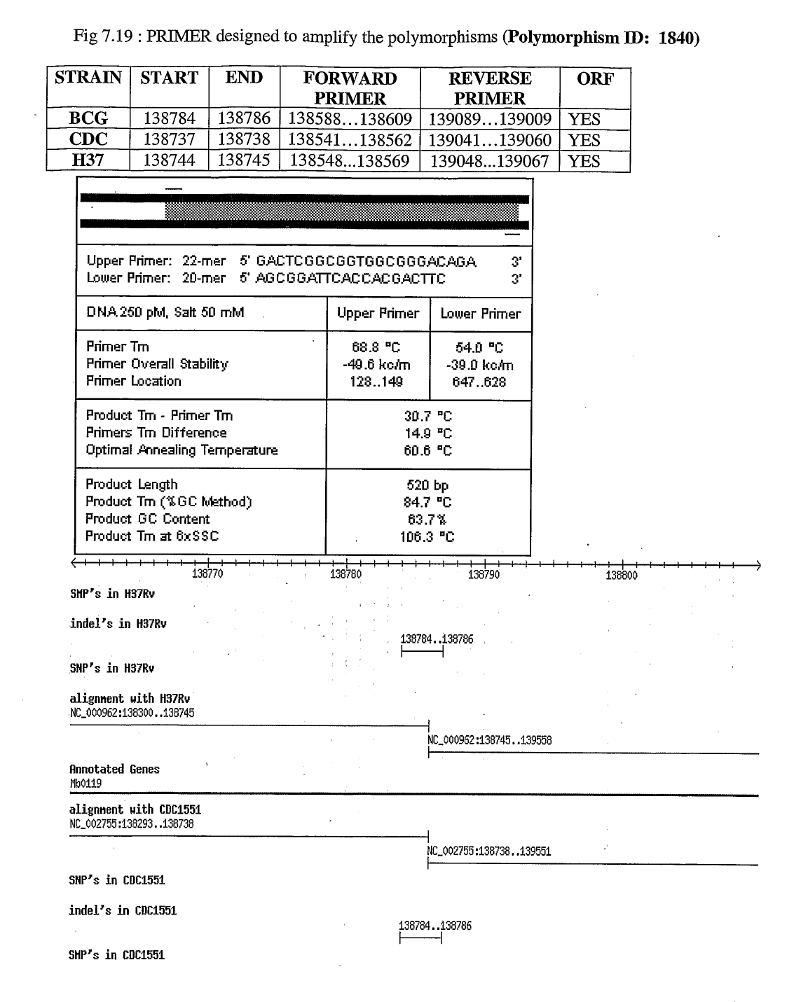

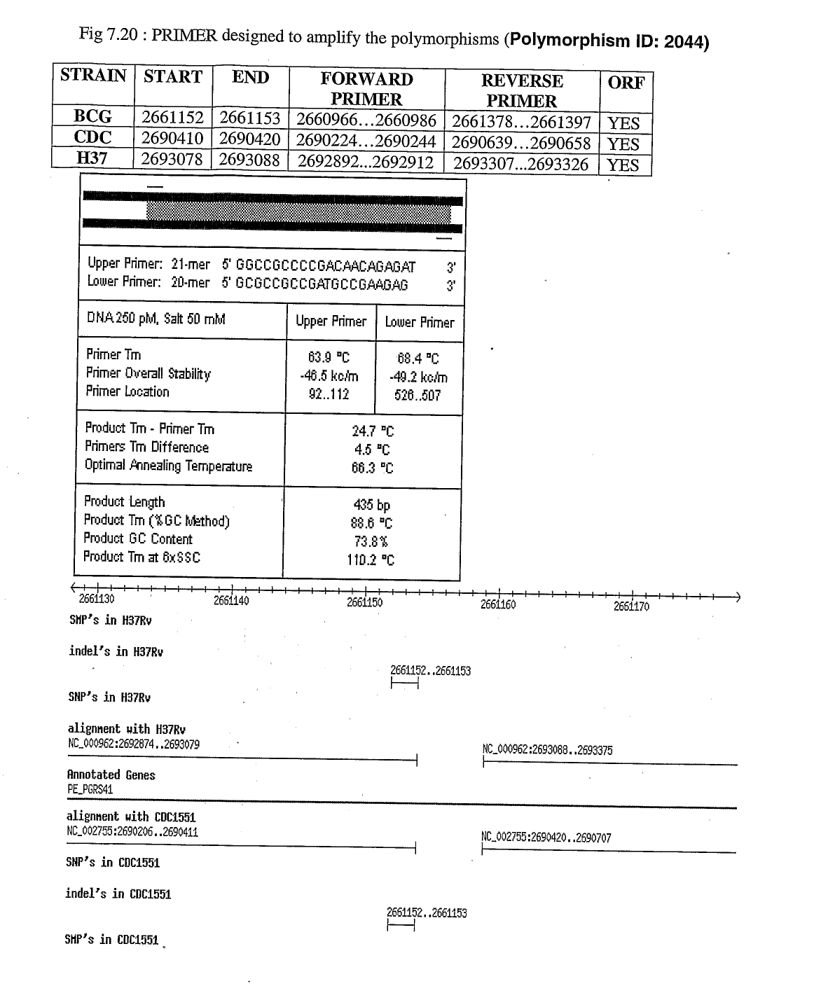

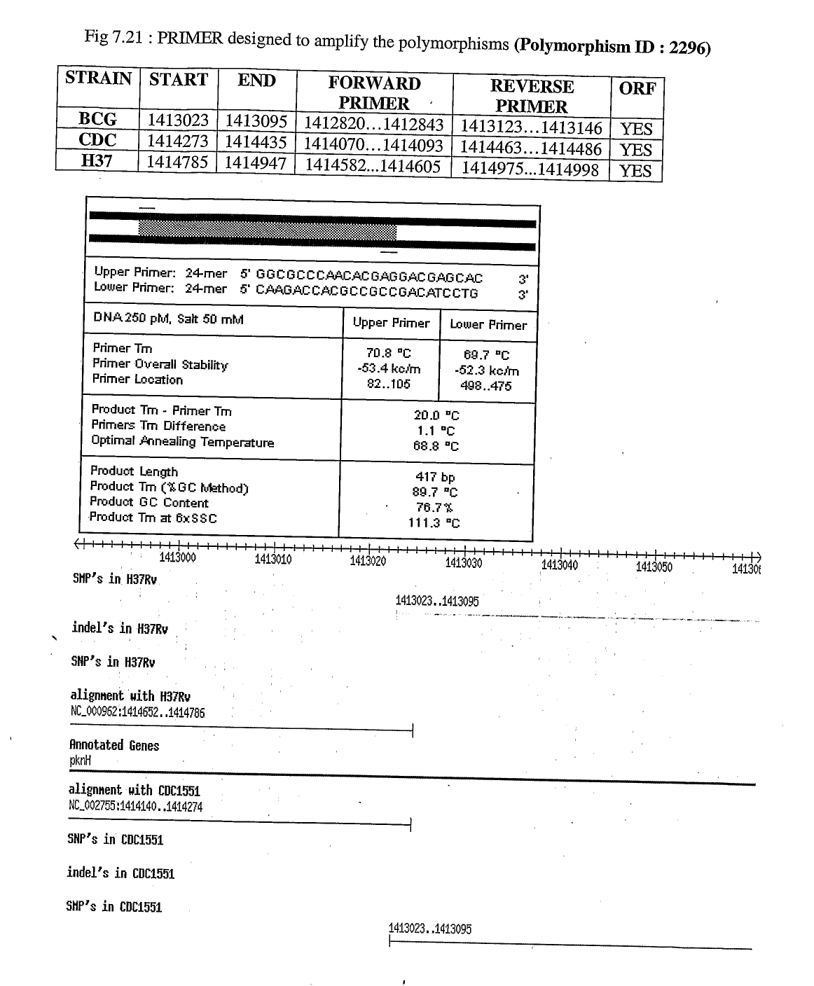

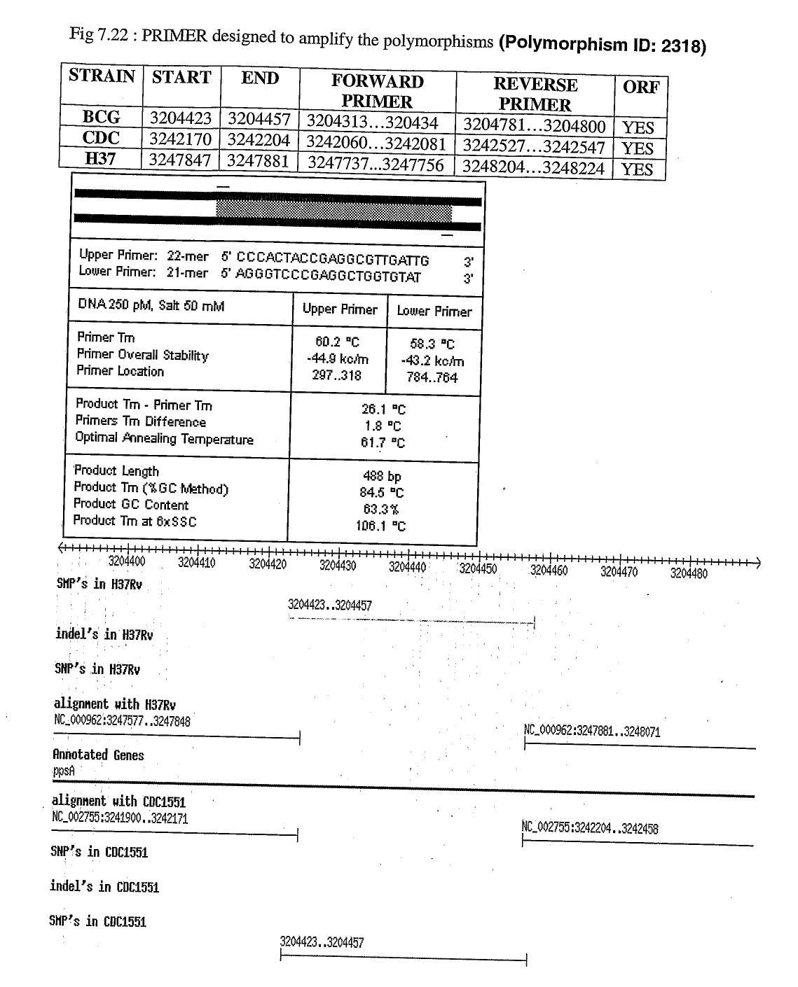

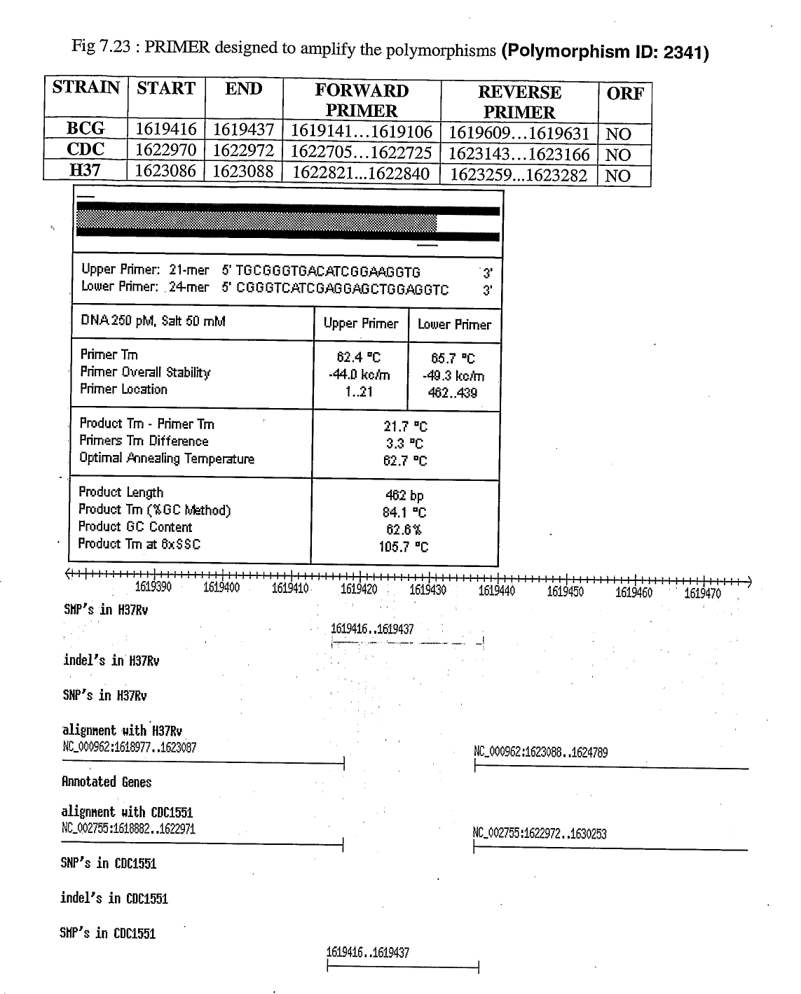

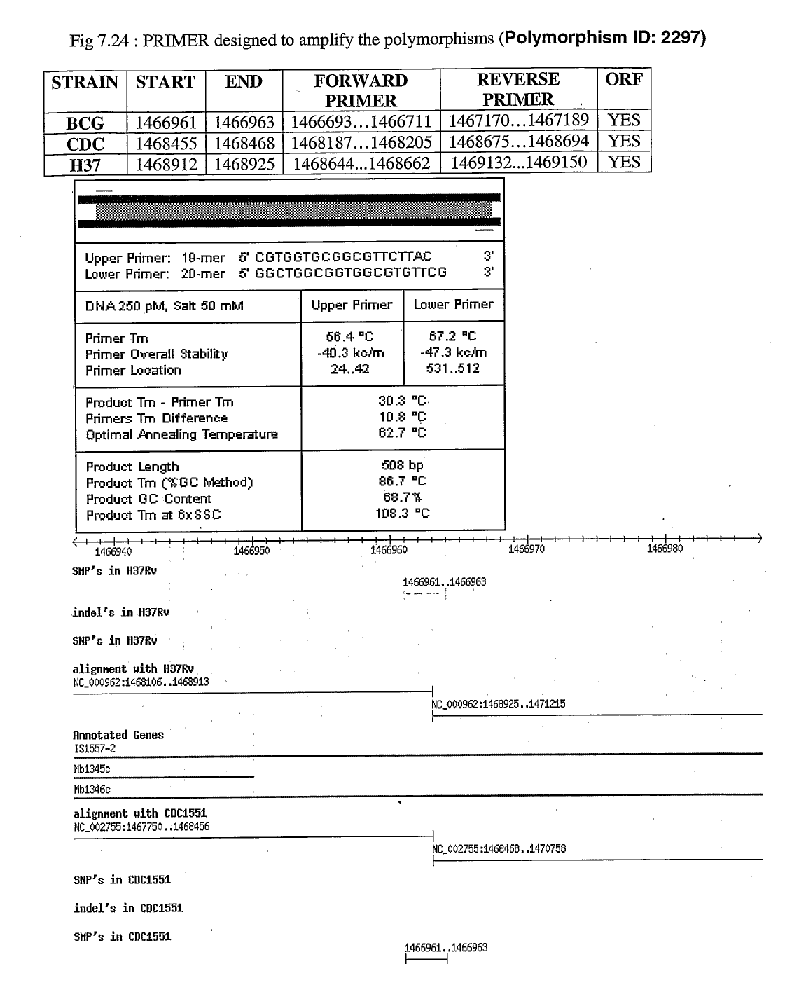

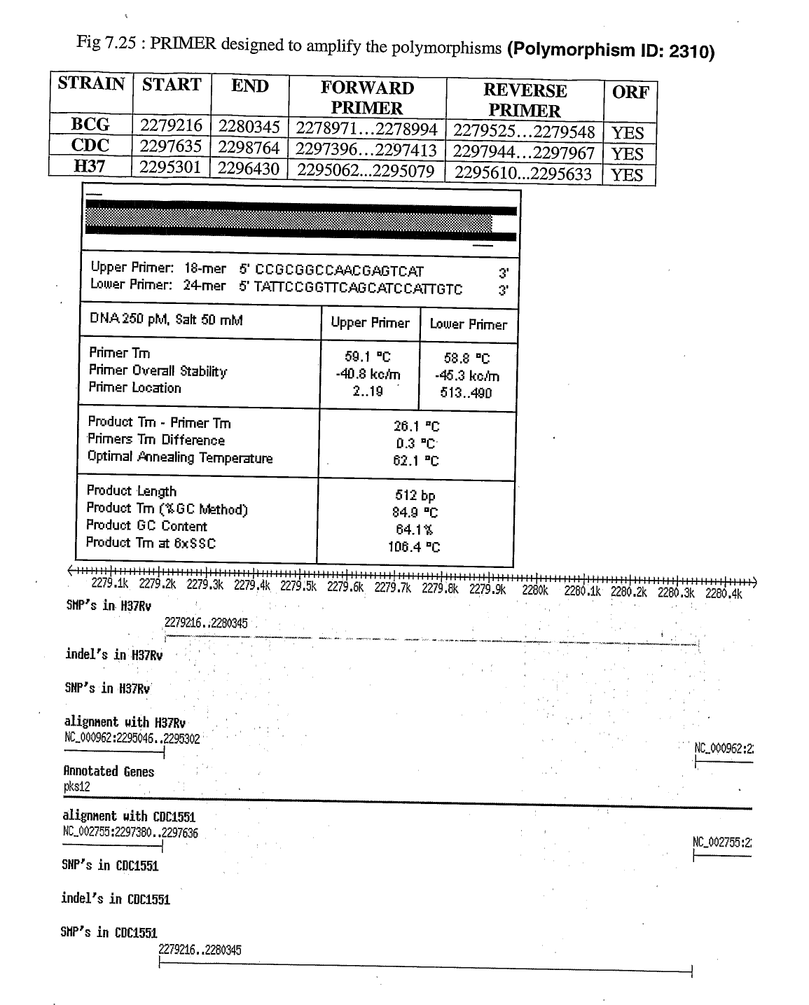

Primers have been designed to encompass the regions of polymorphisms. The list of the primers used for the amplification is given in the FIG. 6.1-6.25

Amplification and sequencing of regions around the polymorphisms: DNA from the five strains has been amplified under optimal conditions determined for each primer pair. The amplified fragments have been sequenced and the sequences obtained from different strains compared.

A few examples are given below:

| | |
|---|---|
| | 60 70 80 90 100 110 |
| | +---------+---------+---------+---------+---------+----- |
| BCG | ACCGATCTCGCCGCGCAGACAATGGCTGGCTCAGCGGCGATGCTGCTGGAGCGGAT |
| H37Rv | ++++++++++++++++++++++++++++++++++++++++++++++++++++++++ |
| CD1551 | ++++++++++++++++++++++++++++++++++++++++++++++++++++++++ |
| SI | ++++++++++++++++++++++++++++++++++++++++++++++++++++++++ |
| NINF | ++++++++++++++++++++++++++++++++++++++++++++++++++++++++ |
| BS | ++++++++++++++++++++++++++++++++++++++++++++++++++++++++ |
| NIF | ++++++++++++++++++++++++++++++++++++++++++++++++++++++++ |

| | |
|---|---|
| | 120 130 140 150 160 170 |
| | ----+---------+---------+---------+---------+---------+ |
| BCG | GGACCAAGACCAGGGTGGCGCCAATGGCGAGCTGATGGGGCTGCGCGTGGACCTT |
| H37Rv | +++G+++++++++++++++++++++++++++++++++++++++++++++++++++ |
| CD1551 | +++G+++++++++++++++++++++++++++++++++++++++++++++++++++ |
| SI | +++G+++++++++++++++++++++++++++++++++++++++++++++++++++ |
| NINF | +++G+++++++++++++++++++++++++++++++++++++++++++++++++++ |
| BS | +++G+++++++++++++++++++++++++++++++++++++++++++++++++++ |
| NIF | +++G+++++++++++++++++++++++++++++++++++++++++++++++++++ |

Sequencing of the region from 1H-590622 to H-591026. Sequences are amplified from different strains. BCG: M.bovis BCG; H37Rv: M. tuberculosis strain H37Rv sequence from NCBI database; CDC: CDC1551; S.I: South Indian strain A2313; NINF: non-lethal North Indian strain; BS: Beijing strain; NIF: Lethal North Indian strain. The gene coding for oxidoreductase activity is a virulence gene which does not show any differences between the M.tuberculosis strains, but has a conservative polymorphism with M.bovis BCG.

| | |
|---|---|
| | 130 140 150 160 170 180 190 200 210 |
| | +--------+---------+---------+---------+---------+---------+---------+---------+ |
| BCG | CCAGGCCTCGATCGACGATCTGGCGTCTCTCGAAGAAGACTTTACCGTTGCACGTCGCCGTCTACCGGCGGGTGATTGCGG |
| H37Rv | +++++++++++++++++++++++++++++++++++++++++++-+++++++++++++++++++++++++++++++++++++ |
| NINF | +++++++++++++++++++++++++++++++++++++++++++-+++++++++++++++++++++++++++++++++++++ |
| BS | +++++++++++++++++++++++++++++++++++++++++++-+++++++++++++++++++++++++++++++++++++ |
| CDC1551 | +++++++++++++++++++++++++++++++++++++++++++-+++++++++++++++++++++++++++++++++++++ |
| SI | +++++++++++++++++++++++++++++++++++++++++++-+++++++++++++++++++++++++++++++++++++ |
| NIF | +++++++++++++++++++++++++++++++++++++++++++-+++++++++++++++++++++++++++++++++++++ |

Sequencing of the region from 11-138548 to 11-139067. Sequences are amplified from different strains. BCG: M.bovis BCG; H37Rv: M.tuberculosis strain H37Rv sequence from NCBI database; CDC: CDC1551; S.I: South Indian strain A2313; BS: Beijing strain; NINF: non-lethal North Indian strain; NIF: Lethal North Indian strain The insertion in BCG leads to a shorter protein with a different carboxyl terminal compared to the transcription factor encoded by the tuberculosis strains.

| | |
|---|---|
| | 10 20 30 40 50 60 70 |
| | +---------+---------+---------+---------+---------+-----+--- |
| BCG | GTGGCGAGCCGGCAAACCCCTGCTGAGCTGGCCAGATGCGACTTGGCTAAGACCGCGGAGCGCG |
| CDC1551 | +++++++++++++++A++++++++++++++++++++++++++++++++++++++++++++++++ |
| H37Rv | +++++++++++++++A++++++++++++++++++++++++++++++++++++++++++++++++ |
| BS | +++++++++++++++-++++++++++++++++++++++++++++++++++++++++++++++++ |
| NTNF | +++++++++++++++-++++++++++++++++++++++++++++++++++++++++++++++++ |
| SI | +++++++++++++++-++++++++++++++++++++++++++++++++++++++++++++++++ |

| | |
|---|---|
| | 80 90 100 110 120 130 |
| | ------+---------+---------+---------+---------+---------\| |
| BCG | AGCACACCCCGACGGCGACTGCGACAACTCCAAGCGTGGCCGGTAACGTGATGCCCA |
| H37Rv | +++++++++++++++++++++++++++++++++++++++++++++++++++++++++ |
| BS | +++++++++++++++++++++++++++++++++++++++++++++++++++++++++ |
| NTNF | +++++++++++++++++++++++++++++++++++++++++++++++++++++++++ |
| SI | +++++++++++++++++++++++++++++++++++++++++++++++++++++++++ |

| | |
|---|---|
| | 131 140 150 160 170 180 190 |
| | \|--------+---------+---------+---------+---------+---------+--- |
| BCG | TGATTGTGCGTTCCCTTCCCGCTGCGTTGCGCGCGTGTGCGCGTCTGCAACCCCATGACCCGG |
| CDC1551 | +++G+++++++++++++++++++++++++++++++++++++++++++++++++++++++++++ |
| H37Rv | +++G+++++++++++++++++++++++++++++++++++++++++++++++++++++++++++ |
| BS | +++G+++++++++++++++++++++++++++++++++++++++++++++++++++++++++++ |
| NTNF | +++G+++++++++++++++++++++++++++++++++++++++++++++++++++++++++++ |
| SI | +++G+++++++++++++++++++++++++++++++++++++++++++++++++++++++++++ |

| | |
|---|---|
| | 200 210 220 230 240 250 |
| | ------+---------+---------+---------+---------+---------+ |
| BCG | CCTTCACGTTTATGGATTACGAACAGGACTGGGACGGCGTTGCGATAACCCTGACGT |
| CDC1551 | +++++++++++++++++++++++++++++++++++++++++++++++++++++++++ |
| H37Rv | +++++++++++++++++++++++++++++++++++++++++++++++++++++++++ |
| BS | +++++++++++++++++++++++++++++++++++++++++++++++++++++++++ |
| NTNF | +++++++++++++++++++++++++++++++++++++++++++++++++++++++++ |
| SI | +++++++++++++++++++++++++++++++++++++++++++++++++++++++++ |

| | |
|---|---|
| | 251 260 270 280 290 300 310 |
| | \|---------\|---------+--------- +---------+---------+---------+-- |
| BCG | GGTCGCAGCTGTATCGGCGAACGCTGAATGTGGCACGGGAGCTGAGCCGTTGTGGTTCCAGGT |
| CDC1551 | +++++++++++++++++++++++++++++++++++++++++++++++++++++++++++C+G |
| H37Rv | +++++++++++++++++++++++++++++++++++++++++++++++++++++++++++C+G |
| BS | +++++++++++++++++++++++++++++++++++++++++++++++++++++++++++C+G |
| NTNF | +++++++++++++++++++++++++++++++++++++++++++++++++++++++++++C+G |
| SI | +++++++++++++++++++++++++++++++++++++++++++++++++++++++++++C+G |

| | |
|---|---|
| | 320 330 340 350 360 370 |
| | -------+---------+---------+---------+---------+---------+ |
| BCG | CGCAGCTGTATCGGCGAACGCTGAATG--TGGCACGGGAGCTGAGCCGTTGTGGTTC |
| CDC1551 | -+TGA+C+CG+-++T++T+T+++CTCCGCA++G++TC+++-+AC+T+++C+CCT+++ |
| H37Rv | -+TGA+C+CG+-++T++T+T+++CTCCGCA++G++TC+++-+AC+T+++C+CCT+++ |
| BS | -+TGA+C+CG+-++T++T+T+++CTCCGCA++G++TC+++-+AC+T+++C+CCT+++ |
| NTNF | -+TGA+C+CG+-++T++T+T+++CTCCGCA++G++TC+++-+AC+T+++C+CCT+++ |
| SI | -+TGA+C+CG+-++T++T+T+++CTCCGCA++G++TC+++-+AC+T+++C+CCT+++ |

Sequencing of the region from H-3283171 to H-3283585. Two SNPs, one indel and a long polymorphism characterize this region. Sequences are amplified from different strains. BCG: M.bovis BCG; H37Rv: M.tuberculosis strain H37Rv sequence from NCBI database; CDC: CDC1551; S.I South Indian strain A2313; BS: Beijing strain; NIF: non-lethal North Indian strain. All the polymorphisms occur in the fadD28, a virulence gene involved in fatty acid synthesis. They result in a non-conservative substitution and probably have an important role in the degree of virulence imparted to the strain.

| | 130 140 150 160 170 180 190 200 210 |
|---|---|
| | +---------+---------+---------+---------+---------+---------+---------+---------+ |
| BCG | TTGGCCCACGTGCTGAACTTGGTGACGTTGGCTGCGGTGACAAACAAGTTCTGATAGGTCGTTGCGCCCGTCGGCCCGAAG |
| H37Rv | +++++++++++++++++++++++++++++++++++++++++++++++C+++++++++++++++++++++++++++++++++ |
| CDC1551 | +++++++++++++++++++++++++++++++++++++++++++++++C+++++++++++++++++++++++++++++++++ |
| NINF | +++++++++++++++++++++++++++++++++++++++++++++++A+++++++++++++++++++++++++++++++++ |
| SI | +++++++++++++++++++++++++++++++++++++++++++++++A+++++++++++++++++++++++++++++++++ |
| BS | +++++++++++++++++++++++++++++++++++++++++++++++A+++++++++++++++++++++++++++++++++ |
| | 211 230 240 250 260 270 280 290 |
| | ---------+---------+---------+---------+---------+---------+---------+--------- |
| BCG | ATGAGTTGGCCCATGAGTTGGGTGTATTGGGTGCTGAGTGTGGCCAGGCCCTGCAGCAGGGTCGGGATGATGTCGAACG |
| H37Rv | +++++++++++++++++++++++++++++++++++++++++++++++++++++++++++++++++++++++++++++++++ |
| CDC1551 | +++++++++++++++++++++++++++++++++++++++++++++++++++++++++++++++++++++++++++++++++ |
| NINF | +++++++++++++++++++++++++++++++++++++++++++++++++++++++++++++++++++++++++++++++++ |
| SI | +++++++++++++++++++++++++++++++++++++++++++++++++++++++++++++++++++++++++++++++++ |
| BS | +++++++++++++++++++++++++++++++++++++++++++++++++++++++++++++++++++++++++++++++++ |
| | 300 310 320 330 340 350 360 370 380 |
| | +---------+---------+---------+---------+---------+---------+---------+---------+ |
| BCG | GAAACTGCGCCGCTGCACTCGAAAGCGCGGTTGTCACCGCATTGGTGCCGCTCGCTAGGGCGGTCGCTTSCCCCGTTGCGG |
| H37Rv | +++++++++++++++++++++++++++++++++++++++++++++++++++++++++++++++++++++G+++++++++++ |
| CDC1551 | +++++++++++++++++++++++++++++++++++++++++++++++++++++++++++++++++++++G+++++++++++ |
| NINF | +++++++++++++++++++++++++++++++++++++++++++++++++++++++++++++++++++++G+++++++++++ |
| SI | +++++++++++++++++++++++++++++++++++++++++++++++++++++++++++++++++++++G+++++++++++ |
| BS | +++++++++++++++++++++++++++++++++++++++++++++++++++++++++++++++++++++G+++++++++++ |

Sequencing of the region from H-2051784 to H-2052209. This region is characterized by a SNP between M.bovis BCG and the tuberculosis strains and a second SNP common to the Asian strains and to BCG, but different from H37Rv and CDC1551. Sequences are amplified from different strains. BCG: M.bovis BCG; H37Rv: M.tuberculosis strain H37Rv sequence from NCBI database; CDC: CDC1551; S.I: South Indian strain A2313; BS: Beijing strain; NINF: non-lethal North Indian strain. The SNP common to all the tuberculosis strains results in a conservative substitution in the PPE33b gene and does not affect the function of this gene. However the A to G substitution results in the truncation of the protein encoded by BCG.

150 160 170 180 190 200 210 220 230 240
+---------+---------+---------+---------+---------+---------+---------+---------+---------+

| | |
|---|---|
| BCG | CATCGTCGCCGGCGCGGGTCACTGGCGCCGCTCCTCCCCATCGCTTTGCTCTGCATCGTCGCCGGCGCGGGTCACTGGCGCCGCTCCTCCC |
| H37Rv | +++++++++++++++++++++---------------------------------------------------------------------- |
| CDC1551 | +++++++++++++++++++++---------------------------------------------------------------------- |
| SI | +++++++++++++++++++++CTGGCGCCGCTCCTCCCCATCGCTTTGCTCTGCATCGTCGCCGGCGCGGGTCACTGGCGCCGCTCCTCCC |
| BS | +++++++++++++++++++++CTGGCGCCGCTCCTCCCCATCGCTTTGCTCTGCATCGTCGCCGGCGCGGGTCACTGGCGCCGCTCCTCCC |
| NINF | +++++++++++++++++++++CTGGCGCCGCTCCTCCCCATCGCTTTGCTCTGCATCGTCGCCGGCGCGGGTCACTGGCGCCGCTCCTCCC |

241 250 260 270 280 290 300
+---------+---------+---------+---------+---------+---------+

| | |
|---|---|
| BCG | CATCGCTTTGCTCTCTGCATCGTCGCCGGCGCGGGTCAATCGAAGATGCCCCGTCGCGTGTC |
| H37Rv | ------------------------------------++++++++++++++++++A+++++ |
| CDC1551 | ------------------------------------++++++++++++++++++H+++++ |
| SI | CATCGCTTTGCTCTGCATCGTCGCCGGCGCGGGTCA++++++++++++++++++A+++++ |
| BS | CATCGCTTTGCTCTGCATCGTCGCCGGCGCGGGTCA++++++++++++++++++A+++++ |
| NINF | CATCGCTTTGCTCTGCATCGTCGCCGGCGCGGGTCA++++++++++++++++++A+++++ |

Sequencing of the region from H-3006917 to H-3007246. Sequences are amplified from different strains. BCG: M.bovis BCG; H37Rv: M.tuberculosis strain H37Rv sequence from NCBI database; CDC: CDC1551; S.I: South Indian strain A2313; BS: Beijing strain; M18: non-lethal North Indian strain. This region encloses a long polymorphism of 106bp inserted into a gene encoding an integral membrane protein in BCG and the Asian strains. This results in a longer integral membrane product in these strains as compared to H37Rv and CDC1551. The SNP also results in the introduction of a stop codon in H37Rv and CDC1551 further reducing the length of the membrane protein encoded by the latter.

| | 40 50 60 70 80 90 100 110 120 |
|---|---|
| | +---------+---------+---------+---------+---------+---------+---------+---------+ |
| BCG | CTGGGTCAGCAGCGGGTGTGCGCTGATTTCGATGAAGGTGTGGTAGGCGCCGTCGGCGCCGCTACCGGCGGAAGCGATGGC |
| BS | +++++++++++++++++++++++++++++++++++++++++++++++++++++++++++++++++++++++++++++++++ |
| NINF | +++++++++++++++++++++++++++++++++++++++++++++++++++++++++++++++++++++++++++++++++ |
| SI | +++++++++++++++++++++++++++++++++++++++++++++++++++++++++++++++++++++++++++++++++ |
| H37Rv | +++++++++++++++++++++++++++++++++++++++++++++++++++++++++++++++++++++++++++++++++ |
| CDC1551 | +++++++++++++++++++++++++++C+++++++++++++++++++++++++++++++++++++++++++++++++++++ |
| | 121 130 140 150 160 170 180 190 200 |
| | ---------+---------+---------+---------+---------+---------+---------+---------+ |
| BCG | CTGGCTGGAAATGCACGGGGTTGCGCATGTTGGTGGCCCAGTGTTCGGCGTCGAAGACCGGTTGGGTGTGCAAGTCTGCGT |
| BS | +++++++++++++++++++++++++++++++++++++++++++++++++++++++++++++++++++++++++++++++++ |
| NINF | +++++++++++++++++++++++++++++++++++++++++++++++++++++++++++++++++++++++++++++++++ |
| SI | +++++++++++++++++++++++++++++++++++++++++++++++++++++++++++++++++++++++++++++++++ |
| H37Rv | +++++++++GC++++++++++++++++++++++++++++++++++++++++++++++++++++++++++++++++++++++ |
| CDC1551 | +++++++++++++++++++++++++++++++++++++++++++++++++++++++++++++++++++++++++++++++++ |
| | 201 210 220 230 240 250 260 270 280 |
| | ---------+---------+---------+---------+---------+---------+---------+---------+ |
| BCG | AGGTGGTGGAGATGATTCCGATGGTGGGGGTCCGTGGGGTCAGATCGGCCAGCTCCGAACGCATCGCCGGCTGCAAAGCA |
| BS | +++++++++++++++++++++++++++++++++++++++++++++++++++++++++++++++++++++++++++++++++ |
| NINF | +++++++++++++++++++++++++++++++++++++++++++++++++++++++++++++++++++++++++++++++++ |
| SI | +++++++++++++++++++++++++++++++++++++++++++++++++++++++++++++++++++++++++++++++++ |
| H37Rv | +++++++++++++++++++++++++++++++++++++++++++++++++++++++++++++++++++++++++++++++++ |
| CDC1551 | +++++++++++++++++++++++++++++++++++++++++++++++++++++++++++++++++++++++++++++++++ |
| | 281 281 300 310 320 330 340 350 360 |
| | ---------+---------+---------+---------+---------+---------+---------+---------+ |
| BCG | TCCATGGCCGGATTGTGCGGGGCCACTTCGATATTGACCCGGCTGGCGAATCGGTCCCTAGCGCGCACGCGAGTGATCAA |
| BS | +++++++++++++++++++++++++++++++++++++++++++++++++++++++++++++++++++++++++++++++++ |
| NINF | +++++++++++++++++++++++++++++++++++++++++++++++++++++++++++++++++++++++++++++++++ |
| SI | +++++++++++++++++++++++++++++++++++++++++++++++++++++++++++++++++++++++++++++++++ |
| H37Rv | +++++++++++++++++++++++++++++++++++++++++++++++++A+++++A++C+++TTTGC++++++++C++G+C++++++ |
| CDC1551 | +++++++++++++++++++++++++++++++++++++++++++++++++A+++++A++C+++TTTGC++++++++C++G+C++++++ |

Sequencing of the region from H-3247737 to H-3248224 Sequences are amplified from different strains. BCG: M.bovis BCG; H37Rv: M.tuberculosis strain H37Rv sequence from NCBI database; CDC: CDC1551; S.I: South Indian strain A2313; BS: Beijing strain; NINF: non-lethal North Indian strain. All the polymorphisms observed occur in ppsA—the polyketide synthase gene and are synonymous substitutions. All the three Asian strains show identity to BCG in this region.

| | 100 110 120 130 140 150 160 |
|---|---|
| | +---------+---------+---------+---------+---------+---------+----- |
| BCG | CGCGGTACACGTGTCGAACGGCGACAAACCCAAGGTTGCCTTGCCCGATACTCAGTTGGGTTCACA |
| H37Rv | ++++++++++++++++++++++++++++++++++++++++++++++++++++++++++++++++++ |
| BS | ++++++++++++++++++++++++++++++++++++++++++++++++++++++++++++++++++ |
| SI | ++++++++++++++++++++++++++++++++++++++++++++++++++++++++++++++++++ |
| NINF | ++++++++++++++++++++++++++++++++++++++++++++++++++++++++++++++++++ |
| CDC1551 | ++++++++++++++++++++++++++++++++++++++++++++++++++++++++++++++++++ |
| NIF | ++++++++++++++++++++++++++++++++++++++++++++++++++++++++++++++++++ |
| | 170 180 190 200 210 220 230 |
| | ----+---------+---------+---------+---------+---------+---------+ |
| BCG | CTCAACGTGATTCGAAATCCACACTGATACTGGAGGTGATTACCGGCTGAAGCAAAGCGCATTGG |
| H37Rv | ++G++++++++++++++++++++++++++++++++++++++++++++++++++++++++++++++ |
| BS | ++G++++++++++++++++++++++++++++++++++++++++++++++++++++++++++++++ |
| SI | ++G++++++++++++++++++++++++++++++++++++++++++++++++++++++++++++++ |
| NINF | ++G++++++++++++++++++++++++++++++++++++++++++++++++++++++++++++++ |
| CDC1551 | ++G++++++++++++++++++++++++++++++++++++++++++++++++++++++++++++++ |
| NIF | ++G++++++++++++++++++++++++++++++++++++++++++++++++++++++++++++++ |

Sequencing of the region from H-2052524 to H-2052863. Sequences are amplified from different strains. BCG: M.bovis BCG; H37Rv: M.tuberculosis strain H37Rv sequence from NCBI database; CDC: CDC1551; S.I: South Indian strain A2313; BS: Beijing strain; N1NF: non-lethal North Indian strain; NIF: Lethal North Indian strain .A single nucleotide polymorphism occurring in the proton transport gene PPF,33b results in the introduction of a stop codon and hence truncation of the protein in BCG.

| | |
|---|---|
| | 190 200 210 220 230 240 |
| | +---------+---------+---------+---------+---------+--------- |
| BCG | CATCGGCCGAAACGTGAGTAATCTGGGCGGCC---------------------------- |
| CDC1551 | ++++++++++++++++++++++++++++++++CGCTCAGCGCCCAGGGCATCGAAGAACA |
| H37Rv | ++++++++++++++++++++++++++++++++CGCTCAGCGCCCAGGGCATCGAAGAACA |
| BS | ++++++++++++++++++++++++++++++++CGCTCAGCGCCCAGGGCATCGAAGAACA |
| NINF | ++++++++++++++++++++++++++++++++CGCTCAGCGCCCAGGGCATCGAAGAACA |
| SI | ++++++++++++++++++++++++++++++++CGCTCAGCGCCCAGGGCATCGAAGAACA |

| | |
|---|---|
| | 250 260 270 280 290 |
| | +---------+---------+---------+---------+ |
| BCG | -------------------GTGGCTCGGGGCGGCCCACACC |
| CDC1551 | AGCCCAGGGTGGCCTTGTC+++++C++++++++++++++++ |
| H37Rv | AGCCCAGGGTGGCCTTGTC+++++C++++++++++++++++ |
| BS | AGCCCAGGGTGGCCTTGTC+++++C++++++++++++++++ |
| NINF | AGCCCAGGGTGGCCTTGTC+++++C++++++++++++++++ |
| SI | AGCCCAGGGTGGCCTTGTC+++++C++++++++++++++++ |

Sequencing of the region from H-1468644 to H-1469150. Sequences are amplified from different strains. BCG: M.bovis BCG; H37Rv: M.tuberculosis strain H37Rv sequence from NCBI database; CDC: CDC1551;S.I: South Indian strain A2313; BS: Beijing strain; NINF: non-lethal North Indian strain. An insertion of 47bp is seen in all the tuberculosis strains in Mbl346c, a gene with DNA binding activity. A second polymorphism (SNP) is also seen immediately adjacent to the insertion in the same gene. The SNP results in splitting the gene into two genes while there is a single long gene in the M.tuberculosis strains.

| | 190 200 210 220 230 240 |
|---|---|
| | +---------+---------+---------+---------+---------+---- |
| BCG | TGTTGGCTTCATCAGCACCCCGAGGTGTGTATTCAGGCGATCCGGGGCAGCG |
| CDC1551 | ++++++++++++++++++++++++++++--++++++++++++++++C+++T++++ |
| H37Rv | ++++++++++++++++++++++++++++--++++++++++++++++C+++T++++ |
| NINF | ++++++++++++++++++++++++++++--++++++++++++++++C+++T++++ |
| SI | ++++++++++++++++++++++++++++--++++++++++++++++C+++T++++ |
| BS | ++++++++++++++++++++++++++++--++++++++++++++++C+++T++++ |

| | 250 260 270 280 290 |
|---|---|
| | -----+---------+---------+---------+---------+ |
| BCG | GGGTCGGGGTGACGCGGTTCCGCCCAAAGGTCC--GTCACCCTGTG |
| CDC1551 | +++++++++++++++++++++++++++++++++AC+++++++++++ |
| H37Rv | +++++++++++++++++++++++++++++++++AC+++++++++++ |
| NINF | +++++++++++++++++++++++++++++++++AC+++++++++++ |
| SI | +++++++++++++++++++++++++++++++++AC+++++++++++ |
| BS | +++++++++++++++++++++++++++++++++AC+++++++++++ |

Sequencing of the region from H-455094 to H-455468. Sequences are amplified from different strains. BCG: M.bovis BCG; H37Rv: M.tuberculosis strain H37Rv sequence from NCBI database; CDC: CDC1551; S.I: South Indian strain A2313; BS: Beijing strain; NINF: non-lethal North Indian strain. The region is characterized by the occurrence of two indels and two SNPs in a transcription regulator. All the tuberculosis strains appear to be identical in this region while BCG, has a different amino-acid sequence in the region.

| | |
|---|---|
| | 60 70 80 90 100 110 120 |
| | +---------+---------+---------+---------+---------+---------+ |
| BCG | CAGATCGGCTCGGTCCGCTTCGCGATTTACCGCTCGGACTATGTGCAGTCGGTGACGGCTC |
| CDC1551 | ++++++++++++++++++++++++++++++++T++++++++++++++++++++++++++++ |
| H37Rv | ++++++++++++++++++++++++++++++++T++++++++++++++++++++++++++++ |
| BS | ++++++++++++++++++++++++++++++++T++++++++++++++++++++++++++++ |
| NTNF | ++++++++++++++++++++++++++++++++T++++++++++++++++++++++++++++ |
| SI | ++++++++++++++++++++++++++++++++T++++++++++++++++++++++++++++ |
| NIF | ++++++++++++++++++++++++++++++++T++++++++++++++++++++++++++++ |

| | |
|---|---|
| | 130 140 150 160 |
| | ---------+---------+---------+---------\| |
| BCG | ++++++++++++++++++++++++++++++A+++++++++ |
| CDC1551 | ++++++++++++++++++++++++++++++A+++++++++ |
| H37Rv | ++++++++++++++++++++++++++++++A+++++++++ |
| BS | ++++++++++++++++++++++++++++++A+++++++++ |
| NTNF | ++++++++++++++++++++++++++++++A+++++++++ |
| SI | ++++++++++++++++++++++++++++++A+++++++++ |
| NIF | ++++++++++++++++++++++++++++++A+++++++++ |

Sequencing of the region from H-466229 to H-466536. Sequences are amplified from different strains. BCG: M.bovis BCG; H37Rv: M. tuberculosis strain H37Rv sequence from NCBI database; CDC: CDC1551; S.I: South Indian strain A2313; BS: Beijing strain; NINF: non-lethal North Indian strain; NIF: Lethal North Indian strain .The C to T transition occurs in a gene of unknown function and results in a synonymous substitution. However, the C to A change occurs in a transcription factor (Mb0393) and is a non-conservative substitution resulting in a slightly different protein in BCG.

| | 130 140 150 160 170 180 190 200 |
|---|---|
| | +---------+---------+---------+---------+---------+---------+---------+ |
| BCG | CCGCCAGGGTTACACCGACGTCGACCAGTTCACACTCGAAAAGTAACCGGACAAAGCGCGCTGGCTACCCA |
| CDC1551 | ++++++++++++++++++++++++++++++++++++G++++++++++++++++++++++++++++++++++ |
| H37Rv | ++++++++++++++++++++++++++++++++++++G++++++++++++++++++++++++++++++++++ |
| NIF | ++++++++++++++++++++++++++++++++++++G++++++++++++++++++++++++++++++++++ |
| NINF | ++++++++++++++++++++++++++++++++++++G++++++++++++++++++++++++++++++++++ |
| BS | ++++++++++++++++++++++++++++++++++++G++++++++++++++++++++++++++++++++++ |
| SI | ++++++++++++++++++++++++++++++++++++G++++++++++++++++++++++++++++++++++ |

Sequencing of the region from H-560625 to H-561248. Sequences are amplified from different strains. BCG: M.bovis BCG; H37Rv: M.tuberculosis strain H37Rv sequence from NCBI database; CDC: CDC1551; S.I: South Indian strain A2313; BS: Beijing strain; NINF: non-lethal North Indian strain; NIF: Lethal North Indian strain. A synonymous SNP occurs in a virulence gene and is identical in all the tuberculosis strains.

| | 150 160 170 180 190 200 |
|---|---|
| | --+---------+---------+---------+---------+---------+ |
| BCG | GGCCCACGATTTGCAATGGTGACGAGTTGGCTGCCTCGGCGCTGGCGTACTAG |
| H37Rv | +++++++++++++++++++++++++++++++++++++++++++++++++++G+ |
| CDC1551 | +++++++++++++++++++++++++++++++++++++++++++++++++++G+ |
| BS | +++++++++++++++++++++++++++++++++++++++++++++++++++G+ |
| SI | +++++++++++++++++++++++++++++++++++++++++++++++++++G+ |
| NINF | +++++++++++++++++++++++++++++++++++++++++++++++++++G+ |
| NIF | +++++++++++++++++++++++++++++++++++++++++++++++++++G+ |

| | 210 220 230 240 250 |
|---|---|
| | ---------+---------+---------+---------+---------+ |
| BCG | GCCGCCCCCGCGCTCATGAGCTGGACGAACTGCTCATGGAATGCGACCGC |
| H37Rv | +++++++++++++++++++++++++++++++++++++++++++++++++ |
| CDC1551 | +++++++++++++++++++++++++++++++++++++++++++++++++ |
| BS | +++++++++++++++++++++++++++++++++++++++++++++++++ |
| SI | +++++++++++++++++++++++++++++++++++++++++++++++++ |
| NINF | +++++++++++++++++++++++++++++++++++++++++++++++++ |
| NIF | +++++++++++++++++++++++++++++++++++++++++++++++++ |

Sequencing of the region from H-2046394 to H-2046928. Sequences are amplified from different strains. BCG: M.bovis BCG; H37Rv: M.tuberculosis strain H37Rv sequence from NCBI database; CDC: CDC1551; S.I: South Indian strain A2313; BS: Beijing strain; NINF: non-lethal North Indian strain; NIF: Lethal North Indian strain. The SNP in BCG results in splitting the gene PE-PGRS32 into two parts with the latter being truncated.

| | 40 50 60 70 80 90 |
|---|---|
| | +---------+---------+---------+---------+---------+----- |
| BCG | ACGATCATCGGTGGTGGTGGAGCCGGTATGGTAGCTACCGCCACGCGGAAGCTGGT |
| CDC1551 | ++++++++++++++++++++++++A+++++++++++++++++++++++++++++ |
| H37Rv | ++++++++++++++++++++++++A+++++++++++++++++++++++++++++ |
| NINF | ++++++++++++++++++++++++A+++++++++++++++++++++++++++++ |
| SI | ++++++++++++++++++++++++A+++++++++++++++++++++++++++++ |
| BS | ++++++++++++++++++++++++A+++++++++++++++++++++++++++++ |
| NIF | ++++++++++++++++++++++++A+++++++++++++++++++++++++++++ |

| | 100 110 120 130 140 150 |
|---|---|
| | ----+---------+---------+---------+---------+---------+ |
| BCG | CGGCGGGCGCTTCATGGCGATGACGACCGGACCGGACAGGTCTATGCCGGACGCG |
| CDC1551 | ++++++++++++++++++++++++++++++++++++++++++++++++++++++ |
| H37Rv | ++++++++++++++++++++++++++++++++++++++++++++++++++++++ |
| NINF | ++++++++++++++++++++++++++++++++++++++++++++++++++++++ |
| SI | ++++++++++++++++++++++++++++++++++++++++++++++++++++++ |
| BS | ++++++++++++++++++++++++++++++++++++++++++++++++++++++ |
| NIF | ++++++++++++++++++++++++++++++++++++++++++++++++++++++ |

| | 151 160 170 180 190 200 |
|---|---|
| | +--------+--------+---------+---------+---------+------ |
| BCG | GCGACCGCGGCCACCGGGGTGATAACGGCGTGCACCGGCGCGGTTCTCCCGGGGAA |
| CDC1551 | +++++++++++++++++++++++++++++++++++++++++++++++++++++++ |
| H37Rv | +++++++++++++++++++++++++++++++++++++++++++++++++++++++ |
| NINF | +++++++++++++++++++++++++++++++++++++++++++++++++++++++ |
| SI | +++++++++++++++++++++++++++++++++++++++++++++++++++++++ |
| BS | +++++++++++++++++++++++++++++++++++++++++++++++++++++++ |
| NIF | +++++++++++++++++++++++++++++++++++++++++++++++++++++++ |

| | 210 220 230 240 250 260 |
|---|---|
| | ---+---------+---------+---------+---------+---------+ |
| BCG | TACCGGAGCCGCGCCGCCGACCGCACTGGCGAATACCAACGGGGCAATCGCTGC |
| CDC1551 | ++++++++++++++C++++++++++++++++++++++++++++++++++++++ |
| H37Rv | ++++++++++++++T++++++++++++++++++++++++++++++++++++++ |
| NINF | ++++++++++++++C++++++++++++++++++++++++++++++++++++++ |
| SI | ++++++++++++++C++++++++++++++++++++++++++++++++++++++ |
| BS | ++++++++++++++C++++++++++++++++++++++++++++++++++++++ |
| NIF | ++++++++++++++C++++++++++++++++++++++++++++++++++++++ |

Sequencing of the region from 11-1373629 to 11-1374101. Sequences are amplified from different strains. BCG: M.bovis BCG; H37Rv: M. tuberculosis strain H37Rv sequence from NCBI database; CDC: CDC1551; S.I: South Indian strain A2313; BS: Beijing strain; NINF: non-lethal North Indian strain; NIF: Lethal North Indian strain. The two polymorphisms observed occur in a transcription factor and result in non-conservative substitutions.

| | 220 230 240 250 260 270 280 |
|---|---|
| | +---------+---------+---------+---------+---------+---------+ |
| BCG | TCTCTCGGTCATTCGTGGTCGCAGGCGCCGCACTCGGTGTCTTCGGGGGGGGGGGGGGGGG |
| H37Rv | ++++++++++++++++++++++++++++++++++++++++++++++T++++--------- |
| CDC1551 | ++++++++++++++++++++++++++++++++++++++++++++++T++++--------- |
| SI | ++++++++++++++++++++++++++++++++++++++++++++++T++++--------- |
| BS | ++++++++++++++++++++++++++++++++++++++++++++++T++++--------- |
| NINF | ++++++++++++++++++++++++++++++++++++++++++++++T++++--------- |
| NIF | ++++++++++++++++++++++++++++++++++++++++++++++T++++--------- |

| | 290 300 310 320 330 340 |
|---|---|
| | ---------+---------+---------+---------+---------+---------+ |
| BCG | GGGGGGGGGGGAAGCGCGACCTCGAAGGCCACTGAAACGCCTTACGGAGACGCGACGAAC |
| H37Rv | -----------++++++++++++++++++++++++++++++++++++++++++++++++ |
| CDC1551 | -----------++++++++++++++++++++++++++++++++++++++++++++++++ |
| SI | -----------++++++++++++++++++++++++++++++++++++++++++++++++ |
| BS | -----------++++++++++++++++++++++++++++++++++++++++++++++++ |
| NINF | -----------++++++++++++++++++++++++++++++++++++++++++++++++ |
| NIF | -----------++++++++++++++++++++++++++++++++++++++++++++++++ |

Sequencing of the region from H-1622821 to H-1623282. Sequences are amplified from different strains. BCG: M.bovis BCG; H37Rv: M.tuberculosis strain H37Rv sequence from NCBI database; CDC: CDC1551; S.I: South Indian strain A2313; BS: Beijing strain; NINF: non-lethal North Indian strain; NIF:North Indian Fatal. The polymorphisms observed occur in a non-coding region outside the ORF.

| | 150 160 170 180 190 200 210 220 230 |
|---|---|
| | +---------+---------+---------+---------+---------+---------+---------+---------+ |
| BCG | TGTGGCGCGCCTGGCTCAGATAACGCAACGCCGCAGGCGCGCGCCGCACGTCAAAAGTGGTGACCGGCAACGGCCGCAGCA |
| CDC1551 | ++++++++++++++++++++++++++++++++++++++++++A++++++++++++++++++++++++++++++++++++++ |
| H37Rv | ++++++++++++++++++++++++++++++++++++++++++A++++++++++++++++++++++++++++++++++++++ |
| SI | ++++++++++++++++++++++++++++++++++++++++++A++++++++++++++++++++++++++++++++++++++ |
| BS | ++++++++++++++++++++++++++++++++++++++++++A++++++++++++++++++++++++++++++++++++++ |
| NINF | ++++++++++++++++++++++++++++++++++++++++++A++++++++++++++++++++++++++++++++++++++ |

Sequencing of the region from 11)2295752 to H-2296046. Sequences are amplified from different strains. BCG: M.bovis BCG; H37Rv: M.tuberculosis strain H37Rv sequence from NCBI database; CDC: CDC1551; S.I: South Indian strain A2313; BS: Beijing strain; NINF: non-lethal North Indian strain. The polymorphism observed occurs in the pks12 gene and results in a non-conservative substitution.

| | 30 40 50 60 70 80 90 |
|---|---|
| | +---------+---------+---------+---------+---------+---------+ |
| BCG | TGGGCCGCTCTAGATGGGCGCCGCCCCGCGCAGATGCTCGAAGATCAGGGACGTCTGGGTA |
| H37Rv | ++++++++++++++++++++++++++++++++++++++++++++++++++++++++++++ |
| CDC1551 | ++T+++++++++++++++++++++++++++++++++++++++++++++++++++++++++ |
| BS | ++++++++++++++++++++++++++++++++++++++++++++++++++++++++++++ |
| SI | ++++++++++++++++++++++++++++++++++++++++++++++++++++++++++++ |
| NINF | ++++++++++++++++++++++++++++++++++++++++++++++++++++++++++++ |

100 110 120 130 140 150
---------+---------+---------+---------+---------+---------+

| | |
|---|---|
| BCG | CCTGCGACGTCGGCGTCGGCATTGAGGTTTTCGACCACGAACGAACGCAGGTCCTCGGTG |
| H37Rv | +++++++++++++++++++++++++++++++++++++++++++++++++++++++++++ |
| CDC1551 | +++++++++++++++++++++++++++++++++++++++++++++++++++++++++++ |
| BS | +++++++++++++++++++++++++++++++++++++++++++++++++++++++++++ |
| SI | +++++++++++++++++++++++++++++++++++++++++++++++++++++++++++ |
| NINF | +++++++++++++++++++++++++++++++++++++++++++++++++++++++++++ |

| | 151 160 170 180 190 200 |
|---|---|
| | +--------+---------+---------+---------+---------+- |
| BCG | TCGCGAGCGGCGACGTGCAAGATGAAATCGTCGGCGCC------------ |
| H37Rv | ++++++++++++++++++++++++++++++++++++++GGCCAGAAAGTAG |
| CDC1551 | ++++++++++++++++++++++++++++++++++++++GGCCAGAAAGTAG |
| BS | ++++++++++++++++++++++++++++++++++++++GGCCAGAAAGTAG |
| SI | ++++++++++++++++++++++++++++++++++++++GGCCAGAAAGTAG |
| NINF | ++++++++++++++++++++++++++++++++++++++GGCCAGAAAGTAG |

| | 210 220 230 240 250 |
|---|---|
| | --------+---------+---------+---------+---------+ |
| BCG | ----------CTGCCGTTTGCGGCGGATCTGCTGGATGAAGCTGCGGA |
| H37Rv | ACATCCATCAC+++++++++++++++++++++++++++++++++++++ |
| CDC1551 | ACATCCATCAC+++++++++++++++++++++++++++++++++++++ |
| BS | ACATCCATCAC+++++++++++++++++++++++++++++++++++++ |
| SI | ACATCCATCAC+++++++++++++++++++++++++++++++++++++ |
| NINF | ACATCCATCAC+++++++++++++++++++++++++++++++++++++ |

Sequencing of the region from H-3086111 to H-3086539. Sequences are amplified from different strains. BCG: M.bovis BCG; H37Rv: M.tuberculosis strain H37Rv sequence from NCBI database; CDC: CDC1551; S.I: South Indian strain A2313; BS: Beijing strain; NINF: non-lethal North Indian strain. The SNP seen in H37Rv occurs in a non-coding region while the deletion in BCG leads to truncation of the transcription regulator.

180 190 200 210 220 230 240 250 260 270
+---------+---------+---------+---------+---------+---------+---------+---------+---------+

| | |
|---|---|
| BCG | CGGTCGCGGGCGAAGCGTTTGAAGTCCACCGTCGCCAGGCCGCTGGTCATGGCGCTGGCCTGATCCCACAGACCCCAGCCCAGGGAGATGG |
| H37Rv | +++++++++++++++++++++++++++++++++++++++++++C++++++++++++++++++++++++++++++++++++++++++++++ |
| CDC1551 | +++++++++++++++++++++++++++++++++++++++++++C++++++++++++++++++++++++++++++++++++++++++++++ |
| SI | +++++++++++++++++++++++++++++++++++++++++++C++++++++++++++++++++++++++++++++++++++++++++++ |
| NIF | +++++++++++++++++++++++++++++++++++++++++++C++++++++++++++++++++++++++++++++++++++++++++++ |
| NINF | +++++++++++++++++++++++++++++++++++++++++++C++++++++++++++++++++++++++++++++++++++++++++++ |
| BS | +++++++++++++++++++++++++++++++++++++++++++C++++++++++++++++++++++++++++++++++++++++++++++ |

Sequencing of the region from H-2295062 to H-2295633. Sequences are amplified from different strains. BCG: M.bovis BCG; H37Rv: M.tuberculosis strain H37Rv sequence from NCBI database; CDC: CDC1551; A2313: South Indian strain A2313; BS: Beijing strain; NINF: non-lethal North Indian strain; NIF:North Indian Fatal. The SNP observed occurs in the pks12 gene and results in a non-conservative substitution.

| | 80 90 100 110 120 130 140 |
|---|---|
| | -+---------+---------+---------+---------+---------+---------+ |
| BCG | CGGCGAGTACAACGACGCTCGGGTCGATGTCCCGGTCCGATGGCTGCACGGCACCG-AGATC |
| H37Rv | ++++++++++++++++++++++++++++++++++++++++++++++++++++++++G+++++ |
| CDC1551 | ++++++++++++++++++++++++++++++++++++++++++++++++++++++++G+++++ |
| BS | ++++++++++++++++++++++++++++++++++++++++++++++++++++++++G+++++ |
| SI | ++++++++++++++++++++++++++++++++++++++++++++++++++++++++G+++++ |
| NINF | ++++++++++++++++++++++++++++++++++++++++++++++++++++++++G+++++ |

| | 150 160 170 180 190 200 |
|---|---|
| | ---------+---------+---------+---------+---------+---------+ |
| BCG | CGGTGATCACGCCCGACCTGCTGGACGGCTATGCCGAGCGGGCCAGCGATTTCGAGGTGG |
| H37Rv | +++++++++++++++++++++++++++++++++++++++++++++++++++++++++++ |
| CDC1551 | +++++++++++++++++++++++++++++++++++++++++++++++++++++++++++ |
| BS | +++++++++++++++++++++++++++++++++++++++++++++++++++++++++++ |
| SI | +++++++++++++++++++++++++++++++++++++++++++++++++++++++++++ |
| NINF | +++++++++++++++++++++++++++++++++++++++++++++++++++++++++++ |

Sequencing of the region from H-162341 to H-162761. Sequences are amplified from different strains. BCG: M.bovis BCG; H37Rv: M.tuberculosis strain H37Rv sequence from NCBI database; CDC: CDC1551; S.I: South Indian strain A2313; BS: Beijing strain; NINF: non-lethal North Indian strain. The deletion in BCG occurs in the region corresponding to a gene with putative enzyme activity and results in a loss of function in BCG.

120 130 140 150 160 170 180 190 200 210
+---------+---------+---------+---------+---------+---------+---------+---------+---------+

| | |
|---|---|
| BCG | CGCCCGCGCCACGACGTCACTACGCACATTCTATTCCGGAGACCCAGGCGAGGCGTCGGGGCGGCACCGTTTGCAGGCCCGGAATCCCTCC |
| H37Rv | ++++++++++++++++++++++++++++++++C++++++++++++++++++++++++++++++++++++++++++++++++++++++++++ |
| CDC1551 | ++++++++++++++++++++++++++++++++C++++++++++++++++++++++++++++++++++++++++++++++++++++++++++ |
| BS | ++++++++++++++++++++++++++++++++C++++++++++++++++++++++++++++++++++++++++++++++++++++++++++ |
| NTNF | ++++++++++++++++++++++++++++++++C++++++++++++++++++++++++++++++++++++++++++++++++++++++++++ |
| SI | ++++++++++++++++++++++++++++++++C++++++++++++++++++++++++++++++++++++++++++++++++++++++++++ |
| NIF | ++++++++++++++++++++++++++++++++C++++++++++++++++++++++++++++++++++++++++++++++++++++++++++ |

211 220 230 240 250 260 270 280 290 300
+---------+---------+---------+---------+---------+---------+---------+---------+---------+

| | |
|---|---|
| BCG | CCCTGAGCGGCCGCCGCAGTCGGCAGGAACCGGACATTGCGCGCGAACGGTGGCCGGACGGGGCAACTCGGCCGGCAGTAGACACCGGTG |
| H37Rv | ++++++++++++++++++++++++++++++++++++++++++++++++++++++++++++++++++++++++++++++++++++++++++ |
| CDC1551 | ++++++++++++++++++++++++++++++++++++++++++++++++++++++++++++++++++++++++++++++++++++++++++ |
| BS | ++++++++++++++++++++++++++++++++++++++++++++++++++++++++++++++++++++++++++++++++++++++++++ |
| NTNF | ++++++++++++++++++++++++++++++++++++++++++++++++++++++++++++++++++++++++++++++++++++++++++ |
| SI | ++++++++++++++++++++++++++++++++++++++++++++++++++++++++++++++++++++++++++++++++++++++++++ |
| NIF | ++++++++++++++++++++++++++++++++++++++++++++++++++++++++++++++++++++++++++++++++++++++++++ |

301 310 320 330 340 350 360 370 380 390
+---------+---------+---------+---------+---------+---------+---------+---------+---------+

| | |
|---|---|
| BCG | GTCAAAACCGCGACGACGAACCAGCCGTCGAACCGGGCGTCTTTGGACTGGACCGCCCGGTAGCAGCGTTCGAAGTCGTCGTGCACCCTT |
| H37Rv | ++++++++++++++++++++++++++++++++++++++++++++++++++++T++++++++++++++++++++++++++++++++++++ |
| CDC1551 | ++++++++++++++++++++++++++++++++++++++++++++++++++++T++++++++++++++++++++++++++++++++++++ |
| BS | ++++++++++++++++++++++++++++++++++++++++++++++++++++T++++++++++++++++++++++++++++++++++++ |
| NTNF | ++++++++++++++++++++++++++++++++++++++++++++++++++++T++++++++++++++++++++++++++++++++++++ |
| SI | ++++++++++++++++++++++++++++++++++++++++++++++++++++T++++++++++++++++++++++++++++++++++++ |
| NIF | ++++++++++++++++++++++++++++++++++++++++++++++++++++T++++++++++++++++++++++++++++++++++++ |

Sequencing of -the region from H-1478664 to H-1479140. Sequences are amplified from different strains. BCG: M.bovis BCG; H37Rv: M.tuberculosis strain H37Rv sequence from NCBI database; CDC: CDC1551; S.I: South Indian strain A2313; BS: Beijing strain; NIN: non-lethal North Indian strain; NIF:North Indian Fatal. The first T to C transition results in the truncation of the bacterial regulatory protein in BCG.

| | 170 180 190 200 210 220 |
|---|---|
| | +---------+---------+---------+---------+---------+----- |
| BCG | CCACCTCGGTGGTGTTCGCCACCGCCCACTACGCGCTGGTGGATTTGGCCGACGTA |
| H37Rv | +++++++++++++++++++++++++++++++++++++++++++++++++++CT+CT |
| CDC1551 | +++++++++++++++++++++++++++++++++++++++++++++++++++CT+CT |
| NINF | +++++++++++++++++++++++++++++++++++++++++++++++++++CT+CT |
| BS | +++++++++++++++++++++++++++++++++++++++++++++++++++CT+CT |
| SI | +++++++++++++++++++++++++++++++++++++++++++++++++++CT+CT |
| NIF | +++++++++++++++++++++++++++++++++++++++++++++++++++CT+CT |

| | 230 240 250 260 270 280 |
|---|---|
| | ----+---------+---------+---------+---------+---------+ |
| BCG | CAACCGGGCCAGCGCGTGTTGATCCATGCCGGCACCGGCGGGGTGGGCATGGCGG |
| CDC1551 | AGGT++++++++++++++++++++++++++++++++++++++++++++++++++ |
| NINF | AGGT++++++++++++++++++++++++++++++++++++++++++++++++++ |
| BS | AGGT++++++++++++++++++++++++++++++++++++++++++++++++++ |
| SI | AGGT++++++++++++++++++++++++++++++++++++++++++++++++++ |
| NIF | AGGT++++++++++++++++++++++++++++++++++++++++++++++++++ |

Sequencing of the region from H-2296260 to H-2296692. Sequences are amplified from different strains. BCG: M.bovis BCG; H37Rv: M.tuberculosis strain H37Rv sequence from NCBI database; CDC: CDC1551; S.I: South Indian strain A2313; BS: Beijing strain; NINF: non-lethal North Indian strain; NIF:North Indian Fatal strain. The long polymorphism observed in the pks12 gene but does not alter the activity of the polyketide synthase enzyme. A total of 2755 polymorphisms including 1779 in ORFs and 313 in regions outside the ORF are being screened for association to virulence and/or infectivity in tuberculosis. A multicomponent analysis to determine the association of polymorphism to the degree. of virulence and infectivity is in progress. The polymorphisms which constitute a set of virulence markers are further being validated in 120 clinical isolates of tuberculosis.

The virulence factors thus identified could be used as:

i. Diagnostic markers in prediction of disease and its progress in the patient.

ii. Drug targets for development of new and effective treatments for TB.

iii. Candidate genes/sequences in DNA vaccine.

iv. In development of SiRNA technology for combating tuberculosis.

TABLE 1
List of SNP's in Mycobacterium tuberculosis/M. bovis BCG.

| | | | | | | | | | | De- | | | |
|---|---|---|---|---|---|---|---|---|---|---|---|---|---|
| | | | | | | | | | | scrip- | | | |
| Poly- | | | | | | | | | | tion | | | |
| mor- | BCG | | | H37Rv | | | CDC | | | of | | | |
| phism | SNP | | | SNP | | | SNP | | | SNP | SNP | | |
| ID | Position | Base | AA | Position | Base | AA | Position | Base | AA | ORF | type | GO ID | Putative Function |
| 1 | 467 | G | R | 467 | A | H | 467 | A | H | Yes | NS, NC | P49993 | nucleotide binding |
| | | | | | | | | | | | | | activity |
| 2 | 1057 | A | I | 1057 | G | V | 1057 | G | V | Yes | NS, C | P49993 | nucleotide binding |
| | | | | | | | | | | | | | activity |
| 3 | 2347 | G | G | 2347 | A | D | 2347 | A | D | Yes | NS, NC | Q50790 | DNA binding activity |
| 4 | 2532 | C | L | 2532 | T | L | 2532 | T | L | Yes | S, NULL | Null | — |
| 5 | 3751 | G | V | 3751 | T | L | 3751 | T | L | Yes | NS, C | Q59586 | DNA binding activity |
| 6 | 4480 | T | L | 4480 | C | S | 4480 | C | S | Yes | NS, NC | P71573 | — |
| 7 | 5752 | A | V | 5752 | G | V | 5752 | G | V | Yes | S, NULL | Null | — |
| 8 | 6406 | T | N | 6406 | C | N | 6406 | C | N | Yes | S, NULL | Null | — |
| 9 | 6446 | T | S | 6446 | G | A | 6446 | G | A | Yes | NS, NC | P41514 | nucleic acid binding |
| | | | | | | | | | | | | | activity |
| 10 | 8285 | T | I | 8285 | C | I | 8285 | C | I | Yes | S, NULL | Null | — |
| 11 | 8741 | T | R | 8741 | C | R | 8741 | C | R | Yes | S, NULL | Null | — |
| 12 | 9143 | C | I | 9143 | T | I | 9143 | T | I | Yes | S, NULL | Null | — |
| 13 | 9217 | C | A | 9217 | A | D | 9217 | A | D | Yes | NS, NC | Q07702 | DNA binding activity |
| 14 | 10727 | G | V | 10727 | A | I | 10727 | A | I | Yes | NS, C | P71575 | integral to membrane |
| 15 | 13197 | C | Null | 13197 | G | Null | 13197 | G | null | Yes | S, NULL | Null | — |
| 16 | 13459 | G | D | 13460 | A | D | 13460 | A | D | Yes | S, NULL | Null | — |
| 17 | 14400 | G | E | 14401 | A | K | 14401 | A | K | Yes | NS, NC | P71582 | integral to membrane |
| 18 | 15116 | G | M | 15117 | C | I | 15117 | C | I | Yes | NS, NC | P71583 | enzyme activity |
| 19 | 17856 | C | T | 17857 | T | T | 17857 | T | T | Yes | S, NULL | Null | — |
| 20 | 21818 | C | A | 21819 | A | S | 21819 | A | S | Yes | NS, NC | P71588 | enzyme activity |
| 21 | 22263 | G | A | 22264 | T | A | 22264 | T | A | Yes | S, NULL | Null | — |
| 22 | 23173 | A | L | 23174 | C | R | 23174 | C | null | Yes | NS, NC | P71588 | enzyme activity |
| 23 | 23713 | T | L | 23714 | C | L | 23714 | C | L | Yes | S, NULL | Null | — |
| 24 | 24293 | T | Q | 24294 | C | R | 24294 | C | R | Yes | NS, NC | P71590 | — |
| 25 | 24533 | C | C | 24534 | T | Y | 24534 | T | Y | Yes | NS, NC | P71590 | — |
| 26 | 24678 | G | H | 24679 | A | Y | 24679 | A | Y | Yes | NS, C | P71590 | — |
| 27 | 24761 | C | G | 24780 | T | D | 24762 | T | D | Yes | NS, NC | P71590 | — |
| 28 | 25287 | G | R | 25306 | C | G | 25288 | C | G | Yes | NS, NC | P71590 | — |
| 29 | 26034 | G | P | 26053 | C | A | 26035 | C | A | Yes | NS, NC | P71591 | electron transport |
| 30 | 27450 | G | Null | 27469 | A | Null | 27451 | A | null | No | nc, NULL | Null | — |
| 31 | 29442 | T | L | 29462 | C | P | 29444 | C | P | Yes | NS, NC | P71595 | — |
| 32 | 29979 | C | P | 29999 | A | Q | 29980 | A | Q | Yes | NS, NC | P71596 | — |
| 33 | 30736 | A | K | 30756 | G | K | 30737 | G | K | Yes | S, NULL | Null | — |
| 34 | 31041 | G | R | 31057 | C | R | 31038 | C | R | Yes | S, NULL | Null | — |
| 35 | 32608 | A | N | 32624 | C | H | 32568 | C | N | Yes | NS, NC | P71599 | — |
| 36 | 33788 | A | Null | 33804 | G | Null | 33748 | G | null | No | nc, NULL | Null | — |
| 37 | 36288 | G | A | 36304 | T | A | 36248 | T | A | Yes | S, NULL | Null | — |
| 38 | 36522 | C | S | 36538 | T | S | 36482 | T | S | Yes | S, NULL | Null | — |
| 39 | 36596 | G | K | 36612 | A | K | 36556 | A | K | Yes | S, NULL | Null | — |
| 40 | 39742 | G | H | 39758 | A | H | 39702 | A | H | Yes | S, NULL | Null | — |
| 41 | 41228 | A | Null | 41244 | C | Null | 41188 | C | null | No | nc, NULL | Null | — |
| 42 | 41437 | T | G | 41453 | C | G | 41397 | C | G | Yes | S, NULL | Null | — |
| 43 | 42265 | A | F | 42281 | C | C | 42225 | C | C | Yes | NS, NC | P71696 | integral to membrane |
| 44 | 43929 | G | V | 43943 | A | V | 43889 | A | V | Yes | S, NULL | Null | — |
| 45 | 45177 | A | A | 45191 | G | A | 45137 | G | A | Yes | S, NULL | Null | — |
| 46 | 49989 | A | Null | 50003 | G | Null | 49949 | G | null | No | nc, NULL | Null | — |
| 47 | 52012 | T | S | 52026 | C | G | 51972 | C | G | Yes | NS, NC | P71705 | integral to membrane |
| 48 | 53663 | T | L | 53677 | C | P | 53623 | C | P | Yes | NS, NC | P71707 | enzyme activity |
| 49 | 59861 | A | Null | 59869 | G | Null | 59815 | G | null | No | nc, NULL | Null | — |
| 50 | 62758 | G | G | 62766 | A | G | 62712 | A | G | Yes | S, NULL | Null | — |
| 51 | 63029 | T | Null | 63037 | C | Null | 62983 | C | null | No | nc, NULL | Null | — |
| 52 | 63049 | G | Null | 63057 | C | Null | 63003 | C | null | No | nc, NULL | Null | — |
| 53 | 65857 | A | I | 65865 | G | V | 65811 | G | V | Yes | NS, C | O53607 | hydrolase activity |
| 54 | 69913 | T | I | 69921 | C | T | 69867 | C | T | Yes | NS, NC | O53609 | molecular_function |
| | | | | | | | | | | | | | unknown |
| 55 | 70082 | G | P | 70090 | A | P | 70036 | A | P | Yes | S, NULL | Null | — |
| 56 | 70257 | T | F | 70265 | G | V | 70211 | G | V | Yes | NS, NC | O53609 | molecular_function |
| | | | | | | | | | | | | | unknown |
| 57 | 71758 | T | Null | 71729 | C | Null | 71712 | C | null | No | nc, NULL | Null | — |
| 58 | 74119 | T | L | 74090 | C | L | 74073 | C | L | Yes | S, NULL | Null | — |
| 59 | 74188 | G | N | 74159 | C | K | 74142 | C | K | Yes | NS, NC | O53611 | isocitrate |
| | | | | | | | | | | | | | dehydrogenase |
| | | | | | | | | | | | | | (NADP+) activity |
| 60 | 78130 | C | G | 78101 | T | E | 78084 | T | E | Yes | NS, NC | O53615 | glycine |
| | | | | | | | | | | | | | hydroxymethyltransferase |
| | | | | | | | | | | | | | activity |
| 61 | 79388 | C | Null | 79359 | G | Null | 79342 | G | null | No | nc, NULL | Null | — |
| 62 | 80169 | T | P | 80131 | C | P | 80123 | C | P | Yes | S, NULL | Null | — |
| 63 | 86899 | G | V | 86862 | A | I | 86854 | A | I | Yes | NS, C | O53623 | DNA binding activity |
| 64 | 89235 | T | V | 89198 | G | G | 89190 | G | G | Yes | NS, C | O53625 | — |
| 65 | 89570 | T | Null | 89533 | C | Null | 89525 | C | null | No | nc, NULL | Null | — |
| 66 | 90964 | T | V | 90927 | C | A | 90919 | C | A | Yes | NS, C | Q10880 | oxidative |
| | | | | | | | | | | | | | phosphorylation |
| 67 | 92357 | T | C | 92320 | A | Null | 92312 | A | null | Yes | nc, NULL | Null | — |
| 68 | 94338 | C | T | 94301 | T | M | 94293 | T | M | Yes | NS, NC | Q10883 | oxidative |
| | | | | | | | | | | | | | phosphorylation |
| 69 | 96136 | A | I | 96099 | G | V | 96091 | G | null | Yes | NS, C | Q10884 | electron transport |
| 70 | 97731 | C | Null | 97694 | T | Null | 97686 | T | null | No | nc, NULL | Null | — |
| 71 | 99336 | T | Null | 99299 | C | Null | 99291 | C | null | Yes | nc, NULL | Null | — |
| 72 | 100624 | G | A | 100587 | A | T | 100579 | A | T | Yes | NS, NC | Q10876 | magnesium ion binding |
| | | | | | | | | | | | | | activity |
| 73 | 103635 | G | R | 103598 | A | C | 103590 | A | C | Yes | NS, NC | Q10890 | integral to membrane |
| 74 | 105903 | T | W | 105865 | C | R | 105857 | C | R | Yes | NS, NC | Q10892 | integral to membrane |
| 75 | 106370 | A | P | 106332 | C | P | 106324 | C | P | Yes | S, NULL | Null | — |
| 76 | 122650 | T | W | 122612 | C | R | 122604 | C | R | Yes | NS, NC | Q10898 | cAMP-dependent |
| | | | | | | | | | | | | | protein kinase complex |
| 77 | 123556 | C | H | 123518 | T | Y | 123510 | T | Y | Yes | NS, C | Q10898 | cAMP-dependent |
| | | | | | | | | | | | | | protein kinase complex |
| 78 | 123878 | T | Null | 123840 | C | Null | 123832 | C | null | No | nc, NULL | Null | — |
| 79 | 126600 | A | S | 126561 | C | A | 126554 | C | A | Yes | NS, NC | Q10900 | magnesium ion binding |
| | | | | | | | | | | | | | activity |
| 80 | 126840 | G | P | 126801 | A | S | 126794 | A | S | Yes | NS, NC | Q10900 | magnesium ion binding |
| | | | | | | | | | | | | | activity |
| 81 | 127447 | G | L | 127408 | C | L | 127401 | C | L | Yes | S, NULL | Null | — |
| 82 | 130172 | A | V | 130133 | G | A | 130126 | G | A | Yes | NS, C | Q10900 | magnesium ion binding |
| | | | | | | | | | | | | | activity |
| 83 | 130237 | T | P | 130198 | C | P | 130191 | C | P | Yes | S, NULL | Null | — |
| 84 | 137223 | A | Q | 137183 | G | Q | 137177 | G | Q | Yes | S, NULL | Null | — |
| 85 | 138339 | A | R | 138299 | G | G | 138292 | G | G | Yes | NS, NC | O53636 | — |
| 86 | 139796 | C | Null | 139754 | T | Null | 139747 | T | null | No | nc, NULL | Null | — |
| 87 | 143247 | C | G | 143205 | T | S | 143198 | T | S | Yes | NS, NC | O53639 | DNA binding activity |
| 88 | 146006 | A | A | 145964 | G | A | 145957 | G | A | Yes | S, NULL | Null | — |
| 89 | 147495 | A | W | 147453 | C | W | 147446 | C | W | Yes | S, NULL | Null | — |
| 90 | 147911 | C | Null | 147871 | A | Null | 147864 | A | null | No | nc, NULL | Null | — |
| 91 | 149987 | G | G | 149947 | C | G | 149940 | C | G | Yes | S, NULL | Null | — |
| 92 | 159370 | A | F | 159177 | G | F | 159350 | G | F | Yes | S, NULL | Null | — |
| 93 | 160535 | T | K | 160342 | C | E | 160515 | C | E | Yes | NS, NC | P96809 | — |
| 94 | 161144 | T | F | 160951 | G | V | 161124 | G | V | Yes | NS, NC | P96810 | N-acetyltransferase |
| | | | | | | | | | | | | | activity |
| 95 | 162499 | A | R | 162306 | G | G | 162479 | G | G | Yes | NS, NC | P96811 | enzyme activity |
| 96 | 162530 | G | G | 162337 | A | D | 162510 | A | D | Yes | NS, NC | P96811 | enzyme activity |
| 97 | 165799 | G | G | 165607 | A | D | 165780 | A | D | Yes | NS, NC | P96815 | — |
| 98 | 166696 | A | H | 166504 | G | R | 166677 | G | R | Yes | NS, NC | P96816 | — |
| 99 | 170273 | G | L | 170081 | A | F | 170254 | A | F | Yes | NS, NC | P96820 | voltage-gated chloride |
| | | | | | | | | | | | | | channel activity |
| 100 | 171097 | C | R | 170905 | A | R | 171078 | A | R | Yes | S, NULL | Null | — |
| 101 | 173091 | A | R | 172899 | C | R | 173072 | C | R | Yes | S, NULL | Null | — |
| 102 | 179424 | T | R | 179232 | C | G | 179405 | C | G | Yes | NS, NC | P96828 | — |
| 103 | 181862 | C | G | 181670 | T | D | 181843 | T | D | Yes | NS, NC | P96830 | protein phosphatase |
| | | | | | | | | | | | | | activity |
| 104 | 184917 | G | C | 184725 | A | Y | 184895 | A | null | Yes | NS, NC | P96833 | — |
| 105 | 188267 | T | T | 188075 | G | P | 188245 | G | P | Yes | NS, NC | O53642 | — |
| 106 | 189999 | T | T | 189807 | C | A | 189977 | C | A | Yes | NS, NC | O86360 | — |
| 107 | 190284 | T | T | 190092 | C | A | 190262 | C | A | Yes | NS, NC | O86360 | — |
| 108 | 192177 | A | L | 191985 | G | L | 192156 | G | L | Yes | S, NULL | Null | — |
| 109 | 195552 | C | A | 195358 | T | V | 195529 | T | V | Yes | NS, C | O07411 | enzyme activity |
| 110 | 195758 | G | A | 195564 | T | S | 195735 | T | S | Yes | NS, NC | O07411 | enzyme activity |
| 111 | 198328 | A | I | 198134 | C | I | 198305 | C | I | Yes | S, NULL | Null | — |
| 112 | 199662 | G | A | 199468 | T | S | 199639 | T | S | Yes | NS, NC | P72013 | pathogenesis |
| 113 | 199800 | T | S | 199606 | C | P | 199777 | C | P | Yes | NS, NC | P72013 | pathogenesis |
| 114 | 200622 | C | T | 200428 | T | I | 200599 | T | I | Yes | NS, NC | O07414 | pathogenesis |
| 115 | 201759 | C | D | 201565 | G | E | 201736 | G | E | Yes | NS, C | O07415 | pathogenesis |
| 116 | 206673 | G | P | 206479 | C | P | 206650 | C | P | Yes | S, NULL | Null | — |
| 117 | 206676 | T | G | 206482 | G | G | 206653 | G | G | Yes | S, NULL | Null | — |
| 118 | 210634 | T | H | 210440 | C | R | 210554 | C | R | Yes | NS, NC | O07423 | — |
| 119 | 212446 | A | Null | 212252 | G | Null | 212366 | G | null | No | nc, NULL | Null | — |
| 120 | 217393 | C | N | 217199 | T | N | 217313 | T | N | Yes | S, NULL | Null | — |
| 121 | 218055 | G | R | 217861 | C | P | 217975 | C | P | Yes | NS, NC | O07430 | hydrolase activity |
| 122 | 225861 | T | S | 225666 | C | G | 225780 | C | G | Yes | NS, NC | O07437 | — |
| 123 | 227215 | G | V | 227020 | A | I | 227134 | A | I | Yes | NS, C | O53645 | nucleotide binding |
| | | | | | | | | | | | | | activity |
| 124 | 227738 | T | M | 227543 | C | T | 227657 | C | T | Yes | NS, NC | O53645 | nucleotide binding |
| | | | | | | | | | | | | | activity |
| 125 | 228053 | T | L | 227858 | C | P | 227972 | C | P | Yes | NS, NC | O53645 | nucleotide binding |
| | | | | | | | | | | | | | activity |
| 126 | 228924 | T | R | 228729 | C | R | 228843 | C | R | Yes | S, NULL | Null | — |
| 127 | 231783 | C | Null | 231587 | T | Null | 231701 | T | null | No | nc, NULL | Null | — |
| 128 | 232188 | G | A | 231992 | C | A | 232106 | C | A | Yes | S, NULL | Null | — |
| 129 | 233552 | C | V | 233356 | A | V | 233470 | A | V | Yes | S, NULL | Null | — |
| 130 | 233558 | C | S | 233362 | G | R | 233476 | G | R | Yes | NS, NC | O53648 | — |
| 131 | 243794 | G | R | 243596 | A | H | 243712 | A | H | Yes | NS, NC | O53656 | integral to membrane |
| 132 | 244589 | C | A | 244391 | T | V | 244507 | T | V | Yes | NS, C | O53656 | integral to membrane |
| 133 | 246117 | C | E | 245919 | G | D | 246035 | G | D | Yes | NS, C | O53657 | membrane |
| 134 | 246365 | T | I | 246167 | A | F | 246283 | A | F | Yes | NS, NC | O53657 | membrane |
| 135 | 249718 | C | A | 249520 | T | V | 249636 | T | V | Yes | NS, C | P96391 | — |
| 136 | 251771 | A | T | 251573 | G | A | 251689 | G | A | Yes | NS, NC | P96392 | — |
| 137 | 251865 | C | Null | 251667 | T | Null | 251783 | T | null | No | nc, NULL | Null | — |
| 138 | 256378 | C | A | 256180 | G | G | 256296 | G | G | Yes | NS, C | P96396 | enzyme activity |
| 139 | 259127 | A | Null | 258900 | C | Null | 259016 | C | null | Yes | nc, NULL | Null | — |
| 140 | 260507 | T | T | 260280 | G | P | 260396 | G | P | Yes | NS, NC | P96399 | enzyme activity |
| 141 | 262385 | A | N | 262158 | G | D | 262274 | G | D | Yes | NS, NC | P96400 | electron transport |
| 142 | 265183 | A | D | 266857 | G | D | 266973 | G | D | Yes | S, NULL | Null | — |
| 143 | 265653 | A | S | 267327 | G | P | 267443 | G | P | Yes | NS, NC | P96405 | metabolism |
| 144 | 266601 | C | L | 268275 | G | F | 268391 | G | F | Yes | NS, NC | P96406 | S-adenosylmethionine- |
| | | | | | | | | | | | | | dependent |
| | | | | | | | | | | | | | methyltransferase |
| | | | | | | | | | | | | | activity |
| 145 | 269989 | T | N | 271663 | C | S | 271779 | C | S | Yes | NS, C | P96409 | — |
| 146 | 271077 | C | R | 272751 | G | S | 272867 | G | S | Yes | NS, NC | P96409 | — |
| 147 | 271882 | G | S | 273556 | C | S | 273672 | C | S | Yes | S, NULL | Null | — |
| 148 | 273691 | T | E | 275365 | G | D | 275481 | G | D | Yes | NS, C | P96413 | zinc ion binding activity |
| 149 | 276186 | T | Null | 277860 | G | Null | 277976 | G | null | No | nc, NULL | Null | — |
| 150 | 282208 | C | A | 283882 | T | T | 283998 | T | T | Yes | NS, NC | P96419 | cell adhesion |
| 151 | 283942 | C | V | 285616 | T | M | 285732 | T | M | Yes | NS, NC | P96419 | cell adhesion |
| 152 | 285894 | T | L | 287568 | G | V | 287684 | G | V | Yes | NS, C | O53660 | hydrolase activity |
| 153 | 287276 | T | T | 288950 | C | T | 289066 | C | T | Yes | S, NULL | Null | — |
| 154 | 287759 | T | S | 289433 | G | A | 289549 | G | A | Yes | NS, NC | O53663 | — |
| 155 | 288778 | T | L | 290452 | C | L | 290568 | C | L | Yes | S, NULL | Null | — |
| 156 | 292523 | G | A | 294196 | T | E | 294313 | T | E | Yes | NS, NC | O53666 | acyl-CoA |
| | | | | | | | | | | | | | dehydrogenase activity |
| 157 | 292778 | C | R | 294451 | T | K | 294568 | T | K | Yes | NS, C | O53666 | acyl-CoA |
| | | | | | | | | | | | | | dehydrogenase activity |
| 158 | 294180 | C | Null | 295853 | A | Null | 295970 | A | null | No | nc, NULL | Null | — |
| 159 | 295519 | T | V | 297192 | C | A | 297309 | C | A | Yes | NS, C | O53668 | — |
| 160 | 300012 | T | Null | 301685 | C | Null | 301802 | C | null | No | nc, NULL | Null | — |
| 161 | 301364 | T | G | 303037 | C | G | 303154 | C | G | Yes | S, NULL | Null | — |
| 162 | 305428 | T | G | 307101 | C | G | 307218 | C | G | Yes | S, NULL | Null | — |
| 163 | 308090 | T | Null | 309763 | C | Null | 309880 | C | null | Yes | nc, NULL | Null | — |
| 164 | 311176 | A | L | 312849 | G | L | 312966 | G | L | Yes | S, NULL | Null | — |
| 165 | 312194 | A | S | 313867 | G | S | 313984 | G | S | Yes | S, NULL | Null | — |
| 166 | 318505 | G | I | 320178 | A | I | 320294 | A | null | Yes | S, NULL | Null | — |
| 167 | 321009 | T | L | 322682 | C | L | 322798 | C | L | Yes | S, NULL | Null | — |
| 168 | 321631 | G | Null | 323304 | A | Null | 323420 | A | null | No | nc, NULL | Null | — |
| 169 | 323830 | C | V | 325503 | T | V | 325619 | T | V | Yes | S, NULL | Null | — |
| 170 | 327543 | A | L | 329216 | G | P | 329332 | G | P | Yes | NS, NC | P95229 | — |
| 171 | 329913 | A | L | 331586 | G | S | 331702 | G | S | Yes | NS, NC | O53681 | — |
| 172 | 331537 | C | Null | 333210 | G | Null | 333326 | G | null | Yes | nc, NULL | Null | — |
| 173 | 331617 | G | Null | 333290 | A | Null | 333406 | A | null | Yes | nc, NULL | Null | — |
| 174 | 331719 | G | Null | 333392 | C | Null | 333508 | C | null | No | nc, NULL | Null | — |
| 175 | 340088 | G | Null | 339084 | A | Null | 339148 | A | null | No | nc, NULL | Null | — |
| 176 | 340090 | C | Null | 339086 | T | Null | 339150 | T | null | No | nc, NULL | Null | — |
| 177 | 340091 | G | Null | 339087 | A | Null | 339151 | A | null | No | nc, NULL | Null | — |
| 178 | 340092 | G | Null | 339088 | C | Null | 339152 | C | null | No | nc, NULL | Null | — |
| 179 | 340097 | C | Null | 339093 | G | Null | 339157 | G | null | No | nc, NULL | Null | — |
| 180 | 343148 | C | A | 342144 | A | E | 342208 | A | null | Yes | NS, NC | O53687 | nuoleotide binding |
| | | | | | | | | | | | | | activity |
| 181 | 344283 | C | A | 343279 | G | A | 343343 | G | A | Yes | S, NULL | Null | — |
| 182 | 351491 | C | A | 350487 | G | A | 350551 | G | A | Yes | S, NULL | Null | — |
| 183 | 355282 | A | T | 354278 | G | A | 354342 | G | A | Yes | NS, NC | O86362 | — |
| 184 | 362163 | C | Null | 361159 | T | Null | 361223 | T | null | No | nc, NULL | Null | — |
| 185 | 362818 | T | F | 361814 | C | F | 361878 | C | F | Yes | S, NULL | Null | — |
| 186 | 364560 | A | N | 363511 | G | S | 363575 | G | null | Yes | NS, C | O07226 | — |
| 187 | 364804 | T | V | 363755 | C | V | 363819 | C | V | Yes | S, NULL | Null | — |
| 188 | 366022 | T | V | 364973 | C | A | 365037 | C | A | Yes | NS, C | O07229 | DNA binding activity |
| 189 | 367778 | C | A | 366729 | T | T | 366793 | T | T | Yes | NS, NC | O07231 | tRNA ligase activity |
| 190 | 368518 | A | F | 367469 | G | S | 367533 | G | S | Yes | NS, NC | O07231 | tRNA ligase activity |
| 191 | 369200 | T | S | 368166 | C | G | 368230 | C | G | Yes | NS, NC | O07231 | tRNA ligase activity |
| 192 | 373180 | A | L | 372147 | G | L | 372211 | G | L | Yes | S, NULL | Null | — |
| 193 | 382060 | G | G | 381028 | A | S | 381091 | A | S | Yes | NS, NC | O07239 | — |
| 194 | 383273 | T | L | 382241 | C | P | 382304 | C | P | Yes | NS, NC | O07239 | — |
| 195 | 383519 | T | Null | 382487 | C | Null | 382550 | C | null | No | nc, NULL | Null | — |
| 196 | 384021 | G | A | 382989 | A | V | 383052 | A | V | Yes | NS, C | O07241 | — |
| 197 | 387090 | C | Null | 386058 | T | Null | 386120 | T | null | No | nc, NULL | Null | — |
| 198 | 390159 | G | C | 389127 | A | Y | 389189 | A | Y | Yes | NS, NC | O07247 | dCTP deaminase |
| | | | | | | | | | | | | | activity |
| 199 | 393291 | C | Q | 392259 | T | * | 392321 | T | * | Yes | NS, TP | O07250 | — |
| 200 | 393536 | G | G | 392504 | A | G | 392566 | A | G | Yes | S, NULL | Null | — |
| 201 | 394778 | A | Y | 393746 | G | H | 393808 | G | H | Yes | NS, C | O08447 | monooxygenase activity |
| 202 | 395965 | G | S | 394933 | C | W | 394995 | C | W | Yes | NS, NC | O07253 | methyltransferase |
| | | | | | | | | | | | | | activity |
| 203 | 398416 | T | Null | 397384 | C | Null | 397446 | C | null | No | nc, NULL | Null | — |
| 204 | 399064 | G | G | 398032 | A | E | 398094 | A | G | Yes | NS, NC | O07256 | — |
| 205 | 402708 | A | H | 401676 | C | P | 401738 | C | P | Yes | NS, NC | O33266 | nucleic acid binding |
| | | | | | | | | | | | | | activity |
| 206 | 406818 | A | V | 405786 | C | V | 405848 | C | V | Yes | S, NULL | Null | — |
| 207 | 406884 | G | Null | 405852 | C | Null | 405914 | C | null | No | nc, NULL | Null | — |
| 208 | 412130 | G | S | 411098 | A | N | 411160 | A | N | Yes | NS, C | O06293 | — |
| 209 | 413310 | G | Q | 412278 | T | H | 412340 | T | H | Yes | NS, NC | O06293 | — |
| 210 | 423408 | T | L | 422377 | C | S | 422439 | C | S | Yes | NS, NC | P32724 | chaperone activity |
| 211 | 423774 | C | G | 422743 | T | G | 422805 | T | G | Yes | S, NULL | Null | — |
| 212 | 425964 | A | F | 424930 | T | I | 425095 | T | I | Yes | NS, NC | O06304 | — |
| 213 | 428488 | G | G | 427469 | T | G | 427619 | T | G | Yes | S, NULL | Null | — |
| 214 | 429715 | T | A | 428696 | C | A | 428786 | C | A | Yes | S, NULL | Null | — |
| 215 | 430077 | C | D | 429058 | T | N | 429148 | T | N | Yes | NS, NC | O06304 | — |
| 216 | 438482 | C | S | 437463 | G | S | 437553 | G | S | Yes | S, NULL | Null | — |
| 217 | 439288 | A | T | 438269 | G | A | 438359 | G | A | Yes | NS, NC | O06309 | metalloendopeptidase |
| | | | | | | | | | | | | | activity |
| 218 | 441762 | C | A | 440743 | T | V | 440833 | T | V | Yes | NS, C | O06312 | cation transport |
| 219 | 443988 | C | L | 442969 | A | I | 443059 | A | I | Yes | NS, C | O06314 | molecular_function |
| | | | | | | | | | | | | | unknown |
| 220 | 444576 | A | V | 443557 | C | V | 443647 | C | V | Yes | S, NULL | Null | — |
| 221 | 446432 | T | S | 445413 | C | G | 445503 | C | G | Yes | NS, NC | O53703 | transporter activity |
| 222 | 446797 | T | H | 445778 | C | R | 445868 | C | R | Yes | NS, NC | O53703 | transporter activity |
| 223 | 448459 | T | E | 447440 | C | G | 447530 | C | G | Yes | NS, NC | O53705 | nucleotide binding |
| | | | | | | | | | | | | | activity |
| 224 | 449922 | T | S | 448903 | C | G | 448993 | C | G | Yes | NS, NC | O53707 | — |
| 225 | 451132 | T | S | 450113 | C | S | 450203 | C | S | Yes | S, NULL | Null | — |
| 226 | 452456 | G | S | 451437 | A | F | 451527 | A | F | Yes | NS, NC | O53708 | electron transport |
| 227 | 452844 | T | A | 451825 | C | A | 451915 | C | A | Yes | S, NULL | Null | — |
| 228 | 456342 | G | R | 455323 | C | P | 455414 | C | P | Yes | NS, NC | O53712 | DNA binding activity |
| 229 | 456346 | C | G | 455327 | T | G | 455418 | T | G | Yes | S, NULL | Null | — |
| 230 | 467343 | C | R | 466324 | T | R | 466415 | T | R | Yes | S, NULL | Null | — |
| 231 | 467402 | C | A | 466383 | A | D | 466474 | A | D | Yes | NS, NC | O53720 | DNA binding activity |
| 232 | 469376 | G | G | 468355 | A | E | 468448 | A | E | Yes | NS, NC | P95197 | purine base |
| | | | | | | | | | | | | | biosynthesis |
| 233 | 470348 | C | T | 469327 | T | I | 469420 | T | I | Yes | NS, NC | P95197 | purine base |
| | | | | | | | | | | | | | biosynthesis |
| 234 | 472937 | G | A | 471916 | A | V | 472009 | A | V | Yes | NS, C | P95200 | electron transport |
| 235 | 474708 | A | A | 473687 | G | A | 473780 | G | A | Yes | S, NULL | Null | — |
| 236 | 476885 | G | T | 475864 | C | T | 475957 | C | T | Yes | S, NULL | Null | — |
| 237 | 482898 | G | P | 481878 | A | L | 481971 | A | L | Yes | NS, NC | P95211 | membrane |
| 238 | 485256 | G | A | 484236 | A | T | 485687 | A | T | Yes | NS, NC | P95213 | enzyme activity |
| 239 | 488897 | G | G | 487876 | T | G | 489327 | T | G | Yes | S, NULL | Null | — |
| 240 | 490019 | T | T | 488998 | C | T | 490449 | C | T | Yes | S, NULL | Null | — |
| 241 | 490878 | G | V | 489857 | A | M | 491308 | A | M | Yes | NS, NC | O86335 | enzyme activity |
| 242 | 492761 | C | F | 491740 | T | F | 493191 | T | F | Yes | S, NULL | Null | — |
| 243 | 493169 | C | A | 492148 | G | G | 493599 | G | G | Yes | NS, C | P96254 | metabolism |
| 244 | 495704 | C | R | 494683 | A | R | 496134 | A | R | Yes | S, NULL | Null | — |
| 245 | 498127 | T | P | 497106 | C | P | 498557 | C | P | Yes | S, NULL | Null | — |
| 246 | 499550 | G | A | 498529 | A | A | 499980 | A | A | Yes | S, NULL | Null | — |
| 247 | 502639 | T | V | 501618 | C | A | 503069 | C | A | Yes | NS, C | P96261 | electron transport |
| 248 | 506993 | A | T | 505972 | G | A | 507423 | G | A | Yes | NS, NC | P96265 | proteolysis and |
| | | | | | | | | | | | | | peptidolysis |
| 249 | 507929 | T | E | 506908 | C | G | 508359 | C | G | Yes | NS, NC | P96266 | — |
| 250 | 515676 | G | A | 514655 | T | E | 516106 | T | E | Yes | NS, NC | P96271 | ATP binding activity |
| 251 | 518377 | C | G | 517356 | T | D | 518807 | T | D | Yes | NS, NC | P96274 | — |
| 252 | 518430 | G | R | 517409 | A | R | 518860 | A | R | Yes | S, NULL | Null | — |
| 253 | 519412 | T | T | 518391 | C | A | 519842 | C | A | Yes | NS, NC | P96275 | protein biosynthesis |
| 254 | 520204 | G | G | 519183 | T | V | 520634 | T | V | Yes | NS, C | P96277 | — |
| 255 | 520350 | G | A | 519329 | A | T | 520780 | A | T | Yes | NS, NC | P96277 | — |
| 256 | 520825 | A | A | 519804 | G | A | 521255 | G | A | Yes | S, NULL | Null | — |
| 257 | 522338 | C | C | 521317 | T | C | 522768 | T | C | Yes | S, NULL | Null | — |
| 258 | 523100 | G | A | 522079 | A | T | 523530 | A | T | Yes | NS, NC | P96280 | ATP-dependent |
| | | | | | | | | | | | | | peptidase activity |
| 259 | 528335 | G | I | 527314 | C | M | 528765 | C | M | Yes | NS, NC | O53725 | Mo-molybdopterin |
| | | | | | | | | | | | | | cofactor biosynthesis |
| 260 | 529373 | A | Null | 528352 | C | Null | 529803 | C | null | Yes | nc, NULL | Null | — |
| 261 | 533211 | T | Null | 532190 | C | Null | 533641 | C | null | Yes | S, NULL | Null | — |
| 262 | 534258 | T | E | 533237 | C | G | 534688 | C | G | Yes | NS, NC | O53729 | — |
| 263 | 534489 | T | D | 533468 | C | G | 534919 | C | G | Yes | NS, NC | O53729 | — |
| 264 | 535446 | A | Null | 534425 | T | Null | 535876 | T | null | No | nc, NULL | Null | — |
| 265 | 540882 | G | S | 539861 | A | S | 541312 | A | S | Yes | S, NULL | Null | — |
| 266 | 540902 | G | L | 539881 | A | L | 541332 | A | L | Yes | S, NULL | Null | — |
| 267 | 541571 | T | T | 540550 | C | A | 542001 | C | A | Yes | NS, NC | O53735 | membrane |
| 268 | 544180 | C | Null | 543159 | T | Null | 544610 | T | null | No | nc, NULL | Null | — |
| 269 | 547376 | G | T | 546355 | A | T | 547806 | A | T | Yes | S, NULL | Null | — |
| 270 | 556349 | T | Q | 555328 | C | R | 556779 | C | R | Yes | NS, NC | O53750 | DNA binding activity |
| 271 | 557010 | G | R | 555989 | A | C | 557440 | A | C | Yes | NS, NC | O53750 | DNA binding activity |
| 272 | 557220 | C | D | 556199 | T | N | 557650 | T | N | Yes | NS, NC | O53750 | DNA binding activity |
| 273 | 558318 | A | Null | 557297 | G | Null | 558748 | G | null | No | nc, NULL | Null | — |
| 274 | 561876 | G | L | 560855 | C | L | 562305 | C | L | Yes | S, NULL | Null | — |
| 275 | 562317 | T | A | 561296 | C | A | 562746 | C | A | Yes | S, NULL | Null | — |
| 276 | 566174 | A | G | 565153 | C | G | 566603 | C | G | Yes | S, NULL | Null | — |
| 277 | 566423 | T | R | 565402 | G | R | 566852 | G | R | Yes | S, NULL | Null | — |
| 278 | 574240 | C | T | 573275 | G | T | 574725 | G | T | Yes | S, NULL | Null | — |
| 279 | 574347 | G | L | 573382 | T | I | 574832 | T | I | Yes | NS, C | Q11150 | metabolism |
| 280 | 582972 | A | F | 581819 | G | L | 583188 | G | L | Yes | NS, NC | Q11157 | electron transporter |
| | | | | | | | | | | | | | activity |
| 281 | 583372 | C | G | 582219 | T | G | 583588 | T | G | Yes | S, NULL | Null | — |
| 282 | 584969 | T | Q | 583816 | C | Q | 585186 | C | Q | Yes | S, NULL | Null | — |
| 283 | 585322 | C | G | 584169 | T | S | 585539 | T | S | Yes | NS, NC | Q11158 | — |
| 284 | 585662 | T | T | 584509 | G | T | 585879 | G | T | Yes | S, NULL | Null | — |
| 285 | 591914 | C | D | 590761 | G | E | 592131 | G | E | Yes | NS, C | Q11141 | pyrroline 5-carboxylate |
| | | | | | | | | | | | | | reductase activity |
| 286 | 598704 | C | H | 597551 | G | D | 598920 | G | D | Yes | NS, NC | Q11171 | membrane |
| 287 | 599874 | T | Y | 598721 | G | D | 600090 | G | D | Yes | NS, NC | Q11171 | membrane |
| 288 | 600514 | G | C | 599361 | C | S | 600730 | C | S | Yes | NS, NC | Q11171 | membrane |
| 289 | 601019 | G | R | 599866 | A | R | 601235 | A | R | Yes | S, NULL | Null | — |
| 290 | 606489 | G | G | 605336 | A | E | 606705 | A | E | Yes | NS, NC | O33357 | porphobilinogen |
| | | | | | | | | | | | | | synthase activity |
| 291 | 610514 | T | H | 609361 | C | H | 610730 | C | H | Yes | S, NULL | Null | — |
| 292 | 611077 | A | D | 609924 | G | G | 611293 | G | G | Yes | NS, NC | O33362 | transferase activity |
| 293 | 612523 | C | Q | 611371 | T | Q | 612740 | T | Q | Yes | S, NULL | Null | — |
| 294 | 622386 | C | S | 621234 | G | S | 622603 | G | S | Yes | S, NULL | Null | — |
| 295 | 624971 | T | G | 623726 | G | G | 625179 | G | G | Yes | S, NULL | Null | — |
| 296 | 625445 | T | G | 624200 | C | G | 625653 | C | G | Yes | S, NULL | Null | — |
| 297 | 631262 | G | Null | 630016 | A | Null | 631469 | A | null | No | nc, NULL | Null | — |
| 298 | 631931 | T | P | 630685 | G | P | 632138 | G | P | Yes | S, NULL | Null | — |
| 299 | 634973 | C | V | 633727 | T | I | 635181 | T | I | Yes | NS, C | O06407 | — |
| 300 | 641373 | T | Null | 640127 | C | Null | 641581 | C | null | No | nc, NULL | Null | — |
| 301 | 644245 | C | R | 643000 | G | R | 644454 | G | R | Yes | S, NULL | Null | — |
| 302 | 645682 | T | A | 644437 | C | A | 645891 | C | A | Yes | S, NULL | Null | — |
| 303 | 649910 | T | D | 648665 | C | D | 650119 | C | D | Yes | S, NULL | Null | — |
| 304 | 650099 | C | G | 648854 | T | G | 650308 | T | G | Yes | S, NULL | Null | — |
| 305 | 654178 | T | G | 652933 | C | G | 654387 | C | G | Yes | S, NULL | Null | — |
| 306 | 657229 | G | Null | 655984 | T | Null | 657438 | T | null | No | nc, NULL | Null | — |
| 307 | 658821 | G | D | 657576 | A | D | 659030 | A | D | Yes | S, NULL | Null | — |
| 308 | 660166 | G | L | 658921 | C | F | 660375 | C | F | Yes | NS, NC | O53764 | methyltransferase |
| | | | | | | | | | | | | | activity |
| 309 | 660584 | C | Null | 659339 | T | Null | 660793 | T | null | No | nc, NULL | Null | — |
| 310 | 664154 | C | A | 662909 | T | A | 664363 | T | A | Yes | S, NULL | Null | — |
| 311 | 668104 | G | F | 666860 | A | F | 668313 | A | F | Yes | S, NULL | Null | — |
| 312 | 669625 | C | K | 668381 | A | N | 669834 | A | N | Yes | NS, NC | O53771 | — |
| 313 | 671788 | A | H | 670543 | G | R | 671996 | G | R | Yes | NS, NC | O53773 | DNA binding activity |
| 314 | 673323 | A | P | 672078 | G | P | 673531 | G | P | Yes | S, NULL | Null | — |
| 315 | 673880 | T | T | 672635 | C | A | 674088 | C | A | Yes | NS, NC | O53775 | subtilase activity |
| 316 | 677219 | G | Null | 675974 | A | Null | 677427 | A | null | Yes | nc, NULL | Null | — |
| 317 | 680416 | C | Null | 679171 | T | Null | 680624 | T | null | No | nc, NULL | Null | — |
| 318 | 680740 | G | G | 679495 | A | D | 680948 | A | D | Yes | NS, NC | O86365 | nitrogen fixation |
| 319 | 685069 | T | I | 683824 | C | M | 685275 | C | I | Yes | NS, NC | O53781 | — |
| 320 | 685533 | G | Null | 684288 | A | Null | 685739 | A | null | Yes | nc, NULL | Null | — |
| 321 | 688596 | C | P | 687351 | T | L | 688802 | T | L | Yes | NS, NC | O07789 | pathogenesis |
| 322 | 690448 | T | M | 689202 | C | T | 690653 | C | T | Yes | NS, NC | O07787 | pathogenesis |
| 323 | 691492 | A | N | 690246 | C | T | 691695 | C | N | Yes | NS, C | O07787 | pathogenesis |
| 324 | 691694 | C | A | 690448 | A | A | 691888 | A | null | Yes | S, NULL | Null | — |
| 325 | 693833 | T | * | 692586 | C | Q | 694036 | C | Q | Yes | NS, TP | O07785 | pathogenesis |
| 326 | 700963 | T | I | 699716 | C | V | 701166 | C | V | Yes | NS, C | O07776 | two-component |
| | | | | | | | | | | | | | response regulator |
| | | | | | | | | | | | | | activity |
| 327 | 701329 | G | A | 700082 | C | P | 701532 | C | null | Yes | NS, NC | O07775 | — |
| 328 | 701386 | A | K | 700139 | G | E | 701589 | G | null | Yes | NS, NC | O07775 | — |
| 329 | 702021 | C | P | 700774 | T | S | 702224 | T | S | Yes | NS, NC | O07774 | — |
| 330 | 706847 | C | S | 705600 | T | N | 707048 | T | N | Yes | NS, C | O07767 | — |
| 331 | 712319 | T | A | 711072 | C | A | 712520 | C | A | Yes | S, NULL | Null | — |
| 332 | 713327 | G | G | 712080 | C | R | 713528 | C | null | Yes | NS, NC | O07759 | UTP-hexose-1- |
| | | | | | | | | | | | | | phosphate |
| | | | | | | | | | | | | | uridylyltransferase |
| | | | | | | | | | | | | | activity |
| 333 | 713374 | C | A | 712127 | G | A | 713575 | G | null | Yes | S, NULL | Null | — |
| 334 | 714556 | C | R | 713308 | T | C | 714756 | T | C | Yes | NS, NC | P96910 | galactokinase activity |
| 335 | 715048 | T | C | 713800 | C | R | 715248 | C | R | Yes | NS, NC | P96910 | galactokinase activity |
| 336 | 716512 | G | W | 715264 | A | Null | 716712 | A | W | Yes | nc, NULL | Null | — |
| 337 | 718834 | G | V | 717586 | A | V | 719034 | A | V | Yes | S, NULL | Null | — |
| 338 | 720904 | T | V | 719656 | C | V | 721104 | C | V | Yes | S, NULL | Null | — |
| 339 | 721373 | C | A | 720125 | T | T | 721573 | T | T | Yes | NS, NC | P96919 | RNA binding activity |
| 340 | 722454 | T | G | 721206 | C | G | 722654 | C | G | Yes | S, NULL | Null | — |
| 341 | 723170 | G | F | 721922 | A | F | 723370 | A | F | Yes | S, NULL | Null | — |
| 342 | 723828 | A | F | 722581 | C | V | 724029 | C | V | Yes | NS, NC | P96920 | DNA binding activity |
| 343 | 726039 | A | L | 724792 | C | V | 726240 | C | V | Yes | NS, C | P96920 | DNA binding activity |
| 344 | 726979 | A | L | 725732 | G | P | 727180 | G | P | Yes | NS, NC | P96921 | helicase activity |
| 345 | 728566 | T | E | 727319 | C | G | 728767 | C | G | Yes | NS, NC | P96921 | helicase activity |
| 346 | 730359 | A | G | 729112 | G | G | 730559 | G | G | Yes | S, NULL | Null | — |
| 347 | 733788 | G | A | 732550 | A | T | 733997 | A | T | Yes | NS, NC | P96927 | cysteine-type |
| | | | | | | | | | | | | | endopeptidase activity |
| 348 | 737101 | G | A | 735863 | A | T | 737310 | A | T | Yes | NS, NC | P96932 | RNA binding activity |
| 349 | 737549 | T | P | 736311 | C | P | 737753 | C | P | Yes | S, NULL | Null | — |
| 350 | 738155 | T | L | 736917 | G | F | 738359 | G | F | Yes | NS, NC | P72028 | methyltransferase |
| | | | | | | | | | | | | | activity |
| 351 | 740056 | A | L | 738818 | C | R | 740260 | C | R | Yes | NS, NC | P72026 | methyltransferase |
| | | | | | | | | | | | | | activity |
| 352 | 742977 | T | T | 741739 | C | A | 743181 | C | A | Yes | NS, NC | P96936 | — |
| 353 | 745086 | T | L | 743315 | C | S | 745290 | C | S | Yes | NS, NC | P96937 | alpha-mannosidase |
| | | | | | | | | | | | | | activity |
| 354 | 746998 | A | P | 745227 | G | P | 747202 | G | P | Yes | S, NULL | Null | — |
| 355 | 747114 | G | R | 745343 | C | P | 747318 | C | P | Yes | NS, NC | P96937 | alpha-mannosidase |
| | | | | | | | | | | | | | activity |
| 356 | 758951 | G | G | 757180 | A | D | 759155 | A | D | Yes | NS, NC | O06776 | metabolism |
| 357 | 764800 | C | A | 763029 | T | A | 765004 | T | A | Yes | S, NULL | Null | — |
| 358 | 770931 | G | P | 769160 | A | S | 771136 | A | null | Yes | NS, NC | O06769 | — |
| 359 | 771448 | C | Null | 769677 | A | Null | 771653 | A | null | No | nc, NULL | Null | — |
| 360 | 772157 | A | A | 770386 | G | A | 772362 | G | A | Yes | S, NULL | Null | — |
| 361 | 774665 | A | L | 772894 | G | L | 774870 | G | L | Yes | S, NULL | Null | — |
| 362 | 777869 | A | I | 776098 | G | T | 778074 | G | T | Yes | NS, NC | O53784 | membrane |
| 363 | 784766 | A | A | 782995 | G | A | 784971 | G | A | Yes | S, NULL | Null | — |
| 364 | 788162 | G | S | 786391 | T | I | 788367 | T | I | Yes | NS, NC | P95032 | — |
| 365 | 800584 | T | Null | 798813 | C | Null | 800788 | C | null | No | nc, NULL | Null | — |
| 366 | 804997 | T | G | 803173 | C | G | 805364 | C | G | Yes | S, NULL | Null | — |
| 367 | 808276 | T | L | 806452 | G | L | 808643 | G | L | Yes | S, NULL | Null | — |
| 368 | 808601 | T | * | 806777 | G | E | 808968 | G | E | Yes | NS, TP | P95059 | metabolism |
| 369 | 811737 | C | Null | 809913 | A | Null | 812104 | A | null | No | nc, NULL | Null | — |
| 370 | 812709 | G | Null | 810885 | A | Null | 813076 | A | null | No | nc, NULL | Null | — |
| 371 | 816925 | T | R | 815101 | C | R | 817293 | C | R | Yes | S, NULL | Null | — |
| 372 | 817058 | C | T | 815234 | T | I | 817426 | T | I | Yes | NS, NC | P95071 | structural constituent of |
| | | | | | | | | | | | | | ribosome |
| 373 | 817673 | G | R | 815849 | A | R | 818041 | A | null | Yes | S, NULL | Null | — |
| 374 | 822574 | T | H | 820750 | C | R | 822942 | C | R | Yes | NS, NC | O86322 | serine biosynthesis |
| 375 | 823729 | C | P | 821905 | T | L | 824097 | T | L | Yes | NS, NC | O53793 | carbohydrate |
| | | | | | | | | | | | | | metabolism |
| 376 | 828003 | C | L | 826179 | G | L | 828371 | G | L | Yes | S, NULL | Null | — |
| 377 | 833390 | G | V | 831564 | A | M | 833756 | A | M | Yes | NS, NC | O53802 | — |
| 378 | 834025 | T | D | 832199 | C | D | 834390 | C | D | Yes | S, NULL | Null | — |
| 379 | 834070 | G | Q | 832244 | A | Q | 834435 | A | Q | Yes | S, NULL | Null | — |
| 380 | 836836 | T | Null | 835010 | A | Null | 837201 | A | null | No | nc, NULL | Null | — |
| 381 | 837652 | T | V | 835826 | C | A | 838017 | C | A | Yes | NS, C | O53809 | — |
| 382 | 839308 | A | N | 837578 | G | S | 839769 | G | S | Yes | NS, C | O53809 | — |
| 383 | 846049 | C | V | 843857 | T | I | 846000 | T | I | Yes | NS, C | O53815 | acyl-CoA |
| | | | | | | | | | | | | | dehydrogenase activity |
| 384 | 846399 | G | A | 844207 | A | V | 846350 | A | V | Yes | NS, C | O53815 | acyl-OcA |
| | | | | | | | | | | | | | dehydrogenase activity |
| 385 | 846819 | C | G | 844627 | A | V | 846770 | A | V | Yes | NS, C | O53816 | metabolism |
| 386 | 850185 | C | Null | 847993 | T | Null | 850136 | T | null | No | nc, NULL | Null | — |
| 387 | 850597 | T | K | 848405 | C | R | 850548 | C | R | Yes | NS, C | O53818 | — |
| 388 | 853294 | T | A | 851102 | C | A | 853245 | C | A | Yes | S, NULL | Null | — |
| 389 | 853752 | A | Null | 851560 | G | Null | 853703 | G | null | No | nc, NULL | Null | — |
| 390 | 854796 | A | I | 852604 | G | I | 854747 | G | I | Yes | S, NULL | Null | — |
| 391 | 854797 | T | I | 852605 | G | I | 854748 | G | I | Yes | S, NULL | Null | — |
| 392 | 856687 | A | F | 854496 | G | F | 856638 | G | F | Yes | S, NULL | Null | — |
| 393 | 864764 | T | V | 862573 | C | A | 864715 | C | A | Yes | NS, C | P71824 | metabolism |
| 394 | 865821 | G | G | 863630 | T | V | 865772 | T | V | Yes | NS, C | P71825 | valine metabolism |
| 395 | 869886 | C | A | 867695 | A | S | 869837 | A | S | Yes | NS, NC | P71829 | enzyme activity |
| 396 | 870116 | G | A | 867925 | T | D | 870067 | T | D | Yes | NS, NC | P71829 | enzyme activity |
| 397 | 870460 | C | A | 868269 | T | A | 870411 | T | A | Yes | S, NULL | Null | — |
| 398 | 873460 | C | P | 871269 | A | T | 873411 | A | T | Yes | NS, NC | P71832 | enzyme activity |
| 399 | 879911 | A | L | 877718 | G | L | 879862 | G | L | Yes | S, NULL | Null | — |
| 400 | 879912 | A | G | 877719 | G | G | 879863 | G | G | Yes | S, NULL | Null | — |
| 401 | 895718 | C | S | 894886 | T | S | 894797 | T | null | Yes | S, NULL | Null | — |
| 402 | 901045 | G | R | 900213 | C | R | 900124 | C | R | Yes | S, NULL | Null | — |
| 403 | 904341 | G | V | 903509 | C | V | 903420 | C | V | Yes | S, NULL | Null | — |
| 404 | 905912 | C | G | 905080 | G | G | 904991 | G | G | Yes | S, NULL | Null | — |
| 405 | 912091 | C | P | 911259 | T | L | 911170 | T | L | Yes | NS, NC | O53830 | two-component |
| | | | | | | | | | | | | | response regulator |
| | | | | | | | | | | | | | activity |
| 406 | 913660 | G | L | 912828 | C | F | 912739 | C | F | Yes | NS, NC | O53832 | nucleotide binding |
| | | | | | | | | | | | | | activity |
| 407 | 914104 | G | S | 913272 | C | S | 913183 | C | S | Yes | S, NULL | Null | — |
| 408 | 916876 | G | P | 916044 | T | H | 915955 | T | H | Yes | NS, NC | O53834 | — |
| 409 | 917180 | A | Null | 916348 | G | Null | 916259 | G | null | No | nc, NULL | Null | — |
| 410 | 917489 | G | L | 916657 | A | F | 916568 | A | F | Yes | NS, NC | O53835 | molecular_function |
| | | | | | | | | | | | | | unknown |
| 411 | 917544 | T | L | 916712 | G | L | 916623 | G | L | Yes | S, NULL | Null | — |
| 412 | 918089 | A | C | 917257 | C | G | 917168 | C | G | Yes | NS, NC | O53835 | molecular_function |
| | | | | | | | | | | | | | unknown |
| 413 | 919629 | C | Null | 918797 | T | Null | 918708 | T | null | No | nc, NULL | Null | — |
| 414 | 920661 | T | R | 919829 | C | R | 919740 | C | R | Yes | S, NULL | Null | — |
| 415 | 920753 | G | G | 919921 | A | E | 919832 | A | E | Yes | NS, NC | O53837 | — |
| 416 | 921344 | A | Q | 920512 | G | R | 920423 | G | R | Yes | NS, NC | O53837 | — |
| 417 | 925130 | A | Null | 924298 | G | Null | 924209 | G | null | No | nc, NULL | Null | — |
| 418 | 928734 | C | Null | 927830 | T | Null | 927741 | T | null | No | nc, NULL | Null | — |
| 419 | 929147 | T | G | 928396 | G | G | 928298 | G | G | Yes | S, NULL | Null | — |
| 420 | 930497 | T | A | 929746 | C | A | 929648 | C | A | Yes | S, NULL | Null | — |
| 421 | 931872 | C | Y | 931121 | T | Y | 931023 | T | Y | Yes | S, NULL | Null | — |
| 422 | 932186 | G | G | 931435 | A | D | 931337 | A | D | Yes | NS, NC | O53846 | — |
| 423 | 933001 | A | P | 932250 | C | Null | 932152 | C | null | Yes | nc, NULL | Null | — |
| 424 | 933029 | C | W | 932278 | T | * | 932180 | T | * | Yes | NS, TP | O53848 | — |
| 425 | 934448 | T | G | 933697 | G | G | 933599 | G | G | Yes | S, NULL | Null | — |
| 426 | 934979 | G | Null | 934228 | C | Null | 934130 | C | null | No | nc, NULL | Null | — |
| 427 | 934982 | A | Null | 934231 | G | Null | 934133 | G | null | No | nc, NULL | Null | — |
| 428 | 935360 | T | Null | 934609 | G | Null | 934511 | G | null | No | nc, NULL | Null | — |
| 429 | 938432 | T | F | 937675 | G | F | 937577 | G | V | Yes | S, NULL | Null | — |
| 430 | 939001 | G | L | 938244 | A | L | 938146 | A | L | Yes | S, NULL | Null | — |
| 431 | 940714 | G | G | 939957 | A | D | 939859 | A | D | Yes | NS, NC | O53855 | metabolism |
| 432 | 941068 | A | D | 940311 | C | A | 940213 | C | A | Yes | NS, NC | O53855 | metabolism |
| 433 | 941645 | G | P | 940888 | C | A | 940790 | C | A | Yes | NS, NC | O53856 | two-component |
| | | | | | | | | | | | | | response regulator |
| | | | | | | | | | | | | | activity |
| 434 | 942600 | A | E | 941843 | C | A | 941745 | C | A | Yes | NS, NC | O53857 | ATP binding activity |
| 435 | 943719 | G | H | 942962 | A | Y | 942864 | A | Y | Yes | NS, C | O53858 | copper ion binding |
| | | | | | | | | | | | | | activity |
| 436 | 945051 | G | Null | 944294 | A | Null | 944196 | A | null | No | nc, NULL | Null | — |
| 437 | 945480 | T | V | 944723 | C | A | 944625 | C | A | Yes | NS, C | O53859 | — |
| 438 | 946102 | G | G | 945345 | T | V | 945247 | T | null | Yes | NS, C | O53860 | amino acid metabolism |
| 439 | 948022 | T | F | 947265 | C | F | 947165 | C | null | Yes | S, NULL | Null | — |
| 440 | 948049 | C | G | 947292 | T | G | 947192 | T | null | Yes | S, NULL | Null | — |
| 441 | 948974 | A | F | 948217 | C | V | 948117 | C | V | Yes | NS, NC | O53863 | metabolism |
| 442 | 949049 | C | G | 948292 | T | S | 948192 | T | S | Yes | NS, NC | O53863 | metabolism |
| 443 | 951897 | A | Null | 951140 | C | Null | 951040 | C | null | No | nc, NULL | Null | — |
| 444 | 953517 | C | Null | 952760 | G | Null | 952660 | G | null | No | nc, NULL | Null | — |
| 445 | 958717 | A | L | 957961 | G | L | 957861 | G | L | Yes | S, NULL | Null | — |
| 446 | 959147 | A | T | 958391 | G | A | 958291 | G | A | Yes | NS, NC | O53872 | enzyme activity |
| 447 | 960133 | G | A | 959377 | A | V | 959277 | A | V | Yes | NS, C | O53873 | nucleic acid binding |
| | | | | | | | | | | | | | activity |
| 448 | 961068 | G | S | 960365 | A | L | 960371 | A | L | Yes | NS, NC | O53874 | — |
| 449 | 967989 | T | S | 967527 | C | G | 967533 | C | G | Yes | NS, NC | O53882 | — |
| 450 | 970372 | A | L | 969904 | G | L | 969919 | G | L | Yes | S, NULL | Null | — |
| 451 | 972368 | A | G | 971900 | G | G | 971915 | G | G | Yes | S, NULL | Null | — |
| 452 | 974604 | G | L | 974136 | A | L | 974150 | A | L | Yes | S, NULL | Null | — |
| 453 | 976327 | C | G | 975859 | T | D | 975873 | T | D | Yes | NS, NC | Q10564 | integral to membrane |
| 454 | 997447 | G | A | 996980 | A | A | 996994 | A | A | Yes | S, NULL | Null | — |
| 455 | 998183 | C | Null | 997716 | G | Null | 997730 | G | null | No | nc, NULL | Null | — |
| 456 | 1001197 | G | A | 1000730 | A | T | 1000744 | A | T | Yes | NS, NC | Q10530 | enzyme activity |
| 457 | 1009407 | C | Null | 1008940 | T | Null | 1008954 | T | null | No | nc, NULL | Null | — |
| 458 | 1010182 | T | G | 1009715 | C | G | 1009729 | C | G | Yes | S, NULL | Null | — |
| 459 | 1010422 | A | L | 1009955 | G | L | 1009969 | G | L | Yes | S, NULL | Null | — |
| 460 | 1011566 | T | L | 1011098 | C | P | 1011113 | C | P | Yes | NS, NC | O05900 | — |
| 461 | 1015281 | G | Q | 1014813 | T | H | 1014828 | T | H | Yes | NS, NC | O05901 | — |
| 462 | 1018494 | A | L | 1018026 | G | P | 1018041 | G | P | Yes | NS, NC | O05905 | — |
| 463 | 1024812 | G | G | 1024344 | A | S | 1024359 | A | S | Yes | NS, NC | O05910 | — |
| 464 | 1026536 | T | E | 1026068 | G | D | 1026083 | G | D | Yes | NS, C | O05912 | — |
| 465 | 1027911 | C | V | 1027443 | G | V | 1027458 | G | V | Yes | S, NULL | Null | — |
| 466 | 1030402 | G | R | 1029934 | A | R | 1029949 | A | R | Yes | S, NULL | Null | — |
| 467 | 1034703 | G | L | 1034236 | A | L | 1034251 | A | L | Yes | S, NULL | Null | — |
| 468 | 1041172 | C | A | 1040704 | A | S | 1040719 | A | S | Yes | NS, NC | O05870 | transporter activity |
| 469 | 1043636 | C | A | 1043167 | T | V | 1043182 | T | V | Yes | NS, C | P15712 | transporter activity |
| 470 | 1048294 | T | L | 1047825 | C | L | 1047840 | C | L | Yes | S, NULL | Null | — |
| 471 | 1054603 | T | T | 1054134 | C | T | 1054149 | C | T | Yes | S, NULL | Null | — |
| 472 | 1055251 | G | G | 1054782 | C | R | 1054797 | C | R | Yes | NS, NC | P71564 | metabolism |
| 473 | 1063212 | C | A | 1062743 | T | V | 1062758 | T | V | Yes | NS, C | P71559 | enzyme activity |
| 474 | 1064232 | A | K | 1063763 | G | R | 1063778 | G | R | Yes | NS, C | P71558 | enzyme activity |
| 475 | 1077356 | T | H | 1076915 | C | R | 1076930 | C | R | Yes | NS, NC | P71545 | — |
| 476 | 1077719 | C | G | 1077278 | A | W | 1077293 | A | W | Yes | NS, NC | P71544 | — |
| 477 | 1080631 | A | N | 1080190 | G | D | 1080205 | G | D | Yes | NS, NC | P77894 | magnesium ion binding |
| | | | | | | | | | | | | | activity |
| 478 | 1083482 | A | H | 1083041 | C | Q | 1083056 | C | Q | Yes | NS, NC | P71539 | acyl-CoA |
| | | | | | | | | | | | | | dehydrogenase activity |
| 479 | 1085532 | A | F | 1085091 | T | I | 1085106 | T | I | Yes | NS, NC | P71538 | ATP binding activity |
| 480 | 1096095 | T | T | 1095642 | C | A | 1095671 | C | A | Yes | NS, NC | O53893 | — |
| 481 | 1096129 | G | G | 1095676 | A | G | 1095705 | A | G | Yes | S, NULL | Null | — |
| 482 | 1096774 | T | Q | 1096321 | G | H | 1096362 | G | H | Yes | NS, NC | O53893 | — |
| 483 | 1097474 | A | S | 1097021 | G | G | 1097062 | G | G | Yes | NS, NC | O53894 | two-component |
| | | | | | | | | | | | | | response regulator |
| | | | | | | | | | | | | | activity |
| 484 | 1098974 | A | H | 1098521 | T | L | 1098562 | T | L | Yes | NS, NC | O53895 | two-component sensor |
| | | | | | | | | | | | | | molecule activity |
| 485 | 1102935 | T | L | 1102482 | G | V | 1102523 | G | V | Yes | NS, C | O53899 | nucleotide binding |
| | | | | | | | | | | | | | activity |
| 486 | 1103991 | T | G | 1103538 | C | G | 1103579 | C | G | Yes | S, NULL | Null | — |
| 487 | 1104263 | A | * | 1103810 | G | W | 1103851 | G | W | Yes | NS, TP | O53900 | membrane |
| 488 | 1105141 | G | V | 1104688 | T | F | 1104729 | T | F | Yes | NS, NC | O53900 | membrane |
| 489 | 1105524 | G | G | 1105071 | A | G | 1105112 | A | G | Yes | S, NULL | Null | — |
| 490 | 1105735 | A | I | 1105282 | G | V | 1105323 | G | V | Yes | NS, C | O86370 | — |
| 491 | 1105969 | G | D | 1105516 | T | Y | 1105557 | T | Y | Yes | NS, NC | O86370 | — |
| 492 | 1108391 | C | A | 1107938 | A | S | 1107979 | A | S | Yes | NS, NC | O05573 | — |
| 493 | 1109614 | G | I | 1109161 | C | M | 1109202 | C | M | Yes | NS, NC | O05575 | enzyme activity |
| 494 | 1111407 | G | G | 1110954 | T | C | 1110995 | T | C | Yes | NS, NC | O05577 | Mo-molybdopterin |
| | | | | | | | | | | | | | cofactor biosynthesis |
| 495 | 1113109 | G | G | 1112656 | A | D | 1112697 | A | D | Yes | NS, NC | O05579 | — |
| 496 | 1113741 | C | Q | 1113288 | G | E | 1113329 | G | E | Yes | NS, NC | O05579 | — |
| 497 | 1120048 | G | L | 1119595 | C | V | 1119635 | C | V | Yes | NS, C | O05586 | mannosyltransferase |
| | | | | | | | | | | | | | activity |
| 498 | 1124048 | G | S | 1123595 | A | L | 1123641 | A | L | Yes | NS, NC | O05591 | biosynthesis |
| 499 | 1124238 | G | S | 1123785 | A | N | 1123831 | A | S | Yes | NS, C | O05592 | — |
| 500 | 1125767 | T | S | 1125314 | C | P | 1125360 | C | P | Yes | NS, NC | O05592 | — |
| 501 | 1129386 | A | E | 1128933 | G | G | 1128979 | G | G | Yes | NS, NC | O05594 | — |
| 502 | 1129611 | T | V | 1129158 | C | A | 1129204 | C | A | Yes | NS, C | O05594 | — |
| 503 | 1131752 | A | N | 1131298 | G | S | 1131344 | G | null | Yes | NS, C | O05597 | — |
| 504 | 1137772 | G | Q | 1137323 | C | E | 1137369 | C | E | Yes | NS, NC | P96382 | UDP-N- |
| | | | | | | | | | | | | | acetylglucosamine |
| | | | | | | | | | | | | | pyrophosphorylase |
| | | | | | | | | | | | | | activity |
| 505 | 1138029 | C | G | 1137580 | T | E | 1137626 | T | E | Yes | NS, NC | P96382 | UDP-N- |
| | | | | | | | | | | | | | acetylglucosamine |
| | | | | | | | | | | | | | pyrophosphorylase |
| | | | | | | | | | | | | | activity |
| 506 | 1139461 | T | L | 1139012 | C | L | 1139058 | C | L | Yes | S, NULL | Null | — |
| 507 | 1144279 | C | P | 1143830 | A | T | 1143876 | A | T | Yes | NS, NC | P96378 | — |
| 508 | 1145032 | G | G | 1144583 | A | R | 1144629 | A | R | Yes | NS, NC | P96377 | phosphopyruvate |
| | | | | | | | | | | | | | hydratase complex |
| 509 | 1148706 | G | Null | 1148257 | A | Null | 1148303 | A | null | Yes | nc, NULL | Null | — |
| 510 | 1149575 | T | L | 1149126 | C | L | 1149172 | C | L | Yes | S, NULL | Null | — |
| 511 | 1151250 | T | D | 1150801 | C | G | 1150847 | C | G | Yes | NS, NC | P96372 | two-component sensor |
| | | | | | | | | | | | | | molecule activity |
| 512 | 1152153 | T | Null | 1151704 | C | Null | 1151750 | C | null | Yes | nc, NULL | Null | — |
| 513 | 1161217 | A | L | 1160768 | T | Q | 1160814 | T | Q | Yes | NS, NC | P96364 | — |
| 514 | 1164196 | A | Null | 1163747 | G | Null | 1163793 | G | null | Yes | nc, NULL | Null | — |
| 515 | 1164719 | T | Null | 1164270 | C | Null | 1164316 | C | null | Yes | nc, NULL | Null | — |
| 516 | 1165018 | G | Null | 1164569 | A | Null | 1164615 | A | null | No | nc, NULL | Null | — |
| 517 | 1165561 | T | N | 1165112 | C | D | 1165158 | C | null | Yes | NS, NC | P96360 | — |
| 518 | 1166468 | A | C | 1166018 | C | G | 1166065 | C | G | Yes | NS, NC | P96358 | serine-type |
| | | | | | | | | | | | | | endopeptidase activity |
| 519 | 1166960 | T | T | 1166510 | C | A | 1166557 | C | A | Yes | NS, NC | P96358 | serine-type |
| | | | | | | | | | | | | | endopeptidase activity |
| 520 | 1168430 | G | G | 1167980 | T | W | 1168027 | T | W | Yes | NS, NC | P96356 | — |
| 521 | 1169896 | A | T | 1169445 | G | A | 1169493 | G | A | Yes | NS, NC | P96354 | peroxidase activity |
| 522 | 1170414 | A | G | 1169963 | G | G | 1170011 | G | G | Yes | S, NULL | Null | — |
| 523 | 1171297 | A | Null | 1170846 | G | Null | 1170894 | G | null | Yes | nc, NULL | Null | — |
| 524 | 1175230 | A | Null | 1174780 | G | Null | 1174828 | G | null | No | nc, NULL | Null | — |
| 525 | 1177201 | C | Null | 1176751 | T | Null | 1176799 | T | null | Yes | nc, NULL | Null | — |
| 526 | 1179589 | G | Null | 1179139 | T | Null | 1179187 | T | null | No | nc, NULL | Null | — |
| 527 | 1189705 | T | M | 1189243 | C | V | 1189291 | C | V | Yes | NS, NC | O53415 | — |
| 528 | 1193177 | C | W | 1191815 | A | L | 1192055 | A | L | Yes | NS, NC | O53416 | — |
| 529 | 1193221 | C | E | 1191859 | T | E | 1192099 | T | E | Yes | S, NULL | Null | — |
| 530 | 1199501 | G | Null | 1198139 | A | Null | 1198376 | A | null | No | nc, NULL | Null | — |
| 531 | 1199502 | A | Null | 1198140 | C | Null | 1198377 | C | null | No | nc, NULL | Null | — |
| 532 | 1199636 | A | G | 1198274 | G | G | 1198511 | G | G | Yes | S, NULL | Null | — |
| 533 | 1205184 | C | T | 1203822 | T | I | 1204059 | T | I | Yes | NS, NC | O53426 | — |
| 534 | 1206868 | A | N | 1205506 | G | N | 1205743 | G | N | Yes | S, NULL | Null | — |
| 535 | 1212729 | C | R | 1211367 | A | S | 1211604 | A | S | Yes | NS, NC | O53434 | metabolism |
| 536 | 1214512 | T | G | 1213168 | C | G | 1213326 | C | null | Yes | S, NULL | Null | — |
| 537 | 1221942 | T | Null | 1220568 | G | Null | 1220117 | G | null | No | nc, NULL | Null | — |
| 538 | 1226570 | T | S | 1225196 | C | P | 1224745 | C | P | Yes | NS, NC | O53444 | carbohydrate |
| | | | | | | | | | | | | | metabolism |
| 539 | 1227449 | A | L | 1226075 | G | P | 1225624 | G | P | Yes | NS, NC | O53445 | — |
| 540 | 1230847 | G | D | 1229473 | T | E | 1229022 | T | E | Yes | NS, C | O53449 | integral to membrane |
| 541 | 1232149 | A | I | 1230776 | G | T | 1230325 | G | T | Yes | NS, NC | O53450 | DNA binding activity |
| 542 | 1236028 | A | D | 1234655 | G | D | 1234205 | G | D | Yes | S, NULL | Null | — |
| 543 | 1236817 | C | S | 1235444 | T | S | 1234994 | T | S | Yes | S, NULL | Null | — |
| 544 | 1239854 | A | I | 1238481 | G | V | 1238031 | G | V | Yes | NS, C | O53459 | GTP binding activity |
| 545 | 1241612 | C | P | 1240239 | T | S | 1239789 | T | S | Yes | NS, NC | O06567 | — |
| 546 | 1244718 | G | A | 1243345 | C | G | 1242895 | C | G | Yes | NS, C | O06572 | guanylate cyclase |
| | | | | | | | | | | | | | activity |
| 547 | 1245764 | G | V | 1244391 | C | V | 1243941 | C | V | Yes | S, NULL | Null | — |
| 548 | 1249753 | G | G | 1248380 | A | S | 1247930 | A | null | Yes | NS, NC | O06577 | — |
| 549 | 1250307 | C | P | 1248934 | G | P | 1248483 | G | P | Yes | S, NULL | Null | — |
| 550 | 1251711 | G | A | 1250338 | A | A | 1249887 | A | A | Yes | S, NULL | Null | — |
| 551 | 1251728 | G | P | 1250355 | T | T | 1249904 | T | T | Yes | NS, NC | O06579 | ATP binding activity |
| 552 | 1255933 | G | G | 1254560 | A | D | 1254109 | A | D | Yes | NS, NC | O06582 | — |
| 553 | 1255953 | C | L | 1254580 | T | F | 1254129 | T | F | Yes | NS, NC | O06582 | — |
| 554 | 1257383 | T | R | 1256010 | G | R | 1255559 | G | R | Yes | S, NULL | Null | — |
| 555 | 1259222 | T | M | 1257849 | C | T | 1257398 | C | T | Yes | NS, NC | O06583 | — |
| 556 | 1261908 | T | L | 1260535 | C | L | 1260084 | C | L | Yes | S, NULL | Null | — |
| 557 | 1282061 | C | G | 1280687 | T | D | 1280179 | T | D | Yes | NS, NC | O06551 | methyltransferase |
| | | | | | | | | | | | | | activity |
| 558 | 1282113 | T | T | 1280739 | C | A | 1280231 | C | A | Yes | NS, NC | O06551 | methyltransferase |
| | | | | | | | | | | | | | activity |
| 559 | 1283143 | C | P | 1281769 | T | S | 1281261 | T | S | Yes | NS, NC | O06553 | — |
| 560 | 1288484 | C | Null | 1287110 | T | Null | 1286600 | T | null | No | nc, NULL | Null | — |
| 561 | 1290071 | C | L | 1288697 | G | V | 1288187 | G | V | Yes | NS, C | O06559 | electron transport |
| 562 | 1291161 | G | G | 1289787 | A | D | 1289277 | A | D | Yes | NS, NC | O06559 | electron transport |
| 563 | 1295376 | T | V | 1294002 | C | A | 1293492 | C | A | Yes | NS, C | O06562 | electron transport |
| 564 | 1295770 | T | D | 1294396 | C | D | 1293886 | C | D | Yes | S, NULL | Null | — |
| 565 | 1303656 | G | H | 1302281 | T | Q | 1301771 | T | Q | Yes | NS, NC | O50428 | — |
| 566 | 1304272 | G | Null | 1302897 | A | Null | 1302387 | A | null | No | nc, NULL | Null | — |
| 567 | 1307530 | C | S | 1306279 | A | I | 1305769 | A | I | Yes | NS, NC | O50431 | electron transport |
| 568 | 1309207 | T | N | 1307956 | G | T | 1307446 | G | T | Yes | NS, C | O50431 | electron transport |
| 569 | 1311565 | T | V | 1310314 | C | A | 1309804 | C | A | Yes | NS, C | O50434 | transaminase activity |
| 570 | 1318177 | C | A | 1316925 | A | D | 1316414 | A | D | Yes | NS, NC | O50437 | alcohol dehydrogenase |
| | | | | | | | | | | | | | activity |
| 571 | 1325815 | A | R | 1324563 | G | R | 1324052 | G | R | Yes | S, NULL | Null | — |
| 572 | 1335064 | T | L | 1333812 | C | L | 1333301 | C | L | Yes | S, NULL | Null | — |
| 573 | 1340081 | C | Null | 1338829 | A | Null | 1338318 | A | null | Yes | nc, NULL | Null | — |
| 574 | 1341302 | T | L | 1340050 | G | R | 1339539 | G | R | Yes | NS, NC | O05298 | — |
| 575 | 1341343 | G | V | 1340091 | A | M | 1339580 | A | M | Yes | NS, NC | O05298 | — |
| 576 | 1341420 | T | A | 1340168 | C | A | 1339657 | C | A | Yes | S, NULL | Null | — |
| 577 | 1341887 | T | Null | 1340638 | C | Null | 1340127 | C | null | No | nc, NULL | Null | — |
| 578 | 1341914 | G | S | 1340665 | A | S | 1340154 | A | S | Yes | S, NULL | Null | — |
| 579 | 1342025 | C | I | 1340776 | T | I | 1340265 | T | I | Yes | S, NULL | Null | — |
| 580 | 1342028 | G | S | 1340779 | C | S | 1340268 | C | S | Yes | S, NULL | Null | — |
| 581 | 1342031 | C | G | 1340782 | T | G | 1340271 | T | G | Yes | S, NULL | Null | — |
| 582 | 1342077 | A | T | 1340828 | G | A | 1340317 | G | A | Yes | NS, NC | O05299 | — |
| 583 | 1342287 | A | D | 1341038 | C | A | 1340527 | C | A | Yes | NS, NC | O05300 | — |
| 584 | 1342456 | T | A | 1341207 | C | A | 1340696 | C | A | Yes | S, NULL | Null | — |
| 585 | 1343724 | A | A | 1342475 | G | A | 1341964 | G | A | Yes | S, NULL | Null | — |
| 586 | 1345988 | G | Y | 1344739 | A | Y | 1344228 | A | Y | Yes | S, NULL | Null | — |
| 587 | 1348420 | T | L | 1347171 | C | L | 1346660 | C | L | Yes | S, NULL | Null | — |
| 588 | 1352419 | G | Null | 1351170 | A | Null | 1350659 | A | null | No | nc, NULL | Null | — |
| 589 | 1356596 | T | I | 1355347 | C | V | 1354836 | C | V | Yes | NS, C | O05313 | biosynthesis |
| 590 | 1357184 | T | F | 1355935 | C | F | 1355424 | C | F | Yes | S, NULL | Null | — |
| 591 | 1360182 | A | S | 1358938 | C | A | 1358427 | C | A | Yes | NS, NC | O05316 | DNA binding activity |
| 592 | 1362617 | T | T | 1361373 | C | A | 1360862 | C | A | Yes | NS, NC | O05318 | — |
| 593 | 1367980 | C | L | 1366734 | T | F | 1366224 | T | F | Yes | NS, NC | O06291 | serine-type |
| | | | | | | | | | | | | | endopeptidase activity |
| 594 | 1368452 | A | N | 1367206 | G | S | 1366696 | G | S | Yes | NS, C | O06291 | serine-type |
| | | | | | | | | | | | | | endopeptidase activity |
| 595 | 1368728 | G | G | 1367482 | T | W | 1366972 | T | W | Yes | NS, NC | O33220 | protein targeting |
| 596 | 1370191 | G | A | 1368945 | A | V | 1368435 | A | V | Yes | NS, C | O33222 | — |
| 597 | 1372850 | C | Null | 1371604 | A | Null | 1371094 | A | null | Yes | nc, NULL | Null | — |
| 598 | 1375153 | G | P | 1373907 | A | S | 1373397 | A | S | Yes | NS, NC | O86313 | transcription factor |
| | | | | | | | | | | | | | activity |
| 599 | 1377868 | T | D | 1376622 | C | G | 1376112 | C | G | Yes | NS, NC | O86316 | — |
| 600 | 1377935 | C | D | 1376689 | T | N | 1376179 | T | N | Yes | NS, NC | O86316 | — |
| 601 | 1378384 | G | E | 1377138 | A | E | 1376628 | A | E | Yes | S, NULL | Null | — |
| 602 | 1383703 | C | R | 1382457 | T | H | 1381947 | T | H | Yes | NS, NC | O50455 | cobalt ion transport |
| 603 | 1388166 | T | G | 1386920 | C | G | 1386410 | C | G | Yes | S, NULL | Null | — |
| 604 | 1388824 | A | K | 1387578 | C | Q | 1387068 | C | Q | Yes | NS, NC | O50459 | — |
| 605 | 1391333 | T | A | 1390087 | C | A | 1389576 | C | A | Yes | S, NULL | Null | — |
| 606 | 1392007 | T | M | 1390761 | C | V | 1390250 | C | V | Yes | NS, NC | O50463 | metabolism |
| 607 | 1394247 | G | Null | 1393001 | T | Null | 1392490 | T | null | Yes | nc, NULL | Null | — |
| 608 | 1394944 | T | T | 1393698 | C | A | 1393187 | C | A | Yes | NS, NC | O50464 | — |
| 609 | 1396254 | G | G | 1395008 | A | R | 1394497 | A | R | Yes | NS, NC | O50465 | transporter activity |
| 610 | 1398445 | G | N | 1397199 | A | N | 1396688 | A | N | Yes | S, NULL | Null | — |
| 611 | 1401640 | G | V | 1400394 | A | M | 1399883 | A | M | Yes | NS, NC | Q11039 | nucleic acid binding |
| | | | | | | | | | | | | | activity |
| 612 | 1408298 | C | R | 1410060 | G | P | 1409548 | G | P | Yes | NS, NC | Q11066 | — |
| 613 | 1410739 | T | C | 1412501 | C | R | 1411989 | C | R | Yes | NS, NC | Q11055 | guanylate cyclase |
| | | | | | | | | | | | | | activity |
| 614 | 1412796 | T | R | 1414558 | C | R | 1414046 | C | R | Yes | S, NULL | Null | — |
| 615 | 1412800 | T | N | 1414562 | A | I | 1414050 | A | I | Yes | NS, NC | Q11053 | protein kinase activity |
| 616 | 1412889 | T | P | 1414651 | C | P | 1414139 | C | P | Yes | S, NULL | Null | — |
| 617 | 1413243 | G | A | 1415095 | C | A | 1414583 | C | A | Yes | S, NULL | Null | — |
| 618 | 1415700 | C | Null | 1417552 | G | Null | 1417040 | G | null | Yes | nc, NULL | Null | — |
| 619 | 1417495 | T | T | 1419347 | C | A | 1418835 | C | A | Yes | NS, NC | Q11049 | membrane |
| 620 | 1421972 | T | A | 1423824 | C | A | 1423312 | C | A | Yes | S, NULL | Null | — |
| 621 | 1422845 | C | P | 1424697 | T | L | 1424185 | T | L | Yes | NS, NC | Q11045 | membrane |
| 622 | 1423465 | A | Null | 1425317 | G | Null | 1424805 | G | null | No | nc, NULL | Null | — |
| 623 | 1423787 | A | S | 1425639 | T | T | 1425127 | T | T | Yes | NS, C | Q11043 | hydrolase activity |
| 624 | 1425622 | T | F | 1427474 | C | F | 1426962 | C | F | Yes | S, NULL | Null | — |
| 625 | 1435584 | A | R | 1437436 | C | R | 1436924 | C | R | Yes | S, NULL | Null | — |
| 626 | 1437785 | T | L | 1439637 | G | V | 1439125 | G | V | Yes | NS, C | Q10600 | sulfate assimilation |
| 627 | 1438084 | T | R | 1439936 | C | R | 1439424 | C | R | Yes | S, NULL | Null | — |
| 628 | 1440727 | G | Null | 1442732 | C | Null | 1442220 | C | null | No | nc, NULL | Null | — |
| 629 | 1452860 | T | N | 1454809 | C | N | 1454352 | C | N | Yes | S, NULL | Null | — |
| 630 | 1456125 | G | G | 1458074 | T | G | 1457617 | T | G | Yes | S, NULL | Null | — |
| 631 | 1456193 | A | Q | 1458142 | C | P | 1457685 | C | P | Yes | NS, NC | Q10618 | molecular_function |
| | | | | | | | | | | | | | unknown |
| 632 | 1457413 | G | G | 1459362 | T | V | 1458905 | T | V | Yes | NS, C | Q10606 | magnesium ion binding |
| | | | | | | | | | | | | | activity |
| 633 | 1458715 | T | V | 1460664 | C | V | 1460207 | C | V | Yes | S, NULL | Null | — |
| 634 | 1460269 | C | V | 1462218 | G | V | 1461761 | G | V | Yes | S, NULL | Null | — |
| 635 | 1465744 | T | V | 1467693 | C | A | 1467236 | C | A | Yes | NS, C | Q10620 | integral to membrane |
| 636 | 1465784 | A | G | 1467733 | G | G | 1467276 | G | G | Yes | S, NULL | Null | — |
| 637 | 1469253 | T | F | 1471215 | C | F | 1470758 | C | F | Yes | S, NULL | Null | — |
| 638 | 1476347 | T | L | 1478310 | C | L | 1477853 | C | L | Yes | S, NULL | Null | — |
| 639 | 1476918 | T | * | 1478881 | C | W | 1478424 | C | W | Yes | NS, TP | Q10630 | DNA binding activity |
| 640 | 1477120 | C | V | 1479083 | T | I | 1478626 | T | I | Yes | NS, C | Q10630 | DNA binding activity |
| 641 | 1487043 | C | A | 1489006 | T | T | 1490222 | T | T | Yes | NS, NC | Q10637 | — |
| 642 | 1488940 | G | P | 1490903 | A | S | 1492119 | A | S | Yes | NS, NC | Q10625 | |
| 643 | 1488946 | A | S | 1490909 | G | P | 1492125 | G | P | Yes | NS, NC | Q10625 | |
| 644 | 1490229 | C | G | 1492192 | T | D | 1493408 | T | D | Yes | NS, NC | Q10625 | |
| 645 | 1490640 | T | A | 1492603 | C | A | 1493819 | C | A | Yes | S, NULL | Null | — |
| 646 | 1494193 | G | G | 1496156 | A | D | 1497372 | A | D | Yes | NS, NC | Q10639 | enzyme activity |
| 647 | 1494324 | T | F | 1496287 | G | V | 1497503 | G | V | Yes | NS, NC | Q10639 | enzyme activity |
| 648 | 1494999 | T | S | 1496962 | G | A | 1498178 | G | A | Yes | NS, NC | Q10639 | enzyme activity |
| 649 | 1497326 | T | G | 1499289 | C | G | 1500505 | C | G | Yes | S, NULL | Null | — |
| 650 | 1497523 | T | K | 1499486 | C | E | 1500702 | C | E | Yes | NS, NC | Q10641 | — |
| 651 | 1499260 | G | R | 1501223 | T | R | 1502439 | T | R | Yes | S, NULL | Null | — |
| 652 | 1500014 | A | T | 1501977 | G | T | 1503193 | G | T | Yes | S, NULL | Null | — |
| 653 | 1503229 | C | G | 1505192 | T | G | 1506408 | T | G | Yes | S, NULL | Null | — |
| 654 | 1504008 | A | R | 1505971 | G | R | 1507187 | G | R | Yes | S, NULL | Null | — |
| 655 | 1505078 | A | S | 1507041 | G | G | 1508257 | G | G | Yes | NS, NC | Q10628 | tRNA binding activity |
| 656 | 1506717 | G | A | 1508680 | T | E | 1509896 | T | E | Yes | NS, NC | Q11013 | integral to membrane |
| 657 | 1521210 | C | A | 1523173 | A | A | 1524390 | A | A | Yes | S, NULL | Null | — |
| 658 | 1521826 | G | P | 1523789 | A | L | 1525006 | A | L | Yes | NS, NC | Q11025 | enzyme activity |
| 659 | 1524738 | T | V | 1526701 | G | G | 1527918 | G | G | Yes | NS, C | Q11028 | DNA binding activity |
| 660 | 1525971 | C | A | 1527934 | A | D | 1529151 | A | D | Yes | NS, NC | Q11028 | DNA binding activity |
| 661 | 1528766 | A | R | 1530729 | G | G | 1531946 | G | G | Yes | NS, NC | Q11029 | guanylate cyclase |
| | | | | | | | | | | | | | activity |
| 662 | 1530691 | T | A | 1532654 | G | A | 1533871 | G | A | Yes | S, NULL | Null | — |
| 663 | 1530694 | T | P | 1532657 | C | P | 1533874 | C | P | Yes | S, NULL | Null | — |
| 664 | 1530695 | G | P | 1532658 | T | P | 1533875 | T | P | Yes | S, NULL | Null | — |
| 665 | 1530763 | T | S | 1532726 | C | S | 1533943 | C | S | Yes | S, NULL | Null | — |
| 666 | 1530890 | T | N | 1532853 | C | S | 1534070 | C | S | Yes | NS, C | Q11031 | — |
| 667 | 1530894 | T | T | 1532857 | C | A | 1534074 | C | A | Yes | NS, NC | Q11031 | — |
| 668 | 1530957 | T | I | 1532920 | G | L | 1534137 | G | L | Yes | NS, C | Q11031 | — |
| 669 | 1531501 | C | G | 1533464 | T | G | 1534681 | T | G | Yes | S, NULL | Null | — |
| 670 | 1531505 | A | V | 1533468 | G | V | 1534685 | G | V | Yes | S, NULL | Null | — |
| 671 | 1531506 | C | V | 1533469 | T | V | 1534686 | T | V | Yes | S, NULL | Null | — |
| 672 | 1531581 | G | Q | 1533544 | T | K | 1534761 | T | K | Yes | NS, NC | Q11031 | — |
| 673 | 1531582 | A | A | 1533545 | C | A | 1534762 | C | A | Yes | S, NULL | Null | — |
| 674 | 1531585 | C | A | 1533548 | G | A | 1534765 | G | A | Yes | S, NULL | Null | — |
| 675 | 1532338 | C | G | 1534301 | A | V | 1535518 | A | V | Yes | NS, C | Q11032 | integral to membrane |
| 676 | 1532964 | T | N | 1534927 | G | T | 1536144 | G | T | Yes | NS, C | Q11033 | integral to membrane |
| 677 | 1534974 | T | T | 1536937 | C | A | 1538155 | C | A | Yes | NS, NC | Q11034 | two-component sensor |
| | | | | | | | | | | | | | molecule activity |
| 678 | 1535961 | T | K | 1537924 | C | E | 1539142 | C | E | Yes | NS, NC | Q11035 | — |
| 679 | 1537543 | A | Null | 1539506 | G | Null | 1540724 | G | null | No | nc, NULL | Null | — |
| 680 | 1538176 | C | V | 1540139 | T | I | 1541357 | T | I | Yes | NS, C | Q11037 | — |
| 681 | 1540933 | T | C | 1544253 | C | C | 1544113 | C | C | Yes | S, NULL | Null | — |
| 682 | 1543382 | T | A | 1546701 | C | A | 1546561 | C | A | Yes | S, NULL | Null | — |
| 683 | 1544766 | A | R | 1548085 | G | G | 1547945 | G | G | Yes | NS, NC | P71803 | — |
| 684 | 1544828 | A | P | 1548147 | G | P | 1548007 | G | P | Yes | S, NULL | Null | — |
| 685 | 1545475 | C | P | 1548794 | G | R | 1548654 | G | R | Yes | NS, NC | P71803 | — |
| 686 | 1546533 | C | P | 1549852 | G | R | 1549712 | G | R | Yes | NS, NC | P71804 | — |
| 687 | 1549309 | T | H | 1552628 | C | R | 1552488 | C | R | Yes | NS, NC | P71806 | — |
| 688 | 1551431 | T | F | 1554750 | G | V | 1554610 | G | V | Yes | NS, NC | P71809 | dihydroorotase activity |
| 689 | 1553002 | G | G | 1556321 | C | A | 1556181 | C | A | Yes | NS, C | P71811 | enzyme activity |
| 690 | 1556241 | A | E | 1559560 | C | A | 1559420 | C | A | Yes | NS, NC | Not | — |
| | | | | | | | | | | | | annotated | |
| 691 | 1556275 | T | H | 1559594 | C | H | 1559454 | C | H | Yes | S, NULL | Null | — |
| 692 | 1558090 | A | Null | 1561409 | G | Null | 1561269 | G | null | No | nc, NULL | Null | — |
| 693 | 1558582 | C | S | 1561901 | A | S | 1561761 | A | S | Yes | S, NULL | Null | — |
| 694 | 1558728 | C | A | 1562047 | T | V | 1561907 | T | V | Yes | NS, C | P71657 | — |
| 695 | 1563681 | G | A | 1567000 | A | T | 1566860 | A | T | Yes | NS, NC | P77899 | magnesium ion binding |
| | | | | | | | | | | | | | activity |
| 696 | 1569266 | T | H | 1572957 | C | R | 1572817 | C | null | Yes | NS, NC | P71664 | integral to membrane |
| 697 | 1570275 | T | Null | 1573966 | C | Null | 1573825 | C | null | No | nc, NULL | Null | — |
| 698 | 1570513 | C | G | 1574204 | T | D | 1574063 | T | D | Yes | NS, NC | P71665 | — |
| 699 | 1578358 | C | Null | 1582049 | T | Null | 1581905 | T | null | No | nc, NULL | Null | — |
| 700 | 1580686 | C | L | 1584377 | A | I | 1584233 | A | I | Yes | NS, C | P71675 | RNA binding activity |
| 701 | 1581590 | C | N | 1585281 | A | K | 1585137 | A | K | Yes | NS, NC | P71677 | enzyme activity |
| 702 | 1581711 | A | T | 1585402 | G | A | 1585258 | G | A | Yes | NS, NC | P71677 | enzyme activity |
| 703 | 1585618 | G | Null | 1589309 | T | Null | 1589165 | T | null | No | nc, NULL | Null | — |
| 704 | 1589763 | T | Null | 1593454 | C | Null | 1593310 | C | null | No | nc, NULL | Null | — |
| 705 | 1593041 | T | R | 1596732 | C | R | 1596588 | C | R | Yes | S, NULL | Null | — |
| 706 | 1593444 | T | V | 1597135 | C | A | 1596991 | C | A | Yes | NS, C | P71691 | — |
| 707 | 1596992 | T | L | 1600683 | C | P | 1600539 | C | P | Yes | NS, NC | P71694 | molecular_function |
| | | | | | | | | | | | | | unknown |
| 708 | 1604583 | C | T | 1608274 | A | N | 1608132 | A | N | Yes | NS, C | O06827 | — |
| 709 | 1605752 | C | Q | 1609443 | A | K | 1609301 | A | K | Yes | NS, NC | O06827 | — |
| 710 | 1607590 | C | Null | 1611281 | T | Null | 1611139 | T | null | No | nc, NULL | Null | — |
| 711 | 1609080 | T | T | 1612750 | C | A | 1612635 | C | A | Yes | NS, NC | O06823 | — |
| 712 | 1614744 | A | G | 1618414 | G | G | 1618299 | G | null | Yes | S, NULL | Null | — |
| 713 | 1614952 | T | N | 1618622 | G | T | 1618507 | G | null | Yes | NS, C | O06818 | structural molecule |
| | | | | | | | | | | | | | activity |
| 714 | 1615306 | C | G | 1618976 | T | D | 1618860 | T | null | Yes | NS, NC | O06818 | structural molecule |
| | | | | | | | | | | | | | activity |
| 715 | 1627846 | G | G | 1631524 | A | G | 1631407 | A | G | Yes | S, NULL | Null | — |
| 716 | 1628460 | G | H | 1632138 | T | N | 1632021 | T | N | Yes | NS, NC | O06810 | — |
| 717 | 1629691 | G | N | 1633342 | A | N | 1633252 | A | N | Yes | S, NULL | Null | — |
| 718 | 1631814 | A | T | 1635228 | G | A | 1635345 | G | A | Yes | NS, NC | O06809 | heme biosynthesis |
| 719 | 1636164 | G | L | 1639416 | A | L | 1639521 | A | L | Yes | S, NULL | Null | — |
| 720 | 1636389 | C | R | 1639641 | T | R | 1639746 | T | R | Yes | S, NULL | Null | — |
| 721 | 1637188 | T | G | 1640440 | C | G | 1640545 | C | G | Yes | S, NULL | Null | — |
| 722 | 1643728 | T | V | 1646980 | C | A | 1647138 | C | A | Yes | NS, C | O53151 | transcription factor |
| | | | | | | | | | | | | | activity |
| 723 | 1643863 | C | G | 1647115 | T | G | 1647273 | T | G | Yes | S, NULL | Null | — |
| 724 | 1656740 | T | S | 1659992 | C | P | 1660150 | C | P | Yes | NS, NC | O53163 | enzyme activity |
| 725 | 1657398 | T | Null | 1660650 | G | Null | 1660808 | G | null | No | nc, NULL | Null | — |
| 726 | 1657399 | T | Null | 1660651 | A | Null | 1660809 | A | null | No | nc, NULL | Null | — |
| 727 | 1658616 | A | Q | 1661868 | G | Q | 1662026 | G | Q | Yes | S, NULL | Null | — |
| 728 | 1659304 | C | P | 1662556 | T | Null | 1662714 | T | S | Yes | nc, NULL | Null | — |
| 729 | 1659465 | C | A | 1662717 | G | A | 1662875 | G | A | Yes | S, NULL | Null | — |
| 730 | 1668404 | T | R | 1671656 | C | R | 1671814 | C | R | Yes | S, NULL | Null | — |
| 731 | 1669135 | C | Null | 1672387 | T | Null | 1672545 | T | null | No | nc, NULL | Null | — |
| 732 | 1678674 | T | V | 1681926 | C | V | 1682084 | C | V | Yes | S, NULL | Null | — |
| 733 | 1681725 | C | S | 1684977 | T | S | 1685135 | T | S | Yes | S, NULL | Null | — |
| 734 | 1683015 | T | Null | 1686267 | C | Null | 1686425 | C | null | No | nc, NULL | Null | — |
| 735 | 1685046 | C | F | 1688298 | T | F | 1688456 | T | F | Yes | S, NULL | Null | — |
| 736 | 1687091 | G | Null | 1690343 | A | Null | 1690501 | A | null | Yes | S, NULL | Null | — |
| 737 | 1690478 | T | C | 1693731 | C | C | 1693889 | C | C | Yes | S, NULL | Null | — |
| 738 | 1690944 | T | K | 1694197 | C | E | 1694355 | C | null | Yes | NS, NC | CAB02017 | — |
| 739 | 1691292 | C | E | 1694545 | A | * | 1694703 | A | null | Yes | NS, TP | CAB02018 | — |
| 740 | 1692419 | A | Y | 1695672 | G | Y | 1695830 | G | Y | Yes | S, NULL | Null | — |
| 741 | 1694454 | A | R | 1710439 | C | S | 1710596 | C | S | Yes | NS, NC | Q50590 | integral to membrane |
| 742 | 1694605 | C | L | 1710590 | A | M | 1710747 | A | M | Yes | NS, NC | Q50590 | integral to membrane |
| 743 | 1696535 | T | I | 1712520 | G | S | 1712677 | G | S | Yes | NS, NC | Q50586 | enzyme activity |
| 744 | 1698303 | T | * | 1714288 | G | S | 1714445 | G | S | Yes | NS, TP | Q50585 | membrane |
| 745 | 1698786 | T | N | 1714771 | C | S | 1714928 | C | S | Yes | NS, C | Q50585 | membrane |
| 746 | 1700485 | G | P | 1716470 | A | S | 1716627 | A | S | Yes | NS, NC | Q50585 | membrane |
| 747 | 1701016 | C | A | 1717001 | T | T | 1717158 | T | T | Yes | NS, NC | Q50585 | membrane |
| 748 | 1703404 | T | F | 1719389 | C | F | 1719546 | C | F | Yes | S, NULL | Null | — |
| 749 | 1708108 | G | L | 1724093 | A | F | 1724250 | A | null | Yes | NS, NC | O53901 | enzyme activity |
| 750 | 1710829 | T | T | 1726814 | G | P | 1726970 | G | null | Yes | NS, NC | O53901 | enzyme activity |
| 751 | 1712677 | T | Null | 1728662 | C | Null | 1728818 | C | null | Yes | nc, NULL | Null | — |
| 752 | 1715811 | A | A | 1731796 | G | A | 1731952 | G | A | Yes | S, NULL | Null | — |
| 753 | 1723307 | G | A | 1739292 | C | P | 1739447 | C | P | Yes | NS, NC | Q10765 | tRNA ligase activity |
| 754 | 1734934 | A | A | 1750919 | C | A | 1751074 | C | A | Yes | S, NULL | Null | — |
| 755 | 1737144 | G | A | 1753129 | A | V | 1753284 | A | V | Yes | NS, C | Q10778 | integral to membrane |
| 756 | 1737486 | C | Null | 1753471 | G | Null | 1753626 | G | null | Yes | nc, NULL | Null | — |
| 757 | 1738210 | A | G | 1754193 | G | G | 1754349 | G | G | Yes | S, NULL | Null | — |
| 758 | 1738587 | C | S | 1754570 | T | L | 1754726 | T | L | Yes | NS, NC | Q10776 | enzyme activity |
| 759 | 1744942 | C | T | 1760921 | T | T | 1761077 | T | T | Yes | S, NULL | Null | — |
| 760 | 1752792 | T | T | 1766618 | C | A | 1766774 | C | A | Yes | NS, NC | Q10769 | hydrolase activity |
| 761 | 1760533 | A | Null | 1775165 | G | Null | 1775321 | G | null | No | nc, NULL | Null | — |
| 762 | 1779600 | A | A | 1794232 | G | A | 1785141 | G | A | Yes | S, NULL | Null | — |
| 763 | 1782943 | G | D | 1797575 | A | N | 1788484 | A | N | Yes | NS, NC | O06594 | nicotinate-nucleotide |
| | | | | | | | | | | | | | pyrophosphorylase |
| | | | | | | | | | | | | | (carboxylating) activity |
| 764 | 1785887 | G | Q | 1800519 | A | Q | 1791428 | A | Q | Yes | S, NULL | Null | — |
| 765 | 1789614 | A | E | 1804246 | G | E | 1795155 | G | E | Yes | S, NULL | Null | — |
| 766 | 1789681 | C | H | 1804313 | T | Y | 1795222 | T | Y | Yes | NS, C | O53907 | inositol/ |
| | | | | | | | | | | | | | phosphatidylinositol |
| | | | | | | | | | | | | | phosphatase |
| | | | | | | | | | | | | | activity |
| 767 | 1790156 | T | L | 1804788 | C | P | 1795697 | C | P | Yes | NS, NC | O53907 | inositol/ |
| | | | | | | | | | | | | | phosphatidylinositol |
| | | | | | | | | | | | | | phosphatase |
| | | | | | | | | | | | | | activity |
| 768 | 1790580 | T | L | 1805212 | C | P | 1796121 | C | P | Yes | NS, NC | O53908 | histidine biosynthesis |
| 769 | 1798626 | C | V | 1813258 | T | V | 1804167 | T | V | Yes | S, NULL | Null | — |
| 770 | 1802214 | T | D | 1816846 | G | E | 1807755 | G | E | Yes | NS, C | O06134 | magnesium ion binding |
| | | | | | | | | | | | | | activity |
| 771 | 1808750 | C | T | 1823382 | G | T | 1814291 | G | T | Yes | S, NULL | Null | — |
| 772 | 1811420 | C | A | 1826052 | T | T | 1816961 | T | T | Yes | NS, NC | O06141 | — |
| 773 | 1813171 | T | V | 1827803 | C | V | 1818712 | C | V | Yes | S, NULL | Null | — |
| 774 | 1813366 | A | Null | 1827998 | G | Null | 1818907 | G | null | No | nc, NULL | Null | — |
| 775 | 1813755 | T | R | 1828387 | C | R | 1819296 | C | R | Yes | S, NULL | Null | — |
| 776 | 1815661 | A | F | 1830293 | G | F | 1821202 | G | F | Yes | S, NULL | Null | — |
| 777 | 1820225 | A | T | 1834857 | G | A | 1825766 | G | A | Yes | NS, NC | O06147 | RNA binding activity |
| 778 | 1822223 | T | Null | 1836855 | G | Null | 1827764 | G | null | No | nc, NULL | Null | — |
| 779 | 1824626 | G | L | 1839258 | T | L | 1830167 | T | L | Yes | S, NULL | Null | — |
| 780 | 1825125 | C | R | 1839757 | G | G | 1830666 | G | G | Yes | NS, NC | O06151 | transporter activity |
| 781 | 1828921 | A | N | 1843553 | G | N | 1834462 | G | N | Yes | S, NULL | Null | — |
| 782 | 1834571 | C | G | 1849203 | T | D | 1840112 | T | D | Yes | NS, NC | P94974 | magnesium ion binding |
| | | | | | | | | | | | | | activity |
| 783 | 1834975 | C | R | 1849607 | T | R | 1840516 | T | R | Yes | S, NULL | Null | — |
| 784 | 1844924 | A | D | 1859557 | C | A | 1850466 | C | A | Yes | NS, NC | P94984 | magnesium ion binding |
| | | | | | | | | | | | | | activity |
| 785 | 1845757 | T | V | 1860390 | C | A | 1851299 | C | A | Yes | NS, C | P94985 | tRNA binding activity |
| 786 | 1850233 | A | V | 1864866 | G | A | 1855775 | G | A | Yes | NS, C | P94986 | — |
| 787 | 1858345 | G | R | 1872957 | A | R | 1863865 | A | R | Yes | S, NULL | Null | — |
| 788 | 1862697 | T | W | 1877309 | C | R | 1868217 | C | R | Yes | NS, NC | P94996 | enzyme activity |
| 789 | 1863620 | A | T | 1878232 | G | T | 1869140 | G | T | Yes | S, NULL | Null | — |
| 790 | 1864215 | C | P | 1878827 | T | S | 1869735 | T | S | Yes | NS, NC | P94996 | enzyme activity |
| 791 | 1867321 | T | Y | 1881933 | G | D | 1872841 | G | D | Yes | NS, NC | O65933 | enzyme activity |
| 792 | 1867566 | T | A | 1882178 | C | A | 1873086 | C | A | Yes | S, NULL | Null | — |
| 793 | 1869512 | T | V | 1884124 | C | A | 1875032 | C | A | Yes | NS, C | O65933 | enzyme activity |
| 794 | 1869897 | A | V | 1884509 | G | V | 1875417 | G | V | Yes | S, NULL | Null | — |
| 795 | 1870867 | G | G | 1885479 | C | R | 1876387 | C | R | Yes | NS, NC | O65933 | enzyme activity |
| 796 | 1871495 | G | C | 1886107 | A | Y | 1877015 | A | Y | Yes | NS, NC | O65933 | enzyme activity |
| 797 | 1873514 | A | T | 1888126 | G | A | 1879034 | G | A | Yes | NS, NC | O06586 | enzyme activity |
| 798 | 1874459 | C | P | 1889071 | G | A | 1879979 | G | A | Yes | NS, NC | O06586 | enzyme activity |
| 799 | 1878859 | A | T | 1893471 | G | T | 1884379 | G | T | Yes | S, NULL | Null | — |
| 800 | 1885417 | T | Null | 1900019 | C | Null | 1890823 | C | null | Yes | nc, NULL | Null | — |
| 801 | 1886196 | G | A | 1900798 | A | V | 1891602 | A | V | Yes | NS, C | O53922 | transcription factor |
| | | | | | | | | | | | | | activity |
| 802 | 1888569 | T | I | 1903171 | C | I | 1893975 | C | I | Yes | S, NULL | Null | — |
| 803 | 1890513 | G | G | 1905115 | C | A | 1895919 | C | A | Yes | NS, C | O33182 | — |
| 804 | 1891732 | G | Null | 1906334 | A | Null | 1897138 | A | null | No | nc, NULL | Null | — |
| 805 | 1897364 | A | F | 1912022 | C | V | 1902826 | C | V | Yes | NS, NC | O33188 | drug transporter activity |
| 806 | 1897922 | A | A | 1912580 | G | A | 1903384 | G | A | Yes | S, NULL | Null | — |
| 807 | 1899910 | C | A | 1914568 | A | A | 1905372 | A | A | Yes | S, NULL | Null | — |
| 808 | 1905462 | T | P | 1920118 | G | P | 1910922 | G | P | Yes | S, NULL | Null | — |
| 809 | 1910301 | C | G | 1924957 | T | G | 1915761 | T | G | Yes | S, NULL | Null | — |
| 810 | 1911052 | G | L | 1925708 | C | F | 1916512 | C | F | Yes | NS, NC | O33199 | — |
| 811 | 1911527 | A | T | 1926183 | G | A | 1916987 | G | A | Yes | NS, NC | O33199 | — |
| 812 | 1915691 | T | A | 1930348 | C | A | 1921152 | C | A | Yes | S, NULL | Null | — |
| 813 | 1916811 | A | Null | 1931468 | G | Null | 1922272 | G | null | No | nc, NULL | Null | — |
| 814 | 1917059 | C | V | 1931716 | G | L | 1922520 | G | L | Yes | NS, C | O33204 | — |
| 815 | 1921036 | G | A | 1935693 | T | S | 1926497 | T | S | Yes | NS, NC | O33206 | sulfate porter activity |
| 816 | 1921535 | A | Q | 1936192 | G | R | 1926996 | G | R | Yes | NS, NC | O33206 | sulfate porter activity |
| 817 | 1921866 | A | T | 1936523 | G | A | 1927327 | G | null | Yes | NS, NC | O33207 | — |
| 818 | 1928563 | A | Null | 1943220 | G | Null | 1934024 | G | null | Yes | S, NULL | Null | — |
| 819 | 1933291 | A | M | 1947949 | G | V | 1938753 | G | V | Yes | NS, NC | P71980 | — |
| 820 | 1934421 | T | R | 1949079 | C | R | 1939883 | C | R | Yes | S, NULL | Null | — |
| 821 | 1937104 | A | Null | 1951762 | G | Null | 1942565 | G | null | No | nc, NULL | Null | — |
| 822 | 1937372 | C | P | 1952030 | G | A | 1942833 | G | A | Yes | NS, NC | P71984 | sugar porter activity |
| 823 | 1938167 | T | Y | 1952825 | C | H | 1943628 | C | H | Yes | NS, C | P71984 | sugar porter activity |
| 824 | 1941920 | A | T | 1956521 | C | T | 1947381 | C | T | Yes | S, NULL | Null | — |
| 825 | 1942743 | C | Null | 1957344 | T | Null | 1948204 | T | null | Yes | nc, NULL | Null | — |
| 826 | 1946179 | C | A | 1960780 | T | T | 1951640 | T | A | Yes | NS, NC | P71992 | — |
| 827 | 1948447 | C | R | 1963048 | T | R | 1953908 | T | R | Yes | S, NULL | Null | — |
| 828 | 1949354 | C | G | 1963955 | T | D | 1954815 | T | D | Yes | NS, NC | P71994 | electron transport |
| 829 | 1949427 | G | H | 1964028 | C | D | 1954888 | C | D | Yes | NS, NC | P71994 | electron transport |
| 830 | 1950831 | G | Null | 1965432 | A | Null | 1956292 | A | null | No | nc, NULL | Null | — |
| 831 | 1953513 | C | T | 1968114 | A | K | 1958974 | A | K | Yes | NS, NC | P71999 | — |
| 832 | 1953569 | T | Null | 1968170 | G | Null | 1959030 | G | null | No | nc, NULL | Null | — |
| 833 | 1954568 | A | D | 1969168 | T | V | 1960028 | T | V | Yes | NS, NC | P72001 | protein kinase activity |
| 834 | 1956427 | T | Q | 1971027 | C | R | 1961887 | C | R | Yes | NS, NC | O06787 | — |
| 835 | 1956859 | T | Null | 1971459 | G | Null | 1962319 | G | null | Yes | S, NULL | Null | — |
| 836 | 1957123 | C | R | 1971723 | G | R | 1962583 | G | R | Yes | S, NULL | Null | — |
| 837 | 1957247 | G | S | 1971847 | A | F | 1962707 | A | F | Yes | NS, NC | P72002 | isopentenyl- |
| | | | | | | | | | | | | | diphosphate delta- |
| | | | | | | | | | | | | | isomerase activity |
| 838 | 1958508 | A | T | 1973108 | G | A | 1963968 | G | A | Yes | NS, NC | P72003 | protein kinase activity |
| 839 | 1961358 | T | S | 1975958 | G | S | 1966818 | G | S | Yes | S, NULL | Null | — |
| 840 | 1965950 | C | T | 1980550 | T | M | 1971410 | T | M | Yes | NS, NC | O65936 | monooxygenase activity |
| 841 | 1981202 | G | G | 1990359 | A | G | 1988183 | A | null | Yes | S, NULL | Null | — |
| 842 | 1982624 | G | G | 1991781 | A | G | 1989605 | A | null | Yes | S, NULL | Null | — |
| 843 | 1985405 | T | T | 1994564 | C | T | 1992387 | C | T | Yes | S, NULL | Null | — |
| 844 | 1989704 | G | R | 1999262 | A | Null | 1996686 | A | null | Yes | nc, NULL | Null | — |
| 845 | 1991672 | C | H | 2001230 | A | N | 1998654 | A | null | Yes | NS, NC | O06801 | — |
| 846 | 1993549 | T | L | 2003089 | C | L | 2000511 | C | L | Yes | S, NULL | Null | — |
| 847 | 1997652 | A | F | 2007192 | G | F | 2004614 | G | F | Yes | S, NULL | Null | — |
| 848 | 2001072 | A | Null | 2010612 | G | Null | 2008034 | G | null | No | nc, NULL | Null | — |
| 849 | 2002085 | G | Q | 2011625 | C | H | 2009047 | C | H | Yes | NS, NC | O33180 | monooxygenase activity |
| 850 | 2002894 | C | R | 2012434 | G | T | 2009856 | G | T | Yes | NS, NC | O33181 | — |
| 851 | 2004047 | T | L | 2013587 | C | L | 2011009 | C | L | Yes | S, NULL | Null | — |
| 852 | 2007749 | C | R | 2017289 | T | R | 2014711 | T | R | Yes | S, NULL | Null | — |
| 853 | 2008319 | A | H | 2017859 | G | R | 2015281 | G | R | Yes | NS, NC | O53933 | — |
| 854 | 2009694 | G | P | 2019234 | T | P | 2016656 | T | P | Yes | S, NULL | Null | — |
| 855 | 2011784 | G | A | 2021324 | A | A | 2018746 | A | A | Yes | S, NULL | Null | — |
| 856 | 2012022 | A | M | 2021562 | G | V | 2018984 | G | V | Yes | NS, NC | O53935 | nucleotide binding |
| | | | | | | | | | | | | | activity |
| 857 | 2017231 | A | Null | 2026774 | G | Null | 2024196 | G | null | Yes | nc, NULL | Null | — |
| 858 | 2018064 | G | A | 2027607 | T | S | 2025029 | T | S | Yes | NS, NC | O53939 | — |
| 859 | 2019447 | T | S | 2028990 | C | P | 2026412 | C | P | Yes | NS, NC | O53940 | — |
| 860 | 2019644 | G | P | 2029187 | A | P | 2026609 | A | P | Yes | S, NULL | Null | — |
| 861 | 2020481 | T | P | 2030024 | C | P | 2027446 | C | P | Yes | S, NULL | Null | — |
| 862 | 2020767 | T | Null | 2030310 | C | Null | 2027732 | C | null | No | nc, NULL | Null | — |
| 863 | 2020942 | T | G | 2030485 | C | G | 2027907 | C | null | Yes | S, NULL | Null | — |
| 864 | 2020943 | C | Q | 2030486 | A | Q | 2027908 | A | null | Yes | S, NULL | Null | — |
| 865 | 2020944 | A | Q | 2030487 | T | Q | 2027909 | T | null | Yes | S, NULL | Null | — |
| 866 | 2020976 | C | Q | 2030519 | T | * | 2027941 | T | null | Yes | NS, TP | Not | — |
| | | | | | | | | | | | | annotated | |
| 867 | 2021631 | T | P | 2031174 | G | P | 2028596 | G | P | Yes | S, NULL | Null | — |
| 868 | 2022570 | G | Null | 2032113 | A | Null | 2029535 | A | null | No | nc, NULL | Null | — |
| 869 | 2023460 | G | A | 2033003 | A | T | 2030425 | A | T | Yes | NS, NC | O53944 | — |
| 870 | 2023476 | A | D | 2033019 | G | G | 2030441 | G | G | Yes | NS, NC | O53944 | — |
| 871 | 2030339 | G | W | 2039882 | T | C | 2037304 | T | C | Yes | NS, NC | O53949 | — |
| 872 | 2030664 | G | V | 2040207 | T | F | 2037629 | T | F | Yes | NS, NC | O53949 | — |
| 873 | 2031415 | C | A | 2040958 | T | V | 2038380 | T | V | Yes | NS, C | O53949 | — |
| 874 | 2037036 | A | * | 2046579 | G | Q | 2044001 | G | Q | Yes | NS, TP | O53952 | — |
| 875 | 2037314 | C | Null | 2046857 | T | Null | 2044279 | T | null | Yes | nc, NULL | Null | — |
| 876 | 2039950 | T | P | 2049493 | C | P | 2046915 | C | P | Yes | S, NULL | Null | — |
| 877 | 2040516 | G | S | 2050059 | A | S | 2047481 | A | S | Yes | S, NULL | Null | — |
| 878 | 2041277 | C | A | 2050820 | G | G | 2048242 | G | G | Yes | NS, C | O53957 | — |
| 879 | 2042298 | T | * | 2051841 | C | Q | 2049263 | C | Q | Yes | NS, TP | O53958 | proton transport |
| 880 | 2043142 | C | S | 2052685 | G | * | 2050107 | G | * | Yes | NS, TP | O53958 | proton transport |
| 881 | 2044762 | A | T | 2054305 | G | T | 2051727 | G | T | Yes | S, NULL | Null | — |
| 882 | 2048737 | G | K | 2058280 | A | K | 2055702 | A | K | Yes | S, NULL | Null | — |
| 883 | 2052442 | T | S | 2061976 | C | G | 2059405 | C | G | Yes | NS, NC | Q50615 | integral to membrane |
| 884 | 2053144 | G | Null | 2062678 | A | Null | 2060017 | A | null | Yes | nc, NULL | Null | — |
| 885 | 2053386 | C | V | 2062920 | T | I | 2060259 | T | I | Yes | NS, C | Q50614 | nucleotide binding |
| | | | | | | | | | | | | | activity |
| 886 | 2054840 | G | A | 2064374 | A | V | 2061713 | A | V | Yes | NS, C | Q50614 | nucleotide binding |
| | | | | | | | | | | | | | activity |
| 887 | 2055511 | A | P | 2065045 | G | P | 2062384 | G | P | Yes | S, NULL | Null | — |
| 888 | 2062654 | C | A | 2072188 | A | E | 2069527 | A | E | Yes | NS, NC | Q50607 | glycine cleavage |
| | | | | | | | | | | | | | system complex |
| 889 | 2064683 | T | C | 2074217 | C | R | 2071556 | C | R | Yes | NS, NC | Q50604 | — |
| 890 | 2065441 | C | Y | 2075087 | T | Y | 2072314 | T | Y | Yes | S, NULL | Null | — |
| 891 | 2066872 | C | A | 2076518 | T | V | 2073745 | T | V | Yes | NS, C | Q50601 | glycine cleavage |
| | | | | | | | | | | | | | system |
| 892 | 2067570 | G | A | 2077216 | A | T | 2074443 | A | T | Yes | NS, NC | Q50601 | glycine cleavage |
| | | | | | | | | | | | | | system |
| 893 | 2068670 | T | V | 2078316 | C | V | 2075543 | C | V | Yes | S, NULL | Null | — |
| 894 | 2070996 | C | A | 2080642 | T | V | 2077869 | T | V | Yes | NS, C | Q50599 | enzyme activity |
| 895 | 2073217 | T | H | 2082863 | C | R | 2080091 | C | R | Yes | NS, NC | Q50597 | integral to membrane |
| 896 | 2074580 | A | C | 2084226 | G | R | 2081454 | G | R | Yes | NS, NC | Q50597 | integral to membrane |
| 897 | 2080993 | G | S | 2090774 | A | L | 2088002 | A | L | Yes | NS, NC | Q50592 | integral to membrane |
| 898 | 2082905 | A | G | 2092686 | G | G | 2089914 | G | G | Yes | S, NULL | Null | — |
| 899 | 2083932 | C | K | 2093713 | T | Null | 2090941 | T | null | Yes | nc, NULL | Null | — |
| 900 | 2086536 | A | Y | 2096324 | C | D | 2093546 | C | D | Yes | NS, NC | P95163 | — |
| 901 | 2087337 | T | N | 2097126 | C | N | 2094348 | C | N | Yes | S, NULL | Null | — |
| 902 | 2087353 | C | L | 2097142 | G | V | 2094364 | G | V | Yes | NS, C | P95162 | enzyme activity |
| 903 | 2090002 | T | L | 2099791 | A | M | 2097013 | A | M | Yes | NS, NC | P50050 | nickel ion binding |
| | | | | | | | | | | | | | activity |
| 904 | 2092402 | A | W | 2102191 | G | R | 2099413 | G | R | Yes | NS, NC | P95160 | electron transport |
| 905 | 2094478 | T | H | 2104268 | C | R | 2101490 | C | R | Yes | NS, NC | P95158 | metabolism |
| 906 | 2094633 | T | A | 2104423 | C | A | 2101645 | C | A | Yes | S, NULL | Null | — |
| 907 | 2097719 | T | M | 2107509 | C | T | 2104731 | C | T | Yes | NS, NC | P95155 | nucleotide binding |
| | | | | | | | | | | | | | activity |
| 908 | 2099098 | C | Null | 2108888 | A | Null | 2106110 | A | null | No | nc, NULL | Null | — |
| 909 | 2099682 | T | Null | 2109472 | C | Null | 2106694 | C | null | No | nc, NULL | Null | — |
| 910 | 2100574 | C | T | 2110363 | A | T | 2107586 | A | T | Yes | S, NULL | Null | — |
| 911 | 2103397 | C | Q | 2113186 | G | E | 2110409 | G | E | Yes | NS, NC | P95149 | DNA binding activity |
| 912 | 2110986 | T | L | 2120775 | C | Null | 2117998 | C | null | Yes | nc, NULL | Null | — |
| 913 | 2111005 | A | L | 2120794 | T | * | 2118017 | T | * | Yes | NS, TP | P95145 | base-excision repair |
| 914 | 2112834 | G | A | 2122623 | A | V | 2119846 | A | V | Yes | NS, C | P95143 | electron transport |
| 915 | 2113185 | G | A | 2122974 | C | G | 2120197 | C | G | Yes | NS, C | P95143 | electron transport |
| 916 | 2113487 | G | G | 2123276 | T | G | 2120499 | T | G | Yes | S, NULL | Null | — |
| 917 | 2113686 | A | N | 2123475 | G | D | 2120698 | G | D | Yes | NS, NC | O07756 | — |
| 918 | 2116072 | T | Null | 2125861 | C | Null | 2123084 | C | null | No | nc, NULL | Null | — |
| 919 | 2118582 | G | T | 2128370 | A | T | 2125593 | A | T | Yes | S, NULL | Null | — |
| 920 | 2119054 | A | T | 2128842 | G | A | 2126065 | G | A | Yes | NS, NC | O07752 | glutamate-ammonia |
| | | | | | | | | | | | | | ligase activity |
| 921 | 2119491 | A | A | 2129279 | C | A | 2126502 | C | A | Yes | S, NULL | Null | — |
| 922 | 2120739 | G | Null | 2130527 | A | Null | 2127750 | A | null | No | nc, NULL | Null | — |
| 923 | 2128138 | A | G | 2137901 | G | G | 2135149 | G | G | Yes | S, NULL | Null | — |
| 924 | 2134872 | A | D | 2144635 | G | D | 2141883 | G | D | Yes | S, NULL | Null | — |
| 925 | 2136113 | G | P | 2145876 | A | L | 2143124 | A | L | Yes | NS, NC | O07733 | — |
| 926 | 2137405 | T | Q | 2147123 | C | R | 2144416 | C | R | Yes | NS, NC | O07732 | guanylate cyclase |
| | | | | | | | | | | | | | activity |
| 927 | 2141458 | T | D | 2151176 | C | D | 2148469 | C | D | Yes | S, NULL | Null | — |
| 928 | 2141625 | T | M | 2151343 | C | T | 2148636 | C | T | Yes | NS, NC | O07728 | — |
| 929 | 2141958 | T | T | 2151676 | G | P | 2148969 | G | P | Yes | NS, NC | O07727 | monooxygenase activity |
| 930 | 2145004 | A | L | 2154722 | C | R | 2152015 | C | R | Yes | NS, NC | Q08129 | catalase activity |
| 931 | 2145783 | A | T | 2155501 | G | T | 2152794 | G | T | Yes | S, NULL | Null | — |
| 932 | 2146305 | T | P | 2156023 | G | P | 2153316 | G | P | Yes | S, NULL | Null | — |
| 933 | 2147148 | T | G | 2156866 | G | G | 2154159 | G | G | Yes | S, NULL | Null | — |
| 934 | 2147202 | T | T | 2156920 | A | T | 2154213 | A | T | Yes | S, NULL | Null | — |
| 935 | 2148389 | C | G | 2158107 | T | D | 2155400 | T | D | Yes | NS, NC | O07721 | NADPH: quinone |
| | | | | | | | | | | | | | reductase activity |
| 936 | 2153341 | A | L | 2163058 | G | L | 2160351 | G | L | Yes | S, NULL | Null | — |
| 937 | 2159560 | G | S | 2167924 | A | L | 2165280 | A | L | Yes | NS, NC | O53960 | — |
| 938 | 2159645 | G | R | 2168009 | T | S | 2165365 | T | S | Yes | NS, NC | O53960 | — |
| 939 | 2159953 | A | I | 2168317 | G | T | 2165673 | G | T | Yes | NS, NC | O53960 | — |
| 940 | 2163647 | C | I | 2172010 | G | M | 2169366 | G | M | Yes | NS, NC | O53962 | metabolism |
| 941 | 2166221 | A | R | 2174584 | C | R | 2171940 | C | R | Yes | S, NULL | Null | — |
| 942 | 2167025 | A | D | 2175388 | G | G | 2172744 | G | G | Yes | NS, NC | P95290 | — |
| 943 | 2167348 | A | N | 2175711 | G | D | 2173067 | G | D | Yes | NS, NC | P95290 | — |
| 944 | 2168708 | C | Null | 2177071 | T | Null | 2174427 | T | null | No | nc, NULL | Null | — |
| 945 | 2180277 | T | C | 2188640 | C | C | 2185973 | C | C | Yes | S, NULL | Null | — |
| 946 | 2188349 | C | L | 2196713 | G | V | 2194046 | G | V | Yes | NS, C | P95269 | — |
| 947 | 2188810 | A | T | 2197174 | G | T | 2194507 | G | T | Yes | S, NULL | Null | — |
| 948 | 2189303 | T | T | 2197667 | C | A | 2195000 | C | A | Yes | NS, NC | P95268 | — |
| 949 | 2190686 | G | R | 2199050 | C | G | 2196383 | C | G | Yes | NS, NC | P95266 | — |
| 950 | 2191050 | G | L | 2199414 | A | F | 2196747 | A | F | Yes | NS, NC | P95265 | — |
| 951 | 2192930 | C | R | 2201294 | T | H | 2198627 | T | null | Yes | NS, NC | P95260 | — |
| 952 | 2200251 | G | G | 2221333 | A | D | 2218667 | A | D | Yes | NS, NC | O53979 | S-adenosylmethionine- |
| | | | | | | | | | | | | | dependent |
| | | | | | | | | | | | | | methyltransferase |
| | | | | | | | | | | | | | activity |
| 953 | 2201224 | C | G | 2222306 | T | D | 2219640 | T | D | Yes | NS, NC | Q10875 | amino acid-polyamine |
| | | | | | | | | | | | | | transporter activity |
| 954 | 2204091 | C | R | 2225173 | A | L | 2222507 | A | L | Yes | NS, NC | Q10840 | ribonucleoside- |
| | | | | | | | | | | | | | diphosphate reductase |
| | | | | | | | | | | | | | activity |
| 955 | 2205817 | A | T | 2226899 | G | A | 2224233 | G | A | Yes | NS, NC | Q10873 | integral to membrane |
| 956 | 2208717 | G | P | 2229799 | C | P | 2227133 | C | P | Yes | S, NULL | Null | — |
| 957 | 2210402 | G | Null | 2231484 | A | Null | 2228818 | A | null | No | nc, NULL | Null | — |
| 958 | 2220658 | G | P | 2241740 | A | P | 2239074 | A | P | Yes | S, NULL | Null | — |
| 959 | 2221950 | G | R | 2243032 | T | S | 2240366 | T | S | Yes | NS, NC | Q10859 | enzyme activity |
| 960 | 2223259 | T | R | 2244341 | G | R | 2241675 | G | R | Yes | S, NULL | Null | — |
| 961 | 2225876 | C | V | 2246958 | G | V | 2244292 | G | V | Yes | S, NULL | Null | — |
| 962 | 2226085 | A | H | 2247167 | G | R | 2244501 | G | R | Yes | NS, NC | Q10856 | — |
| 963 | 2231155 | A | S | 2252237 | G | G | 2249571 | G | G | Yes | NS, NC | Q10850 | carbohydrate |
| | | | | | | | | | | | | | metabolism |
| 964 | 2232015 | C | A | 2253097 | A | A | 2250431 | A | A | Yes | S, NULL | Null | — |
| 965 | 2234651 | C | P | 2255733 | T | L | 2253067 | T | L | Yes | NS, NC | Q10850 | carbohydrate |
| | | | | | | | | | | | | | metabolism |
| 966 | 2237132 | G | D | 2258214 | A | N | 2255548 | A | N | Yes | NS, NC | Q10848 | — |
| 967 | 2239016 | T | Null | 2260098 | C | Null | 2257432 | C | null | No | nc, NULL | Null | — |
| 968 | 2240157 | G | E | 2261239 | A | E | 2258573 | A | E | Yes | S, NULL | Null | — |
| 969 | 2240609 | C | Null | 2261691 | G | Null | 2259025 | G | null | No | nc, NULL | Null | — |
| 970 | 2244542 | G | G | 2265624 | A | D | 2262958 | A | D | Yes | NS, NC | O53464 | — |
| 971 | 2246892 | T | R | 2267974 | G | R | 2265308 | G | R | Yes | S, NULL | Null | — |
| 972 | 2247313 | T | L | 2268395 | C | Null | 2265729 | C | L | Yes | nc, NULL | Null | — |
| 973 | 2253136 | C | V | 2269218 | A | F | 2271552 | A | F | Yes | NS, NC | O53470 | ATP binding activity |
| 974 | 2253292 | A | C | 2269374 | G | R | 2271708 | G | R | Yes | NS, NC | O53470 | ATP binding activity |
| 975 | 2255734 | G | Null | 2271818 | A | Null | 2274152 | A | null | No | nc, NULL | Null | — |
| 976 | 2258377 | C | V | 2274461 | A | L | 2276795 | A | L | Yes | NS, C | O53473 | ATP binding activity |
| 977 | 2263994 | T | L | 2280079 | C | P | 2282413 | C | P | Yes | NS, NC | O53476 | — |
| 978 | 2266289 | T | V | 2282374 | C | V | 2284708 | C | V | Yes | S, NULL | Null | — |
| 979 | 2266290 | T | V | 2282375 | C | V | 2284709 | C | V | Yes | S, NULL | Null | — |
| 980 | 2271395 | A | V | 2287480 | G | A | 2289814 | G | A | Yes | NS, C | O53485 | transporter activity |
| 981 | 2272986 | C | D | 2289071 | G | H | 2291405 | G | H | Yes | NS, NC | Q50575 | enzyme activity |
| 982 | 2275230 | C | P | 2291315 | T | S | 2293649 | T | S | Yes | NS, NC | O53489 | — |
| 983 | 2282106 | T | S | 2298191 | C | G | 2300525 | C | G | Yes | NS, NC | O53489 | enzyme activity |
| 984 | 2285002 | C | L | 2301087 | T | L | 2303421 | T | L | Yes | S, NULL | Null | — |
| 985 | 2304443 | G | A | 2320528 | A | V | 2322862 | A | V | Yes | NS, C | O53498 | biosynthesis |
| 986 | 2312457 | C | M | 2328541 | T | I | 2330875 | T | I | Yes | NS, NC | Q10672 | porphyrin biosynthesis |
| 987 | 2312508 | A | G | 2328592 | G | G | 2330926 | G | G | Yes | S, NULL | Null | — |
| 988 | 2312541 | T | A | 2328625 | C | A | 2330959 | C | A | Yes | S, NULL | Null | — |
| 989 | 2313380 | T | P | 2329464 | C | P | 2331798 | C | P | Yes | S, NULL | Null | — |
| 990 | 2317013 | T | H | 2335125 | C | H | 2337459 | C | H | Yes | S, NULL | Null | — |
| 991 | 2317887 | T | L | 2335999 | C | P | 2338333 | C | P | Yes | NS, NC | Q10687 | — |
| 992 | 2319259 | C | A | 2337371 | T | V | 2339705 | T | V | Yes | NS, C | Q10688 | membrane |
| 993 | 2322578 | T | L | 2340687 | C | L | 2343015 | C | L | Yes | S, NULL | Null | — |
| 994 | 2322919 | A | T | 2341028 | G | A | 2343356 | G | A | Yes | NS, NC | Q10691 | integral to membrane |
| 995 | 2329583 | T | I | 2347738 | C | T | 2350008 | C | T | Yes | NS, NC | Q10699 | DNA binding activity |
| 996 | 2332029 | G | R | 2350184 | A | R | 2352454 | A | R | Yes | S, NULL | Null | — |
| 997 | 2333365 | A | M | 2351520 | G | T | 2353790 | G | T | Yes | NS, NC | Q10701 | nucleic acid binding |
| | | | | | | | | | | | | | activity |
| 998 | 2335359 | A | V | 2353514 | G | A | 2355783 | G | A | Yes | NS, C | Q10704 | — |
| 999 | 2344034 | T | S | 2362191 | G | A | 2364459 | G | A | Yes | NS, NC | O53499 | nucleic acid binding |
| | | | | | | | | | | | | | activity |
| 1000 | 2349668 | C | G | 2369184 | G | R | 2370093 | G | R | Yes | NS, NC | O33244 | endopeptidase activity |
| 1001 | 2353673 | C | P | 2373246 | A | T | 2374098 | A | T | Yes | NS, NC | O33248 | — |
| 1002 | 2353887 | T | L | 2373460 | C | S | 2374312 | C | S | Yes | NS, NC | O33248 | — |
| 1003 | 2354188 | T | Y | 2373761 | C | Y | 2374613 | C | Y | Yes | S, NULL | Null | — |
| 1004 | 2356850 | G | Null | 2376423 | A | Null | 2377275 | A | null | No | nc, NULL | Null | — |
| 1005 | 2360168 | G | Null | 2379741 | C | Null | 2380593 | C | null | Yes | nc, NULL | Null | — |
| 1006 | 2364212 | C | V | 2383812 | T | I | 2382390 | T | I | Yes | NS, C | O33259 | dihydropteroate |
| | | | | | | | | | | | | | synthase activity |
| 1007 | 2365161 | C | R | 2384761 | A | R | 2383339 | A | R | Yes | S, NULL | Null | — |
| 1008 | 2369039 | A | D | 2388639 | G | G | 2387216 | G | null | Yes | NS, NC | O33261 | amino acid-polyamine |
| | | | | | | | | | | | | | transporter activity |
| 1009 | 2369143 | A | S | 2388743 | G | G | 2387320 | G | G | Yes | NS, NC | O33261 | amino acid-polyamine |
| | | | | | | | | | | | | | transporter activity |
| 1010 | 2370697 | G | Null | 2390297 | A | Null | 2388874 | A | null | No | nc, NULL | Null | — |
| 1011 | 2373913 | C | R | 2393513 | T | R | 2392090 | T | R | Yes | S, NULL | Null | — |
| 1012 | 2377026 | C | V | 2396626 | A | L | 2395203 | A | L | Yes | NS, C | O06239 | undecaprenol kinase |
| | | | | | | | | | | | | | activity |
| 1013 | 2384213 | C | L | 2403697 | G | L | 2402390 | G | L | Yes | S, NULL | Null | — |
| 1014 | 2393645 | G | P | 2413129 | A | L | 2411822 | A | L | Yes | NS, NC | O06224 | cytokinesis |
| 1015 | 2393760 | A | L | 2413244 | C | V | 2411937 | C | V | Yes | NS, C | O06224 | cytokinesis |
| 1016 | 2395865 | A | V | 2415349 | C | V | 2414042 | C | V | Yes | S, NULL | Null | — |
| 1017 | 2399656 | G | V | 2419140 | A | V | 2417833 | A | V | Yes | S, NULL | Null | — |
| 1018 | 2402330 | G | A | 2421814 | A | V | 2420507 | A | V | Yes | NS, C | O06217 | — |
| 1019 | 2405256 | G | Null | 2424862 | A | Null | 2423555 | A | null | No | nc, NULL | Null | — |
| 1020 | 2405346 | C | Null | 2424952 | A | Null | 2423645 | A | null | No | nc, NULL | Null | — |
| 1021 | 2405863 | C | R | 2425469 | T | R | 2424162 | T | R | Yes | S, NULL | Null | — |
| 1022 | 2408026 | G | A | 2427632 | A | V | 2426325 | A | V | Yes | NS, C | O06213 | — |
| 1023 | 2413856 | G | Null | 2434820 | A | Null | 2432155 | A | null | No | nc, NULL | Null | — |
| 1024 | 2416293 | T | S | 2437257 | G | A | 2434592 | G | A | Yes | NS, NC | O53508 | — |
| 1025 | 2416871 | A | L | 2437835 | G | P | 2435170 | G | P | Yes | NS, NC | O53509 | — |
| 1026 | 2417128 | G | A | 2438092 | T | S | 2435427 | T | S | Yes | NS, NC | O53510 | protein kinase activity |
| 1027 | 2418638 | T | K | 2439602 | G | T | 2436937 | G | T | Yes | NS, NC | O53511 | DNA binding activity |
| 1028 | 2421783 | T | T | 2442747 | G | P | 2440082 | G | P | Yes | NS, NC | O53514 | — |
| 1029 | 2423986 | C | G | 2444950 | A | G | 2442285 | A | G | Yes | S, NULL | Null | — |
| 1030 | 2426460 | T | I | 2447424 | G | I | 2444759 | G | I | Yes | S, NULL | Null | — |
| 1031 | 2426573 | G | Null | 2447537 | A | Null | 2444872 | A | null | No | nc, NULL | Null | — |
| 1032 | 2427491 | T | S | 2448456 | C | S | 2445791 | C | S | Yes | S, NULL | Null | — |
| 1033 | 2428328 | A | E | 2449293 | G | G | 2446628 | G | G | Yes | NS, NC | O53521 | enzyme activity |
| 1034 | 2433965 | A | T | 2454930 | G | T | 2452265 | G | T | Yes | S, NULL | Null | — |
| 1035 | 2433967 | G | T | 2454932 | A | T | 2452267 | A | T | Yes | S, NULL | Null | — |
| 1036 | 2438267 | C | I | 2459232 | G | M | 2456567 | G | M | Yes | NS, NC | Q10387 | electron transport |
| 1037 | 2440692 | A | S | 2461543 | C | A | 2458821 | C | A | Yes | NS, NC | Q10389 | integral to membrane |
| 1038 | 2447668 | T | V | 2468519 | C | V | 2465797 | C | V | Yes | S, NULL | Null | — |
| 1039 | 2449344 | G | C | 2470195 | A | C | 2467473 | A | C | Yes | S, NULL | Null | — |
| 1040 | 2449738 | C | Null | 2470589 | A | Null | 2467867 | A | null | Yes | S, NULL | Null | — |
| 1041 | 2452103 | C | S | 2472954 | T | L | 2470232 | T | L | Yes | NS, NC | Q10397 | porphyrin biosynthesis |
| 1042 | 2454263 | A | F | 2475114 | G | F | 2472392 | G | F | Yes | S, NULL | Null | — |
| 1043 | 2455035 | G | A | 2475886 | T | E | 2473164 | T | E | Yes | NS, NC | Q10399 | enzyme activity |
| 1044 | 2458114 | G | L | 2478965 | C | F | 2476243 | C | F | Yes | NS, NC | Q10401 | aminopeptidase activity |
| 1045 | 2463948 | G | G | 2484799 | A | D | 2482077 | A | D | Yes | NS, NC | Q10404 | enzyme activity |
| 1046 | 2465601 | C | L | 2486452 | T | F | 2483730 | T | F | Yes | NS, NC | Q10405 | integral to membrane |
| 1047 | 2468463 | G | Null | 2489314 | A | Null | 2486590 | A | null | No | nc, NULL | Null | — |
| 1048 | 2474596 | C | Null | 2495447 | T | Null | 2492723 | T | null | No | nc, NULL | Null | — |
| 1049 | 2474647 | C | L | 2495498 | T | L | 2492774 | T | L | Yes | S, NULL | Null | — |
| 1050 | 2478070 | A | D | 2498921 | G | G | 2496197 | G | G | Yes | NS, NC | Q10510 | — |
| 1051 | 2480295 | C | A | 2501146 | T | A | 2498422 | T | A | Yes | S, NULL | Null | — |
| 1052 | 2480652 | C | L | 2501502 | T | F | 2498778 | T | F | Yes | NS, NC | Q10511 | — |
| 1053 | 2481905 | T | Q | 2502755 | C | R | 2500031 | C | null | Yes | NS, NC | Q10513 | kinesin complex |
| 1054 | 2488510 | A | I | 2509360 | G | T | 2506634 | G | T | Yes | NS, NC | Q10518 | vitamin B12 |
| | | | | | | | | | | | | | biosynthesis |
| 1055 | 2488792 | A | G | 2509642 | G | G | 2506916 | G | G | Yes | S, NULL | Null | — |
| 1056 | 2490860 | T | K | 2511710 | G | T | 2508984 | G | T | Yes | NS, NC | Q10522 | integral to membrane |
| 1057 | 2492611 | C | R | 2513461 | G | G | 2510735 | G | G | Yes | NS, NC | Q10504 | pyruvate |
| | | | | | | | | | | | | | dehydrogenase |
| | | | | | | | | | | | | | (lipoamide) activity |
| 1058 | 2494787 | T | V | 2515637 | G | V | 2512911 | G | V | Yes | S, NULL | Null | — |
| 1059 | 2497280 | T | T | 2518130 | C | T | 2515404 | C | T | Yes | S, NULL | Null | — |
| 1060 | 2501788 | A | E | 2522726 | C | D | 2519999 | C | D | Yes | NS, C | Q10526 | — |
| 1061 | 2504215 | G | A | 2525150 | T | D | 2522412 | T | D | Yes | NS, NC | Q10528 | DNA binding activity |
| 1062 | 2507835 | A | V | 2528771 | G | A | 2526031 | G | A | Yes | NS, C | O53528 | lysine permease activity |
| 1063 | 2508361 | A | Null | 2529297 | G | Null | 2526557 | G | null | Yes | nc, NULL | Null | — |
| 1064 | 2508860 | C | G | 2529796 | G | A | 2527056 | G | A | Yes | NS, C | O53530 | — |
| 1065 | 2509390 | C | R | 2530326 | A | R | 2527586 | A | R | Yes | S, NULL | Null | — |
| 1066 | 2516165 | C | P | 2537209 | G | P | 2534414 | G | P | Yes | S, NULL | Null | — |
| 1067 | 2518574 | A | V | 2539618 | G | V | 2536823 | G | V | Yes | S, NULL | Null | — |
| 1068 | 2520531 | G | Null | 2541575 | A | Null | 2538780 | A | null | No | nc, NULL | Null | — |
| 1069 | 2524106 | T | L | 2545150 | C | P | 2542355 | C | P | Yes | NS, NC | Q50693 | membrane |
| 1070 | 2525002 | G | L | 2546046 | C | L | 2543251 | C | L | Yes | S, NULL | Null | — |
| 1071 | 2525661 | C | M | 2546705 | T | I | 2543910 | T | M | Yes | NS, NC | Q50689 | — |
| 1072 | 2528547 | G | P | 2549591 | A | L | 2546796 | A | L | Yes | NS, NC | Q50687 | glycerol metabolism |
| 1073 | 2533496 | T | Null | 2555898 | G | Null | 2551745 | G | null | No | nc, NULL | Null | — |
| 1074 | 2536766 | A | * | 2559168 | G | Q | 2555015 | G | Q | Yes | NS, TP | Q50679 | protein disulfide |
| | | | | | | | | | | | | | oxidoreductase activity |
| 1075 | 2537259 | G | Null | 2559661 | C | Null | 2555508 | C | null | No | nc, NULL | Null | — |
| 1076 | 2540378 | C | A | 2562781 | T | V | 2558628 | T | V | Yes | NS, C | Q50675 | membrane |
| 1077 | 2541551 | C | A | 2563956 | A | E | 2559803 | A | E | Yes | NS, NC | Q59570 | thiosulfate |
| | | | | | | | | | | | | | sulfurtransferase |
| | | | | | | | | | | | | | activity |
| 1078 | 2544362 | G | Null | 2566766 | A | Null | 2562613 | A | null | No | nc, NULL | Null | — |
| 1079 | 2550055 | A | H | 2572458 | G | H | 2568305 | G | H | Yes | S, NULL | Null | — |
| 1080 | 2553846 | A | D | 2576249 | G | G | 2572096 | G | G | Yes | NS, NC | Q50660 | molecular_function |
| | | | | | | | | | | | | | unknown |
| 1081 | 2554841 | G | V | 2577244 | A | V | 2573091 | A | V | Yes | S, NULL | Null | — |
| 1082 | 2555589 | A | W | 2577992 | G | R | 2573839 | G | R | Yes | NS, NC | Q50658 | enzyme activity |
| 1083 | 2558141 | T | V | 2580544 | G | G | 2576391 | G | G | Yes | NS, C | Q50657 | — |
| 1084 | 2558704 | A | R | 2581107 | C | R | 2576954 | C | R | Yes | S, NULL | Null | — |
| 1085 | 2560796 | A | K | 2583199 | G | Null | 2579046 | G | E | Yes | nc, NULL | Null | — |
| 1086 | 2569172 | G | V | 2591575 | C | L | 2587422 | C | L | Yes | NS, C | P71894 | transporter activity |
| 1087 | 2573528 | T | T | 2595931 | C | A | 2591777 | C | A | Yes | NS, NC | P71889 | — |
| 1088 | 2579779 | C | A | 2602182 | G | A | 2598028 | G | A | Yes | S, NULL | Null | — |
| 1089 | 2582470 | C | L | 2604873 | A | L | 2600719 | A | null | Yes | S, NULL | Null | — |
| 1090 | 2582888 | T | D | 2605291 | G | E | 2601137 | G | E | Yes | NS, C | P71880 | malic enzyme activity |
| 1091 | 2583687 | C | L | 2606090 | A | I | 2601936 | A | I | Yes | NS, C | P71880 | malic enzyme activity |
| 1092 | 2586084 | C | Null | 2608486 | T | Null | 2604332 | T | null | Yes | nc, NULL | Null | — |
| 1093 | 2589179 | T | H | 2611581 | C | H | 2607427 | C | H | Yes | S, NULL | Null | — |
| 1094 | 2589896 | T | R | 2612298 | G | R | 2608144 | G | R | Yes | S, NULL | Null | — |
| 1095 | 2592420 | T | V | 2614821 | C | A | 2610667 | C | A | Yes | NS, C | P95235 | membrane |
| 1096 | 2594770 | A | Null | 2617171 | G | Null | 2613017 | G | null | Yes | S, NULL | Null | — |
| 1097 | 2596868 | C | Null | 2619269 | T | Null | 2615115 | T | null | No | nc, NULL | Null | — |
| 1098 | 2603521 | C | A | 2625922 | T | A | 2621768 | T | A | Yes | S, NULL | Null | — |
| 1099 | 2609911 | G | R | 2641838 | A | H | 2639170 | A | H | Yes | NS, NC | O05839 | transcription factor |
| | | | | | | | | | | | | | activity |
| 1100 | 2611180 | A | F | 2643107 | G | F | 2640439 | G | F | Yes | S, NULL | Null | — |
| 1101 | 2614613 | A | A | 2646540 | G | A | 2643872 | G | A | Yes | S, NULL | Null | — |
| 1102 | 2626747 | A | V | 2658674 | G | A | 2656006 | G | A | Yes | NS, C | O05819 | enzyme activity |
| 1103 | 2627613 | C | G | 2659540 | T | G | 2656872 | T | G | Yes | S, NULL | Null | — |
| 1104 | 2643121 | T | Q | 2675048 | C | R | 2672380 | C | R | Yes | NS, NC | P71717 | enzyme activity |
| 1105 | 2650664 | T | T | 2682591 | C | A | 2679923 | C | A | Yes | NS, NC | P71756 | magnesium ion binding |
| | | | | | | | | | | | | | activity |
| 1106 | 2652240 | G | T | 2684167 | A | I | 2681499 | A | I | Yes | NS, NC | P71754 | — |
| 1107 | 2656797 | C | A | 2688724 | T | A | 2686056 | T | A | Yes | S, NULL | Null | — |
| 1108 | 2657264 | A | Q | 2689191 | G | R | 2686523 | G | R | Yes | NS, NC | P71750 | gamma-glutamyl |
| | | | | | | | | | | | | | transferase activity |
| 1109 | 2659785 | C | P | 2691711 | T | S | 2689043 | T | S | Yes | NS, NC | P71749 | — |
| 1110 | 2660947 | A | N | 2692873 | G | S | 2690205 | G | S | Yes | NS, C | P71748 | oxygen transporter |
| | | | | | | | | | | | | | activity |
| 1111 | 2661446 | C | A | 2693375 | T | A | 2690707 | T | A | Yes | S, NULL | Null | — |
| 1112 | 2661841 | G | S | 2693770 | A | N | 2691102 | A | N | Yes | NS, C | P71748 | oxygen transporter |
| | | | | | | | | | | | | | activity |
| 1113 | 2663078 | T | H | 2695007 | C | R | 2692339 | C | R | Yes | NS, NC | P71746 | transporter activity |
| 1114 | 2668981 | G | L | 2700910 | C | V | 2698242 | C | V | Yes | NS, C | P71740 | — |
| 1115 | 2671087 | T | G | 2703016 | G | G | 2700348 | G | G | Yes | S, NULL | Null | — |
| 1116 | 2671898 | T | A | 2703827 | C | A | 2701157 | C | A | Yes | S, NULL | Null | — |
| 1117 | 2677979 | G | A | 2709793 | A | A | 2706644 | A | A | Yes | S, NULL | Null | — |
| 1118 | 2681098 | A | L | 2712911 | C | R | 2709762 | C | R | Yes | NS, NC | P71728 | DNA binding activity |
| 1119 | 2683310 | C | A | 2715123 | T | T | 2711974 | T | T | Yes | NS, NC | P71727 | — |
| 1120 | 2684991 | C | R | 2716804 | T | R | 2713655 | T | R | Yes | S, NULL | Null | — |
| 1121 | 2685491 | A | V | 2717304 | G | A | 2714155 | G | A | Yes | NS, C | P71724 | enzyme activity |
| 1122 | 2686456 | T | S | 2718269 | C | G | 2715120 | C | G | Yes | NS, NC | O86328 | nicotinate-nucleotide |
| | | | | | | | | | | | | | adenylyltransferase |
| | | | | | | | | | | | | | activity |
| 1123 | 2687242 | A | Null | 2719055 | G | Null | 2715906 | G | null | No | nc, NULL | Null | — |
| 1124 | 2689080 | A | Y | 2720893 | G | H | 2717744 | R | ? | Yes | NS, C | P71924 | DNA binding activity |
| 1125 | 2689137 | C | A | 2720950 | T | T | 2717801 | Y | ? | Yes | NS, NC | P71924 | DNA binding activity |
| 1126 | 2689139 | A | I | 2720952 | G | T | 2717803 | R | ? | Yes | NS, NC | P71924 | DNA binding activity |
| 1127 | 2691689 | C | L | 2723504 | T | L | 2720355 | T | L | Yes | S, NULL | Null | — |
| 1128 | 2692030 | G | L | 2723845 | A | F | 2720696 | A | F | Yes | NS, NC | P71922 | nucleotide binding |
| | | | | | | | | | | | | | activity |
| 1129 | 2693966 | T | V | 2725801 | C | Null | 2722652 | C | V | Yes | nc, NULL | Null | — |
| 1130 | 2702213 | T | V | 2734048 | C | A | 2730899 | C | A | Yes | NS, C | P71913 | ribokinase activity |
| 1131 | 2705180 | A | Null | 2737015 | G | Null | 2733866 | G | null | Yes | nc, NULL | Null | — |
| 1132 | 2708856 | C | Null | 2740691 | T | Null | 2737539 | T | null | Yes | nc, NULL | Null | — |
| 1133 | 2709921 | T | L | 2741756 | G | L | 2738604 | G | L | Yes | S, NULL | Null | — |
| 1134 | 2719463 | A | T | 2751298 | G | T | 2748146 | G | T | Yes | S, NULL | Null | — |
| 1135 | 2720611 | G | D | 2752446 | A | N | 2749294 | A | N | Yes | NS, NC | O53178 | — |
| 1136 | 2721171 | T | Null | 2753006 | C | Null | 2749854 | C | null | No | nc, NULL | Null | — |
| 1137 | 2728310 | T | R | 2760145 | C | R | 2756992 | C | R | Yes | S, NULL | Null | — |
| 1138 | 2729181 | A | I | 2761016 | G | M | 2757863 | G | M | Yes | NS, NC | O53186 | transporter activity |
| 1139 | 2733102 | A | L | 2764937 | G | P | 2761784 | G | P | Yes | NS, NC | O53189 | cytokinesis |
| 1140 | 2736005 | C | Q | 2767840 | T | Q | 2764687 | T | Q | Yes | S, NULL | Null | — |
| 1141 | 2740904 | A | S | 2772739 | C | A | 2769586 | C | A | Yes | NS, NC | O53196 | nucleic acid binding |
| | | | | | | | | | | | | | activity |
| 1142 | 2741117 | C | G | 2772952 | T | S | 2769799 | T | S | Yes | NS, NC | O53196 | nucleic acid binding |
| | | | | | | | | | | | | | activity |
| 1143 | 2742343 | C | S | 2774178 | G | W | 2771025 | G | W | Yes | NS, NC | O53198 | alpha-amylase activity |
| 1144 | 2743386 | T | Null | 2775221 | C | Null | 2772068 | C | null | No | nc, NULL | Null | — |
| 1145 | 2745044 | A | V | 2776879 | G | A | 2773726 | G | A | Yes | NS, C | O53201 | — |
| 1146 | 2751087 | C | T | 2782925 | T | T | 2779772 | T | T | Yes | S, NULL | Null | — |
| 1147 | 2754350 | C | S | 2787546 | T | N | 2783035 | T | N | Yes | NS, C | O53207 | glycerol-3-phosphate O- |
| | | | | | | | | | | | | | acyltransferase activity |
| 1148 | 2757260 | C | G | 2790456 | A | C | 2785945 | A | C | Yes | NS, NC | O53208 | metabolism |
| 1149 | 2757900 | T | D | 2791096 | C | G | 2786585 | C | G | Yes | NS, NC | O53209 | molecular_function |
| | | | | | | | | | | | | | unknown |
| 1150 | 2758277 | G | A | 2791473 | A | A | 2786962 | A | A | Yes | S, NULL | Null | — |
| 1151 | 2762360 | T | T | 2795557 | C | T | 2791046 | C | T | Yes | S, NULL | Null | — |
| 1152 | 2763122 | A | V | 2796320 | G | A | 2791809 | G | A | Yes | NS, C | O53212 | — |
| 1153 | 2763158 | G | T | 2796356 | C | S | 2791845 | C | S | Yes | NS, C | O53212 | — |
| 1154 | 2771624 | A | H | 2804833 | G | H | 2800322 | G | H | Yes | S, NULL | Null | — |
| 1155 | 2774283 | A | D | 2807484 | C | A | 2802973 | C | A | Yes | NS, NC | O53217 | thymidylate synthase |
| | | | | | | | | | | | | | activity |
| 1156 | 2776115 | A | W | 2809316 | G | R | 2804805 | G | R | Yes | NS, NC | O06159 | protein binding activity |
| 1157 | 2777179 | T | S | 2810380 | C | G | 2805869 | C | G | Yes | NS, NC | O06160 | — |
| 1158 | 2779539 | G | G | 2812740 | A | G | 2808229 | A | G | Yes | S, NULL | Null | — |
| 1159 | 2784243 | T | L | 2817444 | G | F | 2812933 | G | F | Yes | NS, NC | O06165 | biotin carboxylase |
| | | | | | | | | | | | | | activity |
| 1160 | 2785043 | T | S | 2818244 | C | G | 2813733 | C | G | Yes | NS, NC | O06165 | biotin carboxylase |
| | | | | | | | | | | | | | activity |
| 1161 | 2789771 | T | P | 2822972 | C | P | 2818461 | C | P | Yes | S, NULL | Null | — |
| 1162 | 2792263 | A | K | 2825464 | G | K | 2820953 | G | K | Yes | S, NULL | Null | — |
| 1163 | 2798279 | G | G | 2831480 | A | G | 2826969 | A | G | Yes | S, NULL | Null | — |
| 1164 | 2802192 | T | Null | 2835393 | C | Null | 2830882 | C | null | No | nc, NULL | Null | — |
| 1165 | 2803099 | C | A | 2836300 | G | A | 2831789 | G | A | Yes | S, NULL | Null | — |
| 1166 | 2803100 | C | A | 2836301 | G | A | 2831790 | G | A | Yes | S, NULL | Null | — |
| 1167 | 2804844 | A | M | 2838045 | G | V | 2833534 | G | V | Yes | NS, NC | O53226 | — |
| 1168 | 2807819 | G | R | 2841020 | A | C | 2836509 | A | C | Yes | NS, NC | P95029 | enzyme activity |
| 1169 | 2814078 | G | D | 2847279 | A | D | 2842768 | A | D | Yes | S, NULL | Null | — |
| 1170 | 2818088 | A | V | 2851289 | G | V | 2846777 | G | V | Yes | S, NULL | Null | — |
| 1171 | 2818694 | G | Q | 2851895 | C | E | 2847383 | C | E | Yes | NS, NC | P95025 | — |
| 1172 | 2819952 | A | E | 2853153 | G | G | 2848641 | G | G | Yes | NS, NC | P95024 | nucleic acid binding |
| | | | | | | | | | | | | | activity |
| 1173 | 2824432 | A | D | 2857633 | G | D | 2853121 | G | D | Yes | S, NULL | Null | — |
| 1174 | 2825466 | T | D | 2858667 | G | A | 2854156 | G | A | Yes | NS, NC | P95020 | RNA binding activity |
| 1175 | 2837839 | T | Null | 2870384 | C | Null | 2866530 | C | null | No | nc, NULL | Null | — |
| 1176 | 2843260 | G | N | 2875806 | C | K | 2871952 | C | K | Yes | NS, NC | O07438 | nucleic acid binding |
| | | | | | | | | | | | | | activity |
| 1177 | 2846432 | T | D | 2878978 | C | G | 2875124 | C | G | Yes | NS, NC | Q50739 | nucleotide binding |
| | | | | | | | | | | | | | activity |
| 1178 | 2849417 | C | T | 2881964 | T | I | 2878109 | T | I | Yes | NS, NC | Q50737 | — |
| 1179 | 2853851 | A | S | 2886398 | G | G | 2882543 | G | G | Yes | NS, NC | Q50732 | — |
| 1180 | 2854021 | G | E | 2886568 | A | E | 2882713 | A | E | Yes | S, NULL | Null | — |
| 1181 | 2854631 | T | S | 2887178 | G | A | 2883323 | G | A | Yes | NS, NC | Q50732 | — |
| 1182 | 2858117 | T | L | 2890666 | C | L | 2886811 | C | L | Yes | S, NULL | Null | — |
| 1183 | 2863708 | A | V | 2896258 | G | A | 2892403 | G | A | Yes | NS, C | Q50649 | nucleic acid binding |
| | | | | | | | | | | | | | activity |
| 1184 | 2865290 | C | Null | 2897840 | G | Null | 2893985 | G | null | No | nc, NULL | Null | — |
| 1185 | 2865319 | G | Null | 2897869 | A | Null | 2894014 | A | null | No | nc, NULL | Null | — |
| 1186 | 2866213 | T | A | 2898763 | C | A | 2894908 | C | A | Yes | S, NULL | Null | — |
| 1187 | 2868616 | A | * | 2901166 | G | W | 2897311 | G | W | Yes | NS, TP | Q50644 | hydrolase activity |
| 1188 | 2871372 | A | T | 2903922 | G | A | 2900067 | G | A | Yes | NS, NC | Q50642 | enzyme activity |
| 1189 | 2871964 | A | Q | 2904514 | G | R | 2900659 | G | R | Yes | NS, NC | Q50642 | enzyme activity |
| 1190 | 2874457 | T | F | 2907007 | G | V | 2903152 | G | V | Yes | NS, NC | Q50639 | peptidyl-prolyl cis-trans |
| | | | | | | | | | | | | | isomerase activity |
| 1191 | 2879964 | A | T | 2912514 | G | T | 2908659 | G | T | Yes | S, NULL | Null | — |
| 1192 | 2880160 | G | P | 2912710 | T | T | 2908855 | T | T | Yes | NS, NC | Q50635 | protein targeting |
| 1193 | 2880535 | T | T | 2913085 | C | A | 2909230 | C | A | Yes | NS, NC | Q50635 | protein targeting |
| 1194 | 2881707 | C | S | 2914257 | T | N | 2910402 | T | N | Yes | NS, C | Q50634 | protein targeting |
| 1195 | 2886244 | A | Y | 2918794 | T | F | 2914939 | T | F | Yes | NS, C | Q50631 | enzyme activity |
| 1196 | 2887118 | A | L | 2919668 | G | L | 2915813 | G | L | Yes | S, NULL | Null | — |
| 1197 | 2888634 | A | T | 2921184 | G | A | 2917329 | G | A | Yes | NS, NC | Q50631 | enzyme activity |
| 1198 | 2890241 | A | S | 2922791 | G | G | 2918936 | G | G | Yes | NS, NC | Q50630 | — |
| 1199 | 2890386 | A | D | 2922936 | G | G | 2919081 | G | G | Yes | NS, NC | Q50630 | — |
| 1200 | 2890432 | C | G | 2922982 | T | G | 2919127 | T | G | Yes | S, NULL | Null | — |
| 1201 | 2893419 | C | R | 2925960 | T | C | 2922105 | T | C | Yes | NS, NC | Q50625 | — |
| 1202 | 2894748 | A | A | 2927289 | G | A | 2923434 | G | A | Yes | S, NULL | Null | — |
| 1203 | 2894968 | T | I | 2927509 | G | S | 2923654 | G | S | Yes | NS, NC | Q50622 | integral to membrane |
| 1204 | 2896114 | A | L | 2928655 | G | L | 2924800 | G | L | Yes | S, NULL | Null | — |
| 1205 | 2900347 | G | L | 2932888 | A | F | 2929033 | A | F | Yes | NS, NC | O06209 | acyl-CoA metabolism |
| 1206 | 2903343 | T | S | 2935884 | C | P | 2932029 | C | P | Yes | NS, NC | O06206 | — |
| 1207 | 2911002 | A | H | 2943543 | G | Null | 2939688 | G | null | Yes | nc, NULL | Null | — |
| 1208 | 2913009 | A | I | 2945541 | G | V | 2941686 | G | V | Yes | NS, C | O06198 | — |
| 1209 | 2913792 | C | Null | 2946324 | T | Null | 2942469 | T | null | No | nc, NULL | Null | — |
| 1210 | 2920364 | A | H | 2952899 | G | H | 2949044 | G | H | Yes | S, NULL | Null | — |
| 1211 | 2920770 | T | Null | 2953305 | G | Null | 2949450 | G | null | No | nc, NULL | Null | — |
| 1212 | 2922696 | T | L | 2955231 | C | S | 2951376 | C | S | Yes | NS, NC | O06184 | — |
| 1213 | 2922723 | G | R | 2955258 | C | P | 2951403 | C | P | Yes | NS, NC | O06184 | — |
| 1214 | 2926786 | G | Null | 2959321 | A | Null | 2955467 | A | null | No | nc, NULL | Null | — |
| 1215 | 2929067 | G | G | 2961602 | C | G | 2957748 | C | G | Yes | S, NULL | Null | — |
| 1216 | 2935735 | C | A | 2968270 | T | V | 2964416 | T | V | Yes | NS, C | P71942 | protein tyrosine |
| | | | | | | | | | | | | | phosphatase activity |
| 1217 | 2935930 | C | Null | 2968465 | G | Null | 2964611 | G | null | Yes | nc, NULL | Null | — |
| 1218 | 2938515 | A | R | 2982032 | G | R | 2976820 | G | R | Yes | S, NULL | Null | — |
| 1219 | 2941411 | C | A | 2984929 | G | G | 2979718 | G | G | Yes | NS, C | P71965 | — |
| 1220 | 2941695 | A | K | 2985213 | G | E | 2980002 | G | E | Yes | NS, NC | P71965 | — |
| 1221 | 2945109 | G | D | 2988627 | C | H | 2983416 | C | H | Yes | NS, NC | P71969 | — |
| 1222 | 2948228 | G | S | 2991691 | C | S | 2986535 | C | S | Yes | S, NULL | Null | — |
| 1223 | 2950721 | C | L | 2994184 | T | L | 2989028 | T | L | Yes | S, NULL | Null | — |
| 1224 | 2950791 | C | G | 2994254 | G | A | 2989098 | G | A | Yes | NS, C | O53231 | uroporphyrinogen |
| | | | | | | | | | | | | | decarboxylase activity |
| 1225 | 2953961 | T | V | 2997322 | C | A | 2992268 | C | A | Yes | NS, C | O07183 | nucleic acid binding |
| | | | | | | | | | | | | | activity |
| 1226 | 2956998 | C | S | 3000359 | T | L | 2995305 | T | L | Yes | NS, NC | O07185 | — |
| 1227 | 2959751 | G | G | 3003112 | C | A | 2998058 | C | A | Yes | NS, C | O07187 | transport |
| 1228 | 2959795 | T | C | 3003156 | G | G | 2998102 | G | G | Yes | NS, NC | O07187 | transport |
| 1229 | 2963534 | A | Y | 3006895 | G | Y | 3001840 | G | Y | Yes | S, NULL | Null | — |
| 1230 | 2963874 | G | R | 3007235 | A | * | 3002180 | A | * | Yes | NS, TP | O07192 | amino acid-polyamine |
| | | | | | | | | | | | | | transporter activity |
| 1231 | 2967056 | G | V | 3010417 | A | I | 3005362 | A | I | Yes | NS, C | O07194 | transport |
| 1232 | 2968202 | T | S | 3011563 | G | R | 3006508 | G | R | Yes | NS, NC | O07196 | nucleic acid binding |
| | | | | | | | | | | | | | activity |
| 1233 | 2969755 | T | N | 3013116 | C | D | 3008061 | C | D | Yes | NS, NC | O07198 | — |
| 1234 | 2978578 | G | S | 3021939 | C | T | 3016884 | C | T | Yes | NS, C | O07210 | — |
| 1235 | 2979168 | A | A | 3022529 | G | A | 3017474 | G | A | Yes | S, NULL | Null | — |
| 1236 | 2979744 | T | V | 3023105 | G | V | 3018050 | G | V | Yes | S, NULL | Null | — |
| 1237 | 2984242 | G | W | 3027603 | C | S | 3022542 | C | S | Yes | NS, NC | O07213 | — |
| 1238 | 2984434 | C | A | 3027795 | T | V | 3022734 | T | V | Yes | NS, C | O07213 | — |
| 1239 | 2984591 | A | L | 3027952 | G | L | 3022891 | G | L | Yes | S, NULL | Null | — |
| 1240 | 2987804 | G | H | 3031165 | A | Y | 3026104 | A | Y | Yes | NS, C | O07218 | cell wall catabolism |
| 1241 | 2988773 | C | A | 3032134 | T | V | 3027073 | T | V | Yes | NS, C | Q50765 | DNA binding activity |
| 1242 | 2993548 | G | T | 3036909 | A | I | 3031848 | A | I | Yes | NS, NC | O33229 | acyl-CoA |
| | | | | | | | | | | | | | dehydrogenase activity |
| 1243 | 2993831 | C | A | 3037192 | G | P | 3032131 | G | P | Yes | NS, NC | O33229 | acyl-CoA |
| | | | | | | | | | | | | | dehydrogenase activity |
| 1244 | 2998193 | C | Null | 3041554 | T | Null | 3036491 | T | null | No | nc, NULL | Null | — |
| 1245 | 2998315 | A | V | 3041676 | G | A | 3036613 | G | A | Yes | NS, C | O33234 | — |
| 1246 | 2998989 | C | L | 3042350 | G | F | 3037287 | G | F | Yes | NS, NC | O33234 | — |
| 1247 | 3001500 | T | V | 3044861 | C | V | 3039798 | C | V | Yes | S, NULL | Null | — |
| 1248 | 3011613 | A | G | 3055059 | C | G | 3049784 | C | G | Yes | S, NULL | Null | — |
| 1249 | 3011639 | A | D | 3055085 | G | G | 3049810 | G | G | Yes | NS, NC | O33284 | — |
| 1250 | 3012496 | G | L | 3055919 | A | L | 3050644 | A | L | Yes | S, NULL | Null | — |
| 1251 | 3014181 | G | Q | 3057604 | T | K | 3052329 | T | K | Yes | NS, NC | P31511 | — |
| 1252 | 3015844 | G | E | 3059267 | A | E | 3053992 | A | null | Yes | S, NULL | Null | — |
| 1253 | 3026379 | C | V | 3069802 | G | V | 3064527 | G | V | Yes | S, NULL | Null | — |
| 1254 | 3029501 | A | * | 3072924 | G | Q | 3067649 | G | Q | Yes | NS, TP | O33304 | — |
| 1255 | 3033865 | C | R | 3077288 | G | P | 3072013 | G | P | Yes | NS, NC | O33310 | — |
| 1256 | 3035400 | A | S | 3078823 | G | P | 3073548 | G | P | Yes | NS, NC | O33311 | — |
| 1257 | 3036408 | A | A | 3079831 | G | A | 3074556 | G | A | Yes | S, NULL | Null | — |
| 1258 | 3036451 | G | S | 3079874 | A | F | 3074599 | A | F | Yes | NS, NC | O33312 | — |
| 1259 | 3038305 | A | I | 3081728 | G | T | 3076453 | G | T | Yes | NS, NC | P72024 | dihydrodipicolinate |
| | | | | | | | | | | | | | reductase activity |
| 1260 | 3038502 | T | E | 3081925 | G | D | 3076650 | G | D | Yes | NS, C | P72024 | dihydrodipicolinate |
| | | | | | | | | | | | | | reductase activity |
| 1261 | 3043278 | T | I | 3086725 | C | M | 3081450 | C | M | Yes | NS, NC | O33321 | DNA binding activity |
| 1262 | 3045174 | G | V | 3088622 | A | V | 3083347 | A | V | Yes | S, NULL | Null | — |
| 1263 | 3049237 | G | Null | 3092685 | A | Null | 3087410 | A | null | No | nc, NULL | Null | — |
| 1264 | 3053917 | C | A | 3097365 | A | D | 3092089 | A | D | Yes | NS, NC | O33330 | transcription factor |
| | | | | | | | | | | | | | activity |
| 1265 | 3054660 | A | G | 3098108 | G | G | 3092832 | G | G | Yes | S, NULL | Null | — |
| 1266 | 3055488 | C | Null | 3098936 | G | Null | 3093660 | G | null | No | nc, NULL | Null | — |
| 1267 | 3058474 | C | A | 3101922 | T | T | 3096643 | T | T | Yes | NS, NC | O33334 | recombinase activity |
| 1268 | 3058662 | A | V | 3102110 | G | A | 3096831 | G | A | Yes | NS, C | O33334 | recombinase activity |
| 1269 | 3060738 | G | C | 3104186 | A | C | 3098907 | A | C | Yes | S, NULL | Null | — |
| 1270 | 3061693 | A | F | 3105141 | C | C | 3099862 | C | C | Yes | NS, NC | P71655 | — |
| 1271 | 3063629 | A | A | 3107077 | G | A | 3101798 | G | A | Yes | S, NULL | Null | — |
| 1272 | 3066230 | C | D | 3109675 | T | D | 3104396 | T | D | Yes | S, NULL | Null | — |
| 1273 | 3069429 | A | V | 3112874 | G | A | 3107595 | G | A | Yes | NS, C | P71647 | — |
| 1274 | 3069475 | T | T | 3112920 | C | A | 3107641 | C | A | Yes | NS, NC | P71647 | — |
| 1275 | 3072095 | C | P | 3115540 | A | T | 3110261 | A | T | Yes | NS, NC | P71642 | — |
| 1276 | 3074999 | G | V | 3118446 | A | I | 3113166 | A | I | Yes | NS, C | P71638 | — |
| 1277 | 3081797 | A | * | 3125232 | G | Q | 3119439 | G | Q | Yes | NS, TP | P71635 | — |
| 1278 | 3084028 | G | R | 3127463 | T | R | 3121670 | T | R | Yes | S, NULL | Null | — |
| 1279 | 3089119 | G | P | 3132545 | T | H | 3126761 | T | H | Yes | NS, NC | P71628 | — |
| 1280 | 3089527 | T | E | 3132953 | G | A | 3127169 | G | A | Yes | NS, NC | P71627 | — |
| 1281 | 3089534 | G | L | 3132960 | A | L | 3127176 | A | L | Yes | S, NULL | Null | — |
| 1282 | 3089536 | T | Q | 3132962 | G | Q | 3127178 | G | Q | Yes | S, NULL | Null | — |
| 1283 | 3089537 | G | Q | 3132963 | T | Q | 3127179 | T | Q | Yes | S, NULL | Null | — |
| 1284 | 3089546 | G | L | 3132972 | C | V | 3127188 | C | V | Yes | NS, C | P71627 | — |
| 1285 | 3089625 | G | C | 3133051 | C | C | 3127267 | C | C | Yes | S, NULL | Null | — |
| 1286 | 3089626 | C | C | 3133052 | G | C | 3127268 | G | C | Yes | S, NULL | Null | — |
| 1287 | 3091410 | A | R | 3134836 | G | R | 3129052 | G | R | Yes | S, NULL | Null | — |
| 1288 | 3092467 | G | G | 3135893 | A | G | 3130109 | A | G | Yes | S, NULL | Null | — |
| 1289 | 3092732 | G | A | 3136158 | T | E | 3130374 | T | E | Yes | NS, NC | P71624 | — |
| 1290 | 3093808 | C | Null | 3137234 | T | Null | 3131450 | T | null | No | nc, NULL | Null | — |
| 1291 | 3094520 | T | C | 3137946 | C | R | 3132162 | C | R | Yes | NS, NC | P71621 | enzyme activity |
| 1292 | 3096227 | G | P | 3139653 | C | A | 3133869 | C | A | Yes | NS, NC | P71619 | transporter activity |
| 1293 | 3096535 | A | L | 3139961 | G | P | 3134175 | G | L | Yes | NS, NC | P71619 | transporter activity |
| 1294 | 3096724 | A | I | 3140150 | C | S | 3134364 | C | I | Yes | NS, NC | P71619 | transporter activity |
| 1295 | 3098942 | T | L | 3142369 | C | L | 3136583 | C | L | Yes | S, NULL | Null | — |
| 1296 | 3099150 | A | L | 3142577 | G | P | 3136791 | G | P | Yes | NS, NC | P71616 | transporter activity |
| 1297 | 3103523 | C | E | 3146950 | T | E | 3141164 | T | E | Yes | S, NULL | Null | — |
| 1298 | 3108827 | T | T | 3152254 | C | A | 3146468 | C | A | Yes | NS, NC | O05814 | tRNA ligase activity |
| 1299 | 3108991 | C | R | 3152418 | T | H | 3146632 | T | H | Yes | NS, NC | O05814 | tRNA ligase activity |
| 1300 | 3111157 | C | R | 3154584 | T | R | 3148798 | T | R | Yes | S, NULL | Null | — |
| 1301 | 3111158 | C | R | 3154585 | G | R | 3148799 | G | R | Yes | S, NULL | Null | — |
| 1302 | 3114301 | G | C | 3157782 | C | W | 3151996 | C | W | Yes | NS, NC | O05810 | ATP binding activity |
| 1303 | 3115235 | T | S | 3158716 | C | G | 3152930 | C | G | Yes | NS, NC | O05809 | nucleotide binding |
| | | | | | | | | | | | | | activity |
| 1304 | 3115753 | T | Q | 3159234 | C | R | 3153448 | C | R | Yes | NS, NC | O05809 | nucleotide binding |
| | | | | | | | | | | | | | activity |
| 1305 | 3119320 | C | N | 3162801 | A | K | 3157013 | A | K | Yes | NS, NC | O05806 | — |
| 1306 | 3121306 | T | D | 3164787 | C | D | 3158999 | C | D | Yes | S, NULL | Null | — |
| 1307 | 3127009 | G | P | 3170490 | C | P | 3164702 | C | P | Yes | S, NULL | Null | — |
| 1308 | 3131012 | G | R | 3174493 | A | C | 3168651 | A | C | Yes | NS, NC | O33344 | — |
| 1309 | 3131107 | G | P | 3174588 | C | R | 3168746 | C | R | Yes | NS, NC | O33344 | — |
| 1310 | 3133059 | A | I | 3176540 | G | I | 3170698 | G | I | Yes | S, NULL | Null | — |
| 1311 | 3135930 | G | A | 3179411 | T | D | 3173569 | T | D | Yes | NS, NC | O33350 | isoprenoid biosynthesis |
| 1312 | 3137504 | A | F | 3180985 | C | V | 3175143 | C | V | Yes | NS, NC | O33351 | metalloendopeptidase |
| | | | | | | | | | | | | | activity |
| 1313 | 3141276 | T | Null | 3184757 | G | Null | 3178914 | G | null | Yes | nc, NULL | Null | — |
| 1314 | 3144308 | C | R | 3187789 | T | W | 3181946 | T | R | Yes | NS, NC | Q10802 | integral to membrane |
| 1315 | 3145285 | G | H | 3188766 | A | Y | 3182923 | A | Y | Yes | NS, C | Q10803 | membrane |
| 1316 | 3145758 | G | A | 3189239 | A | A | 3183396 | A | A | Yes | S, NULL | Null | — |
| 1317 | 3146096 | C | Null | 3189577 | T | Null | 3183734 | T | null | No | nc, NULL | Null | — |
| 1318 | 3146180 | C | K | 3189661 | T | K | 3183818 | T | K | Yes | S, NULL | Null | — |
| 1319 | 3149728 | T | Null | 3193210 | C | Null | 3187366 | C | null | No | nc, NULL | Null | — |
| 1320 | 3150908 | A | I | 3194389 | T | K | 3188545 | T | K | Yes | NS, NC | Q10809 | — |
| 1321 | 3152247 | C | V | 3195728 | T | V | 3189884 | T | V | Yes | S, NULL | Null | — |
| 1322 | 3154848 | A | M | 3198329 | G | T | 3192485 | G | T | Yes | NS, NC | Q10788 | translation elongation |
| | | | | | | | | | | | | | factor activity |
| 1323 | 3156820 | G | V | 3200301 | A | V | 3194457 | A | V | Yes | S, NULL | Null | — |
| 1324 | 3162781 | C | G | 3206262 | T | E | 3200418 | T | E | Yes | NS, NC | Q10817 | DNA mediated |
| | | | | | | | | | | | | | transformation |
| 1325 | 3163766 | T | L | 3207247 | A | L | 3201403 | A | L | Yes | S, NULL | Null | — |
| 1326 | 3169156 | T | H | 3212637 | C | R | 3206793 | C | R | Yes | NS, NC | Q10793 | RNA binding activity |
| 1327 | 3169605 | T | N | 3213086 | C | D | 3207242 | C | D | Yes | NS, NC | Q10789 | proteolysis and |
| | | | | | | | | | | | | | peptidolysis |
| 1328 | 3179819 | T | E | 3223300 | C | E | 3217456 | C | E | Yes | S, NULL | Null | — |
| 1329 | 3185717 | A | W | 3229198 | G | R | 3223352 | G | R | Yes | NS, NC | Q10961 | enzyme activity |
| 1330 | 3186529 | C | R | 3230010 | A | L | 3224164 | A | L | Yes | NS, NC | Q10961 | enzyme activity |
| 1331 | 3187380 | A | D | 3230861 | G | D | 3225015 | G | D | Yes | S, NULL | Null | — |
| 1332 | 3192343 | C | G | 3235712 | G | R | 3230035 | G | R | Yes | NS, NC | Q10970 | ATP-binding cassette |
| | | | | | | | | | | | | | (ABC) transporter |
| | | | | | | | | | | | | | activity |
| 1333 | 3193344 | C | R | 3236713 | A | L | 3231036 | A | L | Yes | NS, NC | Q10970 | ATP-binding cassette |
| | | | | | | | | | | | | | (ABC) transporter |
| | | | | | | | | | | | | | activity |
| 1334 | 3199928 | C | Null | 3243352 | T | Null | 3237675 | T | null | No | nc, NULL | Null | — |
| 1335 | 3200987 | G | V | 3244411 | A | M | 3238734 | A | M | Yes | NS, NC | Q10976 | enzyme activity |
| 1336 | 3203120 | C | T | 3246544 | A | N | 3240867 | A | N | Yes | NS, C | Q10977 | enzyme activity |
| 1337 | 3204152 | A | Q | 3247576 | G | R | 3241899 | G | R | Yes | NS, NC | Q10977 | enzyme activity |
| 1338 | 3211268 | T | A | 3254692 | C | A | 3249015 | C | A | Yes | S, NULL | Null | — |
| 1339 | 3211453 | T | L | 3254877 | G | R | 3249200 | G | R | Yes | NS, NC | Q10978 | enzyme activity |
| 1340 | 3217579 | A | G | 3261003 | C | G | 3255326 | C | G | Yes | S, NULL | Null | — |
| 1341 | 3219201 | A | I | 3262625 | G | M | 3256948 | G | M | Yes | NS, NC | P96203 | enzyme activity |
| 1342 | 3222603 | G | S | 3266027 | A | S | 3260350 | A | S | Yes | S, NULL | Null | — |
| 1343 | 3224288 | C | A | 3267712 | A | E | 3262035 | A | E | Yes | NS, NC | P96203 | enzyme activity |
| 1344 | 3227610 | G | D | 3271034 | A | N | 3265357 | A | N | Yes | NS, NC | P96204 | enzyme activity |
| 1345 | 3229711 | G | D | 3273135 | C | H | 3267458 | C | H | Yes | NS, NC | P96205 | nucleotide binding |
| | | | | | | | | | | | | | activity |
| 1346 | 3230432 | T | L | 3273856 | C | L | 3268179 | C | L | Yes | S, NULL | Null | — |
| 1347 | 3238652 | A | C | 3282076 | T | S | 3276399 | T | S | Yes | NS, NC | P96291 | enzyme activity |
| 1348 | 3239912 | T | I | 3283336 | G | S | 3277659 | G | S | Yes | NS, NC | P96290 | enzyme activity |
| 1349 | 3240065 | G | R | 3283489 | A | Q | 3277812 | A | Q | Yes | NS, NC | P96290 | enzyme activity |
| 1350 | 3240165 | C | G | 3283589 | G | G | 3277912 | G | G | Yes | S, NULL | Null | — |
| 1351 | 3242680 | A | V | 3286104 | C | V | 3280427 | C | V | Yes | S, NULL | Null | — |
| 1352 | 3243139 | T | A | 3286563 | C | A | 3280886 | C | A | Yes | S, NULL | Null | — |
| 1353 | 3248962 | A | V | 3292272 | G | A | 3286595 | G | A | Yes | NS, C | P96285 | alcohol dehydrogenase |
| | | | | | | | | | | | | | activity |
| 1354 | 3249193 | C | G | 3292503 | A | V | 3286826 | A | V | Yes | NS, C | P96285 | alcohol dehydrogenase |
| | | | | | | | | | | | | | activity |
| 1355 | 3250170 | A | A | 3293480 | G | A | 3287803 | G | A | Yes | S, NULL | Null | — |
| 1356 | 3253409 | G | R | 3296718 | C | G | 3291041 | C | G | Yes | NS, NC | P96284 | enzyme activity |
| 1357 | 3257940 | T | T | 3301249 | C | A | 3295572 | C | A | Yes | NS, NC | P95141 | enzyme activity |
| 1358 | 3259371 | T | Null | 3302680 | C | Null | 3297003 | C | null | Yes | nc, NULL | Null | — |
| 1359 | 3265154 | T | H | 3308463 | C | R | 3302785 | C | R | Yes | NS, NC | P95137 | — |
| 1360 | 3265769 | A | E | 3309078 | G | E | 3303400 | G | E | Yes | S, NULL | Null | — |
| 1361 | 3266065 | C | T | 3309374 | T | I | 3303696 | T | I | Yes | NS, NC | P95136 | — |
| 1362 | 3267563 | A | Null | 3310872 | G | Null | 3305194 | G | null | Yes | S, NULL | Null | — |
| 1363 | 3267807 | C | D | 3311116 | G | D | 3305438 | G | D | Yes | S, NULL | Null | — |
| 1364 | 3269417 | T | V | 3312725 | C | V | 3307047 | C | V | Yes | S, NULL | Null | — |
| 1365 | 3271101 | G | A | 3314409 | A | A | 3308731 | A | A | Yes | S, NULL | Null | — |
| 1366 | 3277243 | A | S | 3320551 | G | S | 3314873 | G | S | Yes | S, NULL | Null | — |
| 1367 | 3283243 | C | H | 3326551 | A | N | 3320873 | A | N | Yes | NS, NC | P95124 | — |
| 1368 | 3291984 | C | G | 3335294 | T | D | 3329616 | T | D | Yes | NS, NC | P95116 | recombinase activity |
| 1369 | 3292201 | C | A | 3335511 | T | T | 3329833 | T | T | Yes | NS, NC | P95116 | recombinase activity |
| 1370 | 3292395 | C | R | 3335705 | G | P | 3330027 | G | P | Yes | NS, NC | P95116 | recombinase activity |
| 1371 | 3301607 | T | E | 3345044 | G | D | 3339424 | G | D | Yes | NS, C | O53237 | 3-isopropylmalate |
| | | | | | | | | | | | | | dehydratase activity |
| 1372 | 3303630 | T | E | 3347067 | G | A | 3341447 | G | A | Yes | NS, NC | O53239 | — |
| 1373 | 3304818 | A | T | 3348255 | G | A | 3342635 | G | A | Yes | NS, NC | O53240 | — |
| 1374 | 3309535 | G | L | 3352916 | A | F | 3347295 | A | F | Yes | NS, NC | P95313 | 3-isopropylmalate |
| | | | | | | | | | | | | | dehydrogenase activity |
| 1375 | 3312033 | A | S | 3355414 | T | C | 3349793 | T | C | Yes | NS, NC | O53244 | — |
| 1376 | 3313133 | C | P | 3356514 | T | P | 3350893 | T | P | Yes | S, NULL | Null | — |
| 1377 | 3321979 | A | D | 3365361 | G | G | 3359739 | G | G | Yes | NS, NC | O53253 | — |
| 1378 | 3326484 | T | Null | 3369866 | G | Null | 3364244 | G | null | No | nc, NULL | Null | — |
| 1379 | 3327875 | C | V | 3371257 | T | I | 3365635 | T | I | Yes | NS, C | O53258 | amidase activity |
| 1380 | 3327980 | T | T | 3371362 | C | A | 3365740 | C | A | Yes | NS, NC | O53258 | amidase activity |
| 1381 | 3328016 | A | L | 3371398 | G | L | 3365776 | G | L | Yes | S, NULL | Null | — |
| 1382 | 3333886 | C | V | 3377268 | G | L | 3371647 | G | L | Yes | NS, C | P31500 | — |
| 1383 | 3338640 | T | G | 3382077 | C | G | 3377816 | C | G | Yes | S, NULL | Null | — |
| 1384 | 3339158 | T | T | 3382595 | C | A | 3378334 | C | A | Yes | NS, NC | P96354 | peroxidase activity |
| 1385 | 3343458 | C | Null | 3386895 | T | Null | 3382634 | T | null | Yes | nc, NULL | Null | — |
| 1386 | 3343463 | G | Null | 3386900 | A | Null | 3382639 | A | null | Yes | nc, NULL | Null | — |
| 1387 | 3343657 | A | V | 3387094 | G | A | 3382833 | G | A | Yes | NS, C | O53275 | electron transporter |
| | | | | | | | | | | | | | activity |
| 1388 | 3345242 | C | V | 3388679 | T | V | 3384418 | T | V | Yes | S, NULL | Null | — |
| 1389 | 3353514 | C | R | 3396951 | T | Q | 3392690 | T | Q | Yes | NS, NC | O53283 | — |
| 1390 | 3354831 | G | A | 3398268 | T | D | 3394007 | T | D | Yes | NS, NC | O53284 | — |
| 1391 | 3359515 | A | S | 3402952 | C | A | 3398691 | C | A | Yes | NS, NC | O53289 | phosphoserine |
| | | | | | | | | | | | | | phosphatase activity |
| 1392 | 3362817 | A | Null | 3406254 | G | Null | 3401993 | G | null | No | nc, NULL | Null | — |
| 1393 | 3366744 | A | R | 3410181 | G | R | 3405920 | G | R | Yes | S, NULL | Null | — |
| 1394 | 3371878 | G | Null | 3415329 | A | Null | 3411054 | A | null | Yes | nc, NULL | Null | — |
| 1395 | 3376988 | T | Y | 3420439 | C | Y | 3416164 | C | Y | Yes | S, NULL | Null | — |
| 1396 | 3377371 | G | G | 3420822 | A | D | 3416547 | A | D | Yes | NS, NC | P95099 | monooxygenase activity |
| 1397 | 3384993 | T | C | 3428443 | G | G | 3424236 | G | G | Yes | NS, NC | P95095 | cellular response to |
| | | | | | | | | | | | | | starvation |
| 1398 | 3385588 | C | P | 3429038 | T | L | 3424831 | T | L | Yes | NS, NC | P95095 | cellular response to |
| | | | | | | | | | | | | | starvation |
| 1399 | 3386152 | A | Null | 3429602 | G | Null | 3425395 | G | null | Yes | nc, NULL | Null | — |
| 1400 | 3392209 | G | Null | 3435659 | C | Null | 3431452 | C | null | Yes | nc, NULL | Null | — |
| 1401 | 3394933 | A | I | 3438383 | G | I | 3434176 | G | I | Yes | S, NULL | Null | — |
| 1402 | 3397089 | G | G | 3440539 | A | G | 3436332 | A | G | Yes | S, NULL | Null | — |
| 1403 | 3401113 | A | L | 3444563 | G | L | 3440356 | G | L | Yes | S, NULL | Null | — |
| 1404 | 3401886 | A | V | 3445336 | G | A | 3441129 | G | A | Yes | NS, C | P95078 | protein kinase activity |
| 1405 | 3404010 | A | C | 3447460 | G | R | 3443253 | G | R | Yes | NS, NC | Q06861 | DNA binding activity |
| 1406 | 3405330 | A | I | 3448780 | G | V | 3444573 | G | V | Yes | NS, C | O53300 | — |
| 1407 | 3409943 | G | A | 3453393 | A | T | 3449186 | A | T | Yes | NS, NC | O53304 | molecular_function |
| | | | | | | | | | | | | | unknown |
| 1408 | 3410810 | G | V | 3454260 | C | L | 3450053 | C | L | Yes | NS, C | O53304 | molecular_function |
| | | | | | | | | | | | | | unknown |
| 1409 | 3414061 | C | Null | 3457511 | G | Null | 3453304 | G | null | No | nc, NULL | Null | — |
| 1410 | 3417571 | C | M | 3461021 | T | I | 3456814 | T | I | Yes | NS, NC | O05771 | — |
| 1411 | 3421176 | G | G | 3464626 | A | E | 3460424 | A | E | Yes | NS, NC | O05775 | hydrolase activity |
| 1412 | 3423466 | G | A | 3466916 | C | G | 3462714 | C | G | Yes | NS, C | P77909 | enzyme activity |
| 1413 | 3423862 | C | E | 3467312 | G | D | 3463110 | G | D | Yes | NS, C | O05776 | — |
| 1414 | 3424012 | G | A | 3467462 | C | A | 3463260 | C | A | Yes | S, NULL | Null | — |
| 1415 | 3426715 | C | S | 3470165 | T | N | 3465963 | T | N | Yes | NS, C | P96293 | cytokinesis |
| 1416 | 3430975 | A | I | 3474424 | G | V | 3470223 | G | V | Yes | NS, C | O05783 | ferredoxin-NADP |
| | | | | | | | | | | | | | reductase activity |
| 1417 | 3431707 | G | D | 3475156 | A | N | 3470955 | A | N | Yes | NS, NC | O05783 | ferredoxin-NADP |
| | | | | | | | | | | | | | reductase activity |
| 1418 | 3434037 | G | V | 3477486 | A | I | 3473285 | A | I | Yes | NS, C | O05785 | — |
| 1419 | 3434151 | T | Null | 3477600 | C | Null | 3473399 | C | null | No | nc, NULL | Null | — |
| 1420 | 3435166 | G | A | 3478615 | A | T | 3474414 | A | T | Yes | NS, NC | O05786 | enzyme activity |
| 1421 | 3436346 | A | G | 3479795 | G | G | 3475594 | G | G | Yes | S, NULL | Null | — |
| 1422 | 3436653 | C | C | 3480103 | G | W | 3475902 | G | W | Yes | NS, NC | O05790 | metabolism |
| 1423 | 3437192 | G | R | 3480642 | T | L | 3476441 | T | L | Yes | NS, NC | O05790 | metabolism |
| 1424 | 3437336 | C | P | 3480786 | T | S | 3476585 | T | S | Yes | NS, NC | O05791 | zinc ion binding activity |
| 1425 | 3438022 | A | T | 3481472 | G | A | 3477271 | G | A | Yes | NS, NC | P96354 | peroxidase activity |
| 1426 | 3438540 | A | G | 3481990 | G | G | 3477789 | G | G | Yes | S, NULL | Null | — |
| 1427 | 3442328 | T | S | 3488553 | G | R | 3484352 | G | R | Yes | NS, NC | O07033 | — |
| 1428 | 3442459 | A | Q | 3488684 | G | R | 3484483 | G | R | Yes | NS, NC | O07034 | — |
| 1429 | 3443112 | G | Null | 3489337 | C | Null | 3485136 | C | null | No | nc, NULL | Null | — |
| 1430 | 3445311 | A | V | 3491536 | G | A | 3487335 | G | A | Yes | NS, C | O05798 | — |
| 1431 | 3446202 | G | C | 3492427 | T | F | 3488226 | T | F | Yes | NS, NC | O05800 | — |
| 1432 | 3447457 | C | G | 3493682 | A | V | 3489481 | A | null | Yes | NS, C | Not | — |
| | | | | | | | | | | | | annotated | |
| 1433 | 3452190 | G | T | 3498415 | A | I | 3494214 | A | I | Yes | NS, NC | P95194 | ATP binding activity |
| 1434 | 3456796 | T | Null | 3501684 | C | Null | 3497171 | C | null | Yes | S, NULL | Null | — |
| 1435 | 3459632 | A | N | 3504520 | G | S | 3500007 | G | S | Yes | NS, C | P95188 | lyase activity |
| 1436 | 3460073 | C | S | 3504961 | T | F | 3500448 | T | F | Yes | NS, NC | P95188 | lyase activity |
| 1437 | 3460112 | T | L | 3505000 | C | P | 3500487 | C | P | Yes | NS, NC | P95188 | lyase activity |
| 1438 | 3460114 | C | P | 3505002 | A | T | 3500489 | A | T | Yes | NS, NC | P95188 | lyase activity |
| 1439 | 3464200 | C | Null | 3509088 | G | Null | 3504575 | G | null | No | nc, NULL | Null | — |
| 1440 | 3464257 | C | Q | 3509145 | A | H | 3504632 | A | H | Yes | NS, NC | P95184 | — |
| 1441 | 3465229 | G | Q | 3510117 | T | K | 3505604 | T | K | Yes | NS, NC | P95182 | — |
| 1442 | 3466696 | G | Null | 3511584 | T | Null | 3507071 | T | null | No | nc, NULL | Null | — |
| 1443 | 3467191 | C | G | 3512079 | A | G | 3507566 | A | G | Yes | S, NULL | Null | — |
| 1444 | 3468833 | T | N | 3513721 | C | N | 3509208 | C | N | Yes | S, NULL | Null | — |
| 1445 | 3470134 | T | G | 3515022 | C | G | 3510509 | C | G | Yes | S, NULL | Null | — |
| 1446 | 3472676 | T | N | 3517564 | C | N | 3513051 | C | N | Yes | S, NULL | Null | — |
| 1447 | 3476153 | G | A | 3521041 | A | T | 3516528 | A | T | Yes | NS, NC | P95173 | electron transporter |
| | | | | | | | | | | | | | activity |
| 1448 | 3478232 | A | Y | 3523120 | T | F | 3518607 | T | F | Yes | NS, C | O86350 | oxidative |
| | | | | | | | | | | | | | phosphorylation |
| 1449 | 3480424 | A | D | 3525312 | G | G | 3520799 | G | G | Yes | NS, NC | O53307 | oxidative |
| | | | | | | | | | | | | | phosphorylation |
| 1450 | 3480666 | A | I | 3525554 | G | V | 3521041 | G | V | Yes | NS, C | O53307 | oxidative |
| | | | | | | | | | | | | | phosphorylation |
| 1451 | 3482095 | T | A | 3526983 | A | A | 3522470 | A | A | Yes | S, NULL | Null | — |
| 1452 | 3483661 | T | E | 3528549 | G | D | 3524036 | G | D | Yes | NS, C | O53309 | — |
| 1453 | 3488207 | G | L | 3530952 | C | V | 3528588 | C | V | Yes | NS, C | O53311 | iron ion binding activity |
| 1454 | 3489142 | T | E | 3531888 | G | D | 3529524 | G | D | Yes | NS, C | O53313 | transporter activity |
| 1455 | 3491098 | C | G | 3533844 | T | E | 3531480 | T | E | Yes | NS, NC | O53314 | — |
| 1456 | 3492231 | G | V | 3534977 | C | V | 3532613 | C | V | Yes | S, NULL | Null | — |
| 1457 | 3493545 | C | R | 3536291 | T | W | 3533927 | T | W | Yes | NS, NC | O53318 | — |
| 1458 | 3497395 | A | M | 3540141 | G | T | 3537777 | G | T | Yes | NS, NC | O53321 | enzyme activity |
| 1459 | 3499414 | C | A | 3542160 | T | V | 3539796 | T | V | Yes | NS, C | O53324 | metabolism |
| 1460 | 3500015 | C | L | 3542761 | G | L | 3540397 | G | L | Yes | S, NULL | Null | — |
| 1461 | 3510233 | G | A | 3555696 | A | V | 3550616 | A | V | Yes | NS, C | O53336 | — |
| 1462 | 3515180 | T | Q | 3560642 | C | R | 3555549 | C | R | Yes | NS, NC | O53339 | molecular_function |
| | | | | | | | | | | | | | unknown |
| 1463 | 3519984 | A | E | 3565446 | G | E | 3560353 | G | E | Yes | S, NULL | Null | — |
| 1464 | 3522549 | A | R | 3568001 | C | R | 3562908 | C | R | Yes | S, NULL | Null | — |
| 1465 | 3526379 | G | P | 3571831 | T | Q | 3566738 | T | Q | Yes | NS, NC | O53345 | magnesium ion binding |
| | | | | | | | | | | | | | activity |
| 1466 | 3528181 | C | D | 3573633 | T | N | 3568540 | T | N | Yes | NS, NC | O53346 | potassium channel |
| | | | | | | | | | | | | | activity |
| 1467 | 3532503 | G | A | 3577955 | A | V | 3572862 | A | V | Yes | NS, C | O53348 | DNA binding activity |
| 1468 | 3538731 | T | N | 3584187 | C | D | 3579093 | C | null | Yes | NS, NC | O05859 | proteolysis and |
| | | | | | | | | | | | | | peptidolysis |
| 1469 | 3542388 | C | A | 3587844 | T | V | 3582750 | T | V | Yes | NS, C | O05855 | nucleic acid binding |
| | | | | | | | | | | | | | activity |
| 1470 | 3544683 | C | A | 3590139 | T | V | 3585045 | T | V | Yes | NS, C | O05854 | — |
| 1471 | 3545177 | C | Null | 3590633 | G | Null | 3585539 | G | null | No | nc, NULL | Null | — |
| 1472 | 3545178 | G | Null | 3590634 | C | Null | 3585540 | C | null | No | nc, NULL | Null | — |
| 1473 | 3549450 | A | Q | 3594848 | G | Q | 3589751 | G | Q | Yes | S, NULL | Null | — |
| 1474 | 3550026 | A | R | 3595424 | G | R | 3590327 | G | R | Yes | S, NULL | Null | — |
| 1475 | 3551995 | T | N | 3597393 | C | D | 3592296 | C | D | Yes | NS, NC | O05846 | ATP binding activity |
| 1476 | 3556127 | T | T | 3601524 | C | A | 3596428 | C | A | Yes | NS, NC | O05841 | N-acetyltransferase |
| | | | | | | | | | | | | | activity |
| 1477 | 3558424 | A | H | 3603821 | G | R | 3598725 | G | R | Yes | NS, NC | P22487 | 3-phosphoshikimate 1- |
| | | | | | | | | | | | | | carboxyvinyltransferase |
| | | | | | | | | | | | | | activity |
| 1478 | 3561226 | A | L | 3606623 | G | L | 3601527 | G | L | Yes | S, NULL | Null | — |
| 1479 | 3562541 | A | C | 3607938 | G | R | 3602842 | G | R | Yes | NS, NC | O05875 | electron transporter |
| | | | | | | | | | | | | | activity |
| 1480 | 3563125 | A | V | 3608522 | G | V | 3603426 | G | V | Yes | S, NULL | Null | — |
| 1481 | 3566088 | C | G | 3611484 | G | G | 3606388 | G | G | Yes | S, NULL | Null | — |
| 1482 | 3569604 | T | H | 3615000 | C | R | 3609903 | C | R | Yes | NS, NC | O05884 | enzyme activity |
| 1483 | 3577972 | G | P | 3623368 | C | P | 3618271 | C | P | Yes | S, NULL | Null | — |
| 1484 | 3578330 | A | L | 3623726 | G | S | 3618629 | G | S | Yes | NS, NC | O05889 | — |
| 1485 | 3578950 | A | A | 3624346 | C | A | 3619249 | C | A | Yes | S, NULL | Null | — |
| 1486 | 3579086 | C | G | 3624482 | T | D | 3619385 | T | D | Yes | NS, NC | O05889 | — |
| 1487 | 3579193 | T | V | 3624589 | C | V | 3619492 | C | V | Yes | S, NULL | Null | — |
| 1488 | 3579310 | T | L | 3624706 | C | L | 3619609 | C | L | Yes | S, NULL | Null | — |
| 1489 | 3583683 | T | I | 3629079 | C | V | 3623982 | C | V | Yes | NS, C | O08364 | adenosylhomocysteinase |
| | | | | | | | | | | | | | activity |
| 1490 | 3592693 | A | L | 3638089 | G | S | 3632992 | G | S | Yes | NS, NC | O86374 | carbohydrate |
| | | | | | | | | | | | | | metabolism |
| 1491 | 3601629 | G | P | 3647037 | A | S | 3641940 | A | S | Yes | NS, NC | P96871 | dTDP-4- |
| | | | | | | | | | | | | | dehydrorhamnose |
| | | | | | | | | | | | | | reductase activity |
| 1492 | 3609075 | G | S | 3654483 | A | N | 3649386 | A | N | Yes | NS, C | P96877 | metabolism |
| 1493 | 3609941 | T | F | 3655349 | C | F | 3650252 | C | F | Yes | S, NULL | Null | — |
| 1494 | 3612854 | G | R | 3658262 | C | G | 3653165 | C | G | Yes | NS, NC | P96880 | phospho- |
| | | | | | | | | | | | | | ribosylaminoimidazole |
| | | | | | | | | | | | | | carboxylase |
| | | | | | | | | | | | | | activity |
| 1495 | 3615280 | T | L | 3660688 | C | S | 3655591 | C | S | Yes | NS, NC | P96882 | — |
| 1496 | 3615970 | T | N | 3661378 | C | D | 3656280 | C | D | Yes | NS, NC | P96884 | biotin-apoprotein ligase |
| | | | | | | | | | | | | | activity |
| 1497 | 3617992 | C | A | 3663400 | T | V | 3658302 | T | V | Yes | NS, C | P96885 | biotin carboxylase |
| | | | | | | | | | | | | | activity |
| 1498 | 3619140 | T | S | 3664611 | G | A | 3659450 | G | A | Yes | NS, NC | P96887 | — |
| 1499 | 3619961 | A | D | 3665432 | G | G | 3660271 | G | G | Yes | NS, NC | P96888 | thiosulfate |
| | | | | | | | | | | | | | sulfurtransferase |
| | | | | | | | | | | | | | activity |
| 1500 | 3621718 | T | H | 3667189 | C | H | 3662028 | C | H | Yes | S, NULL | Null | — |
| 1501 | 3622087 | C | G | 3667558 | T | G | 3662397 | T | G | Yes | S, NULL | Null | — |
| 1502 | 3624565 | T | A | 3670036 | C | A | 3664875 | C | A | Yes | S, NULL | Null | — |
| 1503 | 3626758 | A | V | 3672229 | G | A | 3667068 | G | A | Yes | NS, C | P96896 | DNA binding activity |
| 1504 | 3628943 | T | C | 3674414 | G | G | 3669253 | G | G | Yes | NS, NC | P96898 | metabolism |
| 1505 | 3633454 | A | M | 3678925 | G | V | 3673764 | G | V | Yes | NS, NC | P96901 | nucleotide binding |
| | | | | | | | | | | | | | activity |
| 1506 | 3633751 | A | N | 3679222 | G | D | 3674061 | G | D | Yes | NS, NC | P96901 | nucleotide binding |
| | | | | | | | | | | | | | activity |
| 1507 | 3634474 | G | E | 3679945 | A | K | 3674784 | A | K | Yes | NS, NC | P96901 | nucleotide binding |
| | | | | | | | | | | | | | activity |
| 1508 | 3636073 | C | R | 3681544 | A | R | 3676383 | A | R | Yes | S, NULL | Null | — |
| 1509 | 3639879 | T | S | 3685350 | C | G | 3680189 | C | G | Yes | NS, NC | O65931 | enzyme activity |
| 1510 | 3641272 | T | N | 3686743 | C | D | 3681582 | C | D | Yes | NS, NC | O07166 | pseudouridylate |
| | | | | | | | | | | | | | synthase activity |
| 1511 | 3644541 | G | S | 3690012 | A | L | 3684851 | A | L | Yes | NS, NC | O53355 | cytoplasm |
| 1512 | 3645379 | C | A | 3690850 | T | T | 3685689 | T | T | Yes | NS, NC | O53355 | cytoplasm |
| 1513 | 3648206 | C | A | 3693677 | A | A | 3688572 | A | A | Yes | S, NULL | Null | — |
| 1514 | 3650706 | G | G | 3696177 | A | S | 3691072 | A | S | Yes | NS, NC | O53360 | carbohydrate |
| | | | | | | | | | | | | | metabolism |
| 1515 | 3652233 | G | A | 3697704 | A | T | 3692599 | A | T | Yes | NS, NC | O53361 | hydrolase activity |
| 1516 | 3654880 | G | Null | 3700351 | A | Null | 3695243 | A | null | No | nc, NULL | Null | — |
| 1517 | 3657068 | A | W | 3702539 | G | R | 3697430 | G | R | Yes | NS, NC | O53366 | pyrimidine base |
| | | | | | | | | | | | | | metabolism |
| 1518 | 3659623 | A | V | 3705094 | G | V | 3699985 | G | V | Yes | S, NULL | Null | — |
| 1519 | 3660007 | A | E | 3705478 | G | E | 3700369 | G | E | Yes | S, NULL | Null | — |
| 1520 | 3661401 | G | E | 3706872 | C | D | 3701763 | C | D | Yes | NS, C | O53371 | electron transporter |
| | | | | | | | | | | | | | activity |
| 1521 | 3662101 | C | Null | 3707572 | G | Null | 3702463 | G | null | No | nc, NULL | Null | — |
| 1522 | 3662102 | G | Null | 3707573 | C | Null | 3702464 | C | null | No | nc, NULL | Null | — |
| 1523 | 3663410 | A | A | 3708884 | G | A | 3703775 | G | A | Yes | S, NULL | Null | — |
| 1524 | 3671821 | T | L | 3714568 | C | L | 3712186 | C | L | Yes | S, NULL | Null | — |
| 1525 | 3672930 | T | D | 3715677 | C | D | 3713295 | C | D | Yes | S, NULL | Null | — |
| 1526 | 3673148 | A | V | 3715895 | G | V | 3713513 | G | V | Yes | S, NULL | Null | — |
| 1527 | 3687716 | G | T | 3730462 | A | I | 3728072 | A | null | Yes | NS, NC | O53393 | carboxypeptidase A |
| | | | | | | | | | | | | | activity |
| 1528 | 3693443 | G | A | 3740060 | T | E | 3732216 | T | E | Yes | NS, NC | O53395 | — |
| 1529 | 3693692 | A | V | 3740309 | C | G | 3732465 | C | G | Yes | NS, C | O53395 | — |
| 1530 | 3696063 | A | T | 3742525 | G | T | 3734759 | G | T | Yes | S, NULL | Null | — |
| 1531 | 3697083 | G | Null | 3743545 | A | Null | 3735779 | A | null | No | nc, NULL | Null | — |
| 1532 | 3699350 | T | N | 3745812 | C | D | 3738047 | C | null | Yes | NS, NC | O50378 | — |
| 1533 | 3700460 | C | W | 3746922 | T | W | 3739156 | T | null | Yes | S, NULL | Null | — |
| 1534 | 3701886 | G | A | 3748349 | C | G | 3740583 | C | null | Yes | NS, C | O50378 | — |
| 1535 | 3703453 | A | S | 3749916 | G | P | 3742150 | G | null | Yes | NS, NC | O50378 | — |
| 1536 | 3703940 | C | G | 3750403 | G | G | 3742637 | G | null | Yes | S, NULL | Null | — |
| 1537 | 3703950 | T | Y | 3750413 | A | F | 3742647 | A | null | Yes | NS, C | O50378 | — |
| 1538 | 3703954 | C | G | 3750417 | T | S | 3742651 | T | null | Yes | NS, NC | O50378 | — |
| 1539 | 3704386 | G | L | 3750849 | A | L | 3743083 | A | null | Yes | S, NULL | Null | — |
| 1540 | 3704564 | G | G | 3751027 | A | G | 3743261 | A | null | Yes | S, NULL | Null | — |
| 1541 | 3704570 | G | N | 3751033 | A | N | 3743267 | A | null | Yes | S, NULL | Null | — |
| 1542 | 3706162 | T | I | 3752625 | G | L | 3744859 | G | null | Yes | NS, C | O50378 | — |
| 1543 | 3706947 | G | Null | 3753410 | T | Null | 3745645 | T | null | Yes | nc, NULL | Null | — |
| 1544 | 3708970 | A | Null | 3755439 | G | Null | 3747674 | G | null | No | nc, NULL | Null | — |
| 1545 | 3711573 | T | S | 3758042 | C | S | 3750277 | C | null | Yes | S, NULL | Null | — |
| 1546 | 3715389 | A | V | 3761859 | G | A | 3754086 | G | null | Yes | NS, C | O50379 | — |
| 1547 | 3717907 | C | D | 3764377 | T | N | 3756604 | T | null | Yes | NS, NC | O50379 | — |
| 1548 | 3719141 | C | A | 3765611 | G | A | 3757838 | G | null | Yes | S, NULL | Null | — |
| 1549 | 3719324 | T | Null | 3765794 | G | Null | 3758021 | G | null | Yes | S, NULL | Null | — |
| 1550 | 3719936 | G | G | 3766406 | C | G | 3758633 | C | null | Yes | S, NULL | Null | — |
| 1551 | 3720790 | G | Null | 3767260 | A | Null | 3759487 | A | null | No | nc, NULL | Null | — |
| 1552 | 3722971 | C | A | 3769441 | T | V | 3761668 | T | V | Yes | NS, C | O50383 | — |
| 1553 | 3724114 | G | P | 3770584 | T | Q | 3762811 | T | Q | Yes | NS, NC | O50385 | enzyme activity |
| 1554 | 3724214 | T | Null | 3770684 | C | Null | 3762911 | C | null | No | nc, NULL | Null | — |
| 1555 | 3724771 | A | L | 3771241 | G | L | 3763468 | G | L | Yes | S, NULL | Null | — |
| 1556 | 3724938 | G | R | 3771408 | T | R | 3763635 | T | R | Yes | S, NULL | Null | — |
| 1557 | 3725154 | C | I | 3771624 | A | I | 3763851 | A | I | Yes | S, NULL | Null | — |
| 1558 | 3726142 | A | Null | 3772612 | G | Null | 3764839 | G | null | No | nc, NULL | Null | — |
| 1559 | 3729791 | T | R | 3776261 | G | R | 3768488 | G | R | Yes | S, NULL | Null | — |
| 1560 | 3733435 | A | S | 3779791 | G | G | 3772027 | G | G | Yes | NS, NC | O50396 | — |
| 1561 | 3736194 | A | S | 3782550 | G | S | 3774786 | G | S | Yes | S, NULL | Null | — |
| 1562 | 3736445 | T | R | 3782801 | G | R | 3775037 | G | R | Yes | S, NULL | Null | — |
| 1563 | 3739586 | G | G | 3785942 | A | R | 3778178 | A | R | Yes | NS, NC | O50400 | molecular_function |
| | | | | | | | | | | | | | unknown |
| 1564 | 3739673 | G | V | 3786029 | A | I | 3778265 | A | I | Yes | NS, C | O50400 | molecular_function |
| | | | | | | | | | | | | | unknown |
| 1565 | 3742005 | G | G | 3788361 | T | * | 3780597 | T | null | Yes | NS, TP | O50402 | enzyme activity |
| 1566 | 3746351 | T | T | 3792708 | C | T | 3784944 | C | T | Yes | S, NULL | Null | — |
| 1567 | 3747920 | T | Y | 3794277 | C | C | 3786513 | C | null | Yes | NS, NC | O50408 | — |
| 1568 | 3752240 | G | Null | 3799953 | A | Null | 3790831 | A | null | No | nc, NULL | Null | — |
| 1569 | 3755845 | C | A | 3803582 | G | G | 3794460 | G | G | Yes | NS, C | O50415 | — |
| 1570 | 3756614 | A | L | 3804273 | G | P | 3795151 | G | P | Yes | NS, NC | Q11198 | metabolism |
| 1571 | 3760440 | C | S | 3808099 | T | N | 3798977 | T | N | Yes | NS, C | Q11195 | methyltransferase |
| | | | | | | | | | | | | | activity |
| 1572 | 3763664 | T | M | 3811323 | C | V | 3802201 | C | V | Yes | NS, NC | Q50730 | integral to membrane |
| 1573 | 3763966 | A | V | 3811625 | G | A | 3802503 | G | A | Yes | NS, C | Q50730 | integral to membrane |
| 1574 | 3766395 | A | I | 3814054 | G | I | 3804932 | G | I | Yes | S, NULL | Null | — |
| 1575 | 3772351 | A | H | 3820010 | G | R | 3810888 | G | R | Yes | NS, NC | Q50724 | carbohydrate |
| | | | | | | | | | | | | | metabolism |
| 1576 | 3773467 | C | Null | 3820835 | G | Null | 3811715 | G | null | Yes | S, NULL | Null | — |
| 1577 | 3774060 | A | C | 3821428 | G | R | 3812308 | G | R | Yes | NS, NC | Q50723 | — |
| 1578 | 3777374 | A | L | 3824742 | G | L | 3815622 | G | L | Yes | S, NULL | Null | — |
| 1579 | 3783323 | G | A | 3830691 | A | A | 3821571 | A | A | Yes | S, NULL | Null | — |
| 1580 | 3795810 | T | H | 3848104 | C | R | 3834058 | C | R | Yes | NS, NC | O06247 | DNA binding activity |
| 1581 | 3799589 | C | A | 3851883 | A | S | 3837836 | A | S | Yes | NS, NC | O06250 | molecular_function |
| | | | | | | | | | | | | | unknown |
| 1582 | 3799590 | C | A | 3851884 | T | A | 3837837 | T | A | Yes | S, NULL | Null | — |
| 1583 | 3801054 | T | T | 3853348 | C | A | 3839301 | C | A | Yes | NS, NC | O06251 | — |
| 1584 | 3804863 | C | P | 3857157 | T | L | 3843110 | T | L | Yes | NS, NC | O06254 | — |
| 1585 | 3805949 | T | Null | 3858243 | C | Null | 3844196 | C | null | No | nc, NULL | Null | — |
| 1586 | 3812465 | T | S | 3864760 | C | G | 3850712 | C | G | Yes | NS, NC | O06264 | nucleotide binding |
| | | | | | | | | | | | | | activity |
| 1587 | 3816846 | A | D | 3869141 | C | A | 3855093 | C | A | Yes | NS, NC | O33354 | transporter activity |
| 1588 | 3817926 | T | I | 3870221 | C | I | 3856173 | C | I | Yes | S, NULL | Null | — |
| 1589 | 3818947 | C | G | 3871242 | T | G | 3857194 | T | G | Yes | S, NULL | Null | — |
| 1590 | 3823086 | A | I | 3875382 | G | V | 3861333 | G | V | Yes | NS, C | O06321 | — |
| 1591 | 3824956 | T | A | 3877252 | C | A | 3863203 | C | A | Yes | S, NULL | Null | — |
| 1592 | 3826984 | A | N | 3879284 | C | K | 3865235 | C | K | Yes | NS, NC | O06326 | RNA binding activity |
| 1593 | 3831379 | C | V | 3883679 | G | V | 3869630 | G | V | Yes | S, NULL | Null | — |
| 1594 | 3831382 | G | G | 3883682 | T | G | 3869633 | T | G | Yes | S, NULL | Null | — |
| 1595 | 3831386 | A | T | 3883686 | G | A | 3869637 | G | A | Yes | NS, NC | O06331 | — |
| 1596 | 3831403 | C | L | 3883703 | T | L | 3869654 | T | L | Yes | S, NULL | Null | — |
| 1597 | 3831407 | A | T | 3883707 | G | A | 3869658 | G | A | Yes | NS, NC | O06331 | — |
| 1598 | 3831541 | C | G | 3883841 | T | G | 3869792 | T | G | Yes | S, NULL | Null | — |
| 1599 | 3831611 | A | I | 3883911 | G | V | 3869862 | G | V | Yes | NS, C | O06331 | — |
| 1600 | 3832059 | G | E | 3884359 | A | K | 3870310 | A | K | Yes | NS, NC | Q50655 | — |
| 1601 | 3832094 | T | I | 3884394 | C | I | 3870345 | C | I | Yes | S, NULL | Null | — |
| 1602 | 3832393 | G | G | 3884693 | C | A | 3870644 | C | A | Yes | NS, C | Q50655 | — |
| 1603 | 3832444 | A | D | 3884744 | G | G | 3870695 | G | G | Yes | NS, NC | Q50655 | — |
| 1604 | 3832483 | G | R | 3884783 | A | H | 3870734 | A | H | Yes | NS, NC | Q50655 | — |
| 1605 | 3832818 | A | N | 3885118 | G | N | 3880312 | G | N | Yes | S, NULL | Null | — |
| 1606 | 3835237 | T | E | 3887537 | C | G | 3882731 | C | G | Yes | NS, NC | O06335 | — |
| 1607 | 3836573 | C | W | 3888873 | G | C | 3884067 | G | C | Yes | NS, NC | O06336 | nutrient reservoir |
| | | | | | | | | | | | | | activity |
| 1608 | 3839628 | A | V | 3893286 | G | A | 3887122 | G | A | Yes | NS, C | O06339 | transporter activity |
| 1609 | 3839818 | A | F | 3893476 | G | L | 3887312 | G | L | Yes | NS, NC | O06339 | transporter activity |
| 1610 | 3840507 | T | V | 3894165 | C | A | 3888001 | C | A | Yes | NS, C | O06340 | — |
| 1611 | 3842065 | A | Null | 3895723 | C | Null | 3889559 | C | null | Yes | S, NULL | Null | — |
| 1612 | 3842157 | A | Null | 3895815 | G | Null | 3889651 | G | null | Yes | S, NULL | Null | — |
| 1613 | 3844494 | A | N | 3898865 | C | T | 3892701 | C | T | Yes | NS, C | O06342 | enzyme activity |
| 1614 | 3850113 | A | E | 3904486 | G | E | 3898322 | G | E | Yes | S, NULL | Null | — |
| 1615 | 3853581 | A | R | 3907954 | G | G | 3901790 | G | G | Yes | NS, NC | O06351 | — |
| 1616 | 3854858 | C | L | 3909231 | G | V | 3903067 | G | V | Yes | NS, C | O06353 | alpha |
| 1617 | 3858259 | C | G | 3912632 | T | D | 3906467 | T | D | Yes | NS, NC | O53539 | pathogenesis |
| 1618 | 3865062 | A | P | 3919435 | G | P | 3913270 | G | P | Yes | S, NULL | Null | — |
| 1619 | 3867127 | G | A | 3921500 | C | A | 3915336 | C | A | Yes | S, NULL | Null | — |
| 1620 | 3868459 | C | D | 3922832 | T | D | 3916668 | T | D | Yes | S, NULL | Null | — |
| 1621 | 3869973 | T | V | 3924346 | C | A | 3918182 | C | A | Yes | NS, C | O53550 | acyl-CoA |
| | | | | | | | | | | | | | dehydrogenase activity |
| 1622 | 3870019 | A | Q | 3924392 | G | Q | 3918228 | G | Q | Yes | S, NULL | Null | — |
| 1623 | 3874389 | G | G | 3928888 | A | D | 3922733 | A | D | Yes | NS, NC | O53552 | — |
| 1624 | 3875932 | A | G | 3930377 | C | G | 3924213 | C | G | Yes | S, NULL | Null | — |
| 1625 | 3876018 | G | G | 3930463 | A | D | 3924299 | A | D | Yes | NS, NC | O53552 | — |
| 1626 | 3908574 | C | Null | 3965355 | T | Null | 3957501 | T | null | No | nc, NULL | Null | — |
| 1627 | 3910673 | C | L | 3967454 | T | L | 3959600 | T | L | Yes | S, NULL | Null | — |
| 1628 | 3911467 | T | S | 3968248 | C | S | 3960394 | C | S | Yes | S, NULL | Null | — |
| 1629 | 3911833 | A | T | 3968614 | G | T | 3960760 | G | T | Yes | S, NULL | Null | — |
| 1630 | 3921125 | A | L | 3977906 | G | L | 3970052 | G | L | Yes | S, NULL | Null | — |
| 1631 | 3927536 | A | H | 3984317 | G | H | 3976463 | G | H | Yes | S, NULL | Null | — |
| 1632 | 3930860 | C | V | 3987641 | T | M | 3979787 | T | M | Yes | NS, NC | P71853 | metabolism |
| 1633 | 3933129 | T | S | 3989910 | G | A | 3982056 | G | A | Yes | NS, NC | P71850 | metabolism |
| 1634 | 3938113 | G | R | 3994894 | A | W | 3987040 | A | null | Yes | NS, NC | P96837 | — |
| 1635 | 3939862 | C | G | 3996643 | G | G | 3988788 | G | G | Yes | S, NULL | Null | — |
| 1636 | 3942989 | A | V | 3999770 | G | A | 3991914 | G | V | Yes | NS, C | P96841 | metabolism |
| 1637 | 3946062 | A | N | 4002843 | C | T | 3994987 | C | T | Yes | NS, C | P96843 | enzyme activity |
| 1638 | 3947819 | G | R | 4004600 | A | Q | 3996744 | A | Q | Yes | NS, NC | P96845 | structural constituent of |
| | | | | | | | | | | | | | ribosome |
| 1639 | 3948329 | C | S | 4005110 | G | W | 3997254 | G | W | Yes | NS, NC | P96845 | structural constituent of |
| | | | | | | | | | | | | | ribosome |
| 1640 | 3951962 | G | T | 4008744 | A | I | 4000886 | A | I | Yes | NS, NC | P96849 | electron transport |
| 1641 | 3952885 | C | R | 4009667 | T | Q | 4001809 | T | Q | Yes | NS, NC | P96850 | enzyme activity |
| 1642 | 3955434 | C | D | 4012216 | T | N | 4004358 | T | N | Yes | NS, NC | P96852 | — |
| 1643 | 3961924 | A | N | 4018706 | G | D | 4010848 | G | D | Yes | NS, NC | P96858 | — |
| 1644 | 3964308 | T | S | 4021090 | G | A | 4013232 | G | A | Yes | NS, NC | P96860 | arsenite transporter |
| | | | | | | | | | | | | | activity |
| 1645 | 3964705 | G | A | 4021486 | T | E | 4013628 | T | E | Yes | NS, NC | P96861 | RNA binding activity |
| 1646 | 3964973 | T | R | 4021754 | G | R | 4013896 | G | R | Yes | S, NULL | Null | — |
| 1647 | 3965078 | T | M | 4021859 | C | V | 4014001 | C | V | Yes | NS, NC | P96861 | RNA binding activity |
| 1648 | 3967982 | C | A | 4024763 | T | T | 4016905 | T | T | Yes | NS, NC | P96864 | isoprenoid biosynthesis |
| 1649 | 3971967 | G | A | 4028749 | A | T | 4020891 | A | T | Yes | NS, NC | O53571 | DNA binding activity |
| 1650 | 3972140 | T | D | 4028922 | G | E | 4021064 | G | E | Yes | NS, C | O53571 | DNA binding activity |
| 1651 | 3973474 | G | T | 4030256 | A | I | 4022398 | A | I | Yes | NS, NC | O53573 | carbonate dehydratase |
| | | | | | | | | | | | | | activity |
| 1652 | 3978123 | G | S | 4034905 | A | N | 4027047 | A | N | Yes | NS, C | O06155 | — |
| 1653 | 3978196 | A | Q | 4034978 | G | Q | 4027120 | G | Q | Yes | S, NULL | Null | — |
| 1654 | 3981644 | G | L | 4038400 | A | L | 4030533 | A | L | Yes | S, NULL | Null | — |
| 1655 | 3989459 | A | Null | 4046215 | G | Null | 4038347 | G | null | Yes | nc, NULL | Null | — |
| 1656 | 3990280 | C | G | 4047036 | A | V | 4039168 | A | V | Yes | NS, C | O06278 | — |
| 1657 | 3990684 | C | G | 4047440 | A | G | 4039572 | A | G | Yes | S, NULL | Null | — |
| 1658 | 3991234 | A | G | 4047990 | G | G | 4040122 | G | G | Yes | S, NULL | Null | — |
| 1659 | 3997767 | G | G | 4054634 | A | G | 4046877 | A | G | Yes | S, NULL | Null | — |
| 1660 | 3997999 | T | K | 4054866 | C | K | 4047109 | C | K | Yes | S, NULL | Null | — |
| 1661 | 3999048 | G | A | 4055915 | A | V | 4048158 | A | V | Yes | NS, C | O06267 | — |
| 1662 | 3999546 | A | Null | 4056413 | C | Null | 4048656 | C | null | No | nc, NULL | Null | — |
| 1663 | 3999563 | G | Null | 4056430 | A | Null | 4048673 | A | null | No | nc, NULL | Null | — |
| 1664 | 3999617 | C | Null | 4056484 | T | Null | 4048727 | T | null | Yes | nc, NULL | Null | — |
| 1665 | 4004281 | A | V | 4067041 | G | A | 4059284 | G | A | Yes | NS, C | O06380 | serine |
| | | | | | | | | | | | | | carboxypeptidase |
| | | | | | | | | | | | | | activity |
| 1666 | 4004389 | A | V | 4067149 | G | A | 4059392 | G | A | Yes | NS, C | O06380 | serine |
| | | | | | | | | | | | | | carboxypeptidase |
| | | | | | | | | | | | | | activity |
| 1667 | 4006348 | T | Null | 4069108 | C | Null | 4061351 | C | null | No | nc, NULL | Null | — |
| 1668 | 4007724 | T | Null | 4070484 | G | Null | 4062727 | G | null | No | nc, NULL | Null | — |
| 1669 | 4013888 | T | I | 4076648 | C | I | 4068891 | C | null | Yes | S, NULL | Null | — |
| 1670 | 4015529 | G | N | 4078289 | T | K | 4070532 | T | K | Yes | NS, NC | O06368 | — |
| 1671 | 4016100 | T | E | 4078860 | C | G | 4071103 | C | G | Yes | NS, NC | O06367 | — |
| 1672 | 4017100 | G | Null | 4079860 | C | Null | 4072103 | C | null | No | nc, NULL | Null | — |
| 1673 | 4020748 | T | I | 4083508 | C | I | 4075752 | C | I | Yes | S, NULL | Null | — |
| 1674 | 4024907 | T | V | 4087667 | C | V | 4079911 | C | V | Yes | S, NULL | Null | — |
| 1675 | 4026295 | C | P | 4089055 | T | L | 4081299 | T | L | Yes | NS, NC | O06359 | nucleic acid binding |
| | | | | | | | | | | | | | activity |
| 1676 | 4027898 | T | H | 4090658 | C | H | 4082902 | C | H | Yes | S, NULL | Null | — |
| 1677 | 4032641 | C | E | 4095292 | T | E | 4087553 | T | E | Yes | S, NULL | Null | — |
| 1678 | 4033932 | A | A | 4096583 | G | A | 4088843 | G | A | Yes | S, NULL | Null | — |
| 1679 | 4036406 | A | L | 4099057 | G | P | 4091317 | G | null | Yes | NS, NC | O69628 | — |
| 1680 | 4037111 | C | P | 4099761 | T | S | 4092021 | T | S | Yes | NS, NC | O69629 | metabolism |
| 1681 | 4039283 | T | T | 4101933 | C | A | 4094193 | C | A | Yes | NS, NC | O69630 | — |
| 1682 | 4043080 | C | G | 4105730 | T | E | 4097990 | T | E | Yes | NS, NC | O69634 | transporter activity |
| 1683 | 4043104 | T | Q | 4105754 | C | R | 4098014 | C | R | Yes | NS, NC | O69634 | transporter activity |
| 1684 | 4044421 | C | R | 4107071 | T | Q | 4099331 | T | Q | Yes | NS, NC | O69634 | transporter activity |
| 1685 | 4045320 | A | E | 4107970 | G | G | 4100230 | G | G | Yes | NS, NC | O69635 | enzyme activity |
| 1686 | 4049776 | C | V | 4112426 | T | I | 4104686 | T | I | Yes | NS, C | O69639 | serine-type |
| | | | | | | | | | | | | | endopeptidase activity |
| 1687 | 4054029 | C | L | 4116679 | T | L | 4108939 | T | L | Yes | S, NULL | Null | — |
| 1688 | 4054508 | A | Null | 4117158 | C | Null | 4109418 | C | null | No | nc, NULL | Null | — |
| 1689 | 4056593 | C | D | 4119243 | T | D | 4111501 | T | D | Yes | S, NULL | Null | — |
| 1690 | 4058330 | G | Null | 4120980 | A | Null | 4113238 | A | null | No | nc, NULL | Null | — |
| 1691 | 4059022 | T | Null | 4121672 | C | Null | 4113930 | C | null | No | nc, NULL | Null | — |
| 1692 | 4065667 | G | Q | 4128317 | C | E | 4120575 | C | E | Yes | NS, NC | O69653 | monooxygenase activity |
| 1693 | 4066936 | A | Null | 4129586 | C | Null | 4121844 | C | null | Yes | S, NULL | Null | — |
| 1694 | 4068058 | C | L | 4130708 | G | V | 4122966 | G | V | Yes | NS, C | O69657 | — |
| 1695 | 4072777 | T | T | 4135427 | C | T | 4127684 | C | T | Yes | S, NULL | Null | — |
| 1696 | 4073901 | A | L | 4136551 | G | L | 4128808 | G | L | Yes | S, NULL | Null | — |
| 1697 | 4075724 | G | A | 4138374 | A | V | 4130631 | A | V | Yes | NS, C | O69664 | glycerol kinase activity |
| 1698 | 4076324 | G | A | 4138974 | C | G | 4131231 | C | G | Yes | NS, C | O69664 | glycerol kinase activity |
| 1699 | 4077491 | T | R | 4140140 | C | G | 4132397 | C | G | Yes | NS, NC | O69665 | — |
| 1700 | 4077846 | C | R | 4140495 | A | R | 4132752 | A | R | Yes | S, NULL | Null | — |
| 1701 | 4078038 | C | I | 4140687 | G | M | 4132944 | G | M | Yes | NS, NC | O69666 | — |
| 1702 | 4078538 | G | R | 4141187 | C | P | 4133444 | C | P | Yes | NS, NC | O69666 | — |
| 1703 | 4083085 | G | Y | 4145734 | A | Y | 4137991 | A | Y | Yes | S, NULL | Null | — |
| 1704 | 4091572 | A | H | 4154221 | C | P | 4146478 | C | P | Yes | NS, NC | P96420 | enzyme activity |
| 1705 | 4092200 | T | V | 4154849 | G | V | 4147106 | G | V | Yes | S, NULL | Null | — |
| 1706 | 4093758 | G | A | 4156236 | A | V | 4148493 | A | V | Yes | NS, C | O69678 | DNA-directed DNA |
| | | | | | | | | | | | | | polymerase activity |
| 1707 | 4094022 | T | D | 4156500 | C | G | 4148757 | C | G | Yes | NS, NC | O69678 | DNA-directed DNA |
| | | | | | | | | | | | | | polymerase activity |
| 1708 | 4095038 | A | S | 4157575 | G | G | 4149891 | G | G | Yes | NS, NC | O69679 | ATP binding activity |
| 1709 | 4095492 | C | T | 4158029 | A | K | 4150345 | A | K | Yes | NS, NC | O69679 | ATP binding activity |
| 1710 | 4095821 | G | P | 4158358 | A | P | 4150674 | A | P | Yes | S, NULL | Null | — |
| 1711 | 4101540 | A | Q | 4164077 | G | Q | 4156393 | G | Q | Yes | S, NULL | Null | — |
| 1712 | 4107289 | A | V | 4169827 | G | V | 4162142 | G | V | Yes | S, NULL | Null | — |
| 1713 | 4108573 | C | P | 4171110 | T | P | 4163425 | T | P | Yes | S, NULL | Null | — |
| 1714 | 4109633 | C | S | 4172170 | A | R | 4164485 | A | R | Yes | NS, NC | O69693 | alcohol dehydrogenase |
| | | | | | | | | | | | | | activity |
| 1715 | 4110670 | A | M | 4173207 | G | V | 4165522 | G | V | Yes | NS, NC | O69694 | — |
| 1716 | 4113722 | A | R | 4176259 | G | G | 4168574 | G | G | Yes | NS, NC | O69695 | enzyme activity |
| 1717 | 4116252 | A | I | 4178789 | G | V | 4171104 | G | V | Yes | NS, C | O69696 | S-adenosylmethionine- |
| | | | | | | | | | | | | | dependent |
| | | | | | | | | | | | | | methyltransferase |
| | | | | | | | | | | | | | activity |
| 1718 | 4116549 | T | S | 4179086 | C | P | 4171401 | C | P | Yes | NS, NC | O69696 | S-adenosylmethionine- |
| | | | | | | | | | | | | | dependent |
| | | | | | | | | | | | | | methyltransferase |
| | | | | | | | | | | | | | activity |
| 1719 | 4118314 | C | G | 4180851 | G | G | 4173166 | G | G | Yes | S, NULL | Null | — |
| 1720 | 4118737 | A | F | 4181274 | G | F | 4173589 | G | F | Yes | S, NULL | Null | — |
| 1721 | 4121062 | T | A | 4183599 | C | A | 4175914 | C | A | Yes | S, NULL | Null | — |
| 1722 | 4126515 | A | R | 4189052 | C | R | 4181367 | C | R | Yes | S, NULL | Null | — |
| 1723 | 4127210 | A | C | 4190913 | G | R | 4183228 | G | R | Yes | NS, NC | O69707 | molecular_function |
| | | | | | | | | | | | | | unknown |
| 1724 | 4131476 | A | C | 4195179 | G | C | 4187494 | G | C | Yes | S, NULL | Null | — |
| 1725 | 4132036 | T | A | 4195739 | C | A | 4188054 | C | A | Yes | S, NULL | Null | — |
| 1726 | 4133484 | G | Null | 4197186 | A | Null | 4189502 | A | null | Yes | nc, NULL | Null | — |
| 1727 | 4133850 | A | D | 4197552 | G | G | 4189868 | G | null | Yes | NS, NC | O69715 | — |
| 1728 | 4136514 | G | T | 4200217 | A | M | 4192532 | A | M | Yes | NS, NC | O69720 | — |
| 1729 | 4138677 | C | V | 4202380 | A | V | 4194695 | A | V | Yes | S, NULL | Null | — |
| 1730 | 4141141 | A | V | 4204844 | G | A | 4197159 | G | A | Yes | NS, C | O69725 | — |
| 1731 | 4144138 | G | H | 4207841 | A | Y | 4200156 | A | Y | Yes | NS, C | O69728 | — |
| 1732 | 4144689 | C | G | 4208392 | A | V | 4200707 | A | V | Yes | NS, C | O69728 | — |
| 1733 | 4147170 | C | R | 4210873 | T | H | 4203188 | T | H | Yes | NS, NC | O69729 | two-component sensor |
| | | | | | | | | | | | | | molecule activity |
| 1734 | 4148468 | C | Null | 4212171 | T | Null | 4204486 | T | null | No | nc, NULL | Null | — |
| 1735 | 4151295 | A | Null | 4214998 | C | Null | 4207313 | C | null | No | nc, NULL | Null | — |
| 1736 | 4151778 | C | A | 4215481 | G | P | 4207796 | G | P | Yes | NS, NC | P72037 | — |
| 1737 | 4152549 | G | R | 4216252 | C | Null | 4208567 | C | R | Yes | nc, NULL | Null | — |
| 1738 | 4153851 | G | A | 4217554 | A | T | 4209869 | A | T | Yes | NS, NC | P72039 | histidine biosynthesis |
| 1739 | 4158142 | T | A | 4221844 | C | A | 4214159 | C | A | Yes | S, NULL | Null | — |
| 1740 | 4165765 | C | Y | 4229467 | T | Y | 4221782 | T | Y | Yes | S, NULL | Null | — |
| 1741 | 4167728 | G | A | 4231430 | A | A | 4223746 | A | A | Yes | S, NULL | Null | — |
| 1742 | 4168622 | G | R | 4232324 | A | R | 4224640 | A | R | Yes | S, NULL | Null | — |
| 1743 | 4169929 | A | N | 4233631 | G | N | 4225947 | G | N | Yes | S, NULL | Null | — |
| 1744 | 4172445 | T | G | 4236147 | C | G | 4228463 | C | G | Yes | S, NULL | Null | — |
| 1745 | 4176574 | A | L | 4240276 | G | L | 4232591 | G | L | Yes | S, NULL | Null | — |
| 1746 | 4176966 | T | I | 4240668 | C | T | 4232983 | C | T | Yes | NS, NC | P72059 | cell wall |
| 1747 | 4179265 | T | T | 4242967 | C | T | 4235282 | C | T | Yes | S, NULL | Null | — |
| 1748 | 4180515 | T | L | 4244217 | C | L | 4236532 | C | L | Yes | S, NULL | Null | — |
| 1749 | 4182846 | G | S | 4246548 | A | N | 4238863 | A | N | Yes | NS, C | P72030 | cell wall |
| 1750 | 4183159 | T | V | 4246861 | C | V | 4239176 | C | V | Yes | S, NULL | Null | — |
| 1751 | 4183941 | C | A | 4247643 | A | E | 4239958 | A | E | Yes | NS, NC | P72030 | cell wall |
| 1752 | 4187131 | T | I | 4250833 | C | T | 4243148 | C | T | Yes | NS, NC | P72062 | hydrolase activity |
| 1753 | 4187592 | C | G | 4251294 | G | G | 4243609 | G | G | Yes | S, NULL | Null | — |
| 1754 | 4195852 | C | G | 4259576 | G | A | 4251901 | G | A | Yes | NS, C | O53579 | enzyme activity |
| 1755 | 4197772 | A | V | 4261496 | G | A | 4253821 | G | A | Yes | NS, C | O53580 | enzyme activity |
| 1756 | 4199552 | G | Null | 4263276 | A | Null | 4255601 | A | null | No | nc, NULL | Null | — |
| 1757 | 4201999 | C | G | 4265723 | G | G | 4258048 | G | G | Yes | S, NULL | Null | — |
| 1758 | 4204131 | C | V | 4267855 | T | I | 4260180 | T | I | Yes | NS, C | O53582 | — |
| 1759 | 4205624 | A | A | 4269348 | G | A | 4261673 | G | A | Yes | S, NULL | Null | — |
| 1760 | 4205660 | G | D | 4269384 | T | E | 4261709 | T | E | Yes | NS, C | O53583 | membrane |
| 1761 | 4205879 | G | R | 4269603 | A | R | 4261928 | A | R | Yes | S, NULL | Null | — |
| 1762 | 4209847 | G | Null | 4273571 | T | Null | 4265896 | T | null | No | nc, NULL | Null | — |
| 1763 | 4214379 | A | Null | 4278103 | G | Null | 4270428 | G | null | Yes | nc, NULL | Null | — |
| 1764 | 4214597 | C | Null | 4278321 | G | Null | 4270646 | G | null | No | nc, NULL | Null | — |
| 1765 | 4215241 | G | T | 4278965 | A | M | 4271290 | A | M | Yes | NS, NC | O07810 | molecular_function |
| | | | | | | | | | | | | | unknown |
| 1766 | 4217784 | A | L | 4281508 | G | L | 4273833 | G | L | Yes | S, NULL | Null | — |
| 1767 | 4220711 | A | I | 4284435 | G | T | 4276760 | G | T | Yes | NS, NC | O07803 | molecular_function |
| | | | | | | | | | | | | | unknown |
| 1768 | 4222561 | A | N | 4286285 | G | D | 4278610 | G | D | Yes | NS, NC | O07802 | — |
| 1769 | 4222603 | A | T | 4286327 | G | A | 4278652 | G | A | Yes | NS, NC | O07802 | — |
| 1770 | 4223157 | A | Y | 4286881 | G | C | 4279206 | G | C | Yes | NS, NC | O07801 | — |
| 1771 | 4223301 | C | T | 4287025 | A | N | 4279350 | A | N | Yes | NS, C | O07801 | — |
| 1772 | 4223437 | G | G | 4287161 | A | G | 4279486 | A | G | Yes | S, NULL | Null | — |
| 1773 | 4223634 | T | V | 4287358 | C | A | 4279683 | C | A | Yes | NS, C | O07801 | — |
| 1774 | 4226226 | G | A | 4289950 | A | V | 4282275 | A | V | Yes | NS, C | O07800 | membrane |
| 1775 | 4226837 | T | A | 4290561 | G | A | 4282886 | G | A | Yes | S, NULL | Null | — |
| 1776 | 4227100 | G | R | 4290824 | C | G | 4283149 | C | G | Yes | NS, NC | O07800 | membrane |
| 1777 | 4228368 | A | A | 4292092 | C | A | 4284417 | C | A | Yes | S, NULL | Null | — |
| 1778 | 4238583 | C | L | 4302307 | A | L | 4294632 | A | L | Yes | S, NULL | Null | — |
| 1779 | 4241120 | A | D | 4304844 | C | E | 4297169 | C | E | Yes | NS, C | O07794 | — |
| 1780 | 4241350 | A | T | 4305074 | G | T | 4297399 | G | T | Yes | S, NULL | Null | — |
| 1781 | 4247232 | A | Null | 4310955 | G | Null | 4303280 | G | null | No | nc, NULL | Null | — |
| 1782 | 4249402 | T | S | 4313125 | C | P | 4305450 | C | P | Yes | NS, NC | P96239 | — |
| 1783 | 4252596 | T | H | 4316319 | C | R | 4308644 | C | R | Yes | NS, NC | P96235 | — |
| 1784 | 4252840 | G | R | 4316563 | C | G | 4308888 | C | G | Yes | NS, NC | P96235 | — |
| 1785 | 4254698 | G | Null | 4318421 | T | Null | 4310746 | T | null | Yes | nc, NULL | Null | — |
| 1786 | 4257590 | C | T | 4321314 | T | I | 4313640 | T | I | Yes | NS, NC | P17670 | superoxide dismutase |
| | | | | | | | | | | | | | activity |
| 1787 | 4259178 | A | I | 4322902 | G | V | 4315228 | G | V | Yes | NS, C | P96229 | molecular_function |
| | | | | | | | | | | | | | unknown |
| 1788 | 4259279 | A | A | 4323003 | G | A | 4315329 | G | A | Yes | S, NULL | Null | — |
| 1789 | 4266055 | A | Null | 4329779 | G | Null | 4322105 | G | null | Yes | nc, NULL | Null | — |
| 1790 | 4266511 | T | H | 4330235 | C | R | 4322561 | C | R | Yes | NS, NC | P96219 | monooxygenase activity |
| 1791 | 4270698 | C | E | 4334422 | G | Q | 4326748 | G | Q | Yes | NS, NC | P96218 | glutamate biosynthesis |
| 1792 | 4272870 | A | Null | 4336594 | C | Null | 4328920 | C | null | No | nc, NULL | Null | — |
| 1793 | 4273741 | C | A | 4337465 | T | V | 4329791 | T | V | Yes | NS, C | P96217 | — |
| 1794 | 4280361 | T | V | 4344038 | C | A | 4336363 | C | A | Yes | NS, C | O69733 | nucleotide binding |
| | | | | | | | | | | | | | activity |
| 1795 | 4293145 | A | K | 4356822 | G | E | 4349146 | G | E | Yes | NS, NC | O69742 | — |
| 1796 | 4293741 | A | G | 4357418 | G | G | 4349742 | G | G | Yes | S, NULL | Null | — |
| 1797 | 4294795 | A | P | 4358472 | C | P | 4350796 | C | P | Yes | S, NULL | Null | — |
| 1798 | 4300168 | A | L | 4363800 | G | L | 4356106 | G | L | Yes | S, NULL | Null | — |
| 1799 | 4301014 | C | A | 4364646 | T | T | 4356952 | T | T | Yes | NS, NC | O05461 | subtilase activity |
| 1800 | 4304014 | G | Y | 4367646 | A | Y | 4359952 | A | Y | Yes | S, NULL | Null | — |
| 1801 | 4304489 | G | P | 4368121 | A | L | 4360427 | A | L | Yes | NS, NC | O05459 | — |
| 1802 | 4307013 | C | G | 4370645 | T | D | 4362949 | T | D | Yes | NS, NC | O05457 | — |
| 1803 | 4308186 | C | A | 4374225 | T | T | 4366529 | T | T | Yes | NS, NC | O05453 | — |
| 1804 | 4310990 | C | S | 4377030 | G | S | 4369334 | G | S | Yes | S, NULL | Null | — |
| 1805 | 4313166 | T | R | 4379205 | C | R | 4371509 | C | R | Yes | S, NULL | Null | — |
| 1806 | 4314838 | C | G | 4380877 | T | D | 4373181 | T | D | Yes | NS, NC | O05449 | — |
| 1807 | 4316253 | C | V | 4382293 | T | I | 4374597 | T | I | Yes | NS, C | O05448 | — |
| 1808 | 4316561 | C | G | 4382601 | T | D | 4374905 | T | D | Yes | NS, NC | O05448 | — |
| 1809 | 4316925 | T | Null | 4382965 | C | Null | 4375269 | C | null | No | nc, NULL | Null | — |
| 1810 | 4317617 | G | Q | 4383652 | A | * | 4375961 | A | * | Yes | NS, TP | O05447 | — |
| 1811 | 4317969 | G | Null | 4384004 | C | Null | 4376313 | C | null | No | nc, NULL | Null | — |
| 1812 | 4319148 | G | P | 4385184 | A | S | 4377493 | A | S | Yes | NS, NC | O05446 | — |
| 1813 | 4320218 | C | A | 4386254 | G | P | 4378563 | G | P | Yes | NS, NC | O05445 | — |
| 1814 | 4320714 | C | W | 4386750 | A | L | 4379059 | A | L | Yes | NS, NC | O05444 | — |
| 1815 | 4324427 | C | V | 4390463 | T | M | 4382772 | T | M | Yes | NS, NC | O05441 | — |
| 1816 | 4324820 | T | * | 4390856 | C | W | 4383165 | C | W | Yes | NS, TP | O05440 | — |
| 1817 | 4327799 | T | L | 4393835 | C | L | 4386144 | C | L | Yes | S, NULL | Null | — |
| 1818 | 4328171 | C | R | 4394207 | G | G | 4386516 | G | R | Yes | NS, NC | O05436 | — |
| 1819 | 4328226 | C | A | 4394262 | G | G | 4386571 | G | A | Yes | NS, C | O05436 | — |
| 1820 | 4329348 | G | S | 4395384 | A | N | 4387692 | A | N | Yes | NS, C | O05436 | — |
| 1821 | 4329765 | T | V | 4395801 | C | A | 4388109 | C | A | Yes | NS, C | O05436 | — |
| 1822 | 4331996 | C | A | 4398032 | T | V | 4390340 | T | V | Yes | NS, C | O05435 | pathogenesis |
| 1823 | 4334624 | T | S | 4400660 | C | S | 4392968 | G | A | Yes | S, NULL | Null | — |
| 1824 | 4335857 | G | G | 4401894 | A | D | 4394201 | A | D | Yes | NS, NC | P52214 | thioredoxin reductase |
| | | | | | | | | | | | | | (NADPH) activity |
| 1825 | 4339065 | C | R | 4405102 | T | R | 4397409 | T | R | Yes | S, NULL | Null | — |
| 1826 | 4341548 | C | A | 4407585 | T | A | 4399892 | T | A | Yes | S, NULL | Null | — |
| 1827 | 4342530 | A | W | 4408567 | G | R | 4400874 | G | R | Yes | NS, NC | O53598 | nucleic acid binding |
| | | | | | | | | | | | | | activity |
| 1828 | 26940 | G | Null | 26959 | C | Null | Null | Null | Null | No | Null, NC | Null | Null |
| 1829 | 34028 | C | Null | 34044 | T | Null | Null | Null | Null | No | Null, NC | Null | Null |

Table I: List of single nucleotide polymorphisms in Mycobacaterium tuberculosis/M. bovis BCG
Polymorphism ID: The ID by which the polymorphism can be identified
SNP Position: Position of the SNP in the respective genome
Base: The nucleotide occurring in the region of the polymorphism in the respective genome
AA: The aminoacid occurring in the region of the polymorphism in the respective genome
ORF: Indicates whether the polymorphism occurs in an open reading frame (yes) or not (no)
SNP type: Indicates the kind of SNP-S: synonymous SNP which codes for the same amino acid as the reference sequence; NS: non-synonymous SNP which codes for an aminoacid different from the reference sequence: C: conservative SNP coding for an aminoacid of the same family as the reference sequence: NC: nonconservative SNP coding for an aminoacid from a different family as the reference sequence
GO ID: The ID for the sequence in the gene ontology database
Putative function: The putative function of the gene in which the SNP occurs.

TABLE II
List of insertion/deletions in M. tuberculosis/M. bovis BCG

| | BCG | BCG | H37Rv | H37Rv | CDC | CDC | | | |
|---|---|---|---|---|---|---|---|---|---|
| Polymorphism ID | Start | End | start | end | start | end | ORF | GO ID | Putative Function |
| 1830 | 13233 | 13234 | 13233 | 13235 | 13233 | 13235 | YES | P71580 | integral to |
| | | | | | | | | | membrane |
| 1831 | 24719 | 24720 | 24720 | 24739 | 13233 | 13235 | YES | P71590 | — |
| 1832 | 28917 | 28918 | 28936 | 28938 | 13233 | 13235 | YES | P71594 | — |
| 1833 | 30962 | 30967 | 30982 | 30983 | 13233 | 13235 | YES | P71596 | — |
| 1834 | 42578 | 42588 | 42594 | 42595 | 13233 | 13235 | YES | P71697 | — |
| 1835 | 71576 | 71614 | 71584 | 71585 | 13233 | 13235 | YES | Null | — |
| 1836 | 79584 | 79594 | 79555 | 79556 | 13233 | 13235 | YES | O53616 | RNA binding |
| | | | | | | | | | activity |
| 1837 | 82490 | 82491 | 82452 | 82454 | 13233 | 13235 | YES | O53618 | nucleotide binding |
| | | | | | | | | | activity |
| 1838 | 125870 | 125872 | 125832 | 125833 | 13233 | 13235 | YES | Q10900 | magnesium ion |
| | | | | | | | | | binding activity |
| 1839 | 131213 | 131215 | 131174 | 131175 | 13233 | 13235 | YES | Null | — |
| 1840 | 138784 | 138786 | 138744 | 138745 | 13233 | 13235 | YES | O53637 | peroxidase activity |
| 1841 | 139598 | 139600 | 139557 | 139558 | 13233 | 13235 | YES | O53637 | peroxidase activity |
| 1842 | 147495 | 147496 | 147453 | 147455 | 13233 | 13235 | YES | O07170 | translation |
| | | | | | | | | | elongation factor |
| | | | | | | | | | activity |
| 1843 | 147853 | 147854 | 147812 | 147814 | 13233 | 13235 | YES | Null | — |
| 1844 | 150079 | 150080 | 150039 | 150067 | 13233 | 13235 | YES | O07174 | — |
| 1845 | 150906 | 151077 | 150893 | 150894 | 13233 | 13235 | YES | O07174 | — |
| 1846 | 162346 | 162347 | 162153 | 162155 | 13233 | 13235 | YES | P96811 | enzyme activity |
| 1847 | 162451 | 162453 | 162259 | 162260 | 13233 | 13235 | YES | P96811 | enzyme activity |
| 1848 | 162694 | 162695 | 162501 | 162503 | 13233 | 13235 | YES | P96811 | enzyme activity |
| 1849 | 194495 | 194498 | 194303 | 194304 | 13233 | 13235 | YES | O07410 | transcription factor |
| | | | | | | | | | activity |
| 1850 | 208509 | 208510 | 208315 | 208322 | 13233 | 13235 | YES | O07420 | — |
| 1851 | 223943 | 223945 | 223749 | 223750 | 13233 | 13235 | YES | O07436 | — |
| 1852 | 230770 | 230772 | 230575 | 230576 | 13233 | 13235 | YES | Null | — |
| 1853 | 234690 | 234693 | 234494 | 234495 | 13233 | 13235 | YES | O53648 | — |
| 1854 | 257984 | 258014 | 257786 | 257787 | 13233 | 13235 | YES | P96397 | acyl-CoA |
| | | | | | | | | | dehydrogenase |
| | | | | | | | | | activity |
| 1855 | 264979 | 264980 | 264752 | 266645 | 13233 | 13235 | YES | P96403, P96405 | — |
| 1856 | 265066 | 265068 | 266741 | 266742 | 13233 | 13235 | YES | P96405 | metabolism |
| 1857 | 291957 | 291959 | 293631 | 293632 | 13233 | 13235 | YES | Null | — |
| 1858 | 331998 | 331999 | 333671 | 333673 | 13233 | 13235 | YES | P56877 | — |
| 1859 | 332977 | 335748 | 334651 | 334652 | 13233 | 13235 | YES | P56877 | — |
| 1860 | 336706 | 336707 | 335600 | 335657 | 13233 | 13235 | YES | P56877 | — |
| 1861 | 336884 | 336885 | 335844 | 335863 | 13233 | 13235 | YES | P56877 | — |
| 1862 | 338180 | 338181 | 337158 | 337168 | 13233 | 13235 | YES | O53684 | — |
| 1863 | 339540 | 339541 | 338527 | 338537 | 13233 | 13235 | YES | O53684 | — |
| 1864 | 363810 | 363856 | 362806 | 362807 | 13233 | 13235 | YES | O07224 | intracellular |
| 1865 | 369162 | 369163 | 368113 | 368129 | 13233 | 13235 | YES | O07231 | tRNA ligase activity |
| 1866 | 370799 | 370800 | 369765 | 369767 | 13233 | 13235 | YES | O07231 | tRNA ligase activity |
| 1867 | 374314 | 374315 | 373281 | 373283 | 13233 | 13235 | YES | O07232 | — |
| 1868 | 416214 | 416215 | 415182 | 415184 | 13233 | 13235 | YES | O06296 | — |
| 1869 | 425351 | 425353 | 424320 | 424321 | 13233 | 13235 | YES | O06303 | — |
| 1870 | 425821 | 425824 | 424789 | 424790 | 13233 | 13235 | YES | O06304 | — |
| 1871 | 428391 | 428392 | 427357 | 427373 | 13233 | 13235 | YES | O06304 | — |
| 1872 | 482549 | 482550 | 481528 | 481530 | 13233 | 13235 | YES | P95211 | membrane |
| 1873 | 488117 | 488119 | 487097 | 487098 | 13233 | 13235 | YES | O86335 | enzyme activity |
| 1874 | 570941 | 570942 | 569920 | 569961 | 13233 | 13235 | YES | Q11146 | molecular_function |
| | | | | | | | | | unknown |
| 1875 | 578459 | 578500 | 577494 | 577495 | 13233 | 13235 | YES | Null | — |
| 1876 | 581835 | 581956 | 580812 | 580813 | 13233 | 13235 | YES | Q11156 | two-component |
| | | | | | | | | | response regulator |
| | | | | | | | | | activity |
| 1877 | 612063 | 612064 | 610910 | 610912 | 13233 | 13235 | YES | Null | — |
| 1878 | 624447 | 624522 | 623295 | 623296 | 13233 | 13235 | YES | O06398 | — |
| 1879 | 624655 | 624665 | 623419 | 623420 | 13233 | 13235 | YES | O06398 | — |
| 1880 | 625594 | 625596 | 624349 | 624350 | 13233 | 13235 | YES | O06398 | — |
| 1881 | 641609 | 641610 | 640363 | 640365 | 13233 | 13235 | YES | O06415 | — |
| 1882 | 664431 | 664432 | 663186 | 663188 | 13233 | 13235 | YES | O53767 | ribonucleoside- |
| | | | | | | | | | diphosphate |
| | | | | | | | | | reductase activity |
| 1883 | 669950 | 669952 | 668706 | 668707 | 13233 | 13235 | YES | O53772 | monooxygenase |
| | | | | | | | | | activity |
| 1884 | 690039 | 690041 | 688794 | 688795 | 13233 | 13235 | YES | O07788 | pathogenesis |
| 1885 | 693138 | 693140 | 691892 | 691893 | 13233 | 13235 | YES | O07786 | pathogenesis |
| 1886 | 713437 | 713439 | 712190 | 712191 | 13233 | 13235 | YES | O07759, O07758 | — |
| 1887 | 723680 | 723681 | 722432 | 722434 | 13233 | 13235 | YES | P96920 | DNA binding |
| | | | | | | | | | activity |
| 1888 | 731330 | 731331 | 730083 | 730093 | 13233 | 13235 | YES | P96923 | — |
| 1889 | 743870 | 744394 | 742632 | 742633 | 13233 | 13235 | YES | Null | — |
| 1890 | 800911 | 800912 | 799140 | 799142 | 13233 | 13235 | YES | P95044 | — |
| 1891 | 804268 | 804309 | 802498 | 802499 | 13233 | 13235 | YES | Null | — |
| 1892 | 832699 | 832702 | 830875 | 830876 | 13233 | 13235 | YES | O53802 | — |
| 1893 | 838696 | 838697 | 836870 | 836919 | 13233 | 13235 | YES | O53809 | — |
| 1894 | 839071 | 839072 | 837293 | 837342 | 13233 | 13235 | YES | O53809 | — |
| 1895 | 839638 | 839767 | 837908 | 837909 | 13233 | 13235 | YES | O53809 | — |
| 1896 | 841026 | 841185 | 839098 | 839099 | 13233 | 13235 | YES | O53810 | — |
| 1897 | 841398 | 841494 | 839302 | 839303 | 13233 | 13235 | YES | O53810 | — |
| 1898 | 841688 | 841689 | 839487 | 839497 | 13233 | 13235 | YES | O53810 | — |
| 1899 | 856450 | 856451 | 854258 | 854260 | 13233 | 13235 | YES | Null | — |
| 1900 | 877025 | 877028 | 874834 | 874835 | 13233 | 13235 | YES | P71834, P71835 | — |
| 1901 | 881931 | 881932 | 879738 | 879740 | 13233 | 13235 | YES | P71838 | integral to |
| | | | | | | | | | membrane |
| 1902 | 890037 | 890038 | 887845 | 887847 | 13233 | 13235 | YES | O07268 | cytoplasm |
| 1903 | 927816 | 927891 | 926984 | 926985 | 13233 | 13235 | YES | O53844 | — |
| 1904 | 928822 | 928823 | 927918 | 927928 | 13233 | 13235 | YES | O53845 | calcium ion binding |
| | | | | | | | | | activity |
| 1905 | 928975 | 928976 | 928080 | 928215 | 13233 | 13235 | YES | O53845 | calcium ion binding |
| | | | | | | | | | activity |
| 1906 | 936197 | 936204 | 935446 | 935447 | 13233 | 13235 | YES | O53850 | cell wall |
| 1907 | 953566 | 953567 | 952809 | 952811 | 13233 | 13235 | YES | Null | — |
| 1908 | 961024 | 961025 | 960268 | 960309 | 13233 | 13235 | YES | Null | — |
| 1909 | 963656 | 963657 | 962953 | 962955 | 13233 | 13235 | YES | O53876 | Mo-molybdopterin |
| | | | | | | | | | cofactor |
| | | | | | | | | | biosynthesis |
| 1910 | 965541 | 965542 | 964839 | 965070 | 13233 | 13235 | YES | O53879 | — |
| 1911 | 968900 | 968910 | 968438 | 968439 | 13233 | 13235 | YES | O53884 | — |
| 1912 | 969448 | 969449 | 968977 | 968981 | 13233 | 13235 | YES | O53884 | — |
| 1913 | 977362 | 977363 | 976894 | 976896 | 13233 | 13235 | YES | Q10540 | integral to |
| | | | | | | | | | membrane |
| 1914 | 1010671 | 1010673 | 1010204 | 1010205 | 13233 | 13235 | YES | O05900 | — |
| 1915 | 1032449 | 1032450 | 1031981 | 1031983 | 13233 | 13235 | YES | O05917 | — |
| 1916 | 1039551 | 1039553 | 1039084 | 1039085 | 13233 | 13235 | YES | O05871 | protein kinase |
| | | | | | | | | | activity |
| 1917 | 1041920 | 1041922 | 1041452 | 1041453 | 13233 | 13235 | YES | P95302 | nucleotide binding |
| | | | | | | | | | activity |
| 1918 | 1064550 | 1064551 | 1064081 | 1064110 | 13233 | 13235 | YES | Null | — |
| 1919 | 1087886 | 1087887 | 1087445 | 1087447 | 13233 | 13235 | YES | O86319 | acyl-CoA |
| | | | | | | | | | dehydrogenase |
| | | | | | | | | | activity |
| 1920 | 1090629 | 1090631 | 1090189 | 1090190 | 13233 | 13235 | YES | Null | — |
| 1921 | 1131681 | 1131683 | 1131228 | 1131229 | 13233 | 13235 | YES | O05597 | — |
| 1922 | 1135355 | 1135356 | 1134901 | 1134907 | 13233 | 13235 | YES | P96384 | membrane |
| 1923 | 1165969 | 1165971 | 1165520 | 1165521 | 13233 | 13235 | YES | Null | — |
| 1924 | 1169165 | 1169167 | 1168715 | 1168716 | 13233 | 13235 | YES | O86321 | — |
| 1925 | 1173288 | 1173289 | 1172837 | 1172839 | 13233 | 13235 | YES | Null | — |
| 1926 | 1189124 | 1189125 | 1188674 | 1188678 | 13233 | 13235 | YES | O53415 | — |
| 1927 | 1189603 | 1189622 | 1189156 | 1189157 | 13233 | 13235 | YES | O53415 | — |
| 1928 | 1189661 | 1189662 | 1189196 | 1189200 | 13233 | 13235 | YES | O53415 | — |
| 1929 | 1191462 | 1191463 | 1191000 | 1191010 | 13233 | 13235 | YES | O53416 | — |
| 1930 | 1191817 | 1192525 | 1191364 | 1191365 | 13233 | 13235 | YES | O53416 | — |
| 1931 | 1192629 | 1192812 | 1191459 | 1191460 | 13233 | 13235 | YES | O53416 | — |
| 1932 | 1214392 | 1214393 | 1213030 | 1213049 | 13233 | 13235 | YES | O53435 | — |
| 1933 | 1214589 | 1214590 | 1213245 | 1213255 | 13233 | 13235 | YES | O53435 | — |
| 1934 | 1214840 | 1214844 | 1213505 | 1213506 | 13233 | 13235 | YES | O53435 | — |
| 1935 | 1215028 | 1215074 | 1213690 | 1213691 | 13233 | 13235 | YES | O53435 | — |
| 1936 | 1219617 | 1219618 | 1218234 | 1218244 | 13233 | 13235 | YES | O53439 | — |
| 1937 | 1231791 | 1231792 | 1230417 | 1230419 | 13233 | 13235 | YES | O53449 | integral to |
| | | | | | | | | | membrane |
| 1938 | 1274621 | 1274623 | 1273248 | 1273249 | 13233 | 13235 | YES | O06545 | membrane |
| 1939 | 1300681 | 1300683 | 1299307 | 1299308 | 13233 | 13235 | YES | O50424 | — |
| 1940 | 1306903 | 1306904 | 1305528 | 1305643 | 13233 | 13235 | YES | Null | — |
| 1941 | 1314587 | 1314589 | 1313336 | 1313337 | 13233 | 13235 | YES | Null | — |
| 1942 | 1341420 | 1341421 | 1340168 | 1340182 | 13233 | 13235 | YES | O05298 | — |
| 1943 | 1358664 | 1358665 | 1357415 | 1357421 | 13233 | 13235 | YES | O05315 | — |
| 1944 | 1367083 | 1367086 | 1365839 | 1365840 | 13233 | 13235 | YES | O06291 | serine-type |
| | | | | | | | | | endopeptidase |
| | | | | | | | | | activity |
| 1945 | 1404177 | 1404178 | 1402931 | 1405929 | 13233 | 13235 | YES | Q11063, Q11061 | — |
| 1946 | 1407255 | 1407256 | 1409016 | 1409018 | 13233 | 13235 | YES | Q11058 | monooxygenase |
| | | | | | | | | | activity |
| 1947 | 1439690 | 1439691 | 1441542 | 1441686 | 13233 | 13235 | YES | Q10614 | enzyme activity |
| 1948 | 1441478 | 1441519 | 1443483 | 1443484 | 13233 | 13235 | YES | Q10616 | integral to |
| | | | | | | | | | membrane |
| 1949 | 1466163 | 1466164 | 1468112 | 1468115 | 13233 | 13235 | YES | Q10620 | integral to |
| | | | | | | | | | membrane |
| 1950 | 1475063 | 1475064 | 1477025 | 1477027 | 13233 | 13235 | YES | Null | — |
| 1951 | 1539986 | 1539987 | 1541949 | 1543298 | 13233 | 13235 | YES | Null | — |
| 1952 | 1540483 | 1540485 | 1543804 | 1543805 | 13233 | 13235 | YES | P71799 | — |
| 1953 | 1543150 | 1543152 | 1546470 | 1546471 | 13233 | 13235 | YES | P71801 | sulfotransferase |
| | | | | | | | | | activity |
| 1954 | 1569167 | 1569168 | 1572486 | 1572849 | 13233 | 13235 | YES | P71664 | integral to |
| | | | | | | | | | membrane |
| 1955 | 1608954 | 1608976 | 1612645 | 1612646 | 13233 | 13235 | YES | O06823 | — |
| 1956 | 1627336 | 1627337 | 1630987 | 1631015 | 13233 | 13235 | YES | O06810 | — |
| 1957 | 1628863 | 1628891 | 1632541 | 1632542 | 13233 | 13235 | YES | O06810 | — |
| 1958 | 1632753 | 1632882 | 1636167 | 1636168 | 13233 | 13235 | YES | O06808 | — |
| 1959 | 1632905 | 1632909 | 1636181 | 1636182 | 13233 | 13235 | YES | O06808 | — |
| 1960 | 1633457 | 1633467 | 1636730 | 1636731 | 13233 | 13235 | YES | O06808 | — |
| 1961 | 1689986 | 1689987 | 1693238 | 1693240 | 13233 | 13235 | YES | P71783 | — |
| 1962 | 1737536 | 1737538 | 1753521 | 1753522 | 13233 | 13235 | YES | Q10777 | enzyme activity |
| 1963 | 1738035 | 1738037 | 1754019 | 1754020 | 13233 | 13235 | YES | Q10777, Q10776 | — |
| 1964 | 1744186 | 1744191 | 1760169 | 1760170 | 13233 | 13235 | YES | Q10761 | succinate |
| | | | | | | | | | dehydrogenase |
| | | | | | | | | | activity |
| 1965 | 1745810 | 1747954 | 1761789 | 1761790 | 13233 | 13235 | YES | Q10773 | membrane |
| 1966 | 1754245 | 1754246 | 1768071 | 1768868 | 13233 | 13235 | YES | Q10768 | alpha-amylase |
| | | | | | | | | | activity |
| 1967 | 1765829 | 1765830 | 1780461 | 1780463 | 13233 | 13235 | YES | O06615 | — |
| 1968 | 1765952 | 1765954 | 1780585 | 1780586 | 13233 | 13235 | YES | O06615 | — |
| 1969 | 1837548 | 1837549 | 1852180 | 1852182 | 13233 | 13235 | YES | Null | — |
| 1970 | 1850305 | 1850327 | 1864938 | 1864939 | 13233 | 13235 | YES | P94986 | — |
| 1971 | 1879687 | 1879698 | 1894299 | 1894300 | 13233 | 13235 | YES | O53916 | nucleotide binding |
| | | | | | | | | | activity |
| 1972 | 1892915 | 1892916 | 1907517 | 1907558 | 13233 | 13235 | YES | Null | — |
| 1973 | 1900884 | 1900887 | 1915542 | 1915543 | 13233 | 13235 | YES | O33192 | — |
| 1974 | 1914068 | 1914069 | 1928724 | 1928726 | 13233 | 13235 | YES | Null | — |
| 1975 | 1930724 | 1930725 | 1945381 | 1945383 | 13233 | 13235 | YES | P71976 | — |
| 1976 | 1941012 | 1941053 | 1955670 | 1955671 | 13233 | 13235 | YES | Null | — |
| 1977 | 1953648 | 1953650 | 1968249 | 1968250 | 13233 | 13235 | YES | O33271 | — |
| 1978 | 1967611 | 1967752 | 1982211 | 1982212 | 13233 | 13235 | YES | O65937 | — |
| 1979 | 1968448 | 1968449 | 1982898 | 1982967 | 13233 | 13235 | YES | O65937 | — |
| 1980 | 1968664 | 1968665 | 1983192 | 1983261 | 13233 | 13235 | YES | O65937 | — |
| 1981 | 1983171 | 1983172 | 1992328 | 1992330 | 13233 | 13235 | YES | O06794 | — |
| 1982 | 1985312 | 1985313 | 1994470 | 1994472 | 13233 | 13235 | YES | O06795 | molecular_function |
| | | | | | | | | | unknown |
| 1983 | 1992126 | 1992145 | 2001684 | 2001685 | 13233 | 13235 | YES | O06801 | — |
| 1984 | 2016682 | 2016683 | 2026222 | 2026231 | 13233 | 13235 | YES | O86373 | — |
| 1985 | 2051905 | 2051915 | 2061448 | 2061449 | 13233 | 13235 | YES | Q50615 | integral to |
| | | | | | | | | | membrane |
| 1986 | 2064977 | 2064978 | 2074511 | 2074614 | 13233 | 13235 | YES | Null | — |
| 1987 | 2079195 | 2079196 | 2088841 | 2088979 | 13233 | 13235 | YES | Q50594 | integral to |
| | | | | | | | | | membrane |
| 1988 | 2080613 | 2080626 | 2090406 | 2090407 | 13233 | 13235 | YES | Q50593 | integral to |
| | | | | | | | | | membrane |
| 1989 | 2084192 | 2084193 | 2093973 | 2093975 | 13233 | 13235 | YES | P95165 | phosphogluconate |
| | | | | | | | | | dehydrogenase |
| | | | | | | | | | (decarboxylating) |
| | | | | | | | | | activity |
| 1990 | 2085136 | 2085137 | 2094918 | 2094925 | 13233 | 13235 | YES | P95165 | phosphogluconate |
| | | | | | | | | | dehydrogenase |
| | | | | | | | | | (decarboxylating) |
| | | | | | | | | | activity |
| 1991 | 2087040 | 2087041 | 2096828 | 2096830 | 13233 | 13235 | YES | Null | — |
| 1992 | 2093386 | 2093387 | 2103175 | 2103177 | 13233 | 13235 | YES | Null | — |
| 1993 | 2099733 | 2099735 | 2109523 | 2109524 | 13233 | 13235 | YES | Null | — |
| 1994 | 2116913 | 2116915 | 2126702 | 2126703 | 13233 | 13235 | YES | O07753 | transporter activity |
| 1995 | 2123684 | 2123700 | 2133472 | 2133473 | 13233 | 13235 | YES | O07748 | — |
| 1996 | 2127747 | 2127758 | 2137520 | 2137521 | 13233 | 13235 | YES | O07744 | — |
| 1997 | 2133043 | 2133044 | 2142806 | 2142808 | 13233 | 13235 | YES | O07737 | alcohol |
| | | | | | | | | | dehydrogenase |
| | | | | | | | | | activity |
| 1998 | 2133758 | 2133760 | 2143522 | 2143523 | 13233 | 13235 | YES | O07737 | alcohol |
| | | | | | | | | | dehydrogenase |
| | | | | | | | | | activity |
| 1999 | 2136332 | 2136378 | 2146095 | 2146096 | 13233 | 13235 | YES | O07733 | — |
| 2000 | 2151627 | 2151629 | 2161345 | 2161346 | 13233 | 13235 | YES | O07718 | enzyme activity |
| 2001 | 2153548 | 2153549 | 2163265 | 2163278 | 13233 | 13235 | YES | O07716 | enzyme activity |
| 2002 | 2153668 | 2154142 | 2163397 | 2163398 | 13233 | 13235 | YES | O07716 | enzyme activity |
| 2003 | 2154541 | 2154542 | 2163787 | 2163847 | 13233 | 13235 | YES | O07716 | enzyme activity |
| 2004 | 2156236 | 2156602 | 2165551 | 2165552 | 13233 | 13235 | YES | O07716 | enzyme activity |
| 2005 | 2160449 | 2160451 | 2168813 | 2168814 | 13233 | 13235 | YES | O53960 | — |
| 2006 | 2184230 | 2184231 | 2192593 | 2192595 | 13233 | 13235 | YES | P95275 | electron transport |
| 2007 | 2199225 | 2199227 | 2207589 | 2207590 | 13233 | 13235 | YES | Null | — |
| 2008 | 2254439 | 2254440 | 2270521 | 2270531 | 13233 | 13235 | YES | Null | — |
| 2009 | 2260638 | 2260639 | 2276722 | 2276724 | 13233 | 13235 | YES | O53475 | nucleoside |
| | | | | | | | | | metabolism |
| 2010 | 2312077 | 2312079 | 2328162 | 2328163 | 13233 | 13235 | YES | Q10680 | vitamin B12 |
| | | | | | | | | | biosynthesis |
| 2011 | 2313772 | 2313774 | 2329856 | 2329857 | 13233 | 13235 | YES | Q10671 | porphyrin |
| | | | | | | | | | biosynthesis |
| 2012 | 2313988 | 2313989 | 2330071 | 2332091 | 13233 | 13235 | YES | Q10671, Q10683 | — |
| 2013 | 2320088 | 2320092 | 2338200 | 2338201 | 13233 | 13235 | YES | Q10689 | integral to |
| | | | | | | | | | membrane |
| 2014 | 2324539 | 2324551 | 2342648 | 2342649 | 13233 | 13235 | YES | Q10692 | — |
| 2015 | 2329456 | 2329457 | 2347554 | 2347595 | 13233 | 13235 | YES | Q10699 | DNA binding |
| | | | | | | | | | activity |
| 2016 | 2339127 | 2339128 | 2357282 | 2357286 | 13233 | 13235 | YES | Q10707 | — |
| 2017 | 2339871 | 2339873 | 2358029 | 2358030 | 13233 | 13235 | YES | Q10707, Q9ZAE2 | — |
| 2018 | 2347255 | 2347256 | 2365412 | 2366761 | 13233 | 13235 | YES | Null | — |
| 2019 | 2349048 | 2349049 | 2368563 | 2368565 | 13233 | 13235 | YES | Null | — |
| 2020 | 2352985 | 2352986 | 2372501 | 2372542 | 13233 | 13235 | YES | O33247 | molecular_function |
| | | | | | | | | | unknown |
| 2021 | 2361585 | 2361586 | 2381158 | 2381186 | 13233 | 13235 | YES | O33258 | — |
| 2022 | 2378768 | 2378769 | 2398368 | 2398377 | 13233 | 13235 | YES | O06237 | — |
| 2023 | 2382325 | 2382432 | 2401925 | 2401926 | 13233 | 13235 | YES | Null | — |
| 2024 | 2402489 | 2402494 | 2421973 | 2421974 | 13233 | 13235 | YES | O06217 | — |
| 2025 | 2404055 | 2404056 | 2423535 | 2423634 | 13233 | 13235 | YES | O06215 | — |
| 2026 | 2404228 | 2404229 | 2423816 | 2423835 | 13233 | 13235 | YES | O06215 | — |
| 2027 | 2410508 | 2410509 | 2430114 | 2431463 | 13233 | 13235 | YES | Null | — |
| 2028 | 2427464 | 2427465 | 2448428 | 2448430 | 13233 | 13235 | YES | O53521 | enzyme activity |
| 2029 | 2440537 | 2440642 | 2461502 | 2461503 | 13233 | 13235 | YES | Q10389 | integral to |
| | | | | | | | | | membrane |
| 2030 | 2480295 | 2480297 | 2501146 | 2501147 | 13233 | 13235 | YES | Q10511 | — |
| 2031 | 2502267 | 2502271 | 2523205 | 2523206 | 13233 | 13235 | YES | Null | — |
| 2032 | 2504789 | 2504790 | 2525724 | 2525726 | 13233 | 13235 | YES | O53525 | electron transport |
| 2033 | 2511025 | 2511026 | 2531961 | 2532058 | 13233 | 13235 | YES | Null | — |
| 2034 | 2513519 | 2513520 | 2534561 | 2534564 | 13233 | 13235 | YES | O53536 | nitrogen |
| | | | | | | | | | metabolism |
| 2035 | 2528967 | 2528968 | 2550011 | 2551360 | 13233 | 13235 | YES | Q50687 | glycerol |
| | | | | | | | | | metabolism |
| 2036 | 2540310 | 2540311 | 2562712 | 2562714 | 13233 | 13235 | YES | Q50675 | membrane |
| 2037 | 2540853 | 2540854 | 2563256 | 2563259 | 13233 | 13235 | YES | Q59570 | thiosulfate |
| | | | | | | | | | sulfurtransferase |
| | | | | | | | | | activity |
| 2038 | 2541962 | 2541964 | 2564367 | 2564368 | 13233 | 13235 | YES | Q50673 | enzyme activity |
| 2039 | 2544362 | 2544364 | 2566766 | 2566767 | 13233 | 13235 | YES | Null | — |
| 2040 | 2584392 | 2584410 | 2606795 | 2606796 | 13233 | 13235 | YES | P71879 | transporter activity |
| 2041 | 2592156 | 2592158 | 2614558 | 2614559 | 13233 | 13235 | YES | Null | — |
| 2042 | 2607119 | 2607120 | 2639043 | 2639047 | 13233 | 13235 | YES | P95249 | — |
| 2043 | 2658273 | 2658275 | 2690200 | 2690201 | 13233 | 13235 | YES | P71749 | — |
| 2044 | 2661152 | 2661153 | 2693078 | 2693088 | 13233 | 13235 | YES | P71748 | oxygen transporter |
| | | | | | | | | | activity |
| 2045 | 2672954 | 2672976 | 2704883 | 2704884 | 13233 | 13235 | YES | P71736 | — |
| 2046 | 2673694 | 2673779 | 2705602 | 2705603 | 13233 | 13235 | YES | Null | — |
| 2047 | 2679611 | 2679613 | 2711425 | 2711426 | 13233 | 13235 | YES | P71729 | — |
| 2048 | 2689758 | 2689759 | 2721571 | 2721574 | 13233 | 13235 | YES | P71924 | DNA binding |
| | | | | | | | | | activity |
| 2049 | 2692384 | 2692385 | 2724199 | 2724220 | 13233 | 13235 | YES | Null | — |
| 2050 | 2748934 | 2748935 | 2780769 | 2780773 | 13233 | 13235 | YES | O53203 | — |
| 2051 | 2752776 | 2752777 | 2784614 | 2785963 | 13233 | 13235 | YES | Null | — |
| 2052 | 2762072 | 2762073 | 2795268 | 2795270 | 13233 | 13235 | YES | Null | — |
| 2053 | 2762938 | 2762939 | 2796135 | 2796137 | 13233 | 13235 | YES | O53212 | — |
| 2054 | 2763778 | 2763780 | 2796976 | 2796977 | 13233 | 13235 | YES | O53212 | — |
| 2055 | 2768766 | 2768767 | 2801963 | 2801967 | 13233 | 13235 | YES | O53215 | RNA-3′-phosphate |
| | | | | | | | | | cyclase activity |
| 2056 | 2769956 | 2769957 | 2803156 | 2803166 | 13233 | 13235 | YES | O53215 | RNA-3′-phosphate |
| | | | | | | | | | cyclase activity |
| 2057 | 2771738 | 2771747 | 2804947 | 2804948 | 13233 | 13235 | YES | O53215 | RNA-3′-phosphate |
| | | | | | | | | | cyclase activity |
| 2058 | 2834003 | 2834650 | 2867204 | 2867205 | 13233 | 13235 | YES | P95009 | — |
| 2059 | 2839452 | 2839453 | 2871997 | 2871999 | 13233 | 13235 | YES | P95001 | shikimate 5- |
| | | | | | | | | | dehydrogenase |
| | | | | | | | | | activity |
| 2060 | 2849054 | 2849055 | 2881600 | 2881602 | 13233 | 13235 | YES | Q50737 | — |
| 2061 | 2855417 | 2855418 | 2887964 | 2887967 | 13233 | 13235 | YES | Q50732 | — |
| 2062 | 2863468 | 2863469 | 2896017 | 2896019 | 13233 | 13235 | YES | Q50649 | nucleic acid binding |
| | | | | | | | | | activity |
| 2063 | 2890583 | 2890593 | 2923133 | 2923134 | 13233 | 13235 | YES | Q50630 | — |
| 2064 | 2911188 | 2911198 | 2943729 | 2943730 | 13233 | 13235 | YES | O06199 | — |
| 2065 | 2915441 | 2915442 | 2947973 | 2947977 | 13233 | 13235 | YES | O06191 | — |
| 2066 | 2925032 | 2925033 | 2957567 | 2957569 | 13233 | 13235 | YES | P71930 | molecular_function |
| | | | | | | | | | unknown |
| 2067 | 2925801 | 2925803 | 2958337 | 2958338 | 13233 | 13235 | YES | P71930 | molecular_function |
| | | | | | | | | | unknown |
| 2068 | 2938901 | 2938902 | 2982418 | 2982420 | 13233 | 13235 | YES | Null | — |
| 2069 | 2947177 | 2947218 | 2990695 | 2990696 | 13233 | 13235 | YES | Null | — |
| 2070 | 2952702 | 2952795 | 2996165 | 2996166 | 13233 | 13235 | YES | O86317 | — |
| 2071 | 3010580 | 3010581 | 3053941 | 3053943 | 13233 | 13235 | YES | O33284 | — |
| 2072 | 3011358 | 3011359 | 3054720 | 3054795 | 13233 | 13235 | YES | O33284 | — |
| 2073 | 3012343 | 3012367 | 3055789 | 3055790 | 13233 | 13235 | YES | O33285 | — |
| 2074 | 3042938 | 3042939 | 3086361 | 3086386 | 13233 | 13235 | YES | O33321 | DNA binding |
| | | | | | | | | | activity |
| 2075 | 3043634 | 3043635 | 3087081 | 3087083 | 13233 | 13235 | YES | P30234 | alanine |
| | | | | | | | | | dehydrogenase |
| | | | | | | | | | activity |
| 2076 | 3064422 | 3064426 | 3107870 | 3107871 | 13233 | 13235 | YES | P71652 | — |
| 2077 | 3073960 | 3073961 | 3117405 | 3117408 | 13233 | 13235 | YES | P71639 | DNA binding |
| | | | | | | | | | activity |
| 2078 | 3075770 | 3075771 | 3119217 | 3119800 | 13233 | 13235 | YES | Null | — |
| 2079 | 3075914 | 3076356 | 3119953 | 3119954 | 13233 | 13235 | YES | Null | — |
| 2080 | 3076439 | 3076501 | 3120027 | 3120028 | 13233 | 13235 | YES | Null | — |
| 2081 | 3078601 | 3078745 | 3122118 | 3122119 | 13233 | 13235 | YES | Null | — |
| 2082 | 3078967 | 3078968 | 3122331 | 3122394 | 13233 | 13235 | YES | Null | — |
| 2083 | 3088034 | 3088044 | 3131469 | 3131470 | 13233 | 13235 | YES | P71629 | molecular_function |
| | | | | | | | | | unknown |
| 2084 | 3098539 | 3098540 | 3141965 | 3141967 | 13233 | 13235 | YES | P71617 | transporter activity |
| 2085 | 3112605 | 3112606 | 3156032 | 3156073 | 13233 | 13235 | YES | Null | — |
| 2086 | 3146666 | 3146667 | 3190147 | 3190149 | 13233 | 13235 | YES | Q10806, Q10806 | — |
| 2087 | 3150757 | 3150759 | 3194239 | 3194240 | 13233 | 13235 | YES | Q10809 | — |
| 2088 | 3196190 | 3196191 | 3239559 | 3239600 | 13233 | 13235 | YES | Null | — |
| 2089 | 3248018 | 3248123 | 3291442 | 3291443 | 13233 | 13235 | YES | Null | — |
| 2090 | 3253069 | 3253071 | 3296379 | 3296380 | 13233 | 13235 | YES | P96284 | enzyme activity |
| 2091 | 3267820 | 3267822 | 3311129 | 3311130 | 13233 | 13235 | YES | P95134 | metabolism |
| 2092 | 3288055 | 3288056 | 3331363 | 3331366 | 13233 | 13235 | YES | P95120 | aspartic-type |
| | | | | | | | | | endopeptidase |
| | | | | | | | | | activity |
| 2093 | 3293332 | 3293333 | 3336642 | 3336751 | 13233 | 13235 | YES | Null | — |
| 2094 | 3294465 | 3294466 | 3337893 | 3337903 | 13233 | 13235 | YES | P95114 | cell wall |
| 2095 | 3307774 | 3307815 | 3351211 | 3351212 | 13233 | 13235 | YES | Null | — |
| 2096 | 3313357 | 3313358 | 3356738 | 3356740 | 13233 | 13235 | YES | Null | — |
| 2097 | 3336999 | 3337085 | 3380437 | 3380438 | 13233 | 13235 | YES | O53268, O53268 | — |
| 2098 | 3371757 | 3371758 | 3415194 | 3415209 | 13233 | 13235 | YES | Null | — |
| 2099 | 3381643 | 3381645 | 3425094 | 3425095 | 13233 | 13235 | YES | P95097 | acyl-CoA |
| | | | | | | | | | dehydrogenase |
| | | | | | | | | | activity |
| 2100 | 3430544 | 3430546 | 3473994 | 3473995 | 13233 | 13235 | YES | Null | — |
| 2101 | 3436512 | 3436513 | 3479961 | 3479963 | 13233 | 13235 | YES | Null | — |
| 2102 | 3441287 | 3441288 | 3484737 | 3487503 | 13233 | 13235 | YES | O05793, O08362 | — |
| 2103 | 3455437 | 3456765 | 3501662 | 3501663 | 13233 | 13235 | YES | P95191 | receptor activity |
| 2104 | 3484092 | 3484093 | 3528980 | 3528984 | 13233 | 13235 | YES | O53309 | — |
| 2105 | 3484287 | 3486424 | 3529178 | 3529179 | 13233 | 13235 | YES | Null | — |
| 2106 | 3488662 | 3488663 | 3531407 | 3531409 | 13233 | 13235 | YES | O53312 | — |
| 2107 | 3501951 | 3501952 | 3544697 | 3544699 | 13233 | 13235 | YES | O53326 | — |
| 2108 | 3508479 | 3508480 | 3551226 | 3552575 | 13233 | 13235 | YES | Null | — |
| 2109 | 3508604 | 3508605 | 3552709 | 3554058 | 13233 | 13235 | YES | Null | — |
| 2110 | 3513313 | 3513315 | 3558776 | 3558777 | 13233 | 13235 | YES | Null | — |
| 2111 | 3521477 | 3521488 | 3566939 | 3566940 | 13233 | 13235 | YES | Null | — |
| 2112 | 3535183 | 3535184 | 3580635 | 3580637 | 13233 | 13235 | YES | O05863 | enzyme activity |
| 2113 | 3537673 | 3537674 | 3583126 | 3583130 | 13233 | 13235 | YES | O05860 | enzyme activity |
| 2114 | 3545179 | 3545181 | 3590635 | 3590636 | 13233 | 13235 | YES | Null | — |
| 2115 | 3545228 | 3545230 | 3590683 | 3590684 | 13233 | 13235 | YES | Null | — |
| 2116 | 3549001 | 3549042 | 3594455 | 3594456 | 13233 | 13235 | YES | Null | — |
| 2117 | 3552793 | 3552795 | 3598191 | 3598192 | 13233 | 13235 | YES | Null | — |
| 2118 | 3564990 | 3564992 | 3610387 | 3610388 | 13233 | 13235 | YES | O05879 | molecular_function |
| | | | | | | | | | unknown |
| 2119 | 3600628 | 3600629 | 3646024 | 3646037 | 13233 | 13235 | YES | P96870 | transferase activity |
| 2120 | 3618520 | 3618521 | 3663928 | 3663982 | 13233 | 13235 | YES | P96886 | — |
| 2121 | 3662150 | 3662151 | 3707621 | 3707625 | 13233 | 13235 | YES | Null | — |
| 2122 | 3681154 | 3681156 | 3723901 | 3723902 | 13233 | 13235 | YES | O53388 | — |
| 2123 | 3692113 | 3692114 | 3738414 | 3738416 | 13233 | 13235 | YES | O53394, O53395 | — |
| 2124 | 3692228 | 3692247 | 3738530 | 3738531 | 13233 | 13235 | YES | O53395 | — |
| 2125 | 3692363 | 3692364 | 3738647 | 3738758 | 13233 | 13235 | YES | O53395 | — |
| 2126 | 3694087 | 3694165 | 3740704 | 3740705 | 13233 | 13235 | YES | O53395 | — |
| 2127 | 3694390 | 3694391 | 3740920 | 3740930 | 13233 | 13235 | YES | O53395 | — |
| 2128 | 3694743 | 3694812 | 3741282 | 3741283 | 13233 | 13235 | YES | O53395 | — |
| 2129 | 3695504 | 3695505 | 3741965 | 3741967 | 13233 | 13235 | YES | O53395 | — |
| 2130 | 3700589 | 3700590 | 3747051 | 3747053 | 13233 | 13235 | YES | O50378 | — |
| 2131 | 3706947 | 3706948 | 3753410 | 3753680 | 13233 | 13235 | YES | Null | — |
| 2132 | 3708737 | 3708738 | 3755201 | 3755207 | 13233 | 13235 | YES | Null | — |
| 2133 | 3713227 | 3713228 | 3759696 | 3759698 | 13233 | 13235 | YES | O50379 | — |
| 2134 | 3733169 | 3733265 | 3779639 | 3779640 | 13233 | 13235 | YES | O50396 | — |
| 2135 | 3733306 | 3733316 | 3779671 | 3779672 | 13233 | 13235 | YES | O50396 | — |
| 2136 | 3744771 | 3744772 | 3791127 | 3791132 | 13233 | 13235 | YES | O50406 | enzyme activity |
| 2137 | 3748507 | 3748510 | 3794864 | 3794865 | 13233 | 13235 | YES | Null | — |
| 2138 | 3748698 | 3748699 | 3795053 | 3796402 | 13233 | 13235 | YES | Null | — |
| 2139 | 3754439 | 3754440 | 3802152 | 3802218 | 13233 | 13235 | YES | O50415 | — |
| 2140 | 3754539 | 3754580 | 3802327 | 3802328 | 13233 | 13235 | YES | O50415 | — |
| 2141 | 3754972 | 3754973 | 3802700 | 3802710 | 13233 | 13235 | YES | O50415 | — |
| 2142 | 3756095 | 3756164 | 3803832 | 3803833 | 13233 | 13235 | YES | O50415 | — |
| 2143 | 3772838 | 3773120 | 3820497 | 3820498 | 13233 | 13235 | YES | Null | — |
| 2144 | 3795208 | 3795209 | 3842576 | 3847493 | 13233 | 13235 | YES | Q50703, O06246 | — |
| 2145 | 3810175 | 3810176 | 3862469 | 3862471 | 13233 | 13235 | YES | Null | — |
| 2146 | 3822425 | 3822426 | 3874720 | 3874722 | 13233 | 13235 | YES | O06320 | — |
| 2147 | 3826298 | 3826299 | 3878594 | 3878598 | 13233 | 13235 | YES | Null | — |
| 2148 | 3826332 | 3826333 | 3878631 | 3878633 | 13233 | 13235 | YES | Null | — |
| 2149 | 3843412 | 3843413 | 3897070 | 3897774 | 13233 | 13235 | YES | O06342 | enzyme activity |
| 2150 | 3845388 | 3845389 | 3899759 | 3899762 | 13233 | 13235 | YES | O06343 | molecular_function |
| | | | | | | | | | unknown |
| 2151 | 3873325 | 3873326 | 3927698 | 3927779 | 13233 | 13235 | YES | O53552 | — |
| 2152 | 3873813 | 3873814 | 3928276 | 3928317 | 13233 | 13235 | YES | O53552 | — |
| 2153 | 3874278 | 3874297 | 3928795 | 3928796 | 13233 | 13235 | YES | O53552 | — |
| 2154 | 3874602 | 3874665 | 3929101 | 3929102 | 13233 | 13235 | YES | O53552 | — |
| 2155 | 3874830 | 3874831 | 3929257 | 3929276 | 13233 | 13235 | YES | O53552 | — |
| 2156 | 3877295 | 3877305 | 3931731 | 3931732 | 13233 | 13235 | YES | O53553 | — |
| 2157 | 3877742 | 3877743 | 3932169 | 3932328 | 13233 | 13235 | YES | O53553 | — |
| 2158 | 3878312 | 3878340 | 3932781 | 3932782 | 13233 | 13235 | YES | O53553 | — |
| 2159 | 3879828 | 3879829 | 3934873 | 3934922 | 13233 | 13235 | YES | O53553 | — |
| 2160 | 3888742 | 3888752 | 3944049 | 3944050 | 13233 | 13235 | YES | O53557 | hydroxymethylglutaryl- |
| | | | | | | | | | CoA reductase |
| | | | | | | | | | (NADPH) activity |
| 2161 | 3889054 | 3889064 | 3944352 | 3944353 | 13233 | 13235 | YES | O53557 | hydroxymethylglutaryl- |
| | | | | | | | | | CoA reductase |
| | | | | | | | | | (NADPH) activity |
| 2162 | 3892768 | 3892769 | 3947748 | 3948330 | 13233 | 13235 | YES | O53559 | — |
| 2163 | 3898149 | 3898150 | 3954929 | 3954931 | 13233 | 13235 | YES | O53563 | monooxygenase |
| | | | | | | | | | activity |
| 2164 | 3951089 | 3951090 | 4007870 | 4007872 | 13233 | 13235 | YES | P96848 | arylamine N- |
| | | | | | | | | | acetyltransferase |
| | | | | | | | | | activity |
| 2165 | 3964661 | 3964663 | 4021443 | 4021444 | 13233 | 13235 | YES | P96861 | RNA binding |
| | | | | | | | | | activity |
| 2166 | 3968799 | 3968800 | 4025580 | 4025582 | 13233 | 13235 | YES | Null | — |
| 2167 | 3980409 | 3980437 | 4037191 | 4037192 | 13233 | 13235 | YES | O06287 | — |
| 2168 | 3980524 | 3980525 | 4037279 | 4037281 | 13233 | 13235 | YES | O06287 | — |
| 2169 | 3996679 | 3996680 | 4053435 | 4053537 | 13233 | 13235 | YES | O06272, O06271 | — |
| 2170 | 3999617 | 3999619 | 4056484 | 4056485 | 13233 | 13235 | YES | Null | — |
| 2171 | 4031594 | 4031711 | 4094354 | 4094355 | 13233 | 13235 | YES | O69621 | — |
| 2172 | 4031993 | 4031994 | 4094627 | 4094644 | 13233 | 13235 | YES | Null | — |
| 2173 | 4032348 | 4032349 | 4094998 | 4095000 | 13233 | 13235 | YES | O69623 | — |
| 2174 | 4036755 | 4036757 | 4099406 | 4099407 | 13233 | 13235 | YES | Null | — |
| 2175 | 4076537 | 4076539 | 4139187 | 4139188 | 13233 | 13235 | YES | O69664 | glycerol kinase |
| | | | | | | | | | activity |
| 2176 | 4092930 | 4093092 | 4155579 | 4155580 | 13233 | 13235 | YES | P96420 | enzyme activity |
| 2177 | 4094427 | 4094428 | 4156905 | 4156946 | 13233 | 13235 | YES | Null | — |
| 2178 | 4107128 | 4107129 | 4169665 | 4169667 | 13233 | 13235 | YES | O69691 | — |
| 2179 | 4108423 | 4108425 | 4170961 | 4170962 | 13233 | 13235 | YES | O69692 | — |
| 2180 | 4133434 | 4133436 | 4197137 | 4197138 | 13233 | 13235 | YES | Null | — |
| 2181 | 4134910 | 4134911 | 4198612 | 4198614 | 13233 | 13235 | YES | Null | — |
| 2182 | 4154969 | 4154971 | 4218672 | 4218673 | 13233 | 13235 | YES | P72040 | — |
| 2183 | 4247018 | 4247020 | 4310742 | 4310743 | 13233 | 13235 | YES | P96242 | proteolysis and |
| | | | | | | | | | peptidolysis |
| 2184 | 4254698 | 4254699 | 4318421 | 4318691 | 13233 | 13235 | YES | Null | — |
| 2185 | 4274868 | 4274869 | 4338592 | 4338594 | 13233 | 13235 | YES | Null | — |
| 2186 | 4277269 | 4277306 | 4340994 | 4340995 | 13233 | 13235 | YES | P96213 | — |
| 2187 | 4277709 | 4277722 | 4341398 | 4341399 | 13233 | 13235 | YES | P96213 | — |
| 2188 | 4295484 | 4295503 | 4359161 | 4359162 | 13233 | 13235 | YES | O69743 | — |
| 2189 | 4295520 | 4295548 | 4359179 | 4359180 | 13233 | 13235 | YES | O69743 | — |
| 2190 | 4297431 | 4297432 | 4361063 | 4361065 | 13233 | 13235 | YES | Q933K8 | — |
| 2191 | 4297455 | 4297457 | 4361088 | 4361089 | 13233 | 13235 | YES | Q933K8 | — |
| 2192 | 4307387 | 4307388 | 4371019 | 4373416 | 13233 | 13235 | YES | O05457, O05455 | — |
| 2193 | 4307808 | 4307809 | 4373846 | 4373848 | 13233 | 13235 | YES | O05454 | — |
| 2194 | 4308332 | 4308333 | 4374371 | 4374373 | 13233 | 13235 | YES | Null | — |
| 2195 | 4312098 | 4312100 | 4378138 | 4378139 | 13233 | 13235 | YES | O05450 | — |
| 2196 | 4316061 | 4316062 | 4382100 | 4382102 | 13233 | 13235 | YES | O05448 | — |
| 2197 | 4317102 | 4317108 | 4383142 | 4383143 | 13233 | 13235 | YES | O07036 | — |
| 2198 | 4318998 | 4318999 | 4385033 | 4385035 | 13233 | 13235 | YES | O05446 | — |
| 2199 | 4334624 | 4334625 | 4400660 | 4400662 | 13233 | 13235 | YES | O53590 | DNA binding |
| | | | | | | | | | activity |
| 2200 | 3076218 | 3076219 | 3122987 | 3123052 | 13233 | 13235 | YES | Null | — |
| 2201 | 71576 | 71614 | 71584 | 71585 | 147805 | 147807 | NO | NULL | NULL |
| 2202 | 131213 | 131215 | 131174 | 131175 | 578886 | 578887 | NO | NULL | NULL |
| 2203 | 147853 | 147854 | 147812 | 147814 | 582109 | 582110 | NO | NULL | NULL |
| 2204 | 230770 | 230772 | 230575 | 230576 | 596768 | 596769 | NO | NULL | NULL |
| 2205 | 291957 | 291959 | 293631 | 293632 | 612279 | 612281 | NO | NULL | NULL |
| 2206 | 578459 | 578500 | 577494 | 577495 | 664896 | 664897 | NO | NULL | NULL |
| 2207 | 612063 | 612064 | 610910 | 610912 | 704228 | 704229 | NO | NULL | NULL |
| 2208 | 743870 | 744394 | 742632 | 742633 | 730004 | 730005 | NO | NULL | NULL |
| 2209 | 804268 | 804309 | 802498 | 802499 | 737681 | 737682 | NO | NULL | NULL |
| 2210 | 856450 | 856451 | 854258 | 854260 | 804527 | 804680 | NO | NULL | NULL |
| 2211 | 953566 | 953567 | 952809 | 952811 | 815060 | 815062 | NO | NULL | NULL |
| 2212 | 961024 | 961025 | 960268 | 960309 | 960120 | 960270 | NO | NULL | NULL |
| 2213 | 1064550 | 1064551 | 1064081 | 1064110 | 1090204 | 1090205 | NO | NULL | NULL |
| 2214 | 1090629 | 1090631 | 1090189 | 1090190 | 1172885 | 1172887 | NO | NULL | NULL |
| 2215 | 1165969 | 1165971 | 1165520 | 1165521 | 1277359 | 1277361 | NO | NULL | NULL |
| 2216 | 1173288 | 1173289 | 1172837 | 1172839 | 1305018 | 1305133 | NO | NULL | NULL |
| 2217 | 1306903 | 1306904 | 1305528 | 1305643 | 1312879 | 1312880 | NO | NULL | NULL |
| 2218 | 1314587 | 1314589 | 1313336 | 1313337 | 1365329 | 1365330 | NO | NULL | NULL |
| 2219 | 1475063 | 1475064 | 1477025 | 1477027 | 1408866 | 1408867 | NO | NULL | NULL |
| 2220 | 1539986 | 1539987 | 1541949 | 1543298 | 1476568 | 1476570 | NO | NULL | NULL |
| 2221 | 1837548 | 1837549 | 1852180 | 1852182 | 1489302 | 1489303 | NO | NULL | NULL |
| 2222 | 1892915 | 1892916 | 1907517 | 1907558 | 1606135 | 1606137 | NO | NULL | NULL |
| 2223 | 1914068 | 1914069 | 1928724 | 1928726 | 1630252 | 1630253 | NO | NULL | NULL |
| 2224 | 1941012 | 1941053 | 1955670 | 1955671 | 1644464 | 1644505 | NO | NULL | NULL |
| 2225 | 2064977 | 2064978 | 2074511 | 2074614 | 1843089 | 1843091 | NO | NULL | NULL |
| 2226 | 2087040 | 2087041 | 2096828 | 2096830 | 1886393 | 1886394 | NO | NULL | NULL |
| 2227 | 2093386 | 2093387 | 2103175 | 2103177 | 1898321 | 1898362 | NO | NULL | NULL |
| 2228 | 2099733 | 2099735 | 2109523 | 2109524 | 1919528 | 1919530 | NO | NULL | NULL |
| 2229 | 2199225 | 2199227 | 2207589 | 2207590 | 2094050 | 2094052 | NO | NULL | NULL |
| 2230 | 2254439 | 2254440 | 2270521 | 2270531 | 2100397 | 2100399 | NO | NULL | NULL |
| 2231 | 2347255 | 2347256 | 2365412 | 2366761 | 2132111 | 2132112 | NO | NULL | NULL |
| 2232 | 2349048 | 2349049 | 2368563 | 2368565 | 2484768 | 2484769 | NO | NULL | NULL |
| 2233 | 2382325 | 2382432 | 2401925 | 2401926 | 2484875 | 2484876 | NO | NULL | NULL |
| 2234 | 2410508 | 2410509 | 2430114 | 2431463 | 2529221 | 2529262 | NO | NULL | NULL |
| 2235 | 2502267 | 2502271 | 2523205 | 2523206 | 2562613 | 2562614 | NO | NULL | NULL |
| 2236 | 2511025 | 2511026 | 2531961 | 2532058 | 2701263 | 2701264 | NO | NULL | NULL |
| 2237 | 2544362 | 2544364 | 2566766 | 2566767 | 2721050 | 2721071 | NO | NULL | NULL |
| 2238 | 2592156 | 2592158 | 2614558 | 2614559 | 2790757 | 2790759 | NO | NULL | NULL |
| 2239 | 2673694 | 2673779 | 2705602 | 2705603 | 2846431 | 2846432 | NO | NULL | NULL |
| 2240 | 2692384 | 2692385 | 2724199 | 2724220 | 2953712 | 2953714 | NO | NULL | NULL |
| 2241 | 2752776 | 2752777 | 2784614 | 2785963 | 2977206 | 2977208 | NO | NULL | NULL |
| 2242 | 2762072 | 2762073 | 2795268 | 2795270 | 3088594 | 3088595 | NO | NULL | NULL |
| 2243 | 2938901 | 2938902 | 2982418 | 2982420 | 3113937 | 3114520 | NO | NULL | NULL |
| 2244 | 2947177 | 2947218 | 2990695 | 2990696 | 3114673 | 3114674 | NO | NULL | NULL |
| 2245 | 3075770 | 3075771 | 3119217 | 3119800 | 3116761 | 3116762 | NO | NULL | NULL |
| 2246 | 3075914 | 3076356 | 3119953 | 3119954 | 3117201 | 3117264 | NO | NULL | NULL |
| 2247 | 3076439 | 3076501 | 3120027 | 3120028 | 3150246 | 3150287 | NO | NULL | NULL |
| 2248 | 3078601 | 3078745 | 3122118 | 3122119 | 3220467 | 3220468 | NO | NULL | NULL |
| 2249 | 3078967 | 3078968 | 3122331 | 3122394 | 3233882 | 3233923 | NO | NULL | NULL |
| 2250 | 3112605 | 3112606 | 3156032 | 3156073 | 3285765 | 3285766 | NO | NULL | NULL |
| 2251 | 3196190 | 3196191 | 3239559 | 3239600 | 3330159 | 3330160 | NO | NULL | NULL |
| 2252 | 3248018 | 3248123 | 3291442 | 3291443 | 3345553 | 3345554 | NO | NULL | NULL |
| 2253 | 3293332 | 3293333 | 3336642 | 3336751 | 3370822 | 3370824 | NO | NULL | NULL |
| 2254 | 3307774 | 3307815 | 3351211 | 3351212 | 3475760 | 3475762 | NO | NULL | NULL |
| 2255 | 3313357 | 3313358 | 3356738 | 3356740 | 3561846 | 3561847 | NO | NULL | NULL |
| 2256 | 3371757 | 3371758 | 3415194 | 3415209 | 3585541 | 3585542 | NO | NULL | NULL |
| 2257 | 3430544 | 3430546 | 3473994 | 3473995 | 3589158 | 3589159 | NO | NULL | NULL |
| 2258 | 3436512 | 3436513 | 3479961 | 3479963 | 3589214 | 3589215 | NO | NULL | NULL |
| 2259 | 3484287 | 3486424 | 3529178 | 3529179 | 3589361 | 3589363 | NO | NULL | NULL |
| 2260 | 3508479 | 3508480 | 3551226 | 3552575 | 3590883 | 3590885 | NO | NULL | NULL |
| 2261 | 3508604 | 3508605 | 3552709 | 3554058 | 3590911 | 3590912 | NO | NULL | NULL |
| 2262 | 3513313 | 3513315 | 3558776 | 3558777 | 3685808 | 3685849 | NO | NULL | NULL |
| 2263 | 3521477 | 3521488 | 3566939 | 3566940 | 3702512 | 3702516 | NO | NULL | NULL |
| 2264 | 3545179 | 3545181 | 3590635 | 3590636 | 3717525 | 3717526 | NO | NULL | NULL |
| 2265 | 3545228 | 3545230 | 3590683 | 3590684 | 3747436 | 3747442 | NO | NULL | NULL |
| 2266 | 3549001 | 3549042 | 3594455 | 3594456 | 3787100 | 3787101 | NO | NULL | NULL |
| 2267 | 3662150 | 3662151 | 3707621 | 3707625 | 3811352 | 3811353 | NO | NULL | NULL |
| 2268 | 3706947 | 3706948 | 3753410 | 3753680 | 3835096 | 3835097 | NO | NULL | NULL |
| 2269 | 3708737 | 3708738 | 3755201 | 3755207 | 3864545 | 3864549 | NO | NULL | NULL |
| 2270 | 3748507 | 3748510 | 3794864 | 3794865 | 3864582 | 3864584 | NO | NULL | NULL |
| 2271 | 3748698 | 3748699 | 3795053 | 3796402 | 3903645 | 3903646 | NO | NULL | NULL |
| 2272 | 3772838 | 3773120 | 3820497 | 3820498 | 4017722 | 4017724 | NO | NULL | NULL |
| 2273 | 3810175 | 3810176 | 3862469 | 3862471 | 4086889 | 4086906 | NO | NULL | NULL |
| 2274 | 3826298 | 3826299 | 3878594 | 3878598 | 4091666 | 4091667 | NO | NULL | NULL |
| 2275 | 3826332 | 3826333 | 3878631 | 3878633 | 4109424 | 4109425 | NO | NULL | NULL |
| 2276 | 3968799 | 3968800 | 4025580 | 4025582 | 4160762 | 4160763 | NO | NULL | NULL |
| 2277 | 3999617 | 3999619 | 4056484 | 4056485 | 4348926 | 4348927 | NO | NULL | NULL |
| 2278 | 4031993 | 4031994 | 4094627 | 4094644 | 4366675 | 4366677 | NO | NULL | NULL |
| 2279 | 4036755 | 4036757 | 4099406 | 4099407 | | | NO | NULL | NULL |
| 2280 | 4094427 | 4094428 | 4156905 | 4156946 | | | NO | NULL | NULL |
| 2281 | 4133434 | 4133436 | 4197137 | 4197138 | | | NO | NULL | NULL |
| 2282 | 4134910 | 4134911 | 4198612 | 4198614 | | | NO | NULL | NULL |
| 2283 | 4254698 | 4254699 | 4318421 | 4318691 | | | NO | NULL | NULL |
| 2284 | 4274868 | 4274869 | 4338592 | 4338594 | | | NO | NULL | NULL |
| 2285 | 4308332 | 4308333 | 4374371 | 4374373 | | | NO | NULL | NULL |
| 2286 | 3076218 | 3076219 | 3122987 | 3123052 | | | NO | NULL | NULL |

Table II: List of insertion/deletions (indels) in Mycobacaterium tuberculosis/M. bovis BCG
Polymorphism ID: The ID by which the polymorphism can be identified
BCG Start: The position in the genome of M. bovis BCG at which insertion/deletion starts
BCG End: The position in the genome of M. bovis BCG at which insertion/deletion ends
H37Rv Start: The position in the genome of M. tuberculosis H37Rv at which insertion/deletion starts
H37Rv End: The position in the genome of M. tuberculosis H37Rv at which insertion/deletion ends
CDC1551 Start: The position in the genome of M. tuberculosis CDC1551 at which insertion/deletion starts
CDC1551 End: The position in the genome of M. tuberculosis CDC1551 at which insertion/deletion ends
ORF: Indicates whether the polymorphism occurs in an open reading frame (yes) or not (no)
GO ID: The ID for the sequence in the gene ontology database
Putative function: The putative function of the gene in which the SNP occurs.

TABLE 3
List of long polymorphisms in Mycobacterium tuberculosis/M. bovis BCG.

| Polymorphism | BCG | | H37Rv | H37Rv | | | | | |
|---|---|---|---|---|---|---|---|---|---|
| ID | Start | BCG End | start | end | CDC start | CDC end | ORF | GO ID | Putative Function |
| 2287 | 55529 | 55544 | 55543 | 55552 | 103765 | 105054 | Yes | P71707 | enzyme activity |
| 2288 | 103810 | 105100 | 103773 | 105062 | 103765 | 105054 | Yes | Q50655, | — |
| | | | | | | | | Q10891 | |
| 2289 | 337700 | 337733 | 336678 | 336711 | 103765 | 105054 | Yes | O53684 | — |
| 2290 | 339670 | 339722 | 338666 | 338718 | 103765 | 105054 | Yes | O53684 | — |
| 2291 | 468517 | 468610 | 467498 | 467589 | 103765 | 105054 | Yes | O53722 | — |
| 2292 | 840823 | 840895 | 838955 | 838967 | 103765 | 105054 | Yes | O53810 | — |
| 2293 | 891209 | 892235 | 889018 | 891403 | 103765 | 105054 | Yes | O07182 | DNA binding activity |
| 2294 | 928362 | 928365 | 927446 | 927461 | 103765 | 105054 | Yes | O53844 | — |
| 2295 | 1094366 | 1094867 | 1093925 | 1094414 | 103765 | 105054 | Yes | O53891 | — |
| 2296 | 1413023 | 1413095 | 1414785 | 1414947 | 103765 | 105054 | Yes | Q11053 | protein kinase activity |
| 2297 | 1466961 | 1466963 | 1468912 | 1468925 | 103765 | 105054 | Yes | Q10621 | DNA binding activity |
| 2298 | 1530977 | 1531052 | 1532940 | 1533015 | 103765 | 105054 | Yes | Q11031 | — |
| 2299 | 1531093 | 1531199 | 1533056 | 1533162 | 103765 | 105054 | Yes | Q11031 | — |
| 2300 | 1619416 | 1619437 | 1623086 | 1623088 | 103765 | 105054 | Yes | Null | — |
| 2301 | 1629885 | 1631221 | 1633536 | 1634635 | 103765 | 105054 | Yes | O06810 | — |
| 2302 | 1633501 | 1634095 | 1636765 | 1637347 | 103765 | 105054 | Yes | O06808 | — |
| 2303 | 1634927 | 1634959 | 1638179 | 1638211 | 103765 | 105054 | Yes | O06808 | — |
| 2304 | 1773935 | 1775045 | 1788567 | 1789677 | 103765 | 105054 | Yes | O06603, | — |
| | | | | | | | | O06602 | |
| 2305 | 1986939 | 1988741 | 1996098 | 1998299 | 103765 | 105054 | Yes | O06798 | nucleic acid binding |
| | | | | | | | | | activity |
| 2306 | 2156908 | 2157537 | 2165848 | 2165901 | 103765 | 105054 | Yes | O07716 | enzyme activity |
| 2307 | 2241088 | 2241814 | 2262170 | 2262896 | 103765 | 105054 | Yes | O53461 | nucleic acid binding |
| | | | | | | | | | activity |
| 2308 | 2278758 | 2278826 | 2294843 | 2294911 | 103765 | 105054 | Yes | O53490 | enzyme activity |
| 2309 | 2278938 | 2278961 | 2295023 | 2295046 | 103765 | 105054 | Yes | O53490 | enzyme activity |
| 2310 | 2279216 | 2280345 | 2295301 | 2296430 | 103765 | 105054 | Yes | O53490 | enzyme activity |
| 2311 | 2285306 | 2286046 | 2301391 | 2302131 | 103765 | 105054 | Yes | O53490 | enzyme activity |
| 2312 | 2501326 | 2501345 | 2522176 | 2522283 | 103765 | 105054 | Yes | Null | — |
| 2313 | 2604210 | 2605004 | 2635574 | 2636928 | 103765 | 105054 | Yes | P95248 | — |
| 2314 | 2912021 | 2912672 | 2944553 | 2945204 | 103765 | 105054 | Yes | O06199 | — |
| 2315 | 3079181 | 3079369 | 3122617 | 3123099 | 103765 | 105054 | Yes | Null | — |
| 2316 | 3079550 | 3079876 | 3123280 | 3123311 | 103765 | 105054 | Yes | Null | — |
| 2317 | 3189218 | 3189388 | 3232699 | 3232757 | 103765 | 105054 | Yes | Null | — |
| 2318 | 3204423 | 3204457 | 3247847 | 3247881 | 103765 | 105054 | Yes | Q10977 | enzyme activity |
| 2319 | 3334709 | 3334850 | 3378091 | 3378288 | 103765 | 105054 | Yes | P31500 | — |
| 2320 | 3336266 | 3336348 | 3379704 | 3379786 | 103765 | 105054 | Yes | O53268 | — |
| 2321 | 3689443 | 3689509 | 3732189 | 3735810 | 103765 | 105054 | Yes | O53393 | carboxypeptidase A |
| | | | | | | | | | activity |
| 2322 | 3689905 | 3689925 | 3736206 | 3736226 | 103765 | 105054 | Yes | O53393 | carboxypeptidase A |
| | | | | | | | | | activity |
| 2323 | 3692719 | 3692740 | 3739123 | 3739357 | 103765 | 105054 | Yes | O53395 | — |
| 2324 | 3703709 | 3703744 | 3750172 | 3750207 | 103765 | 105054 | Yes | O50378 | — |
| 2325 | 3838472 | 3838474 | 3890772 | 3892132 | 103765 | 105054 | Yes | Null | — |
| 2326 | 3876017 | 3876037 | 3930462 | 3930473 | 103765 | 105054 | Yes | O53552 | — |
| 2327 | 3878151 | 3878280 | 3932746 | 3932749 | 103765 | 105054 | Yes | O53553 | — |
| 2328 | 3879035 | 3879494 | 3933477 | 3934539 | 103765 | 105054 | Yes | O53553 | — |
| 2329 | 3879583 | 3879686 | 3934628 | 3934731 | 103765 | 105054 | Yes | O53553 | — |
| 2330 | 3879863 | 3880576 | 3934956 | 3936335 | 103765 | 105054 | Yes | O53553 | — |
| 2331 | 3885770 | 3886315 | 3941529 | 3941721 | 103765 | 105054 | Yes | O53556, | — |
| | | | | | | | | O53557 | |
| 2332 | 3887733 | 3887868 | 3943139 | 3943175 | 103765 | 105054 | Yes | O53557 | hydroxymethylglutaryl- |
| | | | | | | | | | CoA reductase (NADPH) |
| | | | | | | | | | activity |
| 2333 | 3890973 | 3891602 | 3946262 | 3946876 | 103765 | 105054 | Yes | O53559 | — |
| 2334 | 3891837 | 3892400 | 3947111 | 3947380 | 103765 | 105054 | Yes | O53559 | — |
| 2335 | 3892771 | 3892967 | 3948342 | 3949747 | 103765 | 105054 | Yes | O53559 | — |
| 2336 | 4127053 | 4127055 | 4189590 | 4190758 | 103765 | 105054 | Yes | O69705 | — |
| 2337 | 4189866 | 4189868 | 4253568 | 4253581 | 103765 | 105054 | Yes | Q10621 | DNA binding activity |
| 2338 | 4190616 | 4190621 | 4254329 | 4254345 | 103765 | 105054 | Yes | Null | — |
| 2339 | 1973115 | 1973588 | 2628042 | 2630136 | 103765 | 105054 | Yes | P95245, | — |
| | | | | | | | | P95246 | — |
| 2340 | 3079605 | 3079661 | 3119272 | 3119329 | 103765 | 105054 | Yes | Null | Null |
| 2341 | 1619416 | 1619437 | 1623086 | 1623088 | 1622970 | 1622972 | No | Null | Null |
| 2342 | 2501326 | 2501345 | 2522176 | 2522283 | 2354339 | 2354347 | No | Null | Null |
| 2343 | 3079181 | 3079369 | 3122617 | 3123099 | 2519450 | 2519556 | No | Null | Null |
| 2344 | 3079550 | 3079876 | 3123280 | 3123311 | 2520462 | 2520465 | No | Null | Null |
| 2345 | 3189218 | 3189388 | 3232699 | 3232757 | 2985403 | 2985518 | No | Null | Null |
| 2346 | 3838472 | 3838474 | 3890772 | 3892132 | 3018402 | 3018431 | No | Null | Null |
| 2347 | 4190616 | 4190621 | 4254329 | 4254345 | 3226853 | 3226952 | No | Null | Null |
| 2348 | 3079605 | 3079661 | 3119272 | 3119329 | 3589269 | 3589323 | No | Null | Null |
| 2349 | | | | | 4245189 | 4245204 | No | Null | Null |
| 2350 | | | | | 4246654 | 4246670 | No | Null | Null |
| 2351 | | | | | 3113992 | 3114049 | No | Null | Null |

Table III: List of long polymorphisms in Mycobacaterium tuberculosis/M. bovis BCG
Polymorphism ID: The ID by which the polymorphism can be identified
BCG Start: The position in the genome of M. bovis BCG at which multiple polymorhisms start occurring
BCG End: The position in the genome of M. bovis BCG at which multiple polymorhisms end
H37Rv Start: The position in the genome of M. tuberculosis H37Rv at which multiple polymorhisms start
H37Rv End: The position in the genome of M. tuberculosis H37Rv at multiple polymorhisms end
C1551 Start: The position in the genome of M. tuberculosis CDC1551 at which multiple polymorhisms start
CDC1551 End: The position in the genome of M. tuberculosis CDC1551 at which multiple polymorhisms ends
ORF: Indicates whether the polymorphism occurs in an open reading frame (yes) or not (no)
GO ID: The ID for the sequence in the gene ontology database
Putative function: The putative function of the gene in which the SNP occurs.

TABLE 4
a:
List of Polymorphisms (Single Nucleotide Polymorphisms) in genes involved in cell wall synthesis

| Polymorphism | | BCG | | Query | Query | Query | Query | | Type of | | Putative |
|---|---|---|---|---|---|---|---|---|---|---|---|
| ID | BCG Position | base | BCG AA | name | Position | base | aa | ORF | SNP | GO ID | Function |
| 48 | 53663 | T | L | H37Rv | 53677 | C | P | Yes | NS, NC | P71707 | Cell wall |
| | | | | | | | | | | | synthesis |
| 48 | 53663 | T | L | CDC1551 | 53623 | C | P | Yes | NS, NC | Q8VKS5 | Cell wall |
| | | | | | | | | | | | synthesis |
| 1014 | 2393645 | G | P | H37Rv | 2413129 | A | L | Yes | NS, NC | O06224 | Cell wall |
| | | | | | | | | | | | synthesis |
| 1014 | 2393645 | G | P | CDC1551 | 2411822 | A | L | Yes | NS, NC | O06224 | Cell wall |
| | | | | | | | | | | | synthesis |
| 1015 | 2393760 | A | L | H37Rv | 2413244 | C | V | Yes | NS, C | O06224 | Cell wall |
| | | | | | | | | | | | synthesis |
| 1015 | 2393760 | A | L | CDC1551 | 2411937 | C | V | Yes | NS, C | O06224 | Cell wall |
| | | | | | | | | | | | synthesis |
| 1240 | 2987804 | G | H | H37Rv | 3031165 | A | Y | Yes | NS, C | O07218 | Cell wall |
| | | | | | | | | | | | synthesis |
| 1240 | 2987804 | G | H | CDC1551 | 3026104 | A | Y | Yes | NS, C | O07218 | Cell wall |
| | | | | | | | | | | | synthesis |
| 1746 | 4176966 | T | I | H37Rv | 4240668 | C | T | Yes | NS, NC | P72059 | Cell wall |
| | | | | | | | | | | | synthesis |
| 1746 | 4176966 | T | I | CDC1551 | 4232983 | C | T | Yes | NS, NC | P72059 | Cell wall |
| | | | | | | | | | | | synthesis |
| 1749 | 4182846 | G | S | H37Rv | 4246548 | A | N | Yes | NS, C | P72030 | Cell wall |
| | | | | | | | | | | | synthesis |
| 1749 | 4182846 | G | S | CDC1551 | 4238863 | A | N | Yes | NS, C | P72030 | Cell wall |
| | | | | | | | | | | | synthesis |
| 1751 | 4183941 | C | A | H37Rv | 4247643 | A | E | Yes | NS, NC | P72030 | Cell wall |
| | | | | | | | | | | | synthesis |
| 1751 | 4183941 | C | A | CDC1551 | 4239958 | A | E | Yes | NS, NC | P72030 | Cell wall |
| | | | | | | | | | | | synthesis |

| | | | | | | | | |
|---|---|---|---|---|---|---|---|---|
| Polymorphism | | | | | | | | |
| ID | BCG start | BCG end | Query name | Query start | Query end | ORF | GO ID | Putative Function |

b:
List of Polymorphisms (Insertions/deletions) in genes involved in cell wall synthesis

| | | | | | | | | |
|---|---|---|---|---|---|---|---|---|
| 1906 | 936197 | 936204 | H37Rv | 935446 | 935447 | Yes | O53850 | Cell wall synthesis |
| 1947 | 1439690 | 1439691 | H37Rv | 1441542 | 1441686 | Yes | Q10614 | Cell wall synthesis |
| 2094 | 3294465 | 3294466 | H37Rv | 3337893 | 3337903 | Yes | P95114 | Cell wall synthesis |
| 1906 | 936197 | 936204 | CDC1551 | 935348 | 935349 | Yes | O53850 | Cell wall synthesis |
| 1947 | 1439690 | 1439691 | CDC1551 | 1441030 | 1441174 | Yes | Q10614 | Cell wall synthesis |
| 2094 | 3294465 | 3294466 | CDC1551 | 3332273 | 3332283 | Yes | P95114 | Cell wall synthesis |

c:
List of Polymorphisms (long polymorphisms) in genes involved in cell wall synthesis

| | | | | | | | | |
|---|---|---|---|---|---|---|---|---|
| 2287 | 55529 | 55544 | H37Rv | 55543 | 55552 | Yes | P71707 | Cell wall Synthesis |

Table IV: List of long polymorphisms in genes involved in cell wall synthesis
Polymorphism ID: The ID by which the polymorphism can be identified
BCG Start: The position in the genome of M. bovis BCG at which multiple polymorhisms start occurring
BCG End: The position in the genome of M. bovis BCG at which multiple polymorhisms end
H37Rv Start: The position in the genome of M. tuberculosis H37Rv at which multiple polymorhisms start
H37Rv End: The position in the genome of M. tuberculosis H37Rv at multiple polymorhisms end
C1551 Start: The position in the genome of M. tuberculosis CDC1551 at which multiple polymorhisms start
CDC1551 End: The position in the genome of M. tuberculosis CDC1551 at which multiple polymorhisms ends
ORF: Indicates whether the polymorphism occurs in an open reading frame (yes) or not (no)
GO ID: The ID for the sequence in the gene ontology database
Putative function: The putative function of the gene in which the SNP occurs.

TABLE 5
a: List of Polymorphisms (Single Nucleotide Polymorphisms) in transcription factors.

| Polymorphism | BCG | BCG | BCG | Query | Query | Query | Query | | Type of | | |
|---|---|---|---|---|---|---|---|---|---|---|---|
| ID | Position | base | AA | name | Position | base | aa | ORF | SNP | GO ID | Putative Function |
| 63 | 86899 | G | V | H37Rv | 86862 | A | I | Yes | NS, C | O53623 | Transcription factor |
| 63 | 86899 | G | V | CDC1551 | 86854 | A | I | Yes | NS, C | O53623 | Transcription factor |
| 188 | 366022 | T | V | H37Rv | 364973 | C | A | Yes | NS, C | O07229 | Transcription factor |
| 188 | 366022 | T | V | CDC1551 | 365037 | C | A | Yes | NS, C | O07229 | Transcription factor |
| 228 | 456342 | G | R | H37Rv | 455323 | C | P | Yes | NS, NC | O53712 | Transcription factor |
| 228 | 456342 | G | R | CDC1551 | 455414 | C | P | Yes | NS, NC | O53712 | Transcription factor |
| 231 | 467402 | C | A | H37Rv | 466383 | A | D | Yes | NS, NC | O53720 | Transcription factor |
| 231 | 467402 | C | A | CDC1551 | 466474 | A | D | Yes | NS, NC | O53720 | Transcription factor |
| 299 | 634973 | C | V | CDC1551 | 635181 | T | I | Yes | NS, C | Q8VKJ4 | Transcription factor |
| 313 | 671788 | A | H | H37Rv | 670543 | G | R | Yes | NS, NC | O53773 | Transcription factor |
| 313 | 671788 | A | H | CDC1551 | 671996 | G | R | Yes | NS, NC | O53773 | Transcription factor |
| 326 | 700963 | T | I | H37Rv | 699716 | C | V | Yes | NS, C | O07776 | Transcription factor |
| 326 | 700963 | T | I | CDC1551 | 701166 | C | V | Yes | NS, C | O07776 | Transcription factor |
| 405 | 912091 | C | P | H37Rv | 911259 | T | L | Yes | NS, NC | O53830 | Transcription factor |
| 405 | 912091 | C | P | CDC1551 | 911170 | T | L | Yes | NS, NC | O53830 | Transcription factor |
| 433 | 941645 | G | P | H37Rv | 940888 | C | A | Yes | NS, NC | O53856 | Transcription factor |
| 433 | 941645 | G | P | CDC1551 | 940790 | C | A | Yes | NS, NC | O53856 | Transcription factor |
| 483 | 1097474 | A | S | H37Rv | 1097021 | G | G | Yes | NS, NC | O53894 | Transcription factor |
| 483 | 1097474 | A | S | CDC1551 | 1097062 | G | G | Yes | NS, NC | O53894 | Transcription factor |
| 598 | 1375153 | G | P | H37Rv | 1373907 | A | S | Yes | NS, NC | O86313 | Transcription factor |
| 598 | 1375153 | G | P | CDC1551 | 1373397 | A | S | Yes | NS, NC | O86313 | Transcription factor |
| 611 | 1401640 | G | V | H37Rv | 1400394 | A | M | Yes | NS, NC | Q11039 | Transcription factor |
| 611 | 1401640 | G | V | CDC1551 | 1399883 | A | M | Yes | NS, NC | Q11039 | Transcription factor |
| 639 | 1476918 | T | * | H37Rv | 1478881 | C | W | Yes | NS, TP | Q10630 | Transcription factor |
| 639 | 1476918 | T | * | CDC1551 | 1478424 | C | W | Yes | NS, TP | Q10630 | Transcription factor |
| 640 | 1477120 | C | V | H37Rv | 1479083 | T | I | Yes | NS, C | Q10630 | Transcription factor |
| 640 | 1477120 | C | V | CDC1551 | 1478626 | T | I | Yes | NS, C | Q10630 | Transcription factor |
| 659 | 1524738 | T | V | H37Rv | 1526701 | G | G | Yes | NS, C | Q11028 | Transcription factor |
| 659 | 1524738 | T | V | CDC1551 | 1527918 | G | G | Yes | NS, C | Q8VK33 | Transcription factor |
| 660 | 1525971 | C | A | H37Rv | 1527934 | A | D | Yes | NS, NC | Q11028 | Transcription factor |
| 660 | 1525971 | C | A | CDC1551 | 1529151 | A | D | Yes | NS, NC | Q8VK33 | Transcription factor |
| 677 | 1534974 | T | T | H37Rv | 1536937 | C | A | Yes | NS, NC | Q11034 | Transcription factor |
| 677 | 1534974 | T | T | CDC1551 | 1538155 | C | A | Yes | NS, NC | Q11034 | Transcription factor |
| 700 | 1580686 | C | L | H37Rv | 1584377 | A | I | Yes | NS, C | P71675 | Transcription factor |
| 700 | 1580686 | C | L | CDC1551 | 1584233 | A | I | Yes | NS, C | P71675 | Transcription factor |
| 722 | 1643728 | T | V | H37Rv | 1646980 | C | A | Yes | NS, C | O53151 | Transcription factor |
| 722 | 1643728 | T | V | CDC1551 | 1647138 | C | A | Yes | NS, C | O53151 | Transcription factor |
| 801 | 1886196 | G | A | H37Rv | 1900798 | A | V | Yes | NS, C | O53922 | Transcription factor |
| 801 | 1886196 | G | A | CDC1551 | 1891602 | A | V | Yes | NS, C | O53922 | Transcription factor |
| 1061 | 2504215 | G | A | H37Rv | 2525150 | T | D | Yes | NS, NC | Q10528 | Transcription factor |
| 1061 | 2504215 | G | A | CDC1551 | 2522412 | T | D | Yes | NS, NC | Q10528 | Transcription factor |
| 1099 | 2609911 | G | R | H37Rv | 2641838 | A | H | Yes | NS, NC | O05839 | Transcription factor |
| 1099 | 2609911 | G | R | CDC1551 | 2639170 | A | H | Yes | NS, NC | O05839 | Transcription factor |
| 1174 | 2825466 | T | D | H37Rv | 2858667 | G | A | Yes | NS, NC | P95020 | Transcription factor |
| 1174 | 2825466 | T | D | CDC1551 | 2854156 | G | A | Yes | NS, NC | P95020 | Transcription factor |
| 1241 | 2988773 | C | A | H37Rv | 3032134 | T | V | Yes | NS, C | Q50765 | Transcription factor |
| 1241 | 2988773 | C | A | CDC1551 | 3027073 | T | V | Yes | NS, C | Q50765 | Transcription factor |
| 1261 | 3043278 | T | I | H37Rv | 3086725 | C | M | Yes | NS, NC | O33321 | Transcription factor |
| 1261 | 3043278 | T | I | CDC1551 | 3081450 | C | M | Yes | NS, NC | O33321 | Transcription factor |
| 1264 | 3053917 | C | A | H37Rv | 3097365 | A | D | Yes | NS, NC | O33330 | Transcription factor |
| 1264 | 3053917 | C | A | CDC1551 | 3092089 | A | D | Yes | NS, NC | O33330 | Transcription factor |
| 1405 | 3404010 | A | C | H37Rv | 3447460 | G | R | Yes | NS, NC | Q06861 | Transcription factor |
| 1405 | 3404010 | A | C | CDC1551 | 3443253 | G | R | Yes | NS, NC | Q06861 | Transcription factor |
| 1503 | 3626758 | A | V | H37Rv | 3672229 | G | A | Yes | NS, C | P96896 | Transcription factor |
| 1503 | 3626758 | A | V | CDC1551 | 3667068 | G | A | Yes | NS, C | P96896 | Transcription factor |

b: List of Polymorphisms (Insertions/Deletions) in transcription factors.

| Polymorphism | | | | | | | Functional | Putative |
|---|---|---|---|---|---|---|---|---|
| ID | BCG start | BCG end | Query name | Query start | Query end | ORF | Annotation | Function |
| 1849 | 194495 | 194498 | H37Rv | 194303 | 194304 | Yes | O07410 | Transcription |
| | | | | | | | | Factor |
| 2074 | 3042938 | 3042939 | H37Rv | 3086361 | 3086386 | Yes | O33321 | Transcription |
| | | | | | | | | Factor |
| 2199 | 4334624 | 4334625 | H37Rv | 4400660 | 4400662 | Yes | O53590 | Transcription |
| | | | | | | | | Factor |
| 1902 | 890037 | 890038 | CDC1551 | 889115 | 889117 | Yes | Q8VKD9 | Transcription |
| | | | | | | | | Factor |
| 1945 | 1404177 | 1404178 | CDC1551 | 1402420 | 1405418 | Yes | Q11063 | Transcription |
| | | | | | | | | Factor |
| 2074 | 3042938 | 3042939 | CDC1551 | 3081086 | 3081111 | Yes | O33321 | Transcription |
| | | | | | | | | Factor |

Table V: List of long polymorphisms in Transcription factors
Polymorphism ID The ID by which the polymorphism can be identified
BCG Start The position in the genome of M. bovis BCG at which multiple polymorhisms start occurring
BCG End The position in the genome of M. bovis BCG at which multiple polymorhisms end
H37Rv Start The position in the genome of M. tuberculosis H37Rv at which multiple polymorhisms start
H37Rv End The position in the genome of M. tuberculosis H37Rv at multiple polymorhisms end
C1551 Start The position in the genome of M. tuberculosis CDC1551 at which multiple polymorhisms start
CDC1551 End The position in the genome of M. tuberculosis CDC1551 at which multiple polymorhisms ends
ORF Indicates whether the polymorphism occurs in an open reading frame (yes) or not (no)
GO ID The ID for the sequence in the gene ontology database
Putative function The putative function of the gene in which the SNP occurs.

TABLE 6
a: List of Polymorphisms(Single Nucleotide Polymorphisms) in genes involved in lipid metabolism

| Polymorphism | BCG | BCG | BCG | Query | Query | Query | Query | | Type of | | Putative |
|---|---|---|---|---|---|---|---|---|---|---|---|
| ID | Position | base | AA | name | Position | base | aa | ORF | SNP | GO ID | Function |
| 29 | 26034 | G | P | H37Rv | 26053 | C | A | Yes | NS, NC | P71591 | Transport |
| 29 | 26034 | G | P | CDC1551 | 26035 | C | A | Yes | NS, NC | P71591 | Transport |
| 69 | 96136 | A | I | H37Rv | 96099 | G | V | Yes | NS, C | Q10884 | Transport |
| 72 | 100624 | G | A | H37Rv | 100587 | A | T | Yes | NS, NC | Q10876 | Transport |
| 72 | 100624 | G | A | CDC1551 | 100579 | A | T | Yes | NS, NC | Q10876 | Transport |
| 79 | 126600 | A | S | H37Rv | 126561 | C | A | Yes | NS, NC | Q10900 | Transport |
| 79 | 126600 | A | S | CDC1551 | 126554 | C | A | Yes | NS, NC | Q10900 | Transport |
| 80 | 126840 | G | P | H37Rv | 126801 | A | S | Yes | NS, NC | Q10900 | Transport |
| 80 | 126840 | G | P | CDC1551 | 126794 | A | S | Yes | NS, NC | Q10900 | Transport |
| 82 | 130172 | A | V | H37Rv | 130133 | G | A | Yes | NS, C | Q10900 | Transport |
| 82 | 130172 | A | V | CDC1551 | 130126 | G | A | Yes | NS, C | Q10900 | Transport |
| 99 | 170273 | G | L | H37Rv | 170081 | A | F | Yes | NS, NC | P96820 | Transport |
| 99 | 170273 | G | L | CDC1551 | 170254 | A | F | Yes | NS, NC | P96820 | Transport |
| 123 | 227215 | G | V | H37Rv | 227020 | A | I | Yes | NS, C | O53645 | Transport |
| 123 | 227215 | G | V | CDC1551 | 227134 | A | I | Yes | NS, C | Q8VKP9 | Transport |
| 124 | 227738 | T | M | H37Rv | 227543 | C | T | Yes | NS, NC | O53645 | Transport |
| 124 | 227738 | T | M | CDC1551 | 227657 | C | T | Yes | NS, NC | Q8VKP9 | Transport |
| 125 | 228053 | T | L | H37Rv | 227858 | C | P | Yes | NS, NC | O53645 | Transport |
| 125 | 228053 | T | L | CDC1551 | 227972 | C | P | Yes | NS, NC | Q8VKP9 | Transport |
| 141 | 262385 | A | N | H37Rv | 262158 | G | D | Yes | NS, NC | P96400 | Transport |
| 141 | 262385 | A | N | CDC1551 | 262274 | G | D | Yes | NS, NC | P96400 | Transport |
| 156 | 292523 | G | A | H37Rv | 294196 | T | E | Yes | NS, NC | O53666 | Transport |
| 156 | 292523 | G | A | CDC1551 | 294313 | T | E | Yes | NS, NC | O53666 | Transport |
| 157 | 292778 | C | R | H37Rv | 294451 | T | K | Yes | NS, C | O53666 | Transport |
| 157 | 292778 | C | R | CDC1551 | 294568 | T | K | Yes | NS, C | O53666 | Transport |
| 201 | 394778 | A | Y | H37Rv | 393746 | G | H | Yes | NS, C | O08447 | Transport |
| 201 | 394778 | A | Y | CDC1551 | 393808 | G | H | Yes | NS, C | O08447 | Transport |
| 218 | 441762 | C | A | H37Rv | 440743 | T | V | Yes | NS, C | O06312 | Transport |
| 218 | 441762 | C | A | CDC1551 | 440833 | T | V | Yes | NS, C | O06312 | Transport |
| 221 | 446432 | T | S | H37Rv | 445413 | C | G | Yes | NS, NC | O53703 | Transport |
| 221 | 446432 | T | S | CDC1551 | 445503 | C | G | Yes | NS, NC | O53703 | Transport |
| 222 | 446797 | T | H | H37Rv | 445778 | C | R | Yes | NS, NC | O53703 | Transport |
| 29 | 26034 | G | P | H37Rv | 26053 | C | A | Yes | NS, NC | P71591 | Transport |
| 222 | 446797 | T | H | CDC1551 | 445868 | C | R | Yes | NS, NC | O53703 | Transport |
| 226 | 452456 | G | S | H37Rv | 451437 | A | F | Yes | NS, NC | O53708 | Transport |
| 226 | 452456 | G | S | CDC1551 | 451527 | A | F | Yes | NS, NC | O53708 | Transport |
| 234 | 472937 | G | A | H37Rv | 471916 | A | V | Yes | NS, C | P95200 | Transport |
| 234 | 472937 | G | A | CDC1551 | 472009 | A | V | Yes | NS, C | P95200 | Transport |
| 247 | 502639 | T | V | H37Rv | 501618 | C | A | Yes | NS, C | P96261 | Transport |
| 247 | 502639 | T | V | CDC1551 | 503069 | C | A | Yes | NS, C | P96261 | Transport |
| 250 | 515676 | G | A | H37Rv | 514655 | T | E | Yes | NS, NC | P96271 | Transport |
| 250 | 515676 | G | A | CDC1551 | 516106 | T | E | Yes | NS, NC | P96271 | Transport |
| 280 | 582972 | A | F | H37Rv | 581819 | G | L | Yes | NS, NC | Q11157 | Transport |
| 280 | 582972 | A | F | CDC1551 | 583188 | G | L | Yes | NS, NC | Q11157 | Transport |
| 383 | 846049 | C | V | H37Rv | 843857 | T | I | Yes | NS, C | O53815 | Transport |
| 383 | 846049 | C | V | CDC1551 | 846000 | T | I | Yes | NS, C | O53815 | Transport |
| 384 | 846399 | G | A | H37Rv | 844207 | A | V | Yes | NS, C | O53815 | Transport |
| 384 | 846399 | G | A | CDC1551 | 846350 | A | V | Yes | NS, C | O53815 | Transport |
| 406 | 913660 | G | L | H37Rv | 912828 | C | F | Yes | NS, NC | O53832 | Transport |
| 406 | 913660 | G | L | CDC1551 | 912739 | C | F | Yes | NS, NC | O53832 | Transport |
| 468 | 1041172 | C | A | H37Rv | 1040704 | A | S | Yes | NS, NC | O05870 | Transport |
| 468 | 1041172 | C | A | CDC1551 | 1040719 | A | S | Yes | NS, NC | O05870 | Transport |
| 469 | 1043636 | C | A | H37Rv | 1043167 | T | V | Yes | NS, C | P15712 | Transport |
| 469 | 1043636 | C | A | CDC1551 | 1043182 | T | V | Yes | NS, C | P15712 | Transport |
| 477 | 1080631 | A | N | H37Rv | 1080190 | G | D | Yes | NS, NC | P77894 | Transport |
| 477 | 1080631 | A | N | CDC1551 | 1080205 | G | D | Yes | NS, NC | P77894 | Transport |
| 478 | 1083482 | A | H | H37Rv | 1083041 | C | Q | Yes | NS, NC | P71539 | Transport |
| 478 | 1083482 | A | H | CDC1551 | 1083056 | C | Q | Yes | NS, NC | P71539 | Transport |
| 483 | 1097474 | A | S | H37Rv | 1097021 | G | G | Yes | NS, NC | O53894 | Transport |
| 483 | 1097474 | A | S | CDC1551 | 1097062 | G | G | Yes | NS, NC | O53894 | Transport |
| 485 | 1102935 | T | L | H37Rv | 1102482 | G | V | Yes | NS, C | O53899 | Transport |
| 485 | 1102935 | T | L | CDC1551 | 1102523 | G | V | Yes | NS, C | O53899 | Transport |
| 561 | 1290071 | C | L | H37Rv | 1288697 | G | V | Yes | NS, C | O06559 | Transport |
| 561 | 1290071 | C | L | CDC1551 | 1288187 | G | V | Yes | NS, C | O06559 | Transport |
| 562 | 1291161 | G | G | H37Rv | 1289787 | A | D | Yes | NS, NC | O06559 | Transport |
| 29 | 26034 | G | P | H37Rv | 26053 | C | A | Yes | NS, NC | P71591 | Transport |
| 562 | 1291161 | G | G | CDC1551 | 1289277 | A | D | Yes | NS, NC | O06559 | Transport |
| 563 | 1295376 | T | V | H37Rv | 1294002 | C | A | Yes | NS, C | O06562 | Transport |
| 563 | 1295376 | T | V | CDC1551 | 1293492 | C | A | Yes | NS, C | O06562 | Transport |
| 567 | 1307530 | C | S | H37Rv | 1306279 | A | I | Yes | NS, NC | O50431 | Transport |
| 567 | 1307530 | C | S | CDC1551 | 1305769 | A | I | Yes | NS, NC | O50431 | Transport |
| 568 | 1309207 | T | N | H37Rv | 1307956 | G | T | Yes | NS, C | O50431 | Transport |
| 568 | 1309207 | T | N | CDC1551 | 1307446 | G | T | Yes | NS, C | O50431 | Transport |
| 595 | 1368728 | G | G | H37Rv | 1367482 | T | W | Yes | NS, NC | O33220 | Transport |
| 595 | 1368728 | G | G | CDC1551 | 1366972 | T | W | Yes | NS, NC | O33220 | Transport |
| 602 | 1383703 | C | R | H37Rv | 1382457 | T | H | Yes | NS, NC | O50455 | Transport |
| 602 | 1383703 | C | R | CDC1551 | 1381947 | T | H | Yes | NS, NC | O50455 | Transport |
| 609 | 1396254 | G | G | H37Rv | 1395008 | A | R | Yes | NS, NC | O50465 | Transport |
| 609 | 1396254 | G | G | CDC1551 | 1394497 | A | R | Yes | NS, NC | O50465 | Transport |
| 780 | 1825125 | C | R | H37Rv | 1839757 | G | G | Yes | NS, NC | O06151 | Transport |
| 780 | 1825125 | C | R | CDC1551 | 1830666 | G | G | Yes | NS, NC | O06151 | Transport |
| 805 | 1897364 | A | F | H37Rv | 1912022 | C | V | Yes | NS, NC | O33188 | Transport |
| 805 | 1897364 | A | F | CDC1551 | 1902826 | C | V | Yes | NS, NC | O33188 | Transport |
| 815 | 1921036 | G | A | H37Rv | 1935693 | T | S | Yes | NS, NC | O33206 | Transport |
| 815 | 1921036 | G | A | CDC1551 | 1926497 | T | S | Yes | NS, NC | O33206 | Transport |
| 816 | 1921535 | A | Q | H37Rv | 1936192 | G | R | Yes | NS, NC | O33206 | Transport |
| 816 | 1921535 | A | Q | CDC1551 | 1926996 | G | R | Yes | NS, NC | O33206 | Transport |
| 822 | 1937372 | C | P | H37Rv | 1952030 | G | A | Yes | NS, NC | P71984 | Transport |
| 822 | 1937372 | C | P | CDC1551 | 1942833 | G | A | Yes | NS, NC | P71984 | Transport |
| 823 | 1938167 | T | Y | H37Rv | 1952825 | C | H | Yes | NS, C | P71984 | Transport |
| 823 | 1938167 | T | Y | CDC1551 | 1943628 | C | H | Yes | NS, C | P71984 | Transport |
| 828 | 1949354 | C | G | H37Rv | 1963955 | T | D | Yes | NS, NC | P71994 | Transport |
| 828 | 1949354 | C | G | CDC1551 | 1954815 | T | D | Yes | NS, NC | P71994 | Transport |
| 829 | 1949427 | G | H | H37Rv | 1964028 | C | D | Yes | NS, NC | P71994 | Transport |
| 829 | 1949427 | G | H | CDC1551 | 1954888 | C | D | Yes | NS, NC | P71994 | Transport |
| 840 | 1965950 | C | T | H37Rv | 1980550 | T | M | Yes | NS, NC | O65936 | Transport |
| 840 | 1965950 | C | T | CDC1551 | 1971410 | T | M | Yes | NS, NC | O65936 | Transport |
| 849 | 2002085 | G | Q | H37Rv | 2011625 | C | H | Yes | NS, NC | O33180 | Transport |
| 29 | 26034 | G | P | H37Rv | 26053 | C | A | Yes | NS, NC | P71591 | Transport |
| 849 | 2002085 | G | Q | CDC1551 | 2009047 | C | H | Yes | NS, NC | O33180 | Transport |
| 879 | 2042298 | T | * | H37Rv | 2051841 | C | Q | Yes | NS, TP | O53958 | Transport |
| 879 | 2042298 | T | * | CDC1551 | 2049263 | C | Q | Yes | NS, TP | Q8VJW0 | Transport |
| 880 | 2043142 | C | S | H37Rv | 2052685 | G | * | Yes | NS, TP | O53958 | Transport |
| 880 | 2043142 | C | S | CDC1551 | 2050107 | G | * | Yes | NS, TP | Q8VJW0 | Transport |
| 885 | 2053386 | C | V | H37Rv | 2062920 | T | I | Yes | NS, C | Q50614 | Transport |
| 885 | 2053386 | C | V | CDC1551 | 2060259 | T | I | Yes | NS, C | Q50614 | Transport |
| 886 | 2054840 | G | A | H37Rv | 2064374 | A | V | Yes | NS, C | Q50614 | Transport |
| 886 | 2054840 | G | A | CDC1551 | 2061713 | A | V | Yes | NS, C | Q50614 | Transport |
| 904 | 2092402 | A | W | H37Rv | 2102191 | G | R | Yes | NS, NC | P95160 | Transport |
| 904 | 2092402 | A | W | CDC1551 | 2099413 | G | R | Yes | NS, NC | P95160 | Transport |
| 907 | 2097719 | T | M | H37Rv | 2107509 | C | T | Yes | NS, NC | P95155 | Transport |
| 907 | 2097719 | T | M | CDC1551 | 2104731 | C | T | Yes | NS, NC | P95155 | Transport |
| 914 | 2112834 | G | A | H37Rv | 2122623 | A | V | Yes | NS, C | P95143 | Transport |
| 914 | 2112834 | G | A | CDC1551 | 2119846 | A | V | Yes | NS, C | P95143 | Transport |
| 915 | 2113185 | G | A | H37Rv | 2122974 | C | G | Yes | NS, C | P95143 | Transport |
| 915 | 2113185 | G | A | CDC1551 | 2120197 | C | G | Yes | NS, C | P95143 | Transport |
| 929 | 2141958 | T | T | H37Rv | 2151676 | G | P | Yes | NS, NC | O07727 | Transport |
| 929 | 2141958 | T | T | CDC1551 | 2148969 | G | P | Yes | NS, NC | O07727 | Transport |
| 930 | 2145004 | A | L | H37Rv | 2154722 | C | R | Yes | NS, NC | Q08129 | Transport |
| 930 | 2145004 | A | L | CDC1551 | 2152015 | C | R | Yes | NS, NC | Q08129 | Transport |
| 953 | 2201224 | C | G | H37Rv | 2222306 | T | D | Yes | NS, NC | Q10875 | Transport |
| 953 | 2201224 | C | G | CDC1551 | 2219640 | T | D | Yes | NS, NC | Q10875 | Transport |
| 980 | 2271395 | A | V | H37Rv | 2287480 | G | A | Yes | NS, C | O53485 | Transport |
| 980 | 2271395 | A | V | CDC1551 | 2289814 | G | A | Yes | NS, C | O53485 | Transport |
| 1008 | 2369039 | A | D | H37Rv | 2388639 | G | G | Yes | NS, NC | O33261 | Transport |
| 1009 | 2369143 | A | S | H37Rv | 2388743 | G | G | Yes | NS, NC | O33261 | Transport |
| 1009 | 2369143 | A | S | CDC1551 | 2387320 | G | G | Yes | NS, NC | O33261 | Transport |
| 1036 | 2438267 | C | I | H37Rv | 2459232 | G | M | Yes | NS, NC | Q10387 | Transport |
| 1036 | 2438267 | C | I | CDC1551 | 2456567 | G | M | Yes | NS, NC | Q10387 | Transport |
| 1062 | 2507835 | A | V | H37Rv | 2528771 | G | A | Yes | NS, C | O53528 | Transport |
| 1062 | 2507835 | A | V | CDC1551 | 2526031 | G | A | Yes | NS, C | O53528 | Transport |
| 29 | 26034 | G | P | H37Rv | 26053 | C | A | Yes | NS, NC | P71591 | Transport |
| 1077 | 2541551 | C | A | H37Rv | 2563956 | A | E | Yes | NS, NC | Q59570 | Transport |
| 1077 | 2541551 | C | A | CDC1551 | 2559803 | A | E | Yes | NS, NC | Q59570 | Transport |
| 1086 | 2569172 | G | V | H37Rv | 2591575 | C | L | Yes | NS, C | P71894 | Transport |
| 1086 | 2569172 | G | V | CDC1551 | 2587422 | C | L | Yes | NS, C | Q8VJL6 | Transport |
| 1110 | 2660947 | A | N | H37Rv | 2692873 | G | S | Yes | NS, C | P71748 | Transport |
| 1110 | 2660947 | A | N | CDC1551 | 2690205 | G | S | Yes | NS, C | P71748 | Transport |
| 1112 | 2661841 | G | S | H37Rv | 2693770 | A | N | Yes | NS, C | P71748 | Transport |
| 1112 | 2661841 | G | S | CDC1551 | 2691102 | A | N | Yes | NS, C | P71748 | Transport |
| 1113 | 2663078 | T | H | H37Rv | 2695007 | C | R | Yes | NS, NC | P71746 | Transport |
| 1113 | 2663078 | T | H | CDC1551 | 2692339 | C | R | Yes | NS, NC | P71746 | Transport |
| 1138 | 2729181 | A | I | H37Rv | 2761016 | G | M | Yes | NS, NC | O53186 | Transport |
| 1138 | 2729181 | A | I | CDC1551 | 2757863 | G | M | Yes | NS, NC | O53186 | Transport |
| 1139 | 2733102 | A | L | H37Rv | 2764937 | G | P | Yes | NS, NC | O53189 | Transport |
| 1139 | 2733102 | A | L | CDC1551 | 2761784 | G | P | Yes | NS, NC | O53189 | Transport |
| 1192 | 2880160 | G | P | H37Rv | 2912710 | T | T | Yes | NS, NC | Q50635 | Transport |
| 1192 | 2880160 | G | P | CDC1551 | 2908855 | T | T | Yes | NS, NC | Q50635 | Transport |
| 1193 | 2880535 | T | T | H37Rv | 2913085 | C | A | Yes | NS, NC | Q50635 | Transport |
| 1193 | 2880535 | T | T | CDC1551 | 2909230 | C | A | Yes | NS, NC | Q50635 | Transport |
| 1194 | 2881707 | C | S | H37Rv | 2914257 | T | N | Yes | NS, C | Q50634 | Transport |
| 1194 | 2881707 | C | S | CDC1551 | 2910402 | T | N | Yes | NS, C | Q50634 | Transport |
| 1216 | 2935735 | C | A | H37Rv | 2968270 | T | V | Yes | NS, C | P71942 | Transport |
| 1216 | 2935735 | C | A | CDC1551 | 2964416 | T | V | Yes | NS, C | P71942 | Transport |
| 1227 | 2959751 | G | G | H37Rv | 3003112 | C | A | Yes | NS, C | O07187 | Transport |
| 1227 | 2959751 | G | G | CDC1551 | 2998058 | C | A | Yes | NS, C | O07187 | Transport |
| 1228 | 2959795 | T | C | H37Rv | 3003156 | G | G | Yes | NS, NC | O07187 | Transport |
| 1228 | 2959795 | T | C | CDC1551 | 2998102 | G | G | Yes | NS, NC | O07187 | Transport |
| 1230 | 2963874 | G | R | H37Rv | 3007235 | A | * | Yes | NS, TP | O07192 | Transport |
| 1230 | 2963874 | G | R | CDC1551 | 3002180 | A | * | Yes | NS, TP | O07192 | Transport |
| 1231 | 2967056 | G | V | H37Rv | 3010417 | A | I | Yes | NS, C | O07194 | Transport |
| 1231 | 2967056 | G | V | CDC1551 | 3005362 | A | I | Yes | NS, C | O07194 | Transport |
| 1242 | 2993548 | G | T | H37Rv | 3036909 | A | I | Yes | NS, NC | O33229 | Transport |
| 1242 | 2993548 | G | T | CDC1551 | 3031848 | A | I | Yes | NS, NC | O33229 | Transport |
| 29 | 26034 | G | P | H37Rv | 26053 | C | A | Yes | NS, NC | P71591 | Transport |
| 1243 | 2993831 | C | A | H37Rv | 3037192 | G | P | Yes | NS, NC | O33229 | Transport |
| 1243 | 2993831 | C | A | CDC1551 | 3032131 | G | P | Yes | NS, NC | O33229 | Transport |
| 1292 | 3096227 | G | P | H37Rv | 3139653 | C | A | Yes | NS, NC | P71619 | Transport |
| 1292 | 3096227 | G | P | CDC1551 | 3133869 | C | A | Yes | NS, NC | Q8VJC1 | Transport |
| 1293 | 3096535 | A | L | H37Rv | 3139961 | G | P | Yes | NS, NC | P71619 | Transport |
| 1294 | 3096724 | A | I | H37Rv | 3140150 | C | S | Yes | NS, NC | P71619 | Transport |
| 1296 | 3099150 | A | L | H37Rv | 3142577 | G | P | Yes | NS, NC | P71616 | Transport |
| 1296 | 3099150 | A | L | CDC1551 | 3136791 | G | P | Yes | NS, NC | P71616 | Transport |
| 1332 | 3192343 | C | G | H37Rv | 3235712 | G | R | Yes | NS, NC | Q10970 | Transport |
| 1332 | 3192343 | C | G | CDC1551 | 3230035 | G | R | Yes | NS, NC | Q10970 | Transport |
| 1333 | 3193344 | C | R | H37Rv | 3236713 | A | L | Yes | NS, NC | Q10970 | Transport |
| 1333 | 3193344 | C | R | CDC1551 | 3231036 | A | L | Yes | NS, NC | Q10970 | Transport |
| 1345 | 3229711 | G | D | H37Rv | 3273135 | C | H | Yes | NS, NC | P96205 | Transport |
| 1345 | 3229711 | G | D | CDC1551 | 3267458 | C | H | Yes | NS, NC | P96205 | Transport |
| 1387 | 3343657 | A | V | H37Rv | 3387094 | G | A | Yes | NS, C | O53275 | Transport |
| 1387 | 3343657 | A | V | CDC1551 | 3382833 | G | A | Yes | NS, C | O53275 | Transport |
| 1396 | 3377371 | G | G | H37Rv | 3420822 | A | D | Yes | NS, NC | P95099 | Transport |
| 1396 | 3377371 | G | G | CDC1551 | 3416547 | A | D | Yes | NS, NC | P95099 | Transport |
| 1416 | 3430975 | A | I | H37Rv | 3474424 | G | V | Yes | NS, C | O05783 | Transport |
| 1416 | 3430975 | A | I | CDC1551 | 3470223 | G | V | Yes | NS, C | O05783 | Transport |
| 1417 | 3431707 | G | D | H37Rv | 3475156 | A | N | Yes | NS, NC | O05783 | Transport |
| 1417 | 3431707 | G | D | CDC1551 | 3470955 | A | N | Yes | NS, NC | O05783 | Transport |
| 1447 | 3476153 | G | A | H37Rv | 3521041 | A | T | Yes | NS, NC | P95173 | Transport |
| 1447 | 3476153 | G | A | CDC1551 | 3516528 | A | T | Yes | NS, NC | P95173 | Transport |
| 1453 | 3488207 | G | L | H37Rv | 3530952 | C | V | Yes | NS, C | O53311 | Transport |
| 1453 | 3488207 | G | L | CDC1551 | 3528588 | C | V | Yes | NS, C | O53311 | Transport |
| 1454 | 3489142 | T | E | H37Rv | 3531888 | G | D | Yes | NS, C | O53313 | Transport |
| 1454 | 3489142 | T | E | CDC1551 | 3529524 | G | D | Yes | NS, C | O53313 | Transport |
| 1466 | 3528181 | C | D | H37Rv | 3573633 | T | N | Yes | NS, NC | O53346 | Transport |
| 1466 | 3528181 | C | D | CDC1551 | 3568540 | T | N | Yes | NS, NC | O53346 | Transport |
| 1479 | 3562541 | A | C | H37Rv | 3607938 | G | R | Yes | NS, NC | O05875 | Transport |
| 1479 | 3562541 | A | C | CDC1551 | 3602842 | G | R | Yes | NS, NC | O05875 | Transport |
| 29 | 26034 | G | P | H37Rv | 26053 | C | A | Yes | NS, NC | P71591 | Transport |
| 1482 | 3569604 | T | H | H37Rv | 3615000 | C | R | Yes | NS, NC | O05884 | Transport |
| 1482 | 3569604 | T | H | CDC1551 | 3609903 | C | R | Yes | NS, NC | Q8VJ44 | Transport |
| 1499 | 3619961 | A | D | H37Rv | 3665432 | G | G | Yes | NS, NC | P96888 | Transport |
| 1499 | 3619961 | A | D | CDC1551 | 3660271 | G | G | Yes | NS, NC | P96888 | Transport |
| 1511 | 3644541 | G | S | H37Rv | 3690012 | A | L | Yes | NS, NC | O53355 | Transport |
| 1511 | 3644541 | G | S | CDC1551 | 3684851 | A | L | Yes | NS, NC | Q8VJ36 | Transport |
| 1512 | 3645379 | C | A | H37Rv | 3690850 | T | T | Yes | NS, NC | O53355 | Transport |
| 1512 | 3645379 | C | A | CDC1551 | 3685689 | T | T | Yes | NS, NC | Q8VJ36 | Transport |
| 1520 | 3661401 | G | E | H37Rv | 3706872 | C | D | Yes | NS, C | O53371 | Transport |
| 1520 | 3661401 | G | E | CDC1551 | 3701763 | C | D | Yes | NS, C | O53371 | Transport |
| 1587 | 3816846 | A | D | H37Rv | 3869141 | C | A | Yes | NS, NC | O33354 | Transport |
| 1587 | 3816846 | A | D | CDC1551 | 3855093 | C | A | Yes | NS, NC | O33354 | Transport |
| 1608 | 3839628 | A | V | H37Rv | 3893286 | G | A | Yes | NS, C | O06339 | Transport |
| 1608 | 3839628 | A | V | CDC1551 | 3887122 | G | A | Yes | NS, C | O06339 | Transport |
| 1609 | 3839818 | A | F | H37Rv | 3893476 | G | L | Yes | NS, NC | O06339 | Transport |
| 1609 | 3839818 | A | F | CDC1551 | 3887312 | G | L | Yes | NS, NC | O06339 | Transport |
| 1621 | 3869973 | T | V | H37Rv | 3924346 | C | A | Yes | NS, C | O53550 | Transport |
| 1621 | 3869973 | T | V | CDC1551 | 3918182 | C | A | Yes | NS, C | O53550 | Transport |
| 1638 | 3947819 | G | R | H37Rv | 4004600 | A | Q | Yes | NS, NC | P96845 | Transport |
| 1638 | 3947819 | G | R | CDC1551 | 3996744 | A | Q | Yes | NS, NC | P96845 | Transport |
| 1639 | 3948329 | C | S | H37Rv | 4005110 | G | W | Yes | NS, NC | P96845 | Transport |
| 1639 | 3948329 | C | S | CDC1551 | 3997254 | G | W | Yes | NS, NC | P96845 | Transport |
| 1640 | 3951962 | G | T | H37Rv | 4008744 | A | I | Yes | NS, NC | P96849 | Transport |
| 1640 | 3951962 | G | T | CDC1551 | 4000886 | A | I | Yes | NS, NC | P96849 | Transport |
| 1644 | 3964308 | T | S | H37Rv | 4021090 | G | A | Yes | NS, NC | P96860 | Transport |
| 1644 | 3964308 | T | S | CDC1551 | 4013232 | G | A | Yes | NS, NC | P96860 | Transport |
| 1682 | 4043080 | C | G | H37Rv | 4105730 | T | E | Yes | NS, NC | O69634 | Transport |
| 1682 | 4043080 | C | G | CDC1551 | 4097990 | T | E | Yes | NS, NC | O69634 | Transport |
| 1683 | 4043104 | T | Q | H37Rv | 4105754 | C | R | Yes | NS, NC | O69634 | Transport |
| 1683 | 4043104 | T | Q | CDC1551 | 4098014 | C | R | Yes | NS, NC | O69634 | Transport |
| 1684 | 4044421 | C | R | H37Rv | 4107071 | T | Q | Yes | NS, NC | O69634 | Transport |
| 1684 | 4044421 | C | R | CDC1551 | 4099331 | T | Q | Yes | NS, NC | O69634 | Transport |
| 29 | 26034 | G | P | H37Rv | 26053 | C | A | Yes | NS, NC | P71591 | Transport |
| 1692 | 4065667 | G | Q | H37Rv | 4128317 | C | E | Yes | NS, NC | O69653 | Transport |
| 1692 | 4065667 | G | Q | CDC1551 | 4120575 | C | E | Yes | NS, NC | O69653 | Transport |
| 1716 | 4113722 | A | R | H37Rv | 4176259 | G | G | Yes | NS, NC | O69695 | Transport |
| 1716 | 4113722 | A | R | CDC1551 | 4168574 | G | G | Yes | NS, NC | O69695 | Transport |
| 1790 | 4266511 | T | H | H37Rv | 4330235 | C | R | Yes | NS, NC | P96219 | Transport |
| 1790 | 4266511 | T | H | CDC1551 | 4322561 | C | R | Yes | NS, NC | P96219 | Transport |
| 1824 | 4335857 | G | G | H37Rv | 4401894 | A | D | Yes | NS, NC | P52214 | Transport |
| 1824 | 4335857 | G | G | CDC1551 | 4394201 | A | D | Yes | NS, NC | P52214 | Transport |

b: List of Polymorphisms(Insertions/Deletions) in genes involved in lipid metabolism

| Polymorphism | BCG | BCG | Query | Query | Query | | | Putative |
|---|---|---|---|---|---|---|---|---|
| ID | start | end | name | start | end | ORF | GO ID | Function |
| 1837 | 82490 | 82491 | H37Rv | 82452 | 82454 | Yes | O53618 | Transport |
| 1838 | 125870 | 125872 | H37Rv | 125832 | 125833 | Yes | Q10900 | Transport |
| 1854 | 257984 | 258014 | H37Rv | 257786 | 257787 | Yes | P96397 | Transport |
| 1883 | 669950 | 669952 | H37Rv | 668706 | 668707 | Yes | O53772 | Transport |
| 1902 | 890037 | 890038 | H37Rv | 887845 | 887847 | Yes | O07268 | Transport |
| 1917 | 1041920 | 1041922 | H37Rv | 1041452 | 1041453 | Yes | P95302 | Transport |
| 1919 | 1087886 | 1087887 | H37Rv | 1087445 | 1087447 | Yes | O86319 | Transport |
| 1946 | 1407255 | 1407256 | H37Rv | 1409016 | 1409018 | Yes | Q11058 | Transport |
| 1964 | 1744186 | 1744191 | H37Rv | 1760169 | 1760170 | Yes | Q10761 | Transport |
| 1971 | 1879687 | 1879698 | H37Rv | 1894299 | 1894300 | Yes | O53916 | Transport |
| 1994 | 2116913 | 2116915 | H37Rv | 2126702 | 2126703 | Yes | O07753 | Transport |
| 2006 | 2184230 | 2184231 | H37Rv | 2192593 | 2192595 | Yes | P95275 | Transport |
| 2032 | 2504789 | 2504790 | H37Rv | 2525724 | 2525726 | Yes | O53525 | Transport |
| 2037 | 2540853 | 2540854 | H37Rv | 2563256 | 2563259 | Yes | Q59570 | Transport |
| 2040 | 2584392 | 2584410 | H37Rv | 2606795 | 2606796 | Yes | P71879 | Transport |
| 2044 | 2661152 | 2661153 | H37Rv | 2693078 | 2693088 | Yes | P71748 | Transport |
| 2075 | 3043634 | 3043635 | H37Rv | 3087081 | 3087083 | Yes | P30234 | Transport |
| 2084 | 3098539 | 3098540 | H37Rv | 3141965 | 3141967 | Yes | P71617 | Transport |
| 2099 | 3381643 | 3381645 | H37Rv | 3425094 | 3425095 | Yes | P95097 | Transport |
| 2103 | 3455437 | 3456765 | H37Rv | 3501662 | 3501663 | Yes | P95191 | Transport |
| 2163 | 3898149 | 3898150 | H37Rv | 3954929 | 3954931 | Yes | O53563 | Transport |
| 1837 | 82490 | 82491 | CDC1551 | 82444 | 82446 | Yes | O53618 | Transport |
| 1854 | 257984 | 258014 | CDC1551 | 257902 | 257903 | Yes | P96397 | Transport |
| 1883 | 669950 | 669952 | CDC1551 | 670159 | 670160 | Yes | O53772 | Transport |
| 1902 | 890037 | 890038 | CDC1551 | 889115 | 889117 | Yes | Q8VKD9 | Transport |
| 1917 | 1041920 | 1041922 | CDC1551 | 1041467 | 1041468 | Yes | P95302 | Transport |
| 1919 | 1087886 | 1087887 | CDC1551 | 1087460 | 1087462 | Yes | O86319 | Transport |
| 1946 | 1407255 | 1407256 | CDC1551 | 1408505 | 1408507 | Yes | Q11058 | Transport |
| 1964 | 1744186 | 1744191 | CDC1551 | 1760325 | 1760326 | Yes | Q10761 | Transport |
| 1994 | 2116913 | 2116915 | CDC1551 | 2123925 | 2123926 | Yes | O07753 | Transport |
| 2006 | 2184230 | 2184231 | CDC1551 | 2189926 | 2189928 | Yes | P95275 | Transport |
| 2037 | 2540853 | 2540854 | CDC1551 | 2559103 | 2559106 | Yes | Q59570 | Transport |
| 2040 | 2584392 | 2584410 | CDC1551 | 2602641 | 2602642 | Yes | P71879 | Transport |
| 2044 | 2661152 | 2661153 | CDC1551 | 2690410 | 2690420 | Yes | P71748 | Transport |
| 2075 | 3043634 | 3043635 | CDC1551 | 3081806 | 3081808 | Yes | P30234 | Transport |
| 2084 | 3098539 | 3098540 | CDC1551 | 3136179 | 3136181 | Yes | P71617 | Transport |
| 2099 | 3381643 | 3381645 | CDC1551 | 3420887 | 3420888 | Yes | P95097 | Transport |
| 2102 | 3441287 | 3441288 | CDC1551 | 3480536 | 3483302 | Yes | O05793 | Transport |
| 2163 | 3898149 | 3898150 | CDC1551 | 3947715 | 3947717 | Yes | O53563 | Transport |

TABLE 7
List of Polymorphisms in genes encoding membrane transport proteins

| | BCG | BCG | BCG | Query | Query | Query | Query | | Type of | | Putative |
|---|---|---|---|---|---|---|---|---|---|---|---|
| Polymorphism ID | Position | base | AA | name | Position | base | aa | ORF | SNP | GO ID | Function |
| 632 | 1457413 | G | G | H37Rv | 1459362 | T | V | Yes | NS, C | Q10606 | Lipid |
| | | | | | | | | | | | Metabolism |
| 632 | 1457413 | G | G | CDC1551 | 1458905 | T | V | Yes | NS, C | Q10606 | Lipid |
| | | | | | | | | | | | Metabolism |

Table VII: List of long polymorphisms in genes encoding membrane transport proteins
Polymorphism ID The ID by which the polymorphism can be identified
BCG Start The position in the genome of M. bovis BCG at which multiple polymorhisms start occurring
BCG End The position in the genome of M. bovis BCG at which multiple polymorhisms end
H37Rv Start The position in the genome of M. tuberculosis H37Rv at which multiple polymorhisms start
H37Rv End The position in the genome of M. tuberculosis H37Rv at multiple polymorhisms end
C1551 Start The position in the genome of M. tuberculosis CDC1551 at which multiple polymorhisms start
CDC1551 End The position in the genome of M. tuberculosis CDC1551 at which multiple polymorhisms ends
ORF Indicates whether the polymorphism occurs in an open reading frame (Yes) or not (no)
GO ID The ID for the sequence in the gene ontology database
Putative function The putative function of the gene in which the SNP occurs.

TABLE 8
List of Polymorphisms in genes implicated in virulence

| Polymorphism | Gene | BCG | BCG | BCG | Query | Query | Query | Query | | is | Is non- | | Putative |
|---|---|---|---|---|---|---|---|---|---|---|---|---|---|
| ID | name | Position | base | AA | Name | position | base | AA | ORF | nsSNP | cons | GO ID | Function |
| 285 | proC | 591914 | C | D | H37Rv | 590761 | G | E | Yes | NS | C | Q11141 | oxidoreductase |
| | | | | | | | | | | | | | activity |
| 285 | proC | 591914 | C | D | CDC1551 | 592131 | G | E | Yes | NS | C | Q11141 | oxidoreductase |
| | | | | | | | | | | | | | activity |
| 1348 | fadD28 | 3239912 | T | I | H37Rv | 3283336 | G | S | Yes | NS | NC | P96290 | calcium ion |
| | | | | | | | | | | | | | binding activity |
| 1348 | fadD28 | 3239912 | T | I | CDC1551 | 3277659 | G | S | Yes | NS | NC | P96290 | calcium ion |
| | | | | | | | | | | | | | binding activity |
| 1349 | fadD28 | 3240065 | G | R | H37Rv | 3283489 | A | Q | Yes | NS | NC | P96290 | calcium ion |
| | | | | | | | | | | | | | binding activity |
| 1349 | fadD28 | 3240065 | G | R | CDC1551 | 3277812 | A | Q | Yes | NS | NC | P96290 | calcium ion |
| | | | | | | | | | | | | | binding activity |
| 1350 | fadD28 | 3240165 | C | G | H37Rv | 3283589 | G | G | Yes | S | Null | Null | Null |
| 1350 | fadD28 | 3240165 | C | G | CDC1551 | 3277912 | G | G | Yes | S | Null | Null | Null |
| 1351 | mmpL7 | 3242680 | A | V | H37Rv | 3286104 | C | V | Yes | S | Null | Null | Null |
| 1351 | mmpL7 | 3242680 | A | V | CDC1551 | 3280427 | C | V | Yes | S | Null | Null | Null |
| 1352 | mmpL7 | 3243139 | T | A | H37Rv | 3286563 | C | A | Yes | S | Null | Null | Null |
| 1352 | mmpL7 | 3243139 | T | A | CDC1551 | 3280886 | C | A | Yes | S | Null | Null | Null |
| 274 | pcaA | 561876 | G | L | H37Rv | 560855 | C | L | Yes | S | Null | Null | Null |
| 274 | pcaA | 561876 | G | L | CDC1551 | 562305 | C | L | Yes | S | Null | Null | Null |
| 275 | pcaA | 562317 | T | A | H37Rv | 561296 | C | A | Yes | S | Null | Null | Null |
| 275 | pcaA | 562317 | T | A | CDC1551 | 562746 | C | A | Yes | S | Null | Null | Null |
| 1561 | dnaE2 | 3736194 | A | S | H37Rv | 3782550 | G | S | Yes | S | Null | Null | Null |
| 1561 | dnaE2 | 3736194 | A | S | CDC1551 | 3774786 | G | S | Yes | S | Null | Null | Null |
| 1562 | dnaE2 | 3736445 | T | R | H37Rv | 3782801 | G | R | Yes | S | Null | Null | Null |
| 1562 | dnaE2 | 3736445 | T | R | CDC1551 | 3775037 | G | R | Yes | S | Null | Null | Null |

Table VIII: List of long polymorphisms in genes implicated in virulence
Polymorphism ID The ID by which the polymorphism can be identified
BCG Start The position in the genome of M. bovis BCG at which multiple polymorhisms start occurring
BCG End The position in the genome of M. bovis BCG at which multiple polymorhisms end
H37Rv Start The position in the genome of M. tuberculosis H37Rv at which multiple polymorhisms start
H37Rv End The position in the genome of M. tuberculosis H37Rv at multiple polymorhisms end
C1551 Start The position in the genome of M. tuberculosis CDC1551 at which multiple polymorhisms start
CDC1551 End The position in the genome of M. tuberculosis CDC1551 at which multiple polymorhisms ends
ORF Indicates whether the polymorphism occurs in an open reading frame (Yes) or not (no)
GO ID The ID for the sequence in the gene ontology database
Putative function The putative function of the gene in which the SNP occurs.

1-26. (canceled)

27. A nucleotide sequence having a sequence selected from the group consisting of SEQ ID NOs: 1 to 2531.

28. A method for diagnosis; identification of strains; typing of strains; and/or giving orientation to potential degree of virulence, infectivity and/or latency of all strains of Mycobactenra wherein said method utilizes a nucleotide sequence selected from sequences having SEQ ID NOs: 1 to 2531.

29. The nucleotide sequence as claimed in claim 27 wherein the sequence is a single nucleotide polymorphism having SEQ ID NOs: 1 to 1829.

30. The nucleotide sequence as claimed in claim 27 wherein the sequence is an insertion/deletion (indel) having SEQ ID NOs:1830 to 2286.

31. The nucleotide sequence as claimed in claim 27 wherein the sequence is a region of long polymorphism having a sequence selected from SEQ ID NOs: 2287 to 2531.

32. Primer sequences for amplifying the region around a polymorphism of SEQ ID NOs:1 to 2531.

33. A nucleotide sequence flanking a polymorphism of SEQ ID NOs:1 to 2531 wherein said sequence has a length up to 35 nucleotides.

34. A method for drug design; drug development; gene therapy; and/or vaccine development, wherein said method utilizes a nucleotide sequence having a sequence selected from SEQ ID NOs:1 to 2531 as a target.

35. The method, according to claim 34, wherein said method utilizes a sequence encompassing a single nucleotide polymorphism having a sequence selected from SEQ ID NOs:1 to 1829.

36. The method, according to claim 34, wherein said method utilizes a sequence encompassing insertion/deletion (indel) having a sequence selected from SEQ ID NOs:1830 to 2286.

37. The method, according to claim 34, wherein said method utilizes a region of long polymorphism having a sequence selected from SEQ ID NOs:2287 to 2581.

38. A method for drug design using bioinformatics and/or for development of drugs effective against infectious diseases including tuberculosis wherein said method utilizes a protein, RNA, DNA or metabolite encoded by a region having a sequence selected from SEQ ID NOs: 1 to 2531.

39. A method for vaccine development against infectious diseases including tuberculosis wherein said method utilizes a protein, RNA, DNA or metabolite encoded by the region having a sequence selected from SEQ ID NOs: 1 to 2531.

40. Use of a protein, RNA, DNA and/or metabolite encoded by the region carrying the polymorphisms having a sequence selected from SEQ ID NOs: 1 to 2531 for RNAi technology and antisense technologies.

41. A method for generating and developing a database for identification and selection of polymorphisms, wherein said method utilizes sequences selected from SEQ ID NOs:1 to 2531.

42. The method as claimed in claim 41 wherein said database is generated using the algorithms as herein described.

43. Use of the database as claimed in claim 41, for identification of the polymorphisms across organisms.

44. A diagnostic kit for diagnosis, identification of the strain, typing of the strain and giving orientation to its potential degree of virulence, infectivity and/or latency of all infectious diseases wherein said kit comprise at least one polynucleotide sequence having a sequence selected from SEQ ID NOs: 1 to 2531.

45. The diagnostic kit as claimed in 44 for diagnosis, identification of the strain, typing of the strain and giving orientation to its potential degree of virulence, infectivity and/or latency of all strain of Mycobacteria having SEQ ID NOs: 1 to 2531.

46. The diagnostic kit as claimed in 18 wherein the said sequence is a single nucleotide polymorphism having SEQ ID NOs: 1to 1829.

47. The diagnostic kit as claimed in 44 wherein the said sequence is an insertion/deletion (indel) having SEQ ID NOs: 1830 to 2286.

48. The diagnostic kit as claimed in 44 wherein the said sequence are regions of long polymorphism having a SEQ ID NOs: 2287 to 2531.

49. An assay for the identification of strains for infectious diseases including mycobacterium wherein said method utilizes a nucleotide sequences having a SEQ ID NOs: 1 to 2531 as a probe in an assay.

50. The method as claimed in claim 49 wherein the sequence is a single nucleotide polymorphism having SEQ ID NOs:1 to 1829.

51. The method as claimed in claim 49 wherein the sequence is an insertion/deletion (indel) having SEQ ID NOs:1830 to 2286.

52. The method as claimed in claim 50 wherein the sequence is a region of long polymorphism having a SEQ ID NOs:2287 to 2531.
